(19) United States
(12) Patent Application Publication
(10) Pub. No.: US 2008/0176239 A1
(43) Pub. Date: Jul. 24, 2008

(54) GENES ASSOCIATED WITH SCHIZOPHRENIA

(75) Inventors: Stephanie Chissoe, Durham, NC (US); Margaret G. Ehm, Durham, NC (US); Pamela St. Jean, Morrisville, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 11/970,605

(22) Filed: Jan. 8, 2008

Related U.S. Application Data
(60) Provisional application No. 60/885,650, filed on Jan. 19, 2007.

(51) Int. Cl. C12Q 1/68

(52) U.S. Cl. 435/6

(57) ABSTRACT

Genes associated with schizophrenia are identified. Methods of screening small molecule compounds for use in treating schizophrenia are described.

### CROSS-REFERENCE TO RELATED APPLICATIONS

This application is based on and claims priority to U.S. provisional application No. 60/885,650 filed on Jan. 19, 2007 and is incorporated herein in its entirety.

### FIELD OF THE INVENTION

The present invention relates to identification of genes that are associated with schizophrenia and to screening methods to identify chemical compounds that act on those targets for the treatment of Schizophrenia or its associated pathologies.

### BACKGROUND OF THE INVENTION

The purpose of the present study was to identify genes coding for tractable targets that are associated with schizophrenia, to develop methods of screening compounds to identify those that act on such targets, and to develop such compounds as medicines to treat Schizophrenia and its associated pathologies.

Schizophrenia is a common disorder, affecting 1% of the population, independent of regional origin or cultural background (Thaker, 2001). The socioeconomic impact of the disease is heavy, since direct costs of schizophrenia in western countries range between 1.6% to 2.6% of total health care expenditures. About 75% of these costs are due to inpatient or residential care, while drug therapy represents less than 5% (Barbato, 1998). Therefore, any new medicine could reduce the cost of care for schizophrenia while targeting the most disabled patients in order to improve their independent living skill. Further understanding of schizophrenia is therefore a valuable strategy for the development of innovative drugs to treat the disease and reduce both its social and economical burden.

Although the aetiology of schizophrenia is still unexplained, diverse evidence favours a genetic component. The lifetime morbidity risk for schizophrenia in the general population is close to 1%. This risk increases to about 3-5% in second degree relatives or in half-siblings, and up to 9-12% in siblings and dizygotic twins. Monozygotic twins share a 50% relative risk, as well as the children of two schizophrenic parents. Together these data suggest a heritability of 80%, emphasizing the strong genetic basis of this disorder (Cardno, 1999; Cardno, 2000 and references herein). However, in addition, epigenetic factors clearly play a role in the disease given the lack of complete sharing of the risk in monozygotic twins, and the lack of any major gene identified or reproducibly localised to date in genetic studies (McGue, 1989; Cardno, 2000, Riley, 2000 and references herein). Thus, schizophrenia is a multifactorial disorder, with a high likelihood of involving many genes of mild to moderate contribution in conjunction with environmental risk factors (Risch, 1990; Cardno, 2000). The identification of these genes, even of small and combined effects, will be of considerable therapeutic importance, and will help establish a complete picture of the pathophysiology of the disease. Ultimately, a better understanding of the underlying pathophysiology of the disease would permit more rational drug development.

### SUMMARY OF THE INVENTION

A first aspect of the present invention is a method for screening small molecule compounds for use in treating Schizophrenia by screening a test compound against a gene product target selected from the group consisting of genes with Schizophrenia associated Single Nucleotide Polymorphisms (SNPs) listed in Tables 3-5. Activity against said target indicates the test compound has potential use in treating Schizophrenia.

### DETAILED DESCRIPTION

The present inventors tested Single Nucleotide Polymorphisms (SNPs) within genes and in close proximity to genes (within ˜10 kb) that encode for potential tractable targets to identify genes that are associated with the occurrence of Schizophrenia and to provide methods for screening to identify compounds with potential therapeutic effects in Schizophrenia. Sequence variations or sequence polymorphisms in genes that modify the risk factors for schizophrenia, were identified through genetic analysis of two case-control cohorts from Munich, Germany and from Aberdeen, Scotland. Each cohort comprised roughly 500-550 cases and 500-650 controls.

As used, herein, a ‘tractable target’ or ‘druggable target’ is a biological molecule that is known to be responsive to manipulation by small molecule chemical compounds, e.g., can be activated or inhibited by small molecule chemical compounds. Classes of ‘tractable targets’ include, but are not limited to, 7-transmembrane receptors (7TM receptors), ion channels, nuclear receptors, kinases, proteases and integrins.

### EXAMPLE 1

### Subjects and Methods

### Sample Set

The sample set consisted of Caucasian cases and Caucasian controls collected in Munich (Klinikum der Universität München—Innenstadt, Munich, Germany, Principal Investigator (PI) Hans-Juergen Moeller & Dan Rujescu; The Max Planck Psychiatry Institute, Munich, Germany (PI Florian Holsboer); and the University of Aberdeen, Aberdeen, Scotland (PI David St Clair). All subjects gave informed consent for the use of their DNA in this study. Caucasian is defined as having 3 of 4 grandparents self-reported as Caucasian. The Munich sample set consisted of 511 cases and 500 controls. The Aberdeen sample consisted of 546 cases and 669 controls.

An individual was eligible for inclusion in this study only if all the following criteria applied: was at least 18 years of age at the time of entering the study; was of Caucasian background; and gave voluntary written consent to participate in the study. In addition to the general inclusion criteria, each schizophrenia case (patient) must have had the diagnosis of schizophrenia as defined by the Diagnostic and Statistical Manual of Mental Disorders (DSM-IV) and/or International Classification of Diseases ICD-10, American Psychiatric Association 1994). A schizophrenia case was not eligible for inclusion in this study if any of the following criteria applied: Have been diagnosed/or presented with schizoaffective disorder, or a mood disorder unrelated to schizophrenia; was a monozygotic twin of a case already included in the study and/or had been diagnosed as an intravenous drug user with a lifetime diagnosis of dependency.

### Target Genes

Relatively few human proteins, approximately a hundred in total, are considered to be suitable targets for effective small molecule drugs. It was considered reasonable to include all the members of these families for which a sequence was available. At the time, some of the genes were not exemplified in the public domain and were discovered through the analysis of expressed sequence tags or genomic sequence using a combination of sequence analysis. In addition, genes were selected because they were the targets of effective drugs even though they were not part of large protein families. Finally, disease expertise was employed to select genes whose involvement in Schizophrenia was either proven or suspected. Genes were named accordingly to NCBI ENTREZ gene.

### Single Nucleotide Polymorphisms (SNP) Identification

The genes were automatically assembled and annotated with a region of the gene designated as 5′ and 3′, intron and exon. SNPs were mapped using BLAST to the manually curated genomic sequences. The SNPs were selected up to 10 kb from the start and stop sites of the transcripts with an average intermarker distance of 30 Kb. SNPs with a minor allele frequency (MAF)>5% were selected, but, all known coding SNPs were included irrespective of MAF. Approximately 10% of genes had fewer than 6 SNPs and these were subjected to SNP discovery using 24 primer pairs per gene to amplify 12 DNAs selected from Coriell Cell Repository of female CEPH cell-line samples. (CEPH refers to the Centre d'Etude du Polymorphisme Humain, which collected Northern European DNA samples.) For all of the discovered SNPs a-minor allele frequency was determined using the FAST (Flow Accelerated SNP Typing) (Taylor et al, 2001) technology using multiplex PCR coupled with Single Base Chain Extension (SBCE) and Amplifluor genotyping. A marker selection algorithm was used to remove highly correlated SNPs to reduce the genotyping requirement while maintaining the genetic information content throughout the regions (Meng et al, 2003).

### Sample Preparation and Genotyping

DNA was isolated from whole blood using a basic salting-out procedure. Samples were arrayed and normalized in water to a standard concentration of 5 ng/ul. Twenty nanogram aliquots of the DNA samples were arrayed into 96-well PCR plates. For purposes of quality control, 3.4% of the samples were duplicated on the plates and two negative template control wells received water. The samples were dried and the plates were stored at −20° C. until use. Genotyping was performed by a modification of the single base chain extension (SBCE) assay previously described (Taylor et al. 2001). Assays were designed by a GlaxoSmithKline in-house primer design program and then grouped into multiplexes of 50 reactions for PCR and SBCE. Following genotyping, the data was scored using a modification of Spotfire Decision Site Version 7.0 Genotypes passed quality control if: a) duplicate comparisons were concordant, b) negative template controls did not generate genotypes and c) more than 80% of the samples had valid genotypes. Genotypes for assays passing quality control tests were exported to an analysis database.

### Data Handling

The GSK database of record for analysis-ready data is called SubjectLand. This database contains all genotypes, phenotypes (i.e. clinical data), and pedigree information, where applicable, on all subjects used in the analysis of data for these studies. SubjectLand does not maintain information regarding DNA samples, but is closely integrated with the sample tracking system to maintain the connection between subjects and their samples and phenotypic data at all times. All subjects gave informed consent for the use of their DNA and phenotypic data in this study. The analytical tools used in the analysis process described below interface directly with subject data in SubjectLand. This interface also archives the files used in analysis as well as the results.

Analysis

Only subjects with a subject type (SBTY) of case or control were analyzed. Subjects with a SBTY of affected family member or other SBTY values were excluded from analysis. Subjects were also excluded if he/she, either parent, or more than one grandparent were non-Caucasian as indicated by self-report. In addition, subjects were excluded if their putative gender was inconsistent with SNP genotypes on the X chromosome. Finally, subjects that genotyped on fewer than 75% of the SNPs in a given genotyping experiment were excluded from analysis.

Each marker was examined for Hardy-Weinberg equilibrium (HWE) and minor allele frequency. Markers with a Hardy-Weinberg p-value<0.001 in controls were not analyzed. Genotypic and allelic associations test were then performed using a Fishers Exact Test, followed by identification of the risk allele and risk genotype using chi-square tests. An odds ratio and confidence interval of greater than 95% was calculated for the risk allele and risk genotype. Next, population stratification was evaluated by determining if the number of allelic and genotypic tests observed to be significant at a given threshold was inflated with respect to what would be expected under the null hypothesis of no association. If population stratification was observed, a genomic-control adjustment was applied to the Fast Fishers Exact Test results.

The two cohorts (Munich and Aberdeen) were genotyped and analyzed separately. In each cohort, statistically significant SNPs were identified as those with an allelic or genotypic Fishers Exact P-value≦0.05. A combined assessment was also conducted to identify SNPs significant in both the Munich and Aberdeen sets. In the combined assessment, a SNP was considered statistically significant if it had a Fishers Exact P-value≦0.05 in both the Munich set and in the Aberdeen set.

### Hardy Weinberg Equilibrium

Hardy Weinberg equilibrium (HWE) is a measure of the association between two alleles at an individual locus. A bi-allelic marker is in HWE if the genotype frequencies are p2, 2pq and q2 for the genotypes 1, 1; 1, 2; and 2, 2 where p and q are the frequencies of the 1 and 2 alleles, respectively. The departure from HWE was tested using a Chi square test, by testing the difference between the expected (calculated from the allele frequencies) and observed genotype frequencies. A HWE permutation test was performed when the HWE chi-square p-value<0.05 and when at least one genotype cell had an expected count less than 5 (Zaykin et al, 1995). When these conditions exist, the HWE chi-square test may not be valid and a permutation test to assess departure from HWE is warranted. Markers failing HWE at p≦0.001 in controls were removed from the pooled analysis marker cluster used in association analyses. HWE failure may indicate a non-robust assay.

### Minor Allele Frequency

For minor allele frequency, markers which were monomorphic were removed from the analysis marker cluster used in association analyses.

### Allelic and Genotypic Test of Association

Testing for association in the study data was carried out using the ‘PROC FREQ’ fast Fisher's exact test (FET) procedure in the statistical software package SASv8.2. An exact test is warranted in situations when asymptotic assumptions are not met such as when the sample size is not large or when the distribution is sparse or skewed. Such situations occur for SNPs with rare minor allele frequencies where the number of expected cases and/or controls for the rare homozygote are less than 5. Under these conditions, the asymptotic results many not be valid and the asymptotic p-value may differ substantially from the exact p-value. The classic Fisher's Exact Test computes exact p-values by enumerating all tables as extreme as, or more extreme than, that observed. This direct enumeration approach is very time-consuming and only feasible for small problems. The fast Fisher's Exact test computes exact p-values for general R×C tables using the network algorithm developed by Mehta and Patel (1983). The network algorithm provides substantial advantage over direct enumeration and is rapid and accurate.

Tables I and II show the structure of the genotype and allele contingency tables, respectively.

TABLE I
Generic disease status by genotype contingency table.
Disease Status

| | | Case | Control | Total |
|---|---|---|---|---|
| Genotype | AA | n11 | n12 | n1. |
| | Aa | n21 | n22 | n2. |
| | aa | n31 | n32 | n3. |

| | | | |
|---|---|---|---|
| Total | n.1 | n.2 | N |

TABLE II
Generic disease status by allele contingency table.
Disease Status

| | | Case | Control | Total |
|---|---|---|---|---|
| Allele | A | 2n11 + n21 | 2n12 + n22 | 2n1. + n2. |
| | a | 2n31 + n21 | 2n32 + n22 | 2n3. + n2 |

| | | | |
|---|---|---|---|
| Total | 2n.1 | 2n.2 | 2N |

### Risk Allele and Risk Genotype

The “risk allele” refers to the allele that appeared more frequently in cases than controls. The “risk genotype” was determined after identifying the genotype that had the largest chi-square value when compared against the other two genotypes combined in the genotypic association test. For example, if a SNP had genotypes AA, AG and GG, three chi-square tests were performed contrasting cases and controls: 1) AA vs AG+GG, 2) AG vs AA+GG and 3) GG vs AA+AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

### Odds Ratios and Confidence Intervals

An odds ratio was constructed for the risk allele and risk genotype.

Odds ratio (OR)=(n11*n22)/(n12*n21)

where
n11=cases with risk genotype
n21=cases without risk genotype
n12=controls with risk genotype
n22=controls without risk genotype

In order to avoid division or multiplication by zero, 0.5 was added to each cell in the contingency table (as recommended in “Statistical Methods for Rates and Proportions” by Fleiss, Ch 5.3 p. 64)

A 95% confidence interval for the odds ratio was also calculated as follows:

where
z=97.5th percentile of the standard normal distribution

v=[1/(n11)]+[1/(n12)]+[1/(n21)]+[1/(n22)]

### Evaluation of Population Stratification

In this assessment, cases and control frequencies were compared across a subset of relatively independent markers (markers in low LD) selected from the set of all markers analyzed. Since the vast majority of genes on the gene list are not associated with a specific disease, this constitutes a null data set. If the cases and controls are from the same underlying population, the expectation is to see 5% of the tests significant at the 5% level, 1% significant at the 1% level, etc. If, on the other hand, the cases and controls are from different populations, (for example, cases from Finland and controls from Japan), there would be an inflation in the proportion of tests significant across thresholds due to genetic differences between the two populations that are unrelated to disease. Inflation in the number of observed significant tests over a range of cut-points suggests that the case and control groups are not well matched. Consequently, the inflated number of positive tests may be due to population stratification rather than to association between the associated SNPs and disease.

The probability of ≧m observed number of significant tests out of n total tests at a cut-point p was calculated using the binomial probability as implemented SAS v8.2 with the function ROBNML (p,n,m) computes the probability that an observation from a binomial(n,p) distribution will be less than or equal to m.

### Adjusting for Population Stratification

Using a Genomic Control approach can correct for overdispersion of test statistics caused by population substructure. Devlin et al (2002) propose dividing the tests for association by \hat{λ}

The simplest implementation suggested is to estimate \hat{λ} as follows

\hat{λ}={median (S₁,S₂, . . . ,Sₙ)/X} where Sᵢ refers to the chi-square statistic for loci i=1 to n. X is the median of the chi-square distribution given degrees of freedom (X=0.456 for 1df test and 1.39 for 2 dftest). When evidence of population stratification existed, we adjusted the FET allelic or genotypic p-values. The p-values were 1^{st} converted to a chi-square statistics. \hat{λ} was estimated using the derived chi-square statistics. Next, the derived chi-square statistic was multiplied by \hat{λ} to yield an adjusted chi-square statistic. The adjusted chi-square statistic then was then used to calculate an adjusted FET p-value.

### Linkage Disequilibrium (LD)

The LD between two markers is given by DAB=pAB-pApB, where pA is the allele frequency of A allele of the first marker, pB is the allele frequency of B allele of the second marker, and pAB is the joint frequency of alleles A and B on the same haplotype. LD tends to decline with distance between markers and generally exists for markers that are less than 100 kb apart.

The SAS procedure PROC CORR was used to calculate r using the Pearson product-moment correlation. To determine whether significant LD existed between a pair of markers we made use of the fact that nr2 has an approximate chi square distribution with 1df for biallelic markers. The significance level of pairwise LD was computed in SAS.

### EXAMPLE 2

### Results

The Munich collection of 511 cases and 500 controls was genotyped in May 2005 on a panel of 1,828 genes with 6,591 SNPs. Forty collected subjects were excluded from the study based on sample set quality control (QC) measures. Eighteen were excluded for ethnicity, 6 for gender inconsistency, and 16 that were genotyped on fewer than 75% of the SNPs. The analysis set of 495 cases and 476 controls was reasonably well-matched with respect to gender with the proportion of males being slightly higher among cases.

The Aberdeen collection of 546 cases and 669 controls was genotyped in November 2006 on a panel of 2,098 genes with 12,897 SNPs. 10 collected subjects were excluded from the study based on sample set quality control (QC) measures. Two were excluded for subject type, 7 for gender inconsistency, and 1 that was genotyped on fewer than 75% of the SNPs. The analysis set of 537 cases and 668 controls was well-matched with respect to age and gender

Key demographic characteristics of the Munich and Aberdeen sets are detailed in Table 1.

During SNP marker quality control for the Munich set, 46 SNPs were excluded due to Hardy-Weinberg Equilibrium (HWE); 275 SNPs were excluded because SNPs were monomorphic in cases and controls; 32 SNPs were excluded due to mapping issues and 36 SNPs were excluded as the returned genotypes on fewer than 80% of samples. As a result, 6,202 SNPs were analyzed for association with Schizophrenia of which 6,102 had a gene assignment and 100 did not. In total 1,801 genes were analyzed: 1,734 autosomal, 67 X-linked. The mean number of SNPs per genes was 3.4 with a range of 1-49 SNPs per gene.

During SNP marker quality control for the Aberdeen set, 33 SNPs were excluded due to Hardy-Weinberg Equilibrium (HWE); 550 SNPs were excluded because SNPs were monomorphic in cases and controls; 68 SNPs were excluded due to mapping issues and 16 SNPs were excluded as the returned genotypes on fewer than 80% of samples. As a result, 12,230 SNPs were analyzed for association with Schizophrenia of which 11,540 had a gene assignment and 690 did not. In total 2,062 genes were analyzed: 1991 autosomal, 71 X-linked. The mean number of SNPs per genes was 5.6 with a range of 1-214 SNPs per gene.

See Table 2 for a summary SNP coverage of genes in the Munich collection and Aberdeen Collection.

Five hundred and five SNPs and accompanying genes in Table 3 showed statistical association with Schizophrenia in the Munich collection. These SNPs had a genotypic or allelic Fisher's Exact p-value of ≦0.05. Since there was no evidence of population stratification in the Munich sample, a genomic control adjustment was not applied to the p-values. Table 6 contains a description of genes identified as associated in the Munich collection.

Nine hundred and forty-four SNPs and accompanying genes in Table 4 showed statistical association with Schizophrenia in the Aberdeen collection. These SNPs had a genotypic or allelic Fisher's Exact p-value of ≦0.05. Since there was evidence of slight population stratification in the Aberdeen sample for the genotypic test, a genomic control adjustment was applied to the genotypic exact p-values. The genotypic exact p-value \hat{λ} of 1.063. The allelic test did not show evidence of inflation with an allelic exact p-value \hat{λ} of 1.00. Table 6 contains a description of genes identified as associated in the Aberdeen collection.

An assessment of the results from the Munich and Aberdeen collection, revealed that thirty-four SNPs and their accompanying genes in Table 5 showed statistical evidence in both individual collections. These SNPs had p-values≦0.05 from the genotypic and/or allelic exact tests in the Munich collection and in the Aberdeen collection. These SNPs and their genes have the strongest statistical evidence for association with Schizophrenia. Among these thirty-four SNPs, twenty-two showed concordant test direction(s) between the two collections and demonstrate the strongest evidence of replication. Concordance was assessed by combining the two data sets into a pooled set and assessing which of the 34 SNPs were significant at p-values≦0.05 for either the allelic or genotypic tests of association. However, it is possible, albeit less likely, for the same gene to be a risk factor in two different collections even if test direction is discordant if the gene has undergone a divergent evolutionary history in the two collections. Table 6 contains a description of genes identified as associated in both the Munich and Aberdeen collections.

It is possible that some of the associations are false positives. Statistical association between a polymorphic marker and disease may occur for several reasons. The marker may be a mutation that influences disease susceptibility directly or may be correlated with a mutation that influences disease susceptibility because the marker and disease susceptibility mutation are physically close to one another. Spurious association may result from issues such as confounding or bias although the study design attempts to remove or minimize these factors. The association between a marker and disease may also be due to chance.

For the combined assessment, thirty-four SNPs were significant at p<0.05 in two separate collections, Munich and Aberdeen. These SNPs and their genes have the strongest statistical evidence for association with Schizophrenia. The thirty-four SNPs are RS-GSK8111961, RS1446966, RS2296618, RS2046490, RS1376865, RS889895, RS977162, RS610902, RS2244291, RS1047530, RS2072633, RS38559, RS2976440, RS2976437, RS1440332, RS2664361, RS1996637, RS6144, RS4747082, RS3781730, RS2927493, RS498186, RS1548133, RS2189480, RS1582188, RS1816628, RS1286920, RS2230739, RS1873191, RS3760798, RS2234395, RS2423410, RS6318 and RS994423.

TABLE 1
Collections analysed
Schizophrenia CASES

| | Munich (Germany) | Aberdeen (UK) |
|---|---|---|
| Sites | n = 495 | n = 537 |
| Male (%):Female | 320 (64.6%):175 (35.3%) | 395 (73.5%):142 (26.4%) |
| (%) | | |
| Mean age (+/−sd) | 38.6 (11.4) | 43.6 (13.1) |
| DSM score: | | |
| Disorganised type | 74 (14.9%) | 11 (2%) |
| Catatonic type | 11 (2.2%) | 1 (0.2%) |
| Paranoid type | 384 (77.6%) | 423 (78.8%) |
| Residual type | 3 (0.6%) | 59 (10.6%) |
| Undifferentiated | 23 (4.6%) | 45 (8.4%) |
| type | | |

CONTROL

| | Munich (Germany) | Aberdeen (UK) |
|---|---|---|
| | n = 476 | n = 668 |
| Male (%):Female | 278 (58.4%):198 (41.6%) | 487 (72.9%):181 (27.1%) |
| (%) | | |
| Mean age (+/−sd) | 51.3 (14.2) | 45.4 (12.1) |

TABLE 2
SNP coverage of genes in analysis marker cluster

| No. of genes | | | | | | | |
|---|---|---|---|---|---|---|---|
| with | 1 SNP | 2 SNPs | 3 SNPS | 4-5 SNPs | 6-9 SNPs | 10+ SNPs | Total |
| Munich | 475 | 467 | 318 | 307 | 141 | 93 | 1801 |
| Aberdeen | 257 | 483 | 380 | 407 | 288 | 247 | 2062 |

TABLE 3
Munich Collection: SNPs with a Genotypic or Allelic Fishers Exact P ≦ 0.05.

| | | | Genotypic | | Allelic | | Number |
|---|---|---|---|---|---|---|---|
| | NCBI 36 | Gene | Exact | Risk Genotype & | Exact | Risk Allele & Odd | Cases, |
| Poly Name¹ | position | Symbols⁴ | P² | Odd Ratio (95% CI)³ | P² | Ratio (95% CI) | Controls |
| RS924201 | 01: 016191655 | | 0.03462 | !C,C: 1.58(1.11, 2.27) | 0.02410 | T: 1.24(1.03, 1.49) | 488,474 |
| RS2235917 | 01: 017168304 | PADI2 | 0.03247 | !C,C: 1.62(1.1, 2.39) | 0.25354 | A: 1.11(0.93, 1.34) | 491,474 |
| RS1204891 | 01: 017439921 | | 0.00938 | T,T: 2.76(1.34, 5.66) | 0.01616 | T: 1.33(1.06, 1.68) | 475,464 |
| 6786585 | 01: 026926094 | NR0B2 | 0.00817 | !C,T: 1.57(1.18, 2.1) | 0.01437 | C: 1.38(1.07, 1.77) | 491,472 |
| RS1877477 | 01: 042296373 | GUCA2A | 0.04389 | !G,G: | 0.03442 | A: 1.38(1.03, 1.86) | 487,471 |
| | | | | 3.46(1.04, 11.52) | | | |
| RS7543083 | 01: 046743360 | MKNK1 | 0.07077 | !A,A: 1.27(0.99, 1.64) | 0.02677 | G: 1.25(1.03, 1.51) | 487,470 |
| RS2479408 | 01: 055216209 | PCSK9 | 0.11963 | !C,C: 1.28(0.99, 1.66) | 0.04179 | G: 1.25(1.01, 1.56) | 486,469 |
| 16419703 | 01: 062778665 | ANGPTL3, | 0.04049 | T,T: 2.09(1.04, 4.17) | 0.04242 | T: 2.06(1.03, 4.09) | 491,474 |
| | | DOCK7, | | | | | |
| | | TIG41002 | | | | | |
| RS2780830 | 01: 065057148 | JAK1 | 0.09429 | C,C: 1.3(0.99, 1.72) | 0.03179 | C: 1.22(1.02, 1.46) | 490,475 |
| RS4655589 | 01: 065983807 | PDE4B | 0.12062 | !G,G: 1.34(1.01, 1.77) | 0.04911 | A: 1.28(1, 1.63) | 474,436 |
| RS2310753 | 01: 066108973 | PDE4B | 0.01788 | A,A: | 0.05834 | A: 1.37(0.99, 1.9) | 477,454 |
| | | | | 14.49(0.83, 254.44) | | | |
| RS1883461 | 01: 071171069 | PTGER3 | 0.03302 | !C,G: 1.42(1.09, 1.86) | 0.10787 | C: 1.2(0.97, 1.49) | 493,475 |
| RS880099 | 01: 071232787 | PTGER3 | 0.08801 | !C,G: 1.43(1.04, 1.98) | 0.04760 | G: 1.34(1, 1.79) | 490,475 |
| RS974843 | 01: 086727200 | CLCA4 | 0.04759 | !T,T: 1.37(1.06, 1.76) | 0.01896 | C: 1.26(1.04, 1.53) | 490,474 |
| RS2231595 | 01: 086748967 | CLCA4 | 0.06937 | !C,C: 1.44(1.05, 1.97) | 0.02560 | T: 1.39(1.05, 1.85) | 492,474 |
| RS552101 | 01: 109794515 | GPR61 | 0.10207 | G,G: 1.42(1.02, 1.99) | 0.04669 | G: 1.38(1.01, 1.89) | 486,467 |
| 6370171 | 01: 110932277 | | 0.00688 | !G,G: 1.74(1.19, 2.54) | 0.00228 | A: 1.75(1.22, 2.51) | 489,476 |
| RS572473 | 01: 112156483 | KCND3 | 0.04416 | C,C: 1.98(0.99, 3.95) | 0.01390 | C: 1.36(1.06, 1.73) | 478,452 |
| RS2163945 | 01: 118038795 | | 0.00017 | !T,T: 1.75(1.31, 2.35) | 0.00004 | C: 1.54(1.25, 1.89) | 493,320 |
| RS3765945 | 01: 119763488 | HSD3B1 | 0.08634 | T,T: 1.34(1.03, 1.74) | 0.02870 | T: 1.24(1.03, 1.5) | 470,443 |
| RS2641338 | 01: 120160791 | | 0.08033 | C,C: 1.39(1.04, 1.87) | 0.03312 | C: 1.34(1.03, 1.76) | 488,475 |
| RS1837984 | 01: 143853558 | PRKAB2 | 0.01313 | G,G: 1.68(1.18, 2.38) | 0.02356 | G: 1.24(1.03, 1.48) | 490,474 |
| RS1891502 | 01: 144446358 | | 0.11529 | !G,G: 1.31(1.01, 1.71) | 0.04278 | A: 1.21(1.01, 1.45) | 492,475 |
| RS- | 01: 147572946 | CTSK | 0.02582 | G,G: 2.57(1.09, 6.06) | 0.02679 | G: 2.54(1.08, 5.96) | 478,470 |
| RS6667434 | 01: 151222173 | IL6R | 0.02090 | A,G: 1.43(1.1, 1.85) | 0.28114 | A: 1.11(0.92, 1.33) | 469,458 |
| RS1702184 | 01: 151578323 | KCNN3 | 0.00736 | !C,T: 1.55(1.17, 2.06) | 0.07440 | C: 1.24(0.98, 1.56) | 494,474 |
| RS1955160 | 01: 156366337 | | 0.00420 | A,A: 1.49(1.13, 1.96) | 0.00097 | A: 1.36(1.13, 1.63) | 492,472 |
| RS1562384 | 01: 156375697 | | 0.11467 | !A,A: 1.3(0.99, 1.7) | 0.03683 | C: 1.28(1.02, 1.6) | 478,473 |
| RS1446965 | 01: 156375839 | | 0.01738 | !A,A: 1.5(1.12, 2.01) | 0.00463 | G: 1.46(1.13, 1.9) | 490,471 |
| RS1446966 | 01: 156376556 | APCS | 0.00765 | !G,G: 1.47(1.13, 1.91) | 0.00181 | A: 1.35(1.12, 1.63) | 467,465 |
| RS380518 | 01: 162104393 | RXRG | 0.00018 | !C,C: | 0.00108 | T: 1.56(1.2, 2.03) | 476,469 |
| | | | | 7.78(2.06, 29.32) | | | |
| RS2485506 | 01: 178214415 | CACNA1E | 0.04620 | C,C: 1.44(1.07, 1.92) | 0.03239 | C: 1.22(1.02, 1.46) | 490,476 |
| RS2296618 | 01: 195397889 | PTPRC | 0.00969 | !G,G: 8.1(1.47, 44.6) | 0.32586 | A: 1.14(0.88, 1.48) | 490,474 |
| RS1494485 | 01: 199832889 | ADORA1 | 0.00816 | !T,T: 1.58(1.11, 2.26) | 0.00226 | A: 1.34(1.11, 1.61) | 482,473 |
| 6946405 | 01: 202776201 | AVPR1B | 0.03327 | A,A: 2.09(1.17, 3.76) | 0.06421 | A: 1.22(0.99, 1.5) | 492,475 |
| RS1539243 | 01: 203036182 | IKBKE | 0.04246 | !T,T: 2.15(0.98, 4.72) | 0.85601 | T: 1.03(0.81, 1.3) | 486,474 |
| RS585627 | 01: 206392594 | | 0.01168 | !C,G: 1.39(1.07, 1.81) | 0.75038 | C: 1.04(0.84, 1.28) | 490,476 |
| RS656566 | 01: 206394998 | | 0.01408 | !C,T: 1.38(1.06, 1.79) | 0.75049 | T: 1.04(0.84, 1.28) | 491,474 |
| RS1970589 | 01: 207331720 | KCNH1 | 0.14328 | !A,A: 1.34(0.95, 1.87) | 0.04962 | G: 1.2(1, 1.44) | 481,466 |
| RS2173371 | 01: 213182348 | ESRRG | 0.02269 | A,A: 1.43(1.1, 1.85) | 0.01964 | A: 1.28(1.04, 1.56) | 468,473 |
| RS946469 | 01: 213344186 | ESRRG | 0.09717 | C,C: 1.34(1.01, 1.77) | 0.03107 | C: 1.31(1.03, 1.67) | 448,470 |
| RS697761 | 01: 224499132 | | 0.04480 | C,C: 1.63(1.11, 2.41) | 0.07598 | C: 1.19(0.98, 1.43) | 494,474 |
| RS1933632 | 01: 227224770 | CAPN9 | 0.02614 | C,C: 2.76(1.25, 6.12) | 0.08485 | C: 1.22(0.97, 1.53) | 489,474 |
| RS1421210 | 01: 233779372 | RYR2 | 0.02413 | T,T: 1.65(1.14, 2.39) | 0.11359 | T: 1.16(0.97, 1.39) | 487,474 |
| RS2058935 | 01: 233787906 | RYR2 | 0.01588 | A,A: 1.72(1.16, 2.56) | 0.21837 | A: 1.12(0.93, 1.35) | 485,473 |
| RS1030116 | 01: 233794604 | RYR2 | 0.00299 | A,A: 1.75(1.25, 2.45) | 0.05170 | A: 1.2(1, 1.44) | 474,470 |
| RS2543037 | 01: 233827463 | RYR2 | 0.07435 | C,C: 1.76(1.03, 3.01) | 0.03994 | C: 1.25(1.01, 1.55) | 486,471 |
| 5342981 | 01: 236399267 | CHRM3 | 0.07887 | C,C: 1.37(1.04, 1.82) | 0.04301 | C: 1.21(1.01, 1.46) | 451,465 |
| RS2184857 | 01: 236407788 | CHRM3 | 0.00485 | !G,G: 1.89(1.28, 2.78) | 0.01935 | T: 1.26(1.04, 1.52) | 470,457 |
| RS6689793 | 01: 238040859 | KMO | 0.07376 | C,G: 1.5(1.04, 2.15) | 0.04868 | C: 1.42(1.01, 1.99) | 490,462 |
| RS1389622 | 01: 240541905 | ZNF238 | 0.01421 | T,T: 2.25(1.28, 3.97) | 0.07312 | T: 1.21(0.98, 1.49) | 482,473 |
| RS2357263 | 02: 009797259 | | 0.07683 | T,T: 1.59(1, 2.51) | 0.03344 | T: 1.25(1.02, 1.52) | 489,471 |
| RS2007556 | 02: 026444142 | GPR113 | 0.07455 | G,G: 1.62(0.97, 2.7) | 0.03734 | G: 1.24(1.02, 1.52) | 489,471 |
| RS2083363 | 02: 027386969 | SLC30A3 | 0.01993 | C,C: 2.28(1.16, 4.49) | 0.01926 | C: 1.3(1.05, 1.61) | 490,474 |
| RS4131229 | 02: 043969718 | ABCG5 | 0.06882 | !T,T: 1.3(0.99, 1.71) | 0.02268 | C: 1.25(1.04, 1.52) | 465,428 |
| RS6718187 | 02: 043994013 | ABCG8 | 0.00701 | G,G: 1.55(1.15, 2.09) | 0.10734 | G: 1.16(0.97, 1.39) | 471,472 |
| RS4314040 | 02: 044003071 | ABCG8 | 0.01417 | G,G: 1.53(1.13, 2.06) | 0.07371 | G: 1.18(0.98, 1.41) | 487,463 |
| RS3795860 | 02: 044010506 | ABCG8 | 0.00789 | !A,G: 1.42(1.1, 1.84) | 0.28606 | G: 1.11(0.92, 1.33) | 489,451 |
| RS616381 | 02: 045803359 | PRKCE | 0.04572 | C,C: 1.48(1.07, 2.05) | 0.13794 | C: 1.15(0.96, 1.38) | 484,469 |
| RS2091787 | 02: 049156210 | FSHR | 0.00777 | C,C: 1.56(1.15, 2.13) | 0.11093 | C: 1.16(0.97, 1.39) | 495,474 |
| 6728874 | 02: 068184534 | C1D | 0.01066 | !C,T: 1.46(1.11, 1.92) | 0.87598 | C: 1.02(0.83, 1.25) | 419,448 |
| RS2193407 | 02: 075308721 | TACR1 | 0.01136 | A,A: 4.66(1.46, 14.89) | 0.09804 | A: 1.25(0.96, 1.62) | 489,471 |
| RS1515950 | 02: 086932475 | | 0.12102 | G,G: 1.28(0.99, 1.65) | 0.03810 | G: 1.25(1.01, 1.54) | 476,464 |
| RS2113418 | 02: 095410723 | KCNIP3 | 0.02088 | C,C: 1.54(1.13, 2.1) | 0.04883 | C: 1.2(1, 1.43) | 485,472 |
| RS1143634 | 02: 113306621 | IL1B | 0.03097 | T,T: 2.02(1.08, 3.76) | 0.78762 | T: 1.03(0.83, 1.27) | 491,472 |
| RS2863238 | 02: 113639114 | | 0.00636 | C,T: 1.51(1.15, 2) | 0.30313 | T: 1.13(0.9, 1.42) | 469,464 |
| RS3738912 | 02: 113710647 | LOC654433, | 0.01011 | G,T: 1.48(1.15, 1.91) | 0.71375 | T: 1.04(0.87, 1.24) | 490,463 |
| | | PAX8 | | | | | |
| RS2579620 | 02: 119954593 | SCTR | 0.11969 | C,C: 1.42(1.02, 1.97) | 0.04054 | C: 1.37(1.02, 1.85) | 445,461 |
| RS2228014 | 02: 136706816 | CXCR4 | 0.03210 | C,T: 1.7(1.09, 2.66) | 0.02580 | T: 1.62(1.06, 2.48) | 491,471 |
| RS1861975 | 02: 162754807 | DPP4 | 0.03482 | C,C: 1.68(1.07, 2.63) | 0.51939 | C: 1.07(0.88, 1.3) | 484,476 |
| RS2046490 | 02: 163264993 | KCNH7 | 0.01273 | A,A: 1.56(1.16, 2.09) | 0.02021 | A: 1.24(1.04, 1.48) | 489,476 |
| RS1385858 | 02: 163311206 | KCNH7 | 0.03299 | !C,C: 1.41(1.07, 1.85) | 0.01182 | G: 1.26(1.06, 1.51) | 492,475 |
| RS497692 | 02: 169614523 | ABCB11 | 0.07874 | G,G: 1.38(1.03, 1.85) | 0.03358 | G: 1.22(1.02, 1.46) | 482,456 |
| RS9751090 | 02: 169683073 | ABCB11 | 0.07550 | C,T: 1.61(1.03, 2.51) | 0.04932 | T: 1.51(1, 2.29) | 491,462 |
| RS1376865 | 02: 175450104 | CHRNA1 | 0.08454 | A,A: 1.4(1.04, 1.88) | 0.03482 | A: 1.34(1.03, 1.75) | 488,474 |
| RS2113378 | 02: 207156329 | ADAM23 | 0.04691 | T,T: 1.41(1.07, 1.85) | 0.02227 | T: 1.32(1.04, 1.67) | 492,474 |
| RS2300963 | 02: 207260010 | ADAM23 | 0.04724 | !G,G: | 0.60433 | G: 1.09(0.81, 1.45) | 492,472 |
| | | | | 3.59(0.87, 14.79) | | | |
| RS889895 | 02: 208224435 | CREB1 | 0.03945 | C,T: 1.32(1.01, 1.72) | 0.78145 | C: 1.03(0.83, 1.28) | 487,465 |
| RS2303901 | 02: 222116407 | EPHA4 | 0.02906 | !C,T: 1.41(1.09, 1.82) | 0.74754 | T: 1.03(0.86, 1.24) | 487,463 |
| RS2853447 | 02: 233215423 | XM_065294, | 0.02887 | G,G: 1.41(1.08, 1.84) | 0.04199 | G: 1.24(1.01, 1.52) | 421,458 |
| | | CHRND, | | | | | |
| | | LOC646960 | | | | | |
| RS2138168 | 02: 240026366 | HDAC4 | 0.00825 | C,T: 1.42(1.1, 1.83) | 0.51365 | T: 1.06(0.89, 1.28) | 489,470 |
| 6728567 | 02: 241291355 | GPR35 | 0.00367 | C,C: 1.56(1.2, 2.02) | 0.00186 | C: 1.41(1.14, 1.75) | 493,471 |
| RS4464318 | 02: 242310227 | ATG4B | 0.04160 | !G,G: 1.47(1.08, 2) | 0.08987 | A: 1.17(0.98, 1.4) | 491,465 |
| RS2136152 | 03: 007257402 | GRM7 | 0.10388 | C,C: 1.33(1, 1.76) | 0.03931 | C: 1.31(1.02, 1.69) | 479,469 |
| RS167979 | 03: 007728798 | GRM7 | 0.00954 | !C,T: 1.47(1.14, 1.9) | 0.70853 | C: 1.04(0.87, 1.25) | 493,470 |
| RS346070 | 03: 011278874 | HRH1 | 0.00793 | T,T: 2.69(1.38, 5.23) | 0.08430 | T: 1.24(0.97, 1.58) | 472,467 |
| RS3021408 | 03: 020088834 | PCAF | 0.00312 | !T,T: 1.94(1.28, 2.95) | 0.19637 | C: 1.13(0.94, 1.37) | 477,470 |
| RS2365793 | 03: 020128398 | PCAF | 0.01397 | !A,G: 1.66(1.14, 2.42) | 0.01491 | G: 1.58(1.1, 2.26) | 477,470 |
| RS977162 | 03: 020166510 | PCAF | 0.01251 | G,G: 1.47(1.14, 1.91) | 0.00928 | G: 1.34(1.08, 1.67) | 484,475 |
| CCR3_2NEW | 03: 046281480 | CCR3 | 0.13556 | !A,A: 1.27(0.97, 1.66) | 0.04194 | G: 1.23(1.01, 1.51) | 398,468 |
| R.326080 | | | | | | | |
| RS6773261 | 03: 048650068 | CELSR3 | 0.08183 | !A,G: 1.45(1.04, 2.01) | 0.04885 | A: 1.34(1, 1.78) | 475,452 |
| RS164640 | 03: 052222354 | ALAS1 | 0.02952 | !A,A: 1.46(1.1, 1.93) | 0.04445 | G: 1.2(1.01, 1.44) | 489,475 |
| RS352140 | 03: 052231737 | TLR9 | 0.02185 | !T,T: 1.44(1.09, 1.9) | 0.13154 | C: 1.15(0.96, 1.38) | 493,475 |
| RS9311654 | 03: 057531869 | ARF4 | 0.02969 | C,T: 1.4(1.06, 1.84) | 0.38297 | T: 1.11(0.89, 1.4) | 493,469 |
| RS7625929 | 03: 057547367 | ARF4 | 0.03066 | !A,C: 1.47(1.1, 1.95) | 0.05363 | A: 1.27(1, 1.63) | 494,468 |
| RS6767123 | 03: 057553316 | ARF4 | 0.02968 | !G,T: 1.46(1.1, 1.95) | 0.07933 | G: 1.25(0.98, 1.6) | 491,466 |
| RS4340676 | 03: 057557128 | ARF4 | 0.03072 | !A,G: 1.47(1.1, 1.97) | 0.05900 | G: 1.27(1, 1.63) | 486,465 |
| RS704586 | 03: 099997851 | DCBLD2, | 0.03275 | C,T: 1.38(1.06, 1.78) | 1.00000 | T: 1(0.83, 1.21) | 452,461 |
| | | ST3GAL6 | | | | | |
| RS8833 | 03: 099998259 | DCBLD2 | 0.00706 | C,T: 1.47(1.14, 1.9) | 0.74029 | T: 1.03(0.86, 1.25) | 489,476 |
| RS828616 | 03: 100023806 | DCBLD2 | 0.02000 | A,G: 1.43(1.1, 1.86) | 0.62518 | A: 1.05(0.87, 1.27) | 459,457 |
| RS610902 | 03: 120734475 | CD80 | 0.05701 | C,C: 1.35(1.04, 1.75) | 0.02242 | C: 1.24(1.03, 1.49) | 492,473 |
| RS1880661 | 03: 120761538 | CD80 | 0.13721 | !C,C: 1.31(0.99, 1.73) | 0.04899 | T: 1.21(1, 1.45) | 458,461 |
| RS2715265 | 03: 123329522 | CD86 | 0.00155 | !T,T: 1.85(1.31, 2.61) | 0.00155 | C: 1.34(1.12, 1.61) | 491,473 |
| RS389566 | 03: 149929080 | AGTR1 | 0.00344 | A,T: 1.56(1.2, 2.03) | 0.04815 | A: 1.23(1, 1.51) | 492,473 |
| RS2331449 | 03: 150098780 | CPA3 | 0.00654 | !A,A: 1.72(1.2, 2.47) | 0.15847 | G: 1.15(0.95, 1.38) | 490,467 |
| RS2141630 | 03: 152391542 | MED12L | 0.01579 | !A,G: 1.39(1.06, 1.81) | 0.78745 | A: 1.03(0.84, 1.28) | 494,475 |
| RS1231521 | 03: 152405336 | MED12L, | 0.04170 | !T,T: 1.82(1.13, 2.93) | 0.12233 | A: 1.17(0.96, 1.44) | 446,466 |
| | | GPR171 | | | | | |
| RS2122419 | 03: 157541397 | KCNAB1 | 0.03734 | C,G: 1.38(1.05, 1.8) | 0.44098 | C: 1.09(0.88, 1.35) | 486,469 |
| RS816547 | 03: 157605001 | KCNAB1 | 0.03295 | T,T: 2.22(1.09, 4.5) | 0.81753 | T: 1.03(0.82, 1.29) | 490,476 |
| RS2241294 | 03: 171280789 | GPR160 | 0.04087 | C,C: 1.38(1.07, 1.78) | 0.02613 | C: 1.25(1.03, 1.52) | 493,471 |
| RS4459940 | 03: 182917992 | SOX2 | 0.03248 | A,T: 1.35(1.05, 1.75) | 0.37763 | T: 1.09(0.91, 1.31) | 476,464 |
| RS2284890 | 03: 197276457 | TFRC | 0.06786 | G,G: 1.37(1.05, 1.8) | 0.04086 | G: 1.22(1.01, 1.47) | 462,456 |
| RS6846731 | 04: 005945903 | CRMP1 | 0.01811 | !A,A: 1.87(1.13, 3.1) | 0.00854 | G: 1.91(1.17, 3.13) | 488,450 |
| | | EVC | | | | | |
| RS6848864 | 04: 005974581 | CRMP1 | 0.05884 | !A,A: 1.44(1.04, 1.98) | 0.02399 | G: 1.41(1.05, 1.9) | 489,466 |
| RS2244291 | 04: 053309407 | USP46 | 0.03583 | !A,G: 1.52(1.08, 2.14) | 0.05933 | A: 1.35(0.99, 1.84) | 477,469 |
| RS1355368 | 04: 062608588 | LPHN3 | 0.04139 | !G,G: 1.35(1.05, 1.75) | 0.01116 | A: 1.29(1.06, 1.56) | 485,474 |
| RS1510923 | 04: 062663507 | LPHN3 | 0.04643 | !G,G: 1.37(1.06, 1.76) | 0.01386 | A: 1.28(1.05, 1.56) | 491,474 |
| RS1349449 | 04: 066021649 | EPHA5 | 0.14612 | !C,C: 1.47(0.93, 2.32) | 0.04778 | T: 1.24(1.01, 1.53) | 476,462 |
| RS1038426 | 04: 068432301 | GNRHR | 0.06263 | !C,C: 1.37(1.05, 1.79) | 0.03787 | A: 1.22(1.01, 1.47) | 479,461 |
| RS974483 | 04: 068444266 | GNRHR | 0.07024 | C,C: 1.47(1, 2.17) | 0.02867 | C: 1.24(1.03, 1.49) | 488,474 |
| RS6532083 | 04: 077286380 | CXCL9 | 0.01989 | !G,G: 1.43(1.09, 1.88) | 0.09356 | A: 1.17(0.97, 1.41) | 481,456 |
| RS3921 | 04: 077300122 | CXCL10 | 0.02037 | !C,C: 1.43(1.09, 1.88) | 0.08017 | G: 1.18(0.98, 1.41) | 491,469 |
| RS4257674 | 04: 077302864 | CXCL10 | 0.01770 | !G,G: 1.44(1.1, 1.89) | 0.09360 | A: 1.17(0.98, 1.41) | 477,462 |
| RS4512021 | 04: 077311586 | CXCL11 | 0.03105 | !G,G: 1.42(1.08, 1.86) | 0.07977 | A: 1.18(0.98, 1.41) | 491,465 |
| RS7376907 | 04: 084013653 | SCD5 | 0.02928 | !C,C: 1.47(1.06, 2.04) | 0.00982 | T: 1.27(1.06, 1.52) | 488,470 |
| RS1464953 | 04: 084017958 | SCD5 | 0.01267 | G,G: 1.43(1.1, 1.87) | 0.00217 | G: 1.35(1.11, 1.62) | 474,446 |
| RS1474427 | 04: 084028198 | SCD5 | 0.02300 | !A,G: 1.47(1.11, 1.95) | 0.02837 | G: 1.3(1.03, 1.64) | 486,468 |
| RS1026861 | 04: 087970391 | PTPN13 | 0.07902 | !T,T: 1.35(1.04, 1.75) | 0.04960 | C: 1.24(1, 1.54) | 468,465 |
| RS284779 | 04: 100695439 | ADH7 | 0.04424 | C,C: 1.4(1.02, 1.92) | 0.01722 | C: 1.25(1.04, 1.49) | 483,474 |
| RS7655579 | 04: 111223312 | EGF | 0.03368 | A,G: 1.4(1.08, 1.81) | 0.14221 | G: 1.16(0.96, 1.41) | 486,466 |
| RS9991904 | 04: 111280183 | EGF | 0.02481 | !A,A: 1.42(1.1, 1.84) | 0.02409 | G: 1.26(1.03, 1.55) | 473,455 |
| RS1562640 | 04: 111849973 | | 0.00193 | !C,T: 1.62(1.19, 2.2) | 0.09995 | C: 1.25(0.96, 1.64) | 490,470 |
| RS1476214 | 04: 124170614 | FGF2, | 0.04914 | G,G: 1.32(1.01, 1.73) | 0.01727 | G: 1.25(1.04, 1.5) | 489,468 |
| | | NUDT6 | | | | | |
| RS2356394 | 04: 149492266 | NR3C2 | 0.02429 | !A,A: | 0.01498 | G: 1.46(1.08, 1.98) | 436,455 |
| | | | | 3.49(1.06, 11.51) | | | |
| RS2407410 | 04: 152500273 | TAR5753, | 0.02366 | C,C: 4.91(1.25, 19.19) | 0.57800 | C: 1.08(0.83, 1.42) | 492,473 |
| | | SH3D19 | | | | | |
| RS1346376 | 04: 168267615 | SPOCK3 | 0.04071 | !T,T: 2.57(0.96, 6.92) | 0.77953 | T: 1.04(0.79, 1.37) | 448,468 |
| RS1428023 | 05: 005281239 | ADAMTS16 | 0.03248 | !C,T: 1.42(1.09, 1.85) | 0.39738 | T: 1.09(0.9, 1.33) | 439,451 |
| RS10038285 | 05: 042725297 | GHR | 0.02488 | !A,A: 1.41(1.09, 1.82) | 0.01032 | G: 1.31(1.07, 1.61) | 488,468 |
| RS4866948 | 05: 042743143 | GHR | 0.01357 | !G,G: 1.5(1.14, 1.98) | 0.00970 | A: 1.34(1.07, 1.66) | 480,369 |
| RS1862639 | 05: 052343127 | ITGA2 | 0.03428 | C,T: 1.33(1.01, 1.75) | 0.64277 | T: 1.06(0.84, 1.33) | 486,472 |
| RS999289 | 05: 054316655 | ESM1 | 0.12423 | !G,G: 1.28(0.99, 1.65) | 0.03826 | C: 1.23(1.01, 1.49) | 487,467 |
| RS2228043 | 05: 055287688 | IL6ST | 0.03088 | !G,G: | 0.12169 | C: 1.25(0.95, 1.64) | 489,475 |
| | | | | 3.63(1.26, 10.45) | | | |
| RS164392 | 05: 068445370 | SLC30A5 | 0.01544 | !G,G: 2.6(1.29, 5.23) | 0.31075 | A: 1.12(0.9, 1.4) | 490,468 |
| RS2874178 | 05: 071046773 | CART | 0.01599 | A,A: 1.51(1.09, 2.1) | 0.01035 | A: 1.51(1.11, 2.05) | 492,471 |
| RS1047530 | 05: 075947155 | IQGAP2, | 0.02982 | A,G: 1.4(1.08, 1.8) | 0.61590 | G: 1.05(0.88, 1.25) | 489,473 |
| | | F2RL2 | | | | | |
| RS160278 | 05: 082871480 | CSPG2 | 0.09370 | !A,A: 1.37(1.02, 1.83) | 0.03945 | T: 1.21(1.01, 1.45) | 486,468 |
| RS305661 | 05: 083716793 | EDIL3 | 0.11148 | A,A: 1.6(0.92, 2.78) | 0.03871 | A: 1.25(1.01, 1.55) | 489,461 |
| RS2886924 | 05: 090426691 | GPR98 | 0.03579 | !T,T: 1.48(1.1, 1.99) | 0.01399 | C: 1.41(1.08, 1.85) | 483,475 |
| RS2287695 | 05: 094983432 | GPR150 | 0.04123 | A,T: 1.4(1.03, 1.89) | 0.40130 | T: 1.12(0.87, 1.45) | 476,425 |
| RS39602 | 05: 096390210 | LNPEP | 0.04861 | G,G: 1.4(1.06, 1.84) | 0.08885 | G: 1.17(0.98, 1.4) | 490,472 |
| RS4958202 | 05: 133340250 | VDAC1 | 0.05333 | !A,A: 1.38(1.05, 1.82) | 0.01703 | C: 1.34(1.05, 1.71) | 482,462 |
| RS258747 | 05: 142637006 | NR3C1 | 0.04133 | C,C: 1.4(1.05, 1.86) | 0.01117 | C: 1.27(1.06, 1.52) | 478,463 |
| RS852977 | 05: 142667687 | NR3C1 | 0.13778 | !A,A: 1.23(0.96, 1.59) | 0.04777 | G: 1.22(1, 1.48) | 492,471 |
| RS33388 | 05: 142677488 | NR3C1 | 0.02677 | !T,T: 1.41(1.04, 1.91) | 0.00618 | A: 1.29(1.08, 1.55) | 468,463 |
| RS33389 | 05: 142680692 | NR3C1 | 0.06855 | !C,C: 1.35(1.01, 1.81) | 0.03154 | T: 1.34(1.03, 1.75) | 490,470 |
| RS2918417 | 05: 142706363 | NR3C1 | 0.13489 | !G,G: 1.24(0.96, 1.6) | 0.04656 | A: 1.22(1, 1.49) | 490,474 |
| RS6877893 | 05: 142707386 | NR3C1 | 0.03827 | G,G: 1.41(1.06, 1.87) | 0.01165 | G: 1.26(1.06, 1.51) | 483,469 |
| RS1368384 | 05: 147875141 | HTR4 | 0.05941 | T,T: 1.36(1.05, 1.77) | 0.02517 | T: 1.28(1.04, 1.59) | 491,469 |
| RS1346489 | 05: 151246727 | GLRA1 | 0.00611 | G,G: 1.45(1.11, 1.9) | 0.00203 | G: 1.42(1.14, 1.77) | 406,454 |
| RS512983 | 05: 152875602 | GRIA1 | 0.04407 | A,A: 1.91(1.05, 3.45) | 0.02017 | A: 1.3(1.04, 1.61) | 494,476 |
| RS27341 | 05: 156612154 | ITK | 0.00488 | !C,C: 1.68(1.19, 2.37) | 0.19542 | T: 1.13(0.94, 1.36) | 491,472 |
| RS2863747 | 05: 156863362 | ADAM19 | 0.06309 | C,C: 1.36(1.05, 1.77) | 0.03371 | C: 1.26(1.02, 1.56) | 484,468 |
| 10017459 | 05: 159278200 | ADRA1B | 0.01782 | !C,G: 1.61(1.15, 2.26) | 0.02883 | G: 1.42(1.04, 1.93) | 483,468 |
| RS5369 | 06: 012402244 | EDN1 | 0.03368 | A,G: 1.48(1.09, 2.02) | 0.03514 | A: 1.35(1.02, 1.78) | 490,474 |
| RS4529296 | 06: 026191114 | HFE | 0.03695 | !C,C: 1.38(1.06, 1.8) | 0.10404 | G: 1.17(0.97, 1.4) | 490,476 |
| RS1234844 | 06: 029560115 | MAS1L | 0.01262 | A,A: 1.64(1.14, 2.37) | 0.00545 | A: 1.63(1.16, 2.31) | 491,474 |
| RS1063635 | 06: 031487910 | MICA, | 0.00864 | !G,G: 1.56(1.17, 2.09) | 0.04276 | A: 1.21(1.01, 1.45) | 485,460 |
| | | BAT1 | | | | | |
| RS2516479 | 06: 031636305 | NFKBIL1 | 0.01487 | C,G: 1.46(1.13, 1.88) | 0.67804 | C: 1.04(0.87, 1.25) | 489,463 |
| RS2072633 | 06: 032027557 | CFB, | 0.03243 | !T,T: 1.52(1.05, 2.21) | 0.01286 | C: 1.27(1.05, 1.52) | 488,474 |
| | | RDBP | | | | | |
| RS626156 | 06: 033778460 | C6ORF125 | 0.03391 | A,G: 1.38(1.07, 1.77) | 0.16383 | G: 1.14(0.95, 1.37) | 494,475 |
| RS733590 | 06: 036753181 | CDKN1A | 0.02021 | !C,C: 1.67(1.16, 2.39) | 0.03453 | T: 1.22(1.02, 1.47) | 489,476 |
| RS3827632 | 06: 041361305 | TREM1 | 0.01061 | !G,G: 18(1.04, 312.77) | 0.37687 | C: 1.14(0.85, 1.52) | 485,466 |
| 6728590 | 06: 041812750 | PGC, | 0.02816 | C,G: 1.4(1.04, 1.88) | 0.36195 | C: 1.13(0.87, 1.46) | 491,468 |
| | | TFEB | | | | | |
| RS1234128 | 06: 046940927 | GPR116 | 0.12063 | T,T: 1.35(1.01, 1.82) | 0.04302 | T: 1.32(1.01, 1.72) | 495,474 |
| RS187233 | 06: 049767662 | CRISP2 | 0.02968 | !A,G: 1.4(1.08, 1.8) | 0.61417 | A: 1.05(0.88, 1.25) | 481,471 |
| RS2202801 | 06: 054275716 | | 0.00765 | C,C: 1.59(1.18, 2.14) | 0.00482 | C: 1.47(1.13, 1.92) | 452,456 |
| RS1523936 | 06: 069662403 | BAI3 | 0.03740 | !C,T: 1.47(1.09, 1.99) | 0.17174 | T: 1.19(0.93, 1.52) | 454,455 |
| RS1772203 | 06: 105827231 | PREP | 0.06065 | !C,C: 3.14(0.93, 10.6) | 0.04529 | G: 1.34(1.01, 1.77) | 490,475 |
| RS2400108 | 06: 105917143 | PREP | 0.05836 | T,T: 1.39(1.05, 1.83) | 0.01782 | T: 1.35(1.05, 1.72) | 491,475 |
| RS6912527 | 06: 110560112 | WASF1 | 0.08418 | A,A: 1.41(1.03, 1.92) | 0.02798 | A: 1.38(1.04, 1.85) | 493,469 |
| RS1726132 | 06: 112083227 | FYN | 0.04146 | !A,A: 1.48(1.02, 2.15) | 0.01312 | G: 1.27(1.05, 1.53) | 475,469 |
| RS804192 | 06: 112116712 | FYN | 0.00607 | !G,G: 1.52(1.17, 1.98) | 0.00148 | A: 1.42(1.14, 1.76) | 475,450 |
| RS1616619 | 06: 134545897 | SGK | 0.02934 | C,T: 1.4(1.09, 1.81) | 0.78480 | C: 1.03(0.86, 1.23) | 491,475 |
| RS1360194 | 06: 142794288 | GPR126 | 0.05411 | !G,G: 1.34(1.04, 1.74) | 0.01333 | A: 1.3(1.06, 1.59) | 471,465 |
| RS2881766 | 06: 152211233 | ESR1 | 0.07520 | !G,G: 1.89(1, 3.56) | 0.03152 | T: 1.3(1.03, 1.64) | 489,473 |
| RS2071454 | 06: 152218938 | ESR1 | 0.06614 | T,T: 1.47(1.06, 2.05) | 0.03125 | T: 1.4(1.04, 1.9) | 479,466 |
| RS712221 | 06: 152272355 | ESR1 | 0.09417 | !A,A: 1.31(0.99, 1.72) | 0.03278 | T: 1.23(1.02, 1.48) | 451,464 |
| RS750686 | 06: 152500240 | ESR1 | 0.11620 | !G,G: 1.28(0.99, 1.66) | 0.03925 | A: 1.22(1.01, 1.48) | 487,468 |
| RS756519 | 06: 170768494 | PSMB1 | 0.01125 | !T,T: 1.68(1.16, 2.42) | 0.18620 | C: 1.14(0.94, 1.37) | 477,475 |
| RS1881125 | 07: 000870159 | MGC11257, | 0.01675 | !A,G: 1.44(1.11, 1.86) | 0.19050 | G: 1.13(0.94, 1.36) | 479,467 |
| | | GPR146 | | | | | |
| RS1104888 | 07: 000879253 | MGC1127 | 0.02757 | A,A: 1.44(1.08, 1.9) | 0.10023 | A: 1.16(0.97, 1.39) | 487,475 |
| RS7313 | 07: 028843449 | CPVL | 0.06521 | !T,T: 1.39(1.03, 1.89) | 0.02214 | C: 1.24(1.03, 1.48) | 489,472 |
| RS323178 | 07: 028929699 | CPVL | 0.03654 | T,T: 2.95(1.2, 7.23) | 0.09793 | T: 1.23(0.96, 1.56) | 493,472 |
| RS245873 | 07: 028958085 | CPVL | 0.01498 | C,C: 1.58(1.12, 2.22) | 0.22428 | C: 1.12(0.94, 1.35) | 478,469 |
| RS929377 | 07: 030477399 | CRHR2 | 0.01995 | T,T: 1.43(1.1, 1.86) | 0.00711 | T: 1.31(1.08, 1.59) | 455,441 |
| RS2014663 | 07: 030488813 | CRHR2 | 0.02869 | !G,G: 2.9(0.98, 8.6) | 0.02026 | A: 1.34(1.05, 1.72) | 485,472 |
| RS917195 | 07: 030501692 | CRHR2 | 0.04317 | !C,T: 1.31(1.01, 1.71) | 0.70221 | C: 1.05(0.84, 1.3) | 490,473 |
| RS323922 | 07: 034522022 | NPSR1, | 0.00629 | !C,C: 1.7(1.2, 2.4) | 0.08582 | G: 1.17(0.98, 1.41) | 492,473 |
| | | AAA1 | | | | | |
| RS6956366 | 07: 054875710 | EGFR | 0.04560 | C,C: 1.55(1.04, 2.32) | 0.01654 | C: 1.27(1.05, 1.54) | 481,460 |
| RS7796872 | 07: 054954053 | EGFR | 0.00930 | G,G: 1.5(1.09, 2.05) | 0.00386 | G: 1.52(1.14, 2.03) | 470,457 |
| RS2240466 | 07: 072300920 | BAZ1B | 0.03996 | T,T: 6.18(1.1, 34.72) | 0.16121 | T: 1.24(0.93, 1.66) | 493,473 |
| RS2428469 | 07: 079410273 | GNAI1 | 0.02260 | !A,A: 1.43(1.11, 1.84) | 0.01036 | C: 1.3(1.06, 1.59) | 491,469 |
| RS1949818 | 07: 079881710 | CD36 | 0.05986 | G,G: 1.31(1, 1.7) | 0.01768 | G: 1.25(1.04, 1.51) | 480,454 |
| RS2229952 | 07: 081312119 | | 0.02808 | A,G: 1.61(1.09, 2.39) | 0.03988 | A: 1.48(1.02, 2.14) | 490,475 |
| RS38559 | 07: 081529638 | CACNA2D1 | 0.03697 | G,G: 1.45(1.04, 2.02) | 0.01525 | G: 1.47(1.08, 2) | 490,473 |
| RS38564 | 07: 081535740 | CACNA2D1 | 0.03928 | !G,G: 1.38(1.07, 1.78) | 0.01584 | T: 1.28(1.05, 1.56) | 489,473 |
| RS4355720 | 07: 083323603 | SEMA3A | 0.04587 | !A,A: 1.38(1.07, 1.79) | 0.05027 | G: 1.22(1, 1.49) | 473,443 |
| RS2158785 | 07: 086031640 | GRM3 | 0.11378 | C,C: 1.24(0.96, 1.61) | 0.04755 | C: 1.24(1, 1.53) | 483,470 |
| RS970186 | 07: 086066321 | GRM3 | 0.00395 | !C,C: 2.35(1.25, 4.44) | 0.00199 | G: 1.41(1.14, 1.75) | 481,462 |
| RS17126 | 07: 086116564 | GRM3 | 0.00088 | G,G: 1.52(1.18, 1.97) | 0.00013 | G: 1.49(1.22, 1.84) | 481,467 |
| RS1063964 | 07: 087480120 | SRI | 0.02974 | !C,C: 1.41(1.09, 1.82) | 0.01613 | T: 1.27(1.05, 1.54) | 489,474 |
| RS1048303 | 07: 100397575 | AP1S1, | 0.11654 | !T,T: 1.32(1.01, 1.72) | 0.04852 | C: 1.21(1.01, 1.45) | 483,456 |
| | | VGF | | | | | |
| RS213951 | 07: 116796385 | CFTR | 0.03191 | !A,A: 1.42(1.09, 1.84) | 0.01210 | G: 1.32(1.06, 1.64) | 492,469 |
| 8211019 | 07: 142094221 | TRPV6 | 0.01865 | C,G: 1.56(1.06, 2.32) | 0.20205 | C: 1.27(0.89, 1.82) | 492,474 |
| RS- | 07: 142096825 | TRPV6 | 0.01997 | C,T: 1.56(1.05, 2.3) | 0.20256 | C: 1.27(0.89, 1.82) | 492,472 |
| GSK10018648 | | | | | | | |
| RS2293365 | 07: 142555742 | CLCN1 | 0.03416 | C,C: 1.54(1.1, 2.15) | 0.01793 | C: 1.25(1.04, 1.5) | 490,472 |
| RS2618458 | 08: 011406477 | BLK | 0.07579 | !C,C: 1.32(1, 1.75) | 0.02771 | T: 1.33(1.04, 1.7) | 491,471 |
| RS10112596 | 08: 011617211 | GATA4 | 0.00792 | !G,G: 1.58(1.18, 2.12) | 0.00324 | A: 1.49(1.14, 1.93) | 494,449 |
| RS285 | 08: 019859469 | LPL | 0.06201 | !C,C: 1.39(1.04, 1.85) | 0.02406 | T: 1.23(1.03, 1.48) | 477,468 |
| RS1552286 | 08: 022020747 | NUDT18 | 0.03640 | C,C: 3.15(1.2, 8.28) | 0.07269 | C: 1.27(0.98, 1.66) | 488,471 |
| RS1369836 | 08: 023770067 | STC1 | 0.00675 | !A,A: 1.51(1.17, 1.95) | 0.00352 | G: 1.37(1.11, 1.69) | 492,475 |
| RS2976440 | 08: 024864142 | NEFL | 0.04256 | T,T: 1.34(1.04, 1.74) | 0.01211 | T: 1.27(1.06, 1.53) | 489,469 |
| RS2976437 | 08: 024872047 | NEFL | 0.03035 | A,A: 1.36(1.05, 1.76) | 0.00815 | A: 1.29(1.07, 1.55) | 495,467 |
| RS6185 | 08: 025336717 | GNRH1 | 0.03191 | !C,G: 1.42(1.09, 1.84) | 0.06531 | G: 1.22(0.99, 1.5) | 492,473 |
| RS682734 | 08: 054024038 | | 0.01838 | !A,G: 1.44(1.11, 1.87) | 0.14142 | A: 1.15(0.96, 1.39) | 471,469 |
| RS1457043 | 08: 059572993 | CYP7A1 | 0.01955 | !A,G: 1.43(1.1, 1.84) | 0.88944 | G: 1.01(0.85, 1.22) | 486,463 |
| RS2162459 | 08: 059573596 | CYP7A1 | 0.01335 | !A,G: 1.45(1.12, 1.87) | 0.96323 | G: 1(0.84, 1.2) | 490,468 |
| RS3824260 | 08: 059575744 | CYP7A1 | 0.03401 | !A,G: 1.38(1.07, 1.78) | 0.88918 | A: 1.01(0.85, 1.22) | 494,464 |
| RS7833031 | 08: 066808103 | PDE7A | 0.00832 | !G,G: 1.5(1.16, 1.93) | 0.00295 | A: 1.36(1.11, 1.67) | 491,458 |
| RS3176921 | 08: 067253933 | CRH | 0.00115 | !C,T: 1.96(1.25, 3.08) | 0.34219 | T: 1.2(0.83, 1.75) | 450,463 |
| RS2250178 | 08: 073821473 | KCNB2 | 0.02022 | !C,C: 1.62(1.07, 2.45) | 0.00754 | A: 1.3(1.08, 1.58) | 488,467 |
| RS1440349 | 08: 073835471 | KCNB2 | 0.02513 | !G,G: 1.38(1.06, 1.81) | 0.00675 | A: 1.3(1.08, 1.57) | 475,451 |
| RS1440332 | 08: 073897644 | KCNB2 | 0.00344 | C,T: 1.6(1.22, 2.1) | 0.42543 | T: 1.09(0.89, 1.32) | 469,370 |
| 6883899 | 08: 086576323 | CA2 | 0.01890 | !G,G: 2.7(1.08, 6.75) | 0.79785 | G: 1.04(0.81, 1.33) | 490,471 |
| RS2664361 | 08: 089200925 | MMP16 | 0.10175 | A,A: 1.3(0.99, 1.71) | 0.03105 | A: 1.22(1.02, 1.46) | 489,473 |
| RS1996637 | 08: 089227035 | MMP16 | 0.07935 | T,T: 1.33(1.01, 1.74) | 0.02400 | T: 1.23(1.03, 1.48) | 483,471 |
| RS7461448 | 08: 132057791 | ADCY8 | 0.03079 | !G,G: 1.38(1.02, 1.87) | 0.02105 | A: 1.4(1.06, 1.84) | 484,448 |
| RS2005895 | 08: 140738217 | KCNK9 | 0.08061 | !T,T: 1.32(1.02, 1.7) | 0.03042 | C: 1.24(1.02, 1.5) | 487,470 |
| RS10105905 | 08: 141930077 | PTK2 | 0.08232 | !G,G: 1.42(1.01, 1.99) | 0.03223 | C: 1.22(1.02, 1.47) | 486,466 |
| RS4074812 | 08: 141952711 | PTK2 | 0.11526 | !G,G: 1.36(0.97, 1.91) | 0.03948 | A: 1.21(1.01, 1.46) | 485,457 |
| RS727944 | 08: 142044370 | PTK2 | 0.04441 | !A,A: 1.49(1.06, 2.08) | 0.02066 | T: 1.24(1.04, 1.49) | 490,471 |
| RS7005909 | 08: 142052881 | PTK2 | 0.05254 | !G,G: 1.48(1.05, 2.08) | 0.02253 | A: 1.24(1.03, 1.49) | 486,468 |
| RS3934583 | 08: 142439523 | GPR20 | 0.00928 | !T,T: | 0.15578 | C: 1.23(0.93, 1.63) | 490,473 |
| | | | | 17.91(1.03, 311.22) | | | |
| RS2375994 | 09: 002621092 | VLDLR | 0.09755 | !A,A: 1.33(1.02, 1.73) | 0.04319 | G: 1.21(1.01, 1.46) | 488,462 |
| RS6144 | 09: 002635557 | VLDLR | 0.08361 | A,A: 1.36(1.03, 1.78) | 0.02591 | A: 1.3(1.03, 1.63) | 485,439 |
| RS1487509 | 09: 016976415 | | 0.02210 | G,G: 1.72(1.08, 2.73) | 0.01409 | G: 1.74(1.11, 2.74) | 466,465 |
| RS1487505 | 09: 016990713 | | 0.03245 | !A,A: | 0.07247 | T: 1.34(0.98, 1.82) | 414,473 |
| | | | | 11.53(0.65, 205.22) | | | |
| RS2782416 | 09: 027097517 | TEK | 0.13231 | G,G: 1.3(0.99, 1.71) | 0.04817 | G: 1.27(1.01, 1.61) | 488,474 |
| RS3849904 | 09: 033424026 | AQP3 | 0.03756 | A,A: 1.39(1.07, 1.8) | 0.02784 | A: 1.27(1.03, 1.57) | 491,473 |
| RS567691 | 09: 033429829 | AQP3 | 0.07602 | G,G: 1.32(1, 1.76) | 0.02904 | G: 1.33(1.03, 1.71) | 490,476 |
| RS760098 | 09: 033782265 | PRSS3 | 0.04683 | A,A: 1.37(1.07, 1.77) | 0.02979 | A: 1.25(1.02, 1.52) | 489,476 |
| RS2026118 | 09: 034656093 | CCL27 | 0.00805 | !A,A: | 0.00487 | G: 1.48(1.13, 1.94) | 491,468 |
| | | | | 3.46(1.19, 10.03) | | | |
| RS1324471 | 09: 036027644 | RECK | 0.07454 | A,A: 1.5(1.04, 2.15) | 0.03166 | A: 1.45(1.03, 2.04) | 479,452 |
| RS2035073 | 09: 090688974 | SYK | 0.00084 | T,T: 1.61(1.25, 2.08) | 0.00028 | T: 1.44(1.18, 1.74) | 484,473 |
| RS2387337 | 09: 090689328 | SYK | 0.00198 | A,A: 1.54(1.2, 1.99) | 0.00040 | A: 1.43(1.18, 1.74) | 483,470 |
| RS1007651 | 09: 098342320 | GABBR2 | 0.02627 | !C,C: | 0.46250 | C: 1.16(0.81, 1.67) | 483,468 |
| | | | | 9.37(0.5, 174.48) | | | |
| RS1055187 | 09: 099812853 | STX17 | 0.09813 | T,T: 1.32(1.02, 1.7) | 0.03381 | T: 1.24(1.02, 1.5) | 479,472 |
| RS4149313 | 09: 104666308 | ABCA1 | 0.02551 | A,G: 1.53(1.12, 2.09) | 0.03244 | G: 1.35(1.03, 1.77) | 482,460 |
| RS2275495 | 09: 108723951 | IKBKAP | 0.01582 | C,C: 1.48(1.08, 2.03) | 0.00633 | C: 1.5(1.12, 2.01) | 492,476 |
| RS838827 | 09: 108748207 | IKBKAP | 0.00836 | C,C: 1.71(1.15, 2.54) | 0.00749 | C: 1.68(1.15, 2.46) | 484,474 |
| RS754333 | 09: 108766451 | IKBKAP | 0.04439 | C,C: 3.81(1.17, 12.43) | 0.16053 | C: 1.2(0.93, 1.54) | 485,469 |
| RS2418076 | 09: 110092906 | TXN | 0.04668 | !T,T: 1.94(1.14, 3.33) | 0.13503 | C: 1.17(0.95, 1.45) | 444,461 |
| RS2766999 | 09: 110629723 | MUSK | 0.00569 | !T,T: 1.55(1.17, 2.05) | 0.03099 | C: 1.22(1.02, 1.46) | 486,467 |
| RS955888 | 09: 110717217 | EDG2 | 0.12996 | G,G: 1.26(0.98, 1.63) | 0.04105 | G: 1.24(1.01, 1.52) | 493,458 |
| RS4837805 | 09: 120825809 | C5 | 0.01016 | !G,G: 1.83(1.22, 2.72) | 0.06967 | A: 1.19(0.99, 1.44) | 473,471 |
| RS2236386 | 09: 124257290 | PSMB7 | 0.11485 | !C,C: 1.32(1, 1.74) | 0.04955 | G: 1.2(1, 1.44) | 493,475 |
| RS2387673 | 10: 001449379 | ADARB2 | 0.10297 | !A,A: 1.33(1.02, 1.75) | 0.03692 | G: 1.29(1.02, 1.63) | 489,468 |
| RS2249266 | 10: 001464611 | ADARB2 | 0.07174 | !C,C: 1.34(1.04, 1.73) | 0.03181 | G: 1.24(1.02, 1.51) | 490,475 |
| RS4880531 | 10: 001717178 | ADARB2 | 0.03905 | A,G: 1.45(1, 2.1) | 0.15317 | A: 1.31(0.92, 1.86) | 492,476 |
| RS3736863 | 10: 006054209 | IL15RA | 0.03731 | T,T: 1.39(1.08, 1.8) | 0.02562 | T: 1.27(1.03, 1.57) | 485,471 |
| RS1277733 | 10: 018602544 | CACNB2 | 0.03118 | A,G: 1.4(1.07, 1.85) | 0.33155 | G: 1.12(0.9, 1.41) | 481,471 |
| RS1012227 | 10: 018721172 | CACNB2 | 0.02548 | C,G: 1.38(1.06, 1.78) | 0.84250 | C: 1.02(0.84, 1.24) | 489,475 |
| RS3034 | 10: 030789901 | MAP3K8 | 0.03133 | A,A: 1.48(1.1, 1.99) | 0.00907 | A: 1.42(1.09, 1.85) | 494,474 |
| RS2488335 | 10: 033272962 | ITGB1 | 0.01615 | C,G: 1.46(1.13, 1.89) | 0.22168 | G: 1.13(0.93, 1.37) | 480,463 |
| RS4128662 | 10: 047173952 | | 0.06740 | !A,A: 1.33(0.99, 1.77) | 0.02515 | G: 1.35(1.04, 1.75) | 487,466 |
| RS2813310 | 10: 047574950 | ASAH2 | 0.08680 | !A,A: 1.33(1.03, 1.72) | 0.04608 | G: 1.24(1, 1.52) | 488,467 |
| RS3211105 | 10: 064615370 | JMJD1C | 0.09860 | T,T: 1.34(1.02, 1.75) | 0.04079 | T: 1.28(1.01, 1.6) | 482,475 |
| RS2306264 | 10: 064623965 | JMJD1C | 0.10562 | !G,G: 1.27(0.98, 1.64) | 0.03827 | C: 1.23(1.01, 1.5) | 485,466 |
| RS4746058 | 10: 072130519 | ADAMTS14 | 0.01512 | !C,G: 1.58(1.12, 2.22) | 0.08314 | C: 1.33(0.97, 1.81) | 486,471 |
| RS4747082 | 10: 072136936 | ADAMTS14 | 0.03179 | !A,G: 1.55(1.07, 2.25) | 0.07692 | G: 1.38(0.98, 1.96) | 494,474 |
| RS1572799 | 10: 072181064 | ADAMTS14 | 0.03041 | A,A: 2.54(1.18, 5.46) | 0.03301 | A: 1.31(1.02, 1.68) | 459,457 |
| RS4919432 | 10: 101934376 | SPFH1 | 0.03229 | C,T: 1.51(0.97, 2.36) | 0.29135 | T: 1.26(0.83, 1.91) | 490,473 |
| RS1408578 | 10: 101952519 | CHUK | 0.00672 | A,T: 2.82(1.28, 6.22) | 0.00718 | T: 2.77(1.27, 6.09) | 493,467 |
| RS3853517 | 10: 101973535 | CHUK | 0.01843 | A,G: 2.27(1.09, 4.73) | 0.09098 | G: 1.87(0.94, 3.73) | 490,463 |
| RS927302 | 10: 102712906 | C10ORF6 | 0.04505 | !C,G: 1.4(1.07, 1.83) | 0.12938 | C: 1.19(0.95, 1.48) | 490,475 |
| RS3740487 | 10: 102740773 | PEO1 | 0.03852 | !A,C: 1.4(1.07, 1.84) | 0.19801 | C: 1.16(0.93, 1.45) | 492,473 |
| RS7897 | 10: 103912886 | NOLC1 | 0.08524 | T,T: 1.42(1.03, 1.97) | 0.04809 | T: 1.2(1, 1.44) | 488,473 |
| RS2487987 | 10: 105729904 | SLK | 0.00249 | A,A: 4.09(1.46, 11.5) | 1.00000 | A: 1.01(0.79, 1.29) | 490,449 |
| RS2476959 | 10: 105732933 | SLK | 0.04089 | !A,G: 1.34(1.01, 1.79) | 0.57664 | A: 1.08(0.84, 1.37) | 489,457 |
| RS1612212 | 10: 105738292 | SLK | 0.01907 | !A,G: 1.4(1.05, 1.87) | 0.37876 | G: 1.12(0.87, 1.43) | 478,465 |
| RS2487997 | 10: 105742015 | SLK | 0.04297 | !A,G: 1.39(1.04, 1.85) | 0.28936 | A: 1.15(0.9, 1.46) | 481,467 |
| RS2476955 | 10: 105748455 | SLK | 0.01508 | !C,T: 1.4(1.05, 1.86) | 0.49655 | C: 1.09(0.86, 1.39) | 490,459 |
| RS1774600 | 10: 105764724 | SLK | 0.01175 | A,A: 2.85(1.16, 6.98) | 0.95007 | C: 1.01(0.79, 1.3) | 493,456 |
| RS805681 | 10: 105773787 | SLK | 0.01992 | !A,G: 1.36(1.02, 1.81) | 0.62189 | G: 1.07(0.84, 1.36) | 489,466 |
| RS2282436 | 10: 105787165 | COL17A1 | 0.00852 | !A,C: 1.43(1.08, 1.9) | 0.42629 | A: 1.11(0.87, 1.41) | 491,474 |
| RS876873 | 10: 114161589 | ACSL5 | 0.03991 | !C,C: 3.39(1.17, 9.84) | 0.10331 | T: 1.23(0.96, 1.57) | 488,474 |
| RS1338172 | 10: 120353701 | PRLHR | 0.01553 | !G,G: | 0.04128 | A: 1.35(1.01, 1.8) | 487,473 |
| | | | | 6.63(1.18, 37.29) | | | |
| RS2250868 | 10: 135163684 | ENSG00000148805 | 0.13114 | !C,C: 1.35(1, 1.84) | 0.04725 | G: 1.33(1.01, 1.76) | 488,474 |
| RS800343 | 11: 002386193 | TRPM5 | 0.04014 | !A,A: | 0.16411 | G: 1.3(0.9, 1.87) | 479,469 |
| | | | | 13.45(0.76, 239.41) | | | |
| 10017430 | 11: 003197127 | C11ORF36, | 0.04350 | !C,G: 1.39(1.06, 1.84) | 0.47114 | C: 1.09(0.88, 1.35) | 482,449 |
| | | MRGPRG | | | | | |
| RS2048092 | 11: 006586106 | ILK | 0.10955 | !G,G: 1.35(0.99, 1.83) | 0.03846 | A: 1.22(1.01, 1.46) | 490,420 |
| RS307247 | 11: 013470054 | PTH | 0.03087 | !C,C: 1.36(1.04, 1.78) | 0.01001 | T: 1.32(1.07, 1.62) | 470,378 |
| RS739688 | 11: 017384133 | ABCC8 | 0.04162 | C,C: 1.39(1.07, 1.8) | 0.04643 | C: 1.21(1, 1.46) | 494,474 |
| RS4757738 | 11: 018917724 | | 0.04633 | !G,G: 1.34(1, 1.78) | 0.05873 | C: 1.3(1, 1.7) | 489,473 |
| 6946514 | 11: 019037357 | MRGPRX2 | 0.05221 | !G,G: 1.33(1.03, 1.72) | 0.01438 | T: 1.28(1.05, 1.55) | 487,458 |
| RS1945771 | 11: 020610197 | SLC6A5 | 0.00022 | C,C: 1.6(1.23, 2.08) | 0.00006 | C: 1.47(1.22, 1.78) | 488,462 |
| 6728204 | 11: 027358279 | LGR4 | 0.02610 | !T,T: | 0.04291 | C: 1.5(1.03, 2.2) | 478,470 |
| | | | | 11.31(0.62, 205.06) | | | |
| RS2070850 | 11: 046698071 | F2 | 0.06712 | C,C: 1.37(1.02, 1.85) | 0.02698 | C: 1.36(1.04, 1.79) | 478,468 |
| RS2289612 | 11: 059864046 | MS4A6E | 0.03815 | !T,T: 1.46(1.07, 2) | 0.17013 | G: 1.13(0.95, 1.36) | 492,471 |
| RS2306836 | 11: 060238905 | MS4A8B | 0.10920 | T,T: 1.41(1, 2) | 0.03527 | T: 1.42(1.03, 1.95) | 464,461 |
| RS239253 | 11: 064741319 | FLJ16331 | 0.03487 | !T,T: 1.42(1.08, 1.87) | 0.02274 | C: 1.32(1.04, 1.67) | 485,475 |
| RS- | 11: 068503377 | MRGPRD | 0.08600 | !G,G: 1.35(1.03, 1.75) | 0.04287 | A: 1.26(1.01, 1.57) | 478,467 |
| GSK7003776 | | | | | | | |
| RS2511241 | 11: 072622989 | P2RY2 | 0.02079 | T,T: 1.67(1.12, 2.5) | 0.00761 | T: 1.68(1.15, 2.46) | 475,471 |
| RS3781730 | 11: 075585731 | WNT11 | 0.01691 | !C,T: 1.61(1.15, 2.24) | 0.01507 | C: 1.42(1.07, 1.89) | 418,452 |
| RS2004652 | 11: 075600560 | WNT11 | 0.07403 | A,A: 1.57(1.05, 2.35) | 0.04141 | A: 1.22(1.01, 1.48) | 493,473 |
| RS948977 | 11: 076479630 | CAPN5 | 0.01067 | T,T: 1.81(1.11, 2.94) | 0.80110 | T: 1.03(0.84, 1.25) | 490,470 |
| RS948141 | 11: 083952129 | DLG2 | 0.03979 | T,T: 1.31(1.02, 1.69) | 0.01240 | T: 1.28(1.06, 1.55) | 486,467 |
| 6884040 | 11: 086338534 | FZD4 | 0.09533 | T,T: 1.32(1.02, 1.72) | 0.03474 | T: 1.24(1.02, 1.52) | 441,454 |
| RS217099 | 11: 087662821 | CTSC | 0.06265 | !C,C: 1.39(1.05, 1.83) | 0.03096 | T: 1.31(1.03, 1.67) | 489,464 |
| RS217060 | 11: 087699331 | CTSC | 0.04393 | !T,T: 1.37(1.06, 1.77) | 0.04312 | C: 1.24(1.01, 1.52) | 492,474 |
| RS217062 | 11: 087700147 | CTSC | 0.05308 | !G,G: 1.36(1.05, 1.75) | 0.04825 | A: 1.23(1, 1.51) | 489,470 |
| RS10501668 | 11: 087932994 | GRM5 | 0.01429 | !A,A: 2.12(1.25, 3.59) | 0.02146 | C: 1.28(1.04, 1.58) | 486,456 |
| RS3337 | 11: 087974669 | GRM5 | 0.01117 | !G,G: 2.15(1.27, 3.64) | 0.14662 | A: 1.17(0.95, 1.45) | 478,453 |
| RS2927493 | 11: 087980312 | GRM5 | 0.03057 | !C,C: 1.66(1.11, 2.49) | 0.26313 | T: 1.12(0.93, 1.36) | 491,461 |
| RS1491891 | 11: 087988043 | GRM5 | 0.00516 | !T,T: 2.61(1.42, 4.78) | 0.05745 | C: 1.23(1, 1.53) | 487,475 |
| RS982011 | 11: 088004599 | GRM5 | 0.00547 | !C,C: | 0.82029 | C: 1.04(0.77, 1.4) | 490,458 |
| | | | | 16.3(0.93, 286.13) | | | |
| RS2169661 | 11: 088087395 | GRM5 | 0.02011 | !T,T: 1.9(1.15, 3.14) | 0.43472 | C: 1.09(0.89, 1.34) | 493,474 |
| RS1903843 | 11: 088106461 | GRM5 | 0.00053 | !T,T: 2.25(1.41, 3.6) | 0.24889 | C: 1.12(0.92, 1.37) | 489,470 |
| RS2126739 | 11: 088107985 | GRM5 | 0.00103 | !T,T: 4.02(1.78, 9.11) | 0.07568 | A: 1.24(0.98, 1.56) | 493,470 |
| RS498186 | 11: 102174855 | MMP1 | 0.01535 | !A,A: 1.41(1.08, 1.85) | 0.00469 | C: 1.3(1.08, 1.56) | 491,468 |
| RS1985242 | 11: 113353483 | HTR3A | 0.08122 | !T,T: 1.34(1.04, 1.73) | 0.03600 | A: 1.23(1.01, 1.5) | 477,464 |
| RS678837 | 11: 116614480 | DKFZP547C195 | 0.04695 | G,G: 1.51(1.08, 2.09) | 0.07723 | G: 1.18(0.98, 1.42) | 482,464 |
| RS6649 | 11: 118713138 | C1QTNF5, | 0.00576 | C,T: 1.46(1.1, 1.93) | 0.16878 | T: 1.19(0.93, 1.52) | 487,463 |
| | | MFRP, | | | | | |
| | | RNF26 | | | | | |
| RS4935970 | 11: 125813541 | KIRREL3 | 0.00908 | !C,T: 2(1.28, 3.13) | 0.02337 | C: 1.59(1.09, 2.32) | 476,208 |
| RS878830 | 11: 125815292 | KIRREL3 | 0.10954 | !A,A: 1.25(0.97, 1.61) | 0.04150 | T: 1.23(1.01, 1.49) | 492,471 |
| RS1548133 | 11: 129821890 | ADAMTS15 | 0.05728 | G,G: 1.46(1.03, 2.07) | 0.02017 | G: 1.24(1.03, 1.49) | 495,476 |
| RS1860420 | 12: 005026536 | KCNA5 | 0.02849 | !C,C: 1.44(1.08, 1.92) | 0.01295 | T: 1.26(1.05, 1.51) | 480,469 |
| RS7487539 | 12: 005452380 | NTF3 | 0.02065 | !C,T: 1.36(1.04, 1.77) | 0.69962 | T: 1.04(0.84, 1.3) | 478,456 |
| RS6489627 | 12: 005459260 | NTF3 | 0.00989 | T,T: 2.47(1.27, 4.82) | 0.54051 | T: 1.07(0.86, 1.34) | 479,451 |
| RS1805484 | 12: 013656784 | GRIN2B | 0.03650 | !C,C: 1.33(1.03, 1.72) | 0.01018 | T: 1.29(1.06, 1.56) | 490,474 |
| RS1805543 | 12: 013675580 | GRIN2B | 0.07052 | !A,A: 1.3(1.01, 1.68) | 0.02224 | G: 1.25(1.04, 1.52) | 487,473 |
| RS220594 | 12: 013858677 | GRIN2B | 0.04993 | !A,A: 1.31(1.02, 1.7) | 0.01754 | G: 1.26(1.04, 1.52) | 492,475 |
| RS704176 | 12: 021883391 | ABCC9 | 0.07258 | G,G: 1.37(1.04, 1.82) | 0.03527 | G: 1.21(1.01, 1.45) | 491,471 |
| RS1393413 | 12: 026549435 | ITPR2 | 0.08968 | !T,T: 1.4(0.99, 1.99) | 0.03171 | C: 1.23(1.02, 1.47) | 490,475 |
| RS2189480 | 12: 046550095 | VDR | 0.06632 | C,C: 1.34(1.03, 1.74) | 0.02241 | C: 1.24(1.03, 1.5) | 479,469 |
| RS886589 | 12: 046763464 | SENP1 | 0.03229 | !G,G: 1.41(1.09, 1.82) | 0.01476 | A: 1.26(1.05, 1.53) | 482,471 |
| RS773108 | 12: 054656178 | RAB5B | 0.01754 | !A,A: 1.45(1.12, 1.87) | 0.00631 | G: 1.32(1.08, 1.61) | 478,454 |
| RS705698 | 12: 054670954 | RAB5B | 0.04082 | !T,T: 1.39(1.07, 1.79) | 0.01904 | C: 1.27(1.04, 1.54) | 481,463 |
| RS2292239 | 12: 054768447 | ERBB3 | 0.01432 | !C,C: 1.45(1.13, 1.87) | 0.01121 | A: 1.28(1.06, 1.55) | 492,475 |
| RS2279373 | 12: 055674465 | ADMR | 0.00702 | !C,T: 1.74(1.21, 2.5) | 0.00965 | C: 1.55(1.12, 2.16) | 485,469 |
| RS733629 | 12: 055692711 | TAC3 | 0.00672 | !C,T: 1.75(1.21, 2.52) | 0.00631 | T: 1.61(1.15, 2.26) | 484,466 |
| RS1549976 | 12: 069055071 | KCNMB4 | 0.06057 | !C,C: 1.34(1.04, 1.73) | 0.02060 | T: 1.26(1.04, 1.53) | 469,469 |
| RS763874 | 12: 094922681 | LTA4H | 0.04205 | !A,G: 1.39(1.08, 1.81) | 0.17583 | A: 1.15(0.94, 1.4) | 469,462 |
| RS1399827 | 12: 107239840 | CMKLR1 | 0.06520 | C,C: 1.64(1.06, 2.55) | 0.04094 | C: 1.23(1.01, 1.49) | 484,468 |
| RS278144 | 12: 118573893 | PRKAB1 | 0.01555 | !A,G: 1.42(1.1, 1.84) | 0.58881 | A: 1.05(0.87, 1.28) | 482,474 |
| RS656612 | 12: 120039372 | P2RX7 | 0.03172 | !C,C: 1.8(1.11, 2.92) | 0.02119 | A: 1.26(1.04, 1.54) | 494,469 |
| RS208294 | 12: 120062973 | P2RX7 | 0.01157 | !G,G: 1.46(1.11, 1.93) | 0.00279 | A: 1.32(1.1, 1.58) | 487,466 |
| RS1186055 | 12: 120063249 | P2RX7 | 0.00462 | G,G: 1.47(1.14, 1.89) | 0.00158 | G: 1.38(1.13, 1.69) | 490,473 |
| RS3751142 | 12: 120085139 | P2RX7 | 0.02128 | A,C: 1.49(1.01, 2.18) | 0.37524 | A: 1.18(0.83, 1.68) | 493,463 |
| RS25644 | 12: 120129366 | P2RX4 | 0.00633 | !A,A: 1.71(1.21, 2.39) | 0.00312 | G: 1.61(1.17, 2.2) | 484,405 |
| RS2229840 | 12: 123351342 | NCOR2 | 0.01382 | !A,G: 1.6(1.16, 2.19) | 0.01915 | G: 1.37(1.05, 1.79) | 431,454 |
| RS7300908 | 12: 131071651 | ULK1 | 0.05521 | !C,C: 1.73(1.02, 2.93) | 0.03959 | T: 1.74(1.04, 2.92) | 472,469 |
| RS1933437 | 13: 027522294 | FLT3 | 0.03693 | !C,C: 1.45(1.05, 2) | 0.01156 | T: 1.26(1.05, 1.51) | 489,476 |
| RS2296189 | 13: 027791642 | FLT1 | 0.00130 | G,G: 3.51(1.62, 7.61) | 0.00349 | G: 1.41(1.12, 1.78) | 491,475 |
| RS2182007 | 13: 027879205 | FLT1 | 0.01909 | T,T: 1.46(1.08, 1.98) | 0.00626 | T: 1.29(1.08, 1.54) | 491,474 |
| RS9315047 | 13: 030219289 | ALOX5AP | 0.02965 | A,T: 1.39(1.08, 1.79) | 0.25119 | A: 1.11(0.93, 1.33) | 493,470 |
| RS677702 | 13: 046344499 | HTR2A | 0.04734 | !T,T: 2.24(1.15, 4.36) | 0.15628 | C: 1.18(0.94, 1.47) | 492,462 |
| RS2149434 | 13: 046376345 | HTR2A | 0.03550 | A,A: 1.51(1.07, 2.12) | 0.23903 | A: 1.12(0.93, 1.34) | 475,470 |
| RS1332091 | 13: 091246903 | GPC5 | 0.05182 | G,G: 1.67(1, 2.78) | 0.02252 | G: 1.26(1.03, 1.54) | 489,473 |
| RS1059751 | 13: 094470951 | ABCC4 | 0.00954 | C,T: 1.48(1.14, 1.9) | 0.46518 | C: 1.07(0.9, 1.28) | 489,475 |
| RS1501855 | 13: 096443203 | OXGR1 | 0.06862 | !C,C: 1.36(1.04, 1.77) | 0.02883 | T: 1.23(1.02, 1.47) | 487,469 |
| RS1887703 | 13: 098747984 | PHGDHL1, | 0.02273 | C,C: 1.42(1.1, 1.84) | 0.03606 | C: 1.22(1.01, 1.47) | 490,473 |
| | | EBI2 | | | | | |
| RS946025 | 13: 100776823 | VGCNL1 | 0.01187 | A,G: 1.46(1.13, 1.89) | 0.05581 | A: 1.21(1, 1.47) | 491,467 |
| RS1582188 | 13: 104934457 | G30, | 0.02118 | !C,T: 1.48(1.11, 1.98) | 0.11196 | T: 1.22(0.96, 1.55) | 481,463 |
| | | DAOA | | | | | |
| RS701567 | 13: 104939996 | DAOA, | 0.01403 | G,G: 1.56(1.11, 2.21) | 0.30383 | G: 1.1(0.92, 1.32) | 481,461 |
| | | G30 | | | | | |
| RS2289046 | 13: 109205907 | IRS2 | 0.10822 | A,A: 1.3(1.01, 1.67) | 0.03926 | A: 1.23(1.01, 1.49) | 490,466 |
| RS8011016 | 14: 022694423 | SLC7A8 | 0.04788 | C,C: 1.96(1.06, 3.63) | 0.59394 | C: 1.07(0.85, 1.35) | 435,446 |
| RS2281472 | 14: 023845686 | CIDEB, | 0.02733 | A,A: 1.44(1.1, 1.87) | 0.01311 | A: 1.33(1.06, 1.65) | 473,463 |
| | | C14ORF21 | | | | | |
| RS1053648 | 14: 023846184 | CIDEB, | 0.00709 | C,C: 2.14(1.26, 3.62) | 0.00420 | C: 2.09(1.26, 3.46) | 482,459 |
| | | C14ORF21, | | | | | |
| | | LTB4R2 | | | | | |
| RS2144492 | 14: 023847251 | CIDEB, | 0.02414 | G,G: 2.01(1.17, 3.48) | 0.00890 | G: 2.02(1.2, 3.4) | 495,309 |
| | | C14ORF21, | | | | | |
| | | LTB4R2 | | | | | |
| RS1951594 | 14: 024154472 | GZMH | 0.03774 | !A,A: 1.39(1.08, 1.8) | 0.02333 | C: 1.25(1.03, 1.51) | 491,472 |
| RS1816628 | 14: 051819186 | PTGDR | 0.04539 | G,G: 1.41(1.04, 1.9) | 0.01568 | G: 1.25(1.05, 1.49) | 493,471 |
| RS1255504 | 14: 062493440 | KCNH5 | 0.01116 | !C,T: 1.5(1.15, 1.97) | 0.73457 | C: 1.04(0.86, 1.25) | 467,392 |
| RS10136228 | 14: 069655022 | SLC8A3 | 0.02260 | G,G: 1.45(1.07, 1.97) | 0.00612 | G: 1.29(1.07, 1.54) | 491,470 |
| RS2361295 | 14: 076035661 | ESRRB | 0.00998 | A,G: 1.47(1.12, 1.92) | 0.21989 | G: 1.15(0.92, 1.43) | 477,467 |
| RS176960 | 14: 077254319 | SNW1, | 0.05672 | !A,A: 1.39(1.06, 1.83) | 0.02286 | C: 1.31(1.04, 1.67) | 487,470 |
| | | C14ORF156 | | | | | |
| RS1286920 | 14: 089609412 | KCNK13 | 0.08063 | !C,C: 1.4(1.02, 1.91) | 0.04430 | G: 1.21(1.01, 1.44) | 491,469 |
| RS140685 | 15: 024771205 | GABRA5 | 0.03879 | C,T: 1.36(1.05, 1.75) | 0.43856 | C: 1.08(0.9, 1.29) | 489,475 |
| RS2337980 | 15: 030231488 | CHRNA7 | 0.02645 | !T,T: 1.58(1.13, 2.21) | 0.05214 | C: 1.2(1, 1.45) | 446,463 |
| RS2059956 | 15: 031910824 | RYR3 | 0.08078 | C,C: 1.35(1.02, 1.79) | 0.02905 | C: 1.23(1.02, 1.47) | 467,469 |
| RS2007958 | 15: 056249857 | AQP9 | 0.06623 | G,G: 1.33(1.01, 1.76) | 0.02832 | G: 1.32(1.03, 1.69) | 487,472 |
| RS8033800 | 15: 058476471 | ANXA2 | 0.03360 | !T,T: 1.6(1.12, 2.28) | 0.04835 | A: 1.21(1, 1.45) | 488,455 |
| RS2277547 | 15: 076869486 | ADAMTS7 | 0.02036 | !C,C: 2.02(1.21, 3.39) | 0.17512 | T: 1.15(0.94, 1.41) | 481,473 |
| 6728885 | 15: 083016233 | SEC11L1 | 0.00951 | A,A: 1.61(1.18, 2.2) | 0.01882 | A: 1.25(1.04, 1.49) | 479,470 |
| 6826348 | 15: 083059207 | SEC11L1 | 0.11562 | A,A: 1.28(1, 1.65) | 0.03959 | A: 1.23(1.01, 1.5) | 495,476 |
| RS728879 | 15: 098491800 | ADAMTS17 | 0.12359 | A,A: 1.28(0.97, 1.7) | 0.04539 | A: 1.21(1.01, 1.45) | 464,468 |
| RS- | 16: 001271597 | | 0.03082 | !G,G: 2.2(1.12, 4.36) | 0.01593 | T: 2.25(1.15, 4.4) | 486,472 |
| GSK8117869 | | | | | | | |
| RS886287 | 16: 002856792 | PRSS22 | 0.02449 | !C,C: 1.44(1.1, 1.88) | 0.00994 | G: 1.28(1.06, 1.54) | 485,459 |
| RS129968 | 16: 003731262 | CREBBP | 0.03784 | !A,G: 1.39(1.08, 1.8) | 0.47150 | G: 1.08(0.89, 1.3) | 484,466 |
| RS2230739 | 16: 003973437 | ADCY9 | 0.02187 | !A,A: 1.4(1.08, 1.81) | 0.00520 | G: 1.33(1.09, 1.61) | 487,457 |
| RS212082 | 16: 016134648 | ABCC1 | 0.00011 | T,T: 1.78(1.34, 2.35) | 0.00003 | T: 1.7(1.32, 2.17) | 491,470 |
| RS1634675 | 16: 019801122 | GPRC5B | 0.08892 | !G,G: 1.33(1.02, 1.74) | 0.02885 | A: 1.29(1.03, 1.62) | 491,472 |
| RS743590 | 16: 028515731 | SULT1A2 | 0.04462 | C,C: 1.38(1.06, 1.8) | 0.07618 | C: 1.19(0.99, 1.43) | 468,454 |
| RS3848300 | 16: 054420106 | CES1 | 0.00791 | G,G: 8.15(1.49, 44.56) | 0.28204 | G: 1.18(0.89, 1.56) | 472,451 |
| RS10802 | 16: 056168297 | GPR114 | 0.08701 | !A,A: 1.36(1.03, 1.78) | 0.03395 | G: 1.28(1.02, 1.6) | 482,448 |
| RS152144 | 16: 056543465 | CNGB1 | 0.03903 | C,G: 1.39(1.08, 1.8) | 0.64132 | G: 1.05(0.87, 1.26) | 493,469 |
| RS2292321 | 16: 082683130 | MBTPS1 | 0.10082 | !G,G: 1.3(1.01, 1.67) | 0.03062 | C: 1.24(1.02, 1.52) | 486,474 |
| RS1862835 | 16: 082826112 | KCNG4 | 0.02349 | !A,G: 1.49(1.02, 2.19) | 0.33115 | A: 1.19(0.84, 1.69) | 463,467 |
| RS774195 | 16: 083459301 | CRISPLD2 | 0.03871 | C,T: 1.39(1.08, 1.79) | 0.28395 | T: 1.11(0.92, 1.34) | 490,473 |
| RS903188 | 16: 083469773 | CRISPLD2 | 0.01912 | !G,G: 1.46(1.12, 1.91) | 0.00890 | A: 1.36(1.09, 1.72) | 490,474 |
| RS774199 | 16: 083470380 | CRISPLD2 | 0.00417 | G,G: 1.51(1.14, 1.99) | 0.00114 | G: 1.35(1.13, 1.62) | 489,475 |
| RS2641666 | 16: 083482495 | CRISPLD2 | 0.01489 | A,C: 1.48(1.13, 1.93) | 0.24091 | A: 1.13(0.93, 1.38) | 445,440 |
| RS982994 | 16: 083484845 | CRISPLD2 | 0.00535 | C,T: 1.5(1.16, 1.94) | 0.43570 | T: 1.08(0.89, 1.31) | 489,470 |
| RS2641674 | 16: 083510605 | LOC123862 | 0.02744 | C,T: 1.36(1.06, 1.76) | 0.67328 | T: 1.04(0.87, 1.25) | 491,471 |
| RS1110400 | 16: 088513631 | MC1R, | 0.01416 | T,T: 3.2(1.21, 8.46) | 0.01468 | T: 3.16(1.2, 8.32) | 491,472 |
| | | TUBB3 | | | | | |
| RS395357 | 17: 003382830 | TRPV3 | 0.03629 | C,T: 1.38(1.07, 1.78) | 0.49539 | T: 1.07(0.89, 1.27) | 494,474 |
| RS8071286 | 17: 004586149 | CXCL16 | 0.02099 | !A,G: 1.38(1.07, 1.79) | 0.57742 | A: 1.05(0.88, 1.26) | 489,462 |
| RS2241933 | 17: 004646905 | PSMB6 | 0.03289 | G,T: 1.4(1.09, 1.81) | 0.28491 | T: 1.11(0.92, 1.35) | 492,473 |
| RS2779248 | 17: 023151959 | NOS2A | 0.04922 | !T,T: 1.35(1.04, 1.76) | 0.01467 | C: 1.26(1.05, 1.52) | 477,469 |
| 10017361 | 17: 024033876 | SUPT6H | 0.03071 | C,C: 1.49(1.1, 2.02) | 0.10017 | C: 1.17(0.97, 1.39) | 488,475 |
| RS4795456 | 17: 024096590 | TRAF4 | 0.12328 | !G,G: 1.29(0.98, 1.71) | 0.04546 | A: 1.28(1.01, 1.63) | 490,409 |
| RS2857653 | 17: 029599872 | CCL2 | 0.07536 | C,C: 1.29(0.98, 1.69) | 0.03488 | C: 1.29(1.02, 1.64) | 472,474 |
| RS4796107 | 17: 031126888 | MMP28 | 0.03607 | G,G: 1.57(1.11, 2.21) | 0.01335 | G: 1.5(1.09, 2.05) | 464,455 |
| RS4796108 | 17: 031150204 | MMP28 | 0.07221 | !C,G: 1.46(1.05, 2.02) | 0.04536 | C: 1.35(1.01, 1.81) | 483,467 |
| RS4795369 | 17: 034862646 | PPARBP | 0.01145 | A,G: 1.46(1.12, 1.9) | 0.54403 | G: 1.07(0.88, 1.3) | 489,458 |
| RS2271308 | 17: 035071008 | STARD3 | 0.05262 | !G,G: 1.37(1.06, 1.77) | 0.01862 | A: 1.27(1.04, 1.56) | 478,461 |
| RS2304497 | 17: 037319300 | ACLY | 0.01407 | G,G: 5.63(0.99, 32.1) | 0.54558 | T: 1.1(0.82, 1.47) | 481,471 |
| RS939881 | 17: 037575778 | KCNH4 | 0.06621 | !T,T: 1.48(1.05, 2.06) | 0.02252 | C: 1.45(1.06, 1.98) | 490,462 |
| RS4098685 | 17: 039985138 | FZD2 | 0.06777 | C,C: 1.33(0.98, 1.79) | 0.03271 | C: 1.35(1.03, 1.77) | 487,464 |
| RS242925 | 17: 041244646 | CRHR1 | 0.03290 | A,G: 1.36(1.05, 1.75) | 0.25642 | A: 1.11(0.93, 1.32) | 494,475 |
| RS2015729 | 17: 042709492 | ITGB3 | 0.04373 | !C,C: 1.4(1.07, 1.82) | 0.04093 | T: 1.21(1.01, 1.45) | 485,474 |
| RS2072365 | 17: 046107865 | ABCC3 | 0.02028 | !T,T: 1.7(1.14, 2.52) | 0.19298 | C: 1.13(0.94, 1.37) | 475,469 |
| RS2286650 | 17: 046133297 | ANKRD40 | 0.09204 | T,T: 1.29(1, 1.66) | 0.03177 | T: 1.24(1.02, 1.5) | 492,474 |
| RS10468445 | 17: 064029272 | PRKAR1A | 0.03899 | !C,C: 1.34(1.04, 1.74) | 0.01387 | T: 1.27(1.05, 1.54) | 494,451 |
| RS2302231 | 17: 064037622 | PRKAR1A | 0.01075 | !T,T: 1.4(1.08, 1.81) | 0.00375 | G: 1.33(1.1, 1.61) | 490,458 |
| RS2952270 | 17: 064042744 | FAM20A | 0.00128 | !C,C: 2.23(1.36, 3.65) | 0.00078 | G: 1.41(1.15, 1.72) | 487,469 |
| RS2886632 | 17: 065601497 | KCNJ16 | 0.00123 | T,T: 5(1.8, 13.87) | 0.16866 | T: 1.21(0.93, 1.56) | 491,473 |
| RS998580 | 17: 065638386 | KCNJ16 | 0.04609 | A,A: 1.44(1.06, 1.94) | 0.01756 | A: 1.4(1.07, 1.85) | 471,464 |
| 6728520 | 17: 069881299 | GPR142 | 0.03543 | T,T: 1.65(1.11, 2.43) | 0.16522 | T: 1.14(0.95, 1.38) | 474,457 |
| RS7229968 | 18: 011757420 | GNAL | 0.02849 | !T,T: | 0.71125 | G: 1.07(0.75, 1.54) | 491,474 |
| | | | | 13.64(0.77, 242.77) | | | |
| RS1481280 | 18: 016909449 | ROCK1 | 0.06907 | C,C: 1.36(1.05, 1.78) | 0.03333 | C: 1.22(1.02, 1.47) | 471,468 |
| RS1058424 | 18: 022689543 | AQP4 | 0.02510 | A,T: 1.45(1.09, 1.92) | 0.14654 | A: 1.19(0.94, 1.51) | 457,460 |
| RS151244 | 18: 022700717 | C18ORF16, | 0.05387 | !C,C: 1.68(1.08, 2.63) | 0.03756 | T: 1.23(1.01, 1.49) | 492,475 |
| | | AQP4 | | | | | |
| RS1873191 | 18: 044722249 | SMAD7 | 0.05191 | !A,A: 1.29(1, 1.67) | 0.02331 | G: 1.26(1.03, 1.54) | 489,467 |
| RS9953366 | 18: 044728190 | SMAD7 | 0.13741 | C,C: 1.27(0.98, 1.64) | 0.04806 | C: 1.21(1, 1.46) | 487,469 |
| RS8086404 | 18: 059068104 | BCL2 | 0.00859 | !C,C: 1.52(1.15, 2.01) | 0.00172 | G: 1.47(1.16, 1.88) | 490,463 |
| RS2285855 | 19: 000576081 | POLRMT | 0.08184 | C,C: 1.41(1.03, 1.92) | 0.04412 | C: 1.21(1.01, 1.44) | 485,474 |
| RS2229431 | 19: 007092775 | INSR | 0.00235 | C,C: 1.99(1.28, 3.11) | 0.00196 | C: 1.96(1.27, 3.02) | 491,467 |
| RS3093032 | 19: 010257336 | ICAM4, | 0.01176 | T,T: 4.63(1.45, 14.79) | 0.11187 | T: 1.23(0.96, 1.58) | 488,467 |
| | | ICAM1 | | | | | |
| RS2304165 | 19: 010520659 | ATG4D | 0.04585 | A,A: 2.72(0.92, 8.05) | 0.71775 | G: 1.05(0.79, 1.4) | 481,463 |
| RS1054685 | 19: 019595539 | EDG4 | 0.10762 | !A,A: 1.38(0.98, 1.94) | 0.03588 | G: 1.22(1.02, 1.46) | 493,473 |
| RS880090 | 19: 019601729 | EDG4, | 0.01867 | !C,C: 1.4(1.08, 1.81) | 0.00505 | G: 1.33(1.09, 1.63) | 483,466 |
| | | GMIP | | | | | |
| RS8100085 | 19: 040214959 | SCN1B | 0.09028 | A,A: 1.44(1.01, 2.06) | 0.03458 | A: 1.23(1.02, 1.49) | 490,428 |
| RS2053071 | 19: 044082771 | NFKBIB, | 0.03174 | !C,C: 1.4(1.08, 1.82) | 0.03911 | G: 1.22(1.01, 1.47) | 487,467 |
| | | SIRT2 | | | | | |
| RS2241704 | 19: 044088175 | NFKBIB | 0.12751 | !A,A: 1.27(0.98, 1.66) | 0.04918 | T: 1.26(1, 1.58) | 492,473 |
| RS892117 | 19: 045403173 | MAP3K10 | 0.01313 | !T,T: 1.51(1.14, 2) | 0.04375 | C: 1.21(1.01, 1.44) | 488,470 |
| RS2304234 | 19: 046440593 | AXL | 0.14507 | A,A: 1.31(0.97, 1.78) | 0.04997 | A: 1.23(1, 1.51) | 477,303 |
| RS3760798 | 19: 053649309 | KCNJ14, | 0.04435 | C,C: 6.21(1.11, 34.94) | 0.17268 | C: 1.27(0.91, 1.77) | 487,470 |
| | | GRWD1 | | | | | |
| RS2387583 | 19: 054153117 | BAX | 0.03732 | !T,T: 3.06(1.04, 8.96) | 0.94756 | T: 1.02(0.79, 1.32) | 468,468 |
| RS1035422 | 19: 055531025 | NAPSB | 0.01327 | T,T: 1.64(1.16, 2.32) | 0.00426 | T: 1.6(1.16, 2.22) | 460,459 |
| RS3764574 | 19: 062333910 | USP29 | 0.07310 | T,T: 1.37(1.02, 1.84) | 0.03040 | T: 1.22(1.02, 1.46) | 486,457 |
| RS- | 19: 062339048 | ZIM3 | 0.08539 | G,G: 1.34(1.03, 1.75) | 0.04169 | G: 1.22(1.01, 1.46) | 476,470 |
| GSK7003777 | | | | | | | |
| RS459012 | 20: 000410008 | | 0.00404 | C,T: 1.61(1.21, 2.15) | 0.01100 | C: 1.39(1.08, 1.79) | 488,468 |
| RS2740204 | 20: 003010467 | AVP | 0.03148 | !A,C: 1.4(1.08, 1.81) | 0.66942 | C: 1.04(0.86, 1.25) | 478,466 |
| RS932529 | 20: 003727629 | CDC25B | 0.12633 | T,T: 1.32(0.97, 1.8) | 0.04943 | T: 1.2(1, 1.44) | 487,474 |
| RS1054600 | 20: 004656130 | PRND | 0.06163 | !C,C: 1.37(1.05, 1.78) | 0.02910 | T: 1.28(1.03, 1.6) | 487,467 |
| RS2234395 | 20: 005221745 | PROKR2 | 0.01370 | !C,C: 1.5(1.12, 2) | 0.00413 | T: 1.3(1.09, 1.56) | 492,474 |
| RS2423410 | 20: 009602944 | PAK7 | 0.00276 | !A,C: 1.49(1.15, 1.93) | 0.60409 | A: 1.05(0.88, 1.27) | 481,464 |
| RS6579211 | 20: 033069518 | TRPC4AP | 0.00067 | !A,A: | 0.70769 | G: 1.06(0.79, 1.43) | 480,473 |
| | | | | 23.9(1.4, 406.66) | | | |
| RS717593 | 20: 033116625 | TRPC4AP | 0.00062 | !G,G: | 0.59759 | C: 1.09(0.81, 1.46) | 493,472 |
| | | | | 24.59(1.45, 418.55) | | | |
| RS4337537 | 20: 041645997 | SGK2 | 0.03132 | !C,C: 1.51(1.1, 2.06) | 0.01836 | T: 1.42(1.07, 1.88) | 488,438 |
| 6728089 | 20: 042807273 | KCNK15 | 0.02592 | !C,C: 1.57(1.12, 2.19) | 0.10368 | T: 1.17(0.97, 1.4) | 488,471 |
| 6728094 | 20: 042813372 | RIMS4, | 0.03590 | !G,G: 5.2(0.9, 30.17) | 0.03192 | T: 1.43(1.03, 1.98) | 486,473 |
| | | KCNK15 | | | | | |
| RS2903772 | 20: 043055517 | STK4 | 0.03458 | !G,G: 1.44(1.07, 1.94) | 0.01237 | T: 1.26(1.05, 1.5) | 492,476 |
| RS378114 | 20: 043971834 | PLTP | 0.12834 | !C,C: 1.26(0.96, 1.65) | 0.04524 | T: 1.25(1.01, 1.54) | 399,460 |
| RS4811189 | 20: 049579123 | NFATC2 | 0.11282 | !A,A: 1.28(0.98, 1.67) | 0.03558 | G: 1.27(1.02, 1.59) | 490,466 |
| RS1614845 | 20: 060231063 | HRH3 | 0.07620 | C,C: 1.37(1.04, 1.8) | 0.04747 | C: 1.27(1, 1.61) | 458,455 |
| RS439826 | 21: 026450517 | APP | 0.03724 | !T,T: 1.69(1.12, 2.54) | 0.04892 | G: 1.21(1.01, 1.46) | 490,474 |
| RS2836038 | 21: 038185753 | KCNJ6 | 0.10496 | A,A: 1.42(1.02, 1.96) | 0.03977 | A: 1.37(1.02, 1.86) | 476,469 |
| RS225436 | 21: 042602103 | TFF3 | 0.02257 | !A,G: 1.4(1.08, 1.8) | 0.25333 | A: 1.11(0.93, 1.33) | 489,470 |
| RS1785452 | 21: 044662218 | TRPM2 | 0.01418 | !T,T: 1.97(1.15, 3.38) | 0.71584 | C: 1.04(0.85, 1.28) | 488,475 |
| RS7275998 | 21: 045356797 | ADARB1 | 0.04912 | C,T: 1.49(1.08, 2.06) | 0.10221 | C: 1.27(0.96, 1.69) | 481,448 |
| RS9637192 | 21: 045461841 | ADARB1 | 0.03253 | T,T: 1.48(1.09, 2.02) | 0.01543 | T: 1.25(1.05, 1.5) | 494,469 |
| RS2253763 | 21: 045467192 | ADARB1 | 0.09674 | !C,C: 1.29(0.99, 1.68) | 0.03182 | T: 1.23(1.02, 1.48) | 490,441 |
| RS2337031 | 22: 045203338 | CELSR1 | 0.03175 | !A,A: 1.71(1.12, 2.62) | 0.02684 | T: 1.25(1.03, 1.51) | 476,443 |
| RS2239941 | X: 009952741 | CLCN4 | 0.01294 | !A,A: 2.02(1.24, 3.27) | 0.10410 | G: 1.31(0.95, 1.8) | 493,474 |
| RS5933961 | X: 011931141 | FRMPD4 | 0.56191 | !T,T: 1.26(0.83, 1.9) | 0.04562 | C: 1.3(1.01, 1.67) | 493,469 |
| RS179013 | X: 012661128 | TLR7 | 0.05348 | T,T: 3.88(1.15, 13.09) | 0.01452 | T: 1.38(1.07, 1.8) | 495,474 |
| RS1013151 | X: 012692188 | TLR8 | 0.35672 | C,C: 1.32(0.88, 2) | 0.04224 | C: 1.26(1.01, 1.57) | 492,474 |
| RS897975 | X: 018860704 | GPR64 | 0.12189 | !A,A: | 0.02008 | G: | 491,473 |
| | | | | 4.28(0.69, 26.31) | | 3.65(1.11, 12.01) | |
| RS2040394 | X: 021960775 | PHEX | 0.02298 | !A,G: 1.77(1.17, 2.66) | 0.78304 | G: 1.04(0.83, 1.28) | 494,475 |
| RS1128513 | X: 040267027 | CRSP2 | 0.08717 | T,T: 1.57(1.03, 2.37) | 0.03003 | T: 1.31(1.03, 1.68) | 489,474 |
| RS1150507 | X: 040769960 | USP9X | 0.04765 | G,G: 8.16(0.42, 159.1) | 0.79128 | G: 1.06(0.75, 1.5) | 468,452 |
| RS2159558 | X: 040780564 | USP9X | 0.03776 | !A,C: 1.72(1.13, 2.61) | 0.77951 | C: 1.03(0.83, 1.29) | 479,470 |
| RS2238978 | X: 048525871 | SLC35A2, | 1.00000 | G,G: 2.97(0.12, 73.3) | 0.03629 | G: 1.48(1.03, 2.14) | 491,391 |
| | | PIM2 | | | | | |
| RS232758 | X: 049429973 | | 0.02234 | !A,G: 1.82(1.17, 2.83) | 0.90857 | A: 1.02(0.81, 1.28) | 444,441 |
| RS- | X: 052983611 | GPR173 | 0.04751 | !C,T: 1.76(1.05, 2.94) | 0.71636 | T: 1.06(0.8, 1.41) | 456,460 |
| GSK16417450 | | | | | | | |
| RS3012630 | X: 071301271 | | 0.03528 | A,A: 2.03(1.12, 3.69) | 0.34832 | A: 1.21(0.84, 1.75) | 471,469 |
| RS1935075 | X: 079982855 | | 0.28095 | !A,A: 1.55(0.88, 2.73) | 0.02150 | G: 1.32(1.05, 1.66) | 470,456 |
| RS1053870 | X: 100402965 | | 0.03167 | !T,T: 5.76(1.02, 32.63) | 0.22922 | G: 1.2(0.89, 1.61) | 483,469 |
| RS6318 | X: 113788715 | HTR2C | 0.01142 | !C,C: | 0.00345 | G: 1.58(1.16, 2.14) | 481,458 |
| | | | | 5.88(1.04, 33.31) | | | |
| RS1414324 | X: 113971478 | HTR2C | 0.04769 | !A,A: | 0.02448 | G: 1.43(1.05, 1.94) | 487,472 |
| | | | | 5.09(0.89, 29.25) | | | |
| RS2235306 | X: 128509633 | APLN | 0.23151 | T,T: 2.17(0.61, 7.67) | 0.04182 | T: 2.44(1.04, 5.74) | 481,474 |
| RS413957 | X: 138363036 | F9 | 0.04425 | !C,G: 1.71(1.1, 2.64) | 0.31485 | C: 1.15(0.89, 1.5) | 478,471 |
| RS994423 | X: 151052763 | GABRA3 | 0.00285 | !G,T: 2.22(1.33, 3.71) | 0.07916 | T: 1.32(0.98, 1.78) | 476,464 |

¹PolyName are RS identifiers. SNPs without an RS prefix were identified at GSK.
²Key denoting strength of association
2.78E−05 < p-value ≦ 0.0005 in bold underline
0.0005 < p-value ≦ 0.005 in bold
0.005 < p-value ≦ 0.005 in italic
³For example, if a SNP had genotypes AA, AG and GG, 3 chi-square tests were performed contrasting cases and controls: 1) AA vs AG + GG, 2) AG vs AA + GG and 3) GG vs AA + AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) thisgenotype and 2) a new odds ratio was calculated as the inverse of the original odds ratio. This new odds ratio was reported.
⁴See Table 6 for descriptions for genes listed in this table

TABLE 4
Aberdeen Collection: SNPs with a Genotypic or Allelic Fishers Exact P ≦ 0.05.

| | | | Genotypic | | | | |
|---|---|---|---|---|---|---|---|
| | | | Genomic | | | | |
| | | | Control | | | | |
| | | | Adjusted | | Allelic | | Number |
| | NCBI 36 | Gene | Exact | Risk Genotype & | Exact | Risk Allele & Odd | Cases, |
| Poly Name¹ | position | Symbols | P² | Odd Ratio (95% CI)³ | P² | Ratio (95% CI) | Controls |
| RS604618 | 01: 001222182 | CENTB5 | 0.01730 | A,G: 1.6(1.15, 2.23) | 0.01813 | A: 1.46(1.07, 1.98) | 527, 660 |
| RS307348 | 01: 001251687 | MGC10334, | 0.09065 | !G,G: 1.46(1.04, 2.07) | 0.03406 | C: 1.44(1.04, 2.01) | 527, 660 |
| | | CPSF3L | | | | | |
| RS3789541 | 01: 006063912 | KCNAB2 | 0.04586 | T,T: 6.33(1.09, 36.65) | 0.73147 | T: 1.07(0.76, 1.49) | 527, 659 |
| RS1061622 | 01: 012175542 | TNFRSF1B | 0.12569 | T,T: 1.27(1, 1.6) | 0.03932 | T: 1.22(1.01, 1.48) | 526, 660 |
| RS1472408 | 01: 016351241 | EPHA2 | 0.12764 | !A,A: 1.28(1.01, 1.62) | 0.04681 | G: 1.18(1, 1.4) | 526, 660 |
| RS11580864 | 01: 022324961 | WNT4 | 0.08732 | T,T: 1.33(1.03, 1.72) | 0.02577 | T: 1.3(1.04, 1.63) | 527, 660 |
| RS1076680 | 01: 022330971 | WNT4 | 0.11182 | C,C: 1.3(1, 1.69) | 0.03519 | C: 1.29(1.02, 1.62) | 518, 635 |
| RS309502 | 01: 023104987 | EPHB2 | 0.01094 | A,A: 1.43(1.12, 1.84) | 0.09736 | A: 1.15(0.98, 1.35) | 527, 660 |
| RS309497 | 01: 023109413 | EPHB2 | 0.04284 | A,A: 1.39(1.06, 1.82) | 0.13369 | A: 1.14(0.96, 1.35) | 503, 594 |
| RS1002487 | 01: 026738558 | RPS6KA1 | 0.04535 | G,G: | 0.73406 | A: 1.06(0.76, 1.48) | 527, 660 |
| | | | | 11.36(0.61, 211.38) | | | |
| RS190737 | 01: 026772031 | RPS6KA1 | 0.05678 | G,G: 1.33(1.01, 1.74) | 0.01479 | G: 1.22(1.04, 1.44) | 527, 660 |
| RS1292089 | 01: 027816587 | FGR | 0.04652 | A,T: 1.43(1.03, 1.98) | 0.13987 | A: 1.26(0.93, 1.7) | 527, 659 |
| RS2938867 | 01: 028387879 | PTAFR | 0.04383 | !G,G: 1.6(1.04, 2.46) | 0.68503 | A: 1.04(0.87, 1.24) | 527, 660 |
| RS11583031 | 01: 035701595 | KIAA0319L | 0.06133 | !A,T: 1.64(1.05, 2.58) | 0.04393 | A: 1.56(1.01, 2.41) | 527, 660 |
| RS272814 | 01: 036463457 | THRAP3 | 0.01333 | !T,T: 7.32(1.35, 39.69) | 0.07349 | G: 1.28(0.98, 1.67) | 527, 660 |
| RS882799 | 01: 036539987 | THRAP3 | 0.01353 | !C,C: | 0.07232 | T: 1.28(0.98, 1.66) | 526, 659 |
| | | | | 7.32(1.35, 39.68) | | | |
| RS1981080 | 01: 043532320 | TIE1 | 0.02601 | !G,T: 1.38(1.09, 1.73) | 0.56308 | G: 1.05(0.89, 1.23) | 527, 659 |
| RS2495480 | 01: 055281943 | PCSK9 | 0.05378 | A,A: 1.62(1.04, 2.51) | 0.02759 | A: 1.62(1.05, 2.48) | 527, 660 |
| RS1772626 | 01: 064287967 | ROR1 | 0.02289 | !A,G: 1.46(1.12, 1.91) | 0.01609 | A: 1.33(1.06, 1.69) | 527, 660 |
| RS2256298 | 01: 065103270 | JAK1 | 0.01911 | !C,T: 1.37(1.07, 1.74) | 0.55422 | C: 1.06(0.88, 1.29) | 527, 660 |
| RS310202 | 01: 065122132 | JAK1 | 0.02005 | C,C: 2.06(1.14, 3.72) | 0.00860 | C: 1.31(1.07, 1.6) | 527, 660 |
| RS310199 | 01: 065122710 | JAK1 | 0.02813 | C,C: 1.84(1.17, 2.89) | 0.02995 | C: 1.22(1.02, 1.47) | 527, 660 |
| RS1463884 | 01: 066322691 | PDE4B | 0.00820 | A,A: 1.44(1.12, 1.85) | 0.00183 | A: 1.42(1.14, 1.78) | 527, 660 |
| RS539322 | 01: 066362336 | PDE4B | 0.01759 | !G,G: 1.39(1.1, 1.76) | 0.00420 | A: 1.28(1.08, 1.51) | 527, 659 |
| RS640352 | 01: 066372396 | PDE4B | 0.01305 | G,G: 1.39(1.1, 1.75) | 0.00213 | G: 1.3(1.1, 1.54) | 527, 660 |
| RS673143 | 01: 066392707 | PDE4B | 0.01033 | G,G: 1.4(1.1, 1.76) | 0.00161 | G: 1.31(1.11, 1.55) | 527, 660 |
| RS640493 | 01: 074699382 | TNNI3K | 0.06052 | A,A: 1.32(1.04, 1.68) | 0.01701 | A: 1.22(1.04, 1.44) | 527, 660 |
| RS515419 | 01: 074700860 | TNNI3K | 0.04611 | C,C: 1.38(1.08, 1.76) | 0.04121 | C: 1.19(1.01, 1.4) | 525, 660 |
| RS3765674 | 01: 074710337 | TNNI3K | 0.03989 | G,G: 1.39(1.09, 1.78) | 0.03746 | G: 1.19(1.01, 1.4) | 527, 660 |
| RS274587 | 01: 074716053 | TNNI3K | 0.01567 | G,G: 1.47(1.14, 1.89) | 0.01445 | G: 1.23(1.04, 1.45) | 527, 660 |
| RS274605 | 01: 074731368 | TNNI3K | 0.00429 | G,G: 1.58(1.21, 2.05) | 0.00747 | G: 1.25(1.06, 1.47) | 519, 640 |
| RS274606 | 01: 074734127 | TNNI3K | 0.00981 | G,G: 1.49(1.16, 1.92) | 0.00702 | G: 1.25(1.07, 1.48) | 527, 660 |
| RS1332824 | 01: 079276960 | ELTD1 | 0.12629 | !G,G: 1.24(0.97, 1.57) | 0.03842 | T: 1.19(1.01, 1.41) | 526, 659 |
| RS629301 | 01: 109619829 | CELSR2 | 0.11085 | A,A: 1.24(0.98, 1.57) | 0.03489 | A: 1.24(1.02, 1.51) | 527, 660 |
| RS10858092 | 01: 109745416 | PSMA5 | 0.03169 | T,T: 1.3(1.04, 1.64) | 0.00735 | T: 1.28(1.07, 1.53) | 526, 660 |
| RS2095731 | 01: 111835999 | TIG22692, | 0.05854 | !C,C: 1.3(1.03, 1.63) | 0.01370 | A: 1.25(1.05, 1.49) | 527, 660 |
| | | ADORA3 | | | | | |
| RS682348 | 01: 112183367 | KCND3 | 0.06893 | !T,T: 1.92(0.98, 3.76) | 0.03136 | A: 1.26(1.02, 1.55) | 526, 660 |
| RS3790609 | 01: 112858513 | WNT2B | 0.06079 | !C,T: 1.38(1.06, 1.8) | 0.03896 | C: 1.26(1.01, 1.58) | 527, 660 |
| RS1217412 | 01: 114158734 | PTPN22 | 0.09140 | !A,A: 1.3(1.03, 1.64) | 0.03167 | G: 1.23(1.02, 1.48) | 527, 660 |
| RS1217388 | 01: 114165999 | PTPN22 | 0.09902 | !A,A: 1.3(1.03, 1.64) | 0.03541 | G: 1.23(1.02, 1.48) | 526, 660 |
| RS1217414 | 01: 114214190 | PTPN22 | 0.02157 | C,C: 1.38(1.1, 1.74) | 0.02680 | C: 1.23(1.02, 1.47) | 527, 660 |
| RS2488457 | 01: 114216891 | PTPN22 | 0.12358 | !G,G: 1.28(1.01, 1.62) | 0.03536 | C: 1.24(1.02, 1.5) | 527, 659 |
| RS720899 | 01: 144262279 | KIAA1245, | 0.00134 | T,T: 1.56(1.24, 1.96) | 0.00077 | T: 1.35(1.13, 1.6) | 526, 657 |
| | | ANKRD35 | | | | | |
| RS- | 01: 149026497 | CTSK | 0.02419 | G,G: 2.2(1.11, 4.39) | 0.02546 | G: 2.17(1.1, 4.31) | 526, 650 |
| GSK8111961 | | | | | | | |
| RS3014866 | 01: 151595695 | S100A9 | 0.08868 | C,C: 1.29(1, 1.67) | 0.02559 | C: 1.2(1.02, 1.42) | 527, 660 |
| RS1891806 | 01: 153027692 | KCNN3 | 0.04056 | !A,A: 1.35(1.07, 1.7) | 0.01021 | G: 1.26(1.06, 1.5) | 527, 659 |
| RS3765087 | 01: 153295877 | ADAM15 | 0.00972 | A,A: 1.46(1.09, 1.95) | 0.00291 | A: 1.5(1.15, 1.96) | 526, 659 |
| RS856068 | 01: 157250401 | IFI16 | 0.14014 | G,G: 1.25(0.99, 1.59) | 0.04276 | G: 1.23(1.01, 1.5) | 536, 667 |
| RS1446966 | 01: 157830107 | APCS | 0.08921 | G,G: 1.28(1.01, 1.62) | 0.02516 | G: 1.21(1.02, 1.43) | 527, 660 |
| RS2274229 | 01: 177350891 | ABL2 | 0.03143 | T,T: | 0.05769 | T: 1.36(1, 1.84) | 527, 660 |
| | | | | 11.36(0.61, 211.38) | | | |
| RS3753814 | 01: 178388103 | QSCN6 | 0.10425 | !G,G: 1.27(1.01, 1.6) | 0.02616 | A: 1.22(1.02, 1.45) | 525, 660 |
| RS3753812 | 01: 178392764 | QSCN6 | 0.06631 | T,T: 1.54(1.06, 2.24) | 0.03563 | T: 1.2(1.01, 1.43) | 527, 659 |
| RS12042344 | 01: 185124758 | PLA2G4A | 0.04793 | !C,T: 1.35(1, 1.81) | 0.27459 | T: 1.16(0.89, 1.52) | 537, 667 |
| RS2296618 | 01: 196932855 | PTPRC | 0.12006 | !A,A: 1.32(1.02, 1.71) | 0.03395 | G: 1.28(1.02, 1.61) | 527, 660 |
| RS2296384 | 01: 199325315 | CACNA1S | 0.00899 | !G,G: 1.38(1.09, 1.74) | 0.00185 | A: 1.31(1.11, 1.56) | 527, 660 |
| RS705763 | 01: 200544436 | LGR6 | 0.04151 | A,G: 1.35(1.07, 1.7) | 0.96686 | A: 1(0.85, 1.18) | 527, 660 |
| RS2184030 | 01: 204734064 | AK057159, | 0.09898 | A,A: 1.33(1.03, 1.73) | 0.02856 | A: 1.2(1.02, 1.41) | 527, 660 |
| | | C1ORF147, | | | | | |
| | | IKBKE | | | | | |
| RS3024505 | 01: 205006527 | IL10 | 0.02614 | !C,T: 1.44(1.1, 1.87) | 0.04215 | C: 1.27(1.01, 1.6) | 527, 660 |
| RS3024493 | 01: 205010591 | IL10 | 0.02614 | !G,T: 1.44(1.1, 1.87) | 0.04215 | G: 1.27(1.01, 1.6) | 527, 660 |
| RS1554286 | 01: 205010856 | IL10 | 0.07374 | !C,C: 1.35(1.05, 1.73) | 0.02810 | T: 1.28(1.03, 1.6) | 526, 659 |
| RS3021094 | 01: 205011575 | IL10 | 0.07783 | !A,A: 1.45(1.06, 1.98) | 0.02217 | C: 1.42(1.05, 1.91) | 527, 660 |
| RS596731 | 01: 208060424 | C1ORF107 | 0.00778 | A,G: 1.46(1.16, 1.83) | 0.52963 | A: 1.06(0.9, 1.24) | 527, 660 |
| RS1934611 | 01: 209007493 | KCNH1 | 0.04794 | !A,G: 1.35(1.07, 1.7) | 0.09253 | G: 1.17(0.98, 1.4) | 527, 660 |
| RS2133123 | 01: 214911440 | ESRRG | 0.15513 | C,C: 1.36(0.94, 1.95) | 0.04578 | C: 1.19(1, 1.42) | 527, 660 |
| RS1153942 | 01: 222037368 | TP53BP2 | 0.02552 | !A,A: 3.16(1.12, 8.97) | 0.86363 | A: 1.03(0.82, 1.28) | 527, 660 |
| RS360063 | 01: 224102932 | TMEM63A | 0.01405 | A,A: 1.64(1.19, 2.26) | 0.03611 | A: 1.2(1.01, 1.42) | 527, 660 |
| RS1410142 | 01: 228895061 | COG2 | 0.03183 | T,T: 1.39(1.09, 1.77) | 0.01137 | T: 1.31(1.06, 1.6) | 527, 660 |
| RS633860 | 01: 231826729 | KCNK1 | 0.01905 | T,T: 1.43(1.12, 1.82) | 0.02682 | T: 1.21(1.02, 1.42) | 526, 659 |
| RS2490390 | 01: 235342691 | RYR2 | 0.13801 | !G,G: 1.26(1, 1.59) | 0.04669 | A: 1.22(1.01, 1.47) | 527, 659 |
| RS2026287 | 01: 235717800 | RYR2 | 0.00303 | G,T: 1.41(1.12, 1.79) | 1.00000 | G: 1(0.84, 1.2) | 518, 640 |
| RS1357150 | 01: 235992440 | RYR2 | 0.02419 | A,A: 1.38(1.08, 1.77) | 0.10407 | A: 1.15(0.97, 1.35) | 527, 660 |
| RS1932439 | 01: 239769607 | KMO | 0.04646 | C,G: 1.35(1.07, 1.69) | 0.96698 | G: 1.01(0.86, 1.18) | 527, 660 |
| RS6710065 | 02: 026930061 | DPYSL5 | 0.02356 | !C,T: 1.39(1.1, 1.75) | 0.25160 | C: 1.11(0.94, 1.31) | 527, 660 |
| RS4665919 | 02: 026968808 | DPYSL5 | 0.04588 | !C,T: 1.34(1.07, 1.69) | 0.21870 | T: 1.11(0.94, 1.31) | 527, 660 |
| RS4441421 | 02: 026995595 | DPYSL5 | 0.00014 | !A,G: 1.65(1.31, 2.08) | 0.01385 | A: 1.24(1.05, 1.47) | 527, 660 |
| RS4233714 | 02: 027034958 | | 0.00217 | !A,G: 1.5(1.19, 1.9) | 0.02156 | A: 1.22(1.03, 1.45) | 527, 660 |
| RS4582 | 02: 027457783 | ZNF513, | 0.09991 | !A,A: 1.3(1.03, 1.65) | 0.04169 | G: 1.19(1.01, 1.41) | 527, 660 |
| | | PPM1G | | | | | |
| RS1528424 | 02: 029331334 | ALK | 0.00509 | !C,C: 2.24(1.35, 3.73) | 0.01076 | T: 1.27(1.06, 1.53) | 526, 660 |
| RS11898966 | 02: 029499928 | ALK | 0.03026 | G,G: 1.44(1.09, 1.91) | 0.01009 | G: 1.41(1.09, 1.82) | 527, 660 |
| RS12618159 | 02: 029504240 | ALK | 0.08003 | T,T: 1.31(0.99, 1.72) | 0.01813 | T: 1.22(1.03, 1.43) | 527, 660 |
| RS4533436 | 02: 029533216 | ALK | 0.00395 | A,A: 1.59(1.22, 2.08) | 0.01170 | A: 1.23(1.05, 1.45) | 527, 660 |
| RS4366859 | 02: 029536488 | ALK | 0.00053 | !C,C: 1.65(1.27, 2.14) | 0.00006 | G: 1.62(1.28, 2.05) | 525, 657 |
| RS4666219 | 02: 029549991 | ALK | 0.00842 | !C,T: 1.45(1.15, 1.82) | 0.20755 | C: 1.11(0.94, 1.31) | 527, 660 |
| RS10445908 | 02: 029581061 | ALK | 0.11511 | !G,G: 1.34(0.99, 1.82) | 0.03179 | T: 1.2(1.02, 1.42) | 527, 659 |
| RS6719507 | 02: 029587305 | ALK | 0.07930 | !A,A: 1.35(1.03, 1.78) | 0.02272 | G: 1.21(1.03, 1.42) | 526, 660 |
| RS4666233 | 02: 029601666 | ALK | 0.04689 | !T,T: 1.38(1.03, 1.85) | 0.01687 | C: 1.4(1.07, 1.84) | 526, 659 |
| RS4666289 | 02: 029979097 | ALK | 0.01350 | C,T: 1.36(1.08, 1.72) | 1.00000 | C: 1(0.84, 1.19) | 527, 658 |
| RS479103 | 02: 030822972 | CAPN13 | 0.04543 | !G,G: 1.4(1.08, 1.81) | 0.01590 | A: 1.33(1.06, 1.67) | 525, 659 |
| RS581331 | 02: 030837418 | CAPN13 | 0.00255 | C,G: 1.59(1.23, 2.06) | 0.01193 | G: 1.33(1.07, 1.65) | 527, 660 |
| RS2058863 | 02: 039433881 | MAP4K3 | 0.06747 | C,C: 2.35(1.06, 5.22) | 0.04181 | C: 1.29(1.01, 1.63) | 527, 660 |
| RS1988650 | 02: 042542866 | KCNG3 | 0.04276 | C,C: 1.3(1.03, 1.65) | 0.01358 | C: 1.3(1.06, 1.59) | 526, 660 |
| RS2278582 | 02: 042842948 | OXER1 | 0.04646 | G,G: 1.3(1.02, 1.67) | 0.00984 | G: 1.24(1.05, 1.46) | 524, 659 |
| RS2278586 | 02: 042843605 | OXER1 | 0.03930 | C,G: 1.38(1.08, 1.76) | 0.11494 | C: 1.18(0.96, 1.43) | 516, 656 |
| RS893735 | 02: 042845527 | OXER1 | 0.00539 | !T,T: 1.57(1.13, 2.16) | 0.00099 | C: 1.32(1.12, 1.56) | 527, 659 |
| RS965374 | 02: 042846670 | OXER1, | 0.00529 | !T,T: 1.58(1.14, 2.18) | 0.00099 | G: 1.32(1.12, 1.56) | 526, 660 |
| | | HAAO | | | | | |
| RS6720173 | 02: 043893905 | ABCG5 | 0.01323 | G,G: 1.47(1.14, 1.89) | 0.00287 | G: 1.41(1.12, 1.76) | 527, 660 |
| RS6740545 | 02: 043949643 | ABCG8 | 0.01002 | !A,A: 1.37(1.09, 1.72) | 0.00243 | G: 1.32(1.11, 1.58) | 527, 658 |
| RS6544718 | 02: 043958429 | ABCG8 | 0.03123 | T,T: 2.16(1.22, 3.83) | 0.07297 | T: 1.2(0.99, 1.46) | 527, 660 |
| RS1837705 | 02: 044323253 | PPM1B | 0.04254 | A,A: 1.82(1.07, 3.09) | 0.76317 | A: 1.03(0.85, 1.26) | 527, 660 |
| RS2303424 | 02: 047593055 | KCNK12 | 0.03962 | !C,C: 1.54(1.11, 2.14) | 0.08202 | T: 1.16(0.98, 1.37) | 527, 660 |
| RS374640 | 02: 070607390 | TGFA | 0.02111 | !T,T: 1.41(1.11, 1.8) | 0.01345 | C: 1.3(1.06, 1.6) | 527, 660 |
| RS2236937 | 02: 101810171 | MAP4K4 | 0.05119 | C,C: 1.87(1, 3.47) | 0.01927 | C: 1.27(1.04, 1.56) | 527, 660 |
| RS2110726 | 02: 102160714 | IL1R1 | 0.02322 | C,C: 1.32(1.04, 1.68) | 0.00529 | C: 1.27(1.08, 1.5) | 526, 660 |
| RS4849160 | 02: 113650556 | PSD4 | 0.06350 | G,G: 1.33(1.05, 1.68) | 0.02234 | G: 1.22(1.03, 1.45) | 526, 660 |
| RS1015755 | 02: 113684778 | PAX8 | 0.00211 | A,A: 2.43(1.36, 4.32) | 0.80327 | A: 1.03(0.85, 1.25) | 527, 660 |
| RS4849178 | 02: 113699079 | PAX8 | 0.00181 | A,A: 2.49(1.38, 4.49) | 0.84116 | A: 1.02(0.84, 1.25) | 527, 660 |
| RS2587693 | 02: 119928084 | SCTR | 0.03068 | !A,A: 1.33(1.03, 1.73) | 0.01440 | G: 1.35(1.07, 1.7) | 527, 660 |
| RS2579629 | 02: 119944232 | SCTR | 0.04650 | C,C: 2.17(1.12, 4.2) | 0.03698 | C: 1.25(1.02, 1.54) | 527, 660 |
| RS2734871 | 02: 136586343 | CXCR4 | 0.03975 | T,T: 1.84(1.13, 3.02) | 0.03367 | T: 1.22(1.02, 1.47) | 527, 660 |
| RS2925754 | 02: 160768561 | ITGB6 | 0.05253 | A,A: 1.35(1.06, 1.71) | 0.01504 | A: 1.29(1.05, 1.58) | 527, 660 |
| RS2046490 | 02: 163147732 | KCNH7 | 0.02409 | A,G: 1.37(1.09, 1.73) | 0.61964 | G: 1.05(0.89, 1.23) | 527, 660 |
| RS1824551 | 02: 166626965 | SCN1A | 0.02285 | !C,T: 1.33(1.06, 1.68) | 0.60876 | C: 1.05(0.89, 1.24) | 527, 660 |
| RS10180790 | 02: 167002099 | SCN7A | 0.07864 | C,C: 1.26(1, 1.59) | 0.02408 | C: 1.22(1.03, 1.45) | 527, 659 |
| RS10208151 | 02: 173134198 | PDK1 | 0.03378 | !A,G: 1.41(1.09, 1.82) | 0.03536 | G: 1.27(1.02, 1.58) | 527, 659 |
| RS6710129 | 02: 173161000 | PDK1 | 0.03657 | !C,T: 1.39(1.08, 1.78) | 0.16189 | T: 1.16(0.94, 1.42) | 527, 660 |
| RS1075638 | 02: 173173442 | PDK1 | 0.04099 | !C,G: 1.38(1.07, 1.77) | 0.17819 | C: 1.15(0.94, 1.41) | 527, 659 |
| RS1376865 | 02: 175332843 | CHRNA1 | 0.02607 | !G,G: 2.45(1.06, 5.64) | 0.00833 | A: 1.39(1.09, 1.78) | 527, 659 |
| RS212343 | 02: 175654683 | ATF2 | 0.08131 | !G,G: 1.32(1.04, 1.68) | 0.02968 | C: 1.26(1.03, 1.54) | 527, 660 |
| RS2300583 | 02: 175661575 | ATF2 | 0.04746 | C,C: | 0.06335 | C: 1.47(0.98, 2.19) | 526, 660 |
| | | | | 11.38(0.61, 211.79) | | | |
| RS268214 | 02: 175741461 | ATF2 | 0.12559 | !T,T: 1.29(1.01, 1.63) | 0.04128 | C: 1.24(1.01, 1.52) | 527, 660 |
| RS7775 | 02: 183407829 | FRZB | 0.00425 | !C,G: 1.82(1.25, 2.65) | 0.00441 | C: 1.67(1.18, 2.39) | 527, 659 |
| RS9333290 | 02: 187227583 | ITGAV | 0.01177 | !G,T: 1.42(1.12, 1.79) | 0.49135 | G: 1.07(0.89, 1.28) | 526, 659 |
| RS7581626 | 02: 196709152 | STK17B | 0.08500 | A,A: 1.24(0.99, 1.57) | 0.02959 | A: 1.21(1.02, 1.44) | 526, 656 |
| RS1054537 | 02: 196712684 | STK17B | 0.10240 | C,C: 1.25(0.99, 1.57) | 0.03254 | C: 1.21(1.02, 1.43) | 527, 660 |
| RS2271887 | 02: 196718772 | STK17B | 0.08775 | C,C: 1.26(1, 1.59) | 0.02856 | C: 1.21(1.02, 1.44) | 525, 656 |
| RS3792232 | 02: 196728884 | STK17B | 0.02517 | G,G: 1.31(1.04, 1.66) | 0.00806 | G: 1.26(1.06, 1.49) | 527, 660 |
| RS1519602 | 02: 196736037 | STK17B | 0.13377 | G,G: 1.24(0.98, 1.56) | 0.04447 | G: 1.19(1, 1.42) | 527, 659 |
| RS4850676 | 02: 196741807 | STK17B | 0.09301 | T,T: 1.26(1, 1.59) | 0.02952 | T: 1.21(1.02, 1.44) | 527, 660 |
| RS926169 | 02: 204430997 | CTLA4 | 0.04606 | !G,T: 1.34(1.06, 1.69) | 1.00000 | G: 1(0.85, 1.18) | 527, 660 |
| RS231770 | 02: 204437398 | CTLA4 | 0.04433 | !C,T: 1.34(1.07, 1.69) | 0.96677 | T: 1(0.85, 1.18) | 527, 660 |
| RS2551639 | 02: 208104432 | CREB1 | 0.05521 | !A,A: 1.34(1.05, 1.71) | 0.01425 | G: 1.3(1.06, 1.6) | 527, 660 |
| RS889895 | 02: 208107174 | CREB1 | 0.05415 | !T,T: 1.34(1.05, 1.71) | 0.01235 | C: 1.3(1.06, 1.61) | 526, 659 |
| RS2551920 | 02: 208140075 | CREB1 | 0.05521 | !C,C: 1.34(1.05, 1.71) | 0.01425 | G: 1.3(1.06, 1.6) | 527, 660 |
| RS2551922 | 02: 208146805 | CREB1 | 0.00095 | !A,G: 2.24(1.35, 3.74) | 0.01239 | G: 1.84(1.14, 2.95) | 527, 660 |
| RS2709388 | 02: 208151256 | CREB1 | 0.06073 | !G,G: 1.33(1.04, 1.69) | 0.01444 | A: 1.3(1.05, 1.6) | 527, 660 |
| RS2551926 | 02: 208154612 | CREB1 | 0.01357 | !G,T: 1.99(1.19, 3.34) | 0.01334 | G: 1.85(1.13, 3.03) | 527, 660 |
| RS4673615 | 02: 211975105 | ERBB4 | 0.01291 | C,T: 1.39(1.1, 1.75) | 0.10427 | C: 1.15(0.97, 1.35) | 525, 660 |
| RS4597456 | 02: 212033473 | ERBB4 | 0.00518 | A,G: 1.43(1.13, 1.79) | 0.24663 | G: 1.1(0.94, 1.3) | 527, 659 |
| RS2272024 | 02: 212252325 | ERBB4 | 0.04382 | !G,G: 6.21(1.13, 34.2) | 0.31297 | A: 1.15(0.88, 1.5) | 527, 660 |
| RS10175273 | 02: 212279939 | ERBB4 | 0.04654 | !C,C: 1.39(1.05, 1.84) | 0.23015 | T: 1.11(0.94, 1.3) | 527, 660 |
| RS7592809 | 02: 212568665 | ERBB4 | 0.06446 | T,T: 1.56(1.02, 2.37) | 0.03490 | T: 1.55(1.03, 2.33) | 527, 660 |
| RS939223 | 02: 212746669 | ERBB4 | 0.04296 | G,T: 1.35(1.08, 1.7) | 0.38610 | G: 1.08(0.91, 1.28) | 526, 660 |
| RS3856551 | 02: 219454359 | WNT10A | 0.00261 | !A,G: 1.62(1.24, 2.13) | 0.00399 | G: 1.4(1.11, 1.76) | 527, 659 |
| RS885854 | 02: 219821972 | TUBA1, | 0.08198 | !C,C: 1.32(1.04, 1.68) | 0.04391 | T: 1.24(1.01, 1.53) | 527, 660 |
| | | STK16 | | | | | |
| RS1510662 | 02: 220099018 | ACCN4 | 0.03378 | G,G: 1.34(1.05, 1.7) | 0.00684 | G: 1.32(1.08, 1.62) | 527, 660 |
| RS927594 | 02: 230354942 | TRIP12 | 0.03375 | G,G: 1.46(1.11, 1.92) | 0.03486 | G: 1.19(1.02, 1.4) | 527, 659 |
| RS7607338 | 02: 231696199 | HTR2B, | 0.00421 | !A,A: 2.01(1.33, 3.05) | 0.01213 | C: 1.26(1.05, 1.51) | 527, 660 |
| | | PSMD1 | | | | | |
| RS2077386 | 02: 232503997 | NPPC | 0.03296 | !C,G: 1.45(1.09, 1.94) | 0.04434 | G: 1.31(1.01, 1.69) | 527, 660 |
| RS1448430 | 02: 239757676 | HDAC4 | 0.15383 | !A,A: 1.27(0.98, 1.64) | 0.04992 | G: 1.25(1, 1.57) | 527, 660 |
| RS4676407 | 02: 241227210 | | 0.06915 | G,G: 1.64(1.05, 2.54) | 0.02960 | G: 1.23(1.02, 1.48) | 527, 660 |
| RS869943 | 02: 242080258 | FARP2 | 0.04366 | G,G: 2.63(1.15, 6.02) | 0.78051 | G: 1.04(0.83, 1.29) | 527, 660 |
| RS1995496 | 02: 242094009 | STK25 | 0.02450 | !A,G: 1.38(1.1, 1.74) | 0.64956 | A: 1.04(0.89, 1.22) | 527, 660 |
| RS7421 | 02: 242260607 | ATG4B | 0.01368 | G,G: 1.36(1.08, 1.72) | 0.00360 | G: 1.28(1.09, 1.52) | 527, 660 |
| RS1018107 | 03: 004802309 | ITPR1 | 0.02337 | A,G: 1.34(1.07, 1.69) | 0.23103 | A: 1.11(0.94, 1.3) | 527, 660 |
| RS655620 | 03: 007000186 | GRM7 | 0.03323 | !G,G: 1.37(1.07, 1.76) | 0.00933 | A: 1.34(1.08, 1.67) | 527, 657 |
| RS1154359 | 03: 007011256 | GRM7 | 0.02065 | G,G: 1.43(1.1, 1.85) | 0.00719 | G: 1.25(1.06, 1.47) | 527, 660 |
| RS1144911 | 03: 010209065 | IRAK2 | 0.01607 | C,T: 14(1.11, 1.76) | 0.12915 | T: 1.14(0.96, 1.34) | 527, 660 |
| RS35681 | 03: 010304377 | GHRL, | 0.04701 | !G,G: 1.42(1.08, 1.86) | 0.04729 | A: 1.18(1, 1.39) | 527, 659 |
| | | C3ORF42 | | | | | |
| RS26802 | 03: 010307365 | GHRL | 0.07272 | !G,G: 1.54(1.02, 2.32) | 0.03910 | T: 1.2(1.01, 1.43) | 527, 660 |
| RS40608 | 03: 010316836 | SEC13L1 | 0.01330 | !G,G: 2.14(1.23, 3.7) | 0.01224 | A: 1.28(1.06, 1.55) | 527, 659 |
| RS1170695 | 03: 011030338 | SLC6A1 | 0.06340 | T,T: 1.33(1.06, 1.67) | 0.02546 | T: 1.23(1.03, 1.47) | 527, 660 |
| RS1946620 | 03: 013833800 | WNT7A | 0.02654 | T,T: 1.5(1.07, 2.1) | 0.00667 | T: 1.26(1.07, 1.5) | 527, 660 |
| RS12053930 | 03: 013845463 | WNT7A | 0.02941 | !C,T: 1.4(1.1, 1.79) | 0.10410 | T: 1.18(0.97, 1.45) | 527, 660 |
| RS826426 | 03: 015234869 | CAPN7 | 0.10520 | !T,T: 1.35(1.01, 1.8) | 0.02679 | C: 1.34(1.04, 1.75) | 527, 660 |
| RS826425 | 03: 015243010 | CAPN7 | 0.01417 | !A,A: 2.29(1.22, 4.32) | 0.00901 | G: 1.31(1.07, 1.61) | 527, 660 |
| RS977162 | 03: 020166510 | PCAF | 0.10395 | !G,G: 1.28(1.01, 1.63) | 0.02676 | A: 1.26(1.03, 1.54) | 527, 660 |
| RS1286762 | 03: 025573913 | RARB | 0.03039 | C,T: 1.3(1.02, 1.67) | 0.54925 | T: 1.07(0.86, 1.32) | 527, 660 |
| RS267538 | 03: 037560743 | ITGA9 | 0.04807 | !G,T: 1.33(1.06, 1.68) | 1.00000 | T: 1(0.85, 1.18) | 527, 660 |
| RS868891 | 03: 049924075 | MON1A | 0.07777 | C,C: 1.41(1.04, 1.91) | 0.03256 | C: 1.2(1.02, 1.41) | 527, 660 |
| RS2282751 | 03: 050266789 | GNAI2 | 0.09691 | G,G: 1.33(1.02, 1.74) | 0.02505 | G: 1.32(1.04, 1.68) | 527, 660 |
| RS4687771 | 03: 051730152 | GRM2 | 0.01912 | !T,T: 1.45(1.11, 1.88) | 0.00440 | A: 1.41(1.11, 1.79) | 525, 660 |
| RS3774531 | 03: 053712615 | CACNA1D | 0.03461 | !C,C: 1.34(1.03, 1.74) | 0.00921 | T: 1.24(1.06, 1.46) | 527, 660 |
| RS2360026 | 03: 054782360 | CACNA2D3 | 0.06374 | !C,C: 1.37(1.06, 1.77) | 0.04248 | T: 1.18(1.01, 1.39) | 527, 660 |
| RS1913914 | 03: 054952530 | CACNA2D3 | 0.02979 | !A,T: 1.44(1.1, 1.88) | 0.07048 | T: 1.24(0.99, 1.57) | 527, 660 |
| RS301935 | 03: 098838920 | EPHA6 | 0.04815 | !C,T: 1.4(1.03, 1.9) | 0.16115 | T: 1.22(0.93, 1.61) | 526, 659 |
| RS7650099 | 03: 098932207 | EPHA6 | 0.03133 | A,G: 1.37(1.09, 1.73) | 1.00000 | A: 1(0.85, 1.18) | 527, 660 |
| RS9864542 | 03: 098938598 | EPHA6 | 0.01578 | A,C: 1.42(1.13, 1.78) | 1.00000 | C: 1(0.85, 1.18) | 527, 660 |
| RS2454685 | 03: 100069756 | DCBLD2 | 0.08261 | C,C: 1.45(1.05, 2) | 0.04972 | C: 1.19(1, 1.4) | 527, 660 |
| RS1520648 | 03: 101854561 | GPR128 | 0.06971 | G,G: 1.38(1.05, 1.81) | 0.01681 | G: 1.35(1.05, 1.72) | 524, 657 |
| RS1143781 | 03: 101883423 | GPR128 | 0.06244 | G,G: 1.49(1.07, 2.08) | 0.04559 | G: 1.19(1.01, 1.4) | 527, 659 |
| RS2030515 | 03: 106759285 | ALCAM | 0.03467 | !G,G: 1.49(1.09, 2.03) | 0.15437 | A: 1.13(0.96, 1.33) | 527, 659 |
| RS2629396 | 03: 120731657 | CD80 | 0.09444 | !A,A: 1.3(1.03, 1.64) | 0.03182 | C: 1.21(1.02, 1.43) | 527, 660 |
| RS610902 | 03: 120734475 | CD80 | 0.08851 | C,C: 1.31(1.04, 1.66) | 0.04107 | C: 1.19(1.01, 1.41) | 527, 660 |
| RS1393200 | 03: 123479573 | CASR | 0.00396 | A,G: 1.42(1.13, 1.79) | 0.89578 | A: 1.01(0.85, 1.2) | 526, 658 |
| RS3792298 | 03: 169024676 | SERPINI1 | 0.09264 | !C,C: 1.31(1.03, 1.65) | 0.04835 | A: 1.22(1.01, 1.49) | 537, 667 |
| RS1875446 | 03: 172433618 | TNIK | 0.00450 | C,C: 1.38(1.1, 1.73) | 0.00105 | C: 1.35(1.13, 1.61) | 527, 660 |
| RS6783565 | 03: 172460392 | TNIK | 0.07963 | G,G: 1.26(1, 1.58) | 0.02383 | G: 1.24(1.03, 1.5) | 527, 660 |
| RS4894672 | 03: 172467502 | TNIK | 0.04414 | !G,G: 1.7(1.07, 2.7) | 0.01967 | A: 1.25(1.04, 1.5) | 527, 660 |
| RS4894686 | 03: 172470611 | TNIK | 0.04111 | !T,T: 1.41(1.08, 1.83) | 0.05172 | A: 1.18(1, 1.39) | 526, 660 |
| RS9829436 | 03: 172521131 | TNIK | 0.01096 | !G,G: 1.76(1.17, 2.66) | 0.39468 | A: 1.08(0.91, 1.29) | 527, 660 |
| RS9855285 | 03: 172525629 | TNIK | 0.04547 | A,T: 1.34(1.07, 1.69) | 1.00000 | A: 1(0.85, 1.18) | 526, 660 |
| RS11919795 | 03: 185373562 | AP2M1, | 0.00947 | G,G: 2.02(1.29, 3.18) | 0.10301 | G: 1.17(0.97, 1.4) | 527, 660 |
| | | DVL3 | | | | | |
| RS710446 | 03: 187942621 | KNG1 | 0.03812 | G,G: 1.57(1.12, 2.21) | 0.04565 | G: 1.19(1, 1.4) | 527, 660 |
| RS266762 | 03: 187945537 | KNG1 | 0.06910 | A,A: 1.31(1.03, 1.67) | 0.02272 | A: 1.21(1.03, 1.43) | 527, 660 |
| RS2241766 | 03: 188053586 | ADIPOQ | 0.00679 | !T,T: 1.58(1.2, 2.08) | 0.00129 | G: 1.52(1.18, 1.95) | 526, 658 |
| RS3774262 | 03: 188054508 | ADIPOQ | 0.00988 | !G,G: 1.54(1.17, 2.04) | 0.00155 | A: 1.5(1.17, 1.93) | 526, 657 |
| RS1365113 | 03: 188875422 | SST | 0.02913 | T,T: 1.49(1.11, 1.98) | 0.00825 | T: 1.43(1.09, 1.86) | 527, 658 |
| RS2278034 | 03: 197081304 | TNK2 | 0.03055 | G,G: 1.37(1.07, 1.76) | 0.00701 | G: 1.25(1.07, 1.48) | 527, 660 |
| RS843532 | 03: 197966608 | PAK2 | 0.04933 | !C,T: 1.39(1.07, 1.81) | 0.09234 | T: 1.22(0.97, 1.54) | 527, 660 |
| RS7646247 | 03: 198003606 | PAK2 | 0.03923 | !C,T: 1.36(1.07, 1.73) | 0.23679 | C: 1.13(0.93, 1.37) | 527, 660 |
| RS4916554 | 03: 198021092 | PAK2 | 0.01870 | !C,G: 1.44(1.11, 1.86) | 0.07993 | G: 1.21(0.98, 1.51) | 527, 660 |
| RS873785 | 04: 000915149 | GAK, | 0.02215 | !A,G: 1.39(1.11, 1.75) | 0.35471 | A: 1.08(0.92, 1.28) | 525, 660 |
| | | MGC4618 | | | | | |
| RS2301293 | 04: 001771664 | FGFR3 | 0.04961 | !T,T: 2.44(1, 5.97) | 0.90297 | T: 1.02(0.8, 1.29) | 527, 660 |
| RS6446402 | 04: 005918854 | CRMP1 | 0.04262 | C,C: 1.33(1.05, 1.68) | 0.01195 | C: 1.29(1.06, 1.56) | 527, 660 |
| RS2192356 | 04: 015033689 | C1QTNF7 | 0.03640 | !A,A: 1.8(1.15, 2.81) | 0.06926 | G: 1.18(0.99, 1.42) | 527, 659 |
| RS768695 | 04: 023407916 | PPARGC1A | 0.08736 | A,A: 1.34(1.02, 1.76) | 0.02832 | A: 1.2(1.02, 1.41) | 527, 660 |
| RS4861327 | 04: 040051471 | CHRNA9 | 0.02693 | A,A: | 0.03722 | A: 1.47(1.03, 2.09) | 527, 660 |
| | | | | 11.36(0.61, 211.38) | | | |
| RS346005 | 04: 053149495 | FLJ11850 | 0.06098 | A,A: 1.32(1.05, 1.66) | 0.02115 | A: 1.23(1.03, 1.47) | 527, 659 |
| RS2244291 | 04: 053163236 | USP46 | 0.00936 | A,A: 1.56(1.17, 2.08) | 0.00165 | A: 1.53(1.17, 1.99) | 527, 660 |
| RS7677751 | 04: 054819217 | PDGFRA | 0.02370 | C,T: 1.46(1.12, 1.91) | 0.01886 | T: 1.33(1.05, 1.68) | 527, 660 |
| RS2307049 | 04: 054824911 | PDGFRA | 0.02690 | A,G: 1.41(1.08, 1.84) | 0.11650 | A: 1.21(0.96, 1.53) | 528, 643 |
| RS1547904 | 04: 054841146 | PDGFRA | 0.01717 | !C,C: 1.43(1.11, 1.84) | 0.00367 | T: 1.39(1.12, 1.74) | 527, 660 |
| RS3690 | 04: 054856570 | PDGFRA | 0.02120 | !A,A: 1.43(1.1, 1.85) | 0.00476 | C: 1.4(1.11, 1.76) | 527, 658 |
| RS8022 | 04: 055301184 | KIT | 0.11039 | !G,G: 1.29(0.99, 1.68) | 0.03989 | T: 1.29(1.02, 1.63) | 527, 660 |
| RS3796776 | 04: 055301967 | KIT | 0.09983 | !C,C: 1.31(1.01, 1.7) | 0.03036 | G: 1.3(1.03, 1.64) | 527, 660 |
| RS1434568 | 04: 073425704 | ADAMTS3 | 0.11508 | T,T: 1.6(0.95, 2.69) | 0.04494 | T: 1.23(1.01, 1.49) | 523, 645 |
| RS3733228 | 04: 083801088 | SCD5 | 0.03390 | !A,C: 1.34(1.06, 1.68) | 1.00000 | C: 1(0.85, 1.19) | 527, 660 |
| RS6535369 | 04: 083806003 | SCD5 | 0.00209 | !G,T: 1.44(1.13, 1.83) | 0.73701 | G: 1.04(0.86, 1.25) | 527, 660 |
| RS4693508 | 04: 083875498 | SCD5 | 0.04190 | !C,C: 1.44(1.08, 1.93) | 0.02017 | T: 1.22(1.03, 1.43) | 527, 660 |
| RS17703261 | 04: 088277361 | AFF1 | 0.00144 | A,T: 1.59(1.23, 2.06) | 0.04968 | T: 1.24(1, 1.53) | 526, 659 |
| RS4693851 | 04: 088796778 | DMP1 | 0.06952 | A,A: 1.31(1.03, 1.66) | 0.01511 | A: 1.28(1.05, 1.57) | 527, 660 |
| RS6812310 | 04: 088966535 | MEPE | 0.06147 | T,T: 1.32(1.04, 1.67) | 0.01368 | T: 1.27(1.05, 1.54) | 522, 645 |
| RS7356228 | 04: 090962878 | SNCA | 0.01799 | C,G: 1.41(1.12, 1.77) | 0.55328 | G: 1.05(0.89, 1.24) | 527, 660 |
| RS1372519 | 04: 090976332 | SNCA, | 0.05260 | G,G: 1.36(1.07, 1.73) | 0.01501 | G: 1.29(1.05, 1.59) | 525, 659 |
| | | LOC644248 | | | | | |
| RS1108563 | 04: 094773785 | GRID2 | 0.04355 | !C,C: 3.16(1.12, 8.97) | 0.76162 | A: 1.04(0.82, 1.32) | 527, 660 |
| RS4699673 | 04: 095907626 | BMPR1B | 0.04957 | !A,A: 1.36(1.07, 1.72) | 0.02614 | G: 1.26(1.03, 1.54) | 527, 659 |
| RS997903 | 04: 095943664 | BMPR1B | 0.08990 | C,C: 1.27(1.01, 1.6) | 0.02091 | C: 1.24(1.03, 1.49) | 527, 660 |
| RS7699554 | 04: 096012581 | BMPR1B | 0.10743 | G,G: 1.28(0.99, 1.65) | 0.03686 | G: 1.28(1.02, 1.61) | 527, 660 |
| RS9307150 | 04: 096086090 | BMPR1B | 0.04796 | !T,T: 1.77(1.11, 2.8) | 0.21832 | C: 1.13(0.94, 1.36) | 527, 660 |
| RS230514 | 04: 103690968 | NFKB1 | 0.03363 | !C,T: 1.31(1.04, 1.65) | 0.66883 | C: 1.04(0.88, 1.23) | 527, 660 |
| RS1800627 | 04: 110833107 | CASP6 | 0.03127 | !T,T: 1.44(1.09, 1.9) | 0.01033 | C: 1.24(1.05, 1.46) | 527, 660 |
| RS4541508 | 04: 110901936 | CFI | 0.08183 | !T,T: 1.52(1.04, 2.21) | 0.04327 | C: 1.2(1.01, 1.42) | 527, 660 |
| RS7670019 | 04: 114826199 | CAMK2D | 0.02624 | A,A: 2.39(1.27, 4.48) | 0.15541 | A: 1.16(0.95, 1.43) | 525, 660 |
| RS1479924 | 04: 123607050 | IL2 | 0.04504 | A,G: 1.35(1.07, 1.7) | 0.40959 | G: 1.08(0.9, 1.29) | 527, 660 |
| RS308428 | 04: 123983958 | FGF2 | 0.04299 | G,G: 2.64(1.09, 6.35) | 0.02915 | G: 1.31(1.03, 1.65) | 527, 660 |
| RS3811741 | 04: 129022609 | PLK4 | 0.01132 | T,T: 1.44(1.14, 1.81) | 0.00397 | T: 1.3(1.09, 1.55) | 527, 660 |
| RS3811740 | 04: 129026669 | PLK4 | 0.00678 | T,T: 1.45(1.15, 1.83) | 0.00126 | T: 1.32(1.12, 1.57) | 527, 660 |
| RS17012739 | 04: 129034414 | PLK4 | 0.00678 | T,T: 1.45(1.15, 1.83) | 0.00126 | T: 1.32(1.12, 1.57) | 527, 660 |
| RS1589241 | 04: 142784539 | IL15 | 0.00239 | !T,T: 2.01(1.29, 3.11) | 0.00117 | C: 1.35(1.13, 1.61) | 526, 660 |
| RS990851 | 04: 142787053 | IL15 | 0.00223 | !C,C: 2.01(1.29, 3.12) | 0.00100 | G: 1.35(1.13, 1.61) | 527, 660 |
| RS1519552 | 04: 142795473 | IL15 | 0.00223 | !A,A: 2.01(1.29, 3.12) | 0.00100 | G: 1.35(1.13, 1.61) | 527, 660 |
| RS1040257 | 04: 142850562 | IL15 | 0.00104 | !C,C: 2.1(1.36, 3.26) | 0.00068 | T: 1.36(1.14, 1.62) | 527, 660 |
| RS1607595 | 04: 142855098 | IL15 | 0.00610 | A,G: 1.54(1.19, 2) | 0.02121 | A: 1.3(1.04, 1.63) | 522, 646 |
| RS2254514 | 04: 142859988 | IL15 | 0.00114 | !T,T: 2.1(1.36, 3.26) | 0.00080 | C: 1.36(1.14, 1.62) | 527, 660 |
| RS906124 | 04: 149209810 | ARHGAP10 | 0.07684 | !T,T: 1.31(1.04, 1.66) | 0.04975 | C: 1.19(1, 1.41) | 527, 659 |
| RS2289318 | 04: 154853184 | RNF175 | 0.13806 | C,C: 1.26(0.99, 1.6) | 0.04156 | C: 1.23(1.01, 1.51) | 527, 660 |
| RS9044 | 04: 157065537 | CTSO | 0.11218 | T,T: 1.3(1.01, 1.67) | 0.04163 | T: 1.19(1.01, 1.4) | 526, 660 |
| RS6856354 | 04: 157068548 | CTSO | 0.06061 | C,C: 1.5(1.07, 2.1) | 0.03112 | C: 1.21(1.02, 1.43) | 527, 658 |
| RS9764 | 04: 164464855 | NPY1R | 0.01696 | !T,T: 1.41(1.12, 1.77) | 0.00557 | C: 1.3(1.08, 1.56) | 527, 660 |
| RS1438113 | 04: 166516630 | CPE | 0.04636 | A,G: 1.35(1.07, 1.69) | 0.83637 | G: 1.02(0.87, 1.2) | 527, 660 |
| RS4429751 | 04: 168089332 | SPOCK3 | 0.03312 | !G,G: 1.44(1.08, 1.92) | 0.21368 | T: 1.11(0.95, 1.31) | 527, 660 |
| RS7683288 | 04: 168239183 | SPOCK3 | 0.09408 | !T,T: 1.37(1.04, 1.81) | 0.03519 | C: 1.32(1.02, 1.71) | 527, 660 |
| RS9996873 | 04: 170793421 | CLCN3 | 0.01032 | !G,G: 2.19(1.18, 4.05) | 0.87929 | T: 1.02(0.83, 1.24) | 527, 660 |
| RS10866341 | 04: 170805364 | CLCN3 | 0.03750 | A,A: 1.61(1.08, 2.4) | 0.01530 | A: 1.25(1.05, 1.49) | 527, 660 |
| RS11732927 | 04: 185544039 | IRF2 | 0.04180 | !C,T: 1.43(1.09, 1.89) | 0.02288 | C: 1.33(1.04, 1.7) | 527, 660 |
| RS3775554 | 04: 185567394 | IRF2 | 0.04397 | !C,C: | 0.22123 | G: 1.17(0.91, 1.51) | 536, 666 |
| | | | | 3.87(1.21, 12.32) | | | |
| RS13114710 | 04: 185572813 | IRF2 | 0.00719 | !G,G: 1.48(1.1, 1.97) | 0.00217 | T: 1.52(1.16, 1.98) | 537, 668 |
| RS7667268 | 04: 185627070 | IRF2 | 0.06106 | A,A: 1.3(1.03, 1.64) | 0.01672 | A: 1.24(1.04, 1.48) | 526, 660 |
| RS6553010 | 04: 187693337 | MTNR1A | 0.07298 | !G,G: 1.33(1.05, 1.68) | 0.02244 | A: 1.26(1.04, 1.54) | 527, 659 |
| RS2042449 | 05: 001469646 | SLC6A3 | 0.09257 | C,C: 1.25(0.98, 1.58) | 0.03640 | C: 1.24(1.02, 1.52) | 525, 658 |
| RS299608 | 05: 034060142 | C1QTNF3 | 0.08193 | T,T: 1.26(1, 1.58) | 0.02524 | T: 1.22(1.03, 1.45) | 527, 659 |
| RS299602 | 05: 034061668 | C1QTNF3 | 0.09415 | C,C: 1.24(0.99, 1.57) | 0.03148 | C: 1.21(1.02, 1.44) | 527, 660 |
| RS1478438 | 05: 052238855 | ITGA1 | 0.00914 | !A,A: 3.09(1.29, 7.39) | 0.95584 | T: 1.01(0.81, 1.25) | 527, 660 |
| RS16889865 | 05: 058790977 | PDE4D | 0.08084 | !A,A: 1.38(1.05, 1.82) | 0.02836 | G: 1.33(1.03, 1.7) | 526, 660 |
| RS7711077 | 05: 065122383 | NLN | 0.12591 | !T,T: 1.36(1.01, 1.83) | 0.03910 | G: 1.35(1.02, 1.78) | 527, 660 |
| RS6896574 | 05: 065123931 | NLN | 0.06983 | G,G: 1.38(1.06, 1.8) | 0.02551 | G: 1.32(1.04, 1.68) | 526, 660 |
| RS2930951 | 05: 068571372 | CDK7 | 0.03493 | C,G: 1.36(1.07, 1.72) | 0.30499 | G: 1.1(0.93, 1.3) | 514, 595 |
| RS1047530 | 05: 075947155 | IQGAP2, | 0.00549 | !A,A: 1.58(1.2, 2.08) | 0.01480 | G: 1.23(1.04, 1.44) | 527, 660 |
| | | F2RL2 | | | | | |
| RS6452557 | 05: 083324573 | EDIL3 | 0.00834 | T,T: 1.49(1.16, 1.91) | 0.00207 | T: 1.4(1.13, 1.74) | 526, 658 |
| RS2009272 | 05: 083408964 | EDIL3 | 0.02648 | !T,T: 1.87(1.19, 2.94) | 0.07313 | A: 1.18(0.99, 1.42) | 528, 638 |
| RS3822634 | 05: 083572771 | EDIL3 | 0.10811 | !G,G: 1.26(1, 1.59) | 0.03323 | A: 1.23(1.02, 1.49) | 527, 660 |
| RS1373959 | 05: 083588782 | EDIL3 | 0.11760 | !C,C: 1.26(1, 1.59) | 0.03755 | G: 1.23(1.02, 1.48) | 527, 660 |
| RS2063244 | 05: 090275784 | GPR98 | 0.04890 | A,A: 1.4(1.06, 1.85) | 0.01445 | A: 1.23(1.04, 1.44) | 527, 660 |
| RS2973442 | 05: 090457972 | GPR98 | 0.02597 | !A,G: 1.46(1.12, 1.91) | 0.03042 | G: 1.3(1.03, 1.63) | 525, 660 |
| RS26345 | 05: 096109094 | CAST | 0.06058 | G,G: 1.53(1.08, 2.19) | 0.02375 | G: 1.48(1.06, 2.08) | 527, 660 |
| RS3805601 | 05: 108113642 | FER, | 0.03434 | !C,C: 1.37(1.08, 1.74) | 0.02370 | G: 1.27(1.04, 1.55) | 527, 660 |
| | | LOC643524, | | | | | |
| | | LOC647747 | | | | | |
| RS1469249 | 05: 113865097 | KCNN2 | 0.02554 | G,G: 1.4(1.1, 1.77) | 0.00844 | G: 1.3(1.07, 1.59) | 525, 660 |
| RS806100 | 05: 127496076 | SLC12A2 | 0.02892 | !C,C: 1.38(1.07, 1.77) | 0.00685 | T: 1.25(1.06, 1.47) | 525, 659 |
| RS2243289 | 05: 132046031 | IL4 | 0.03805 | G,G: 2.65(0.95, 7.38) | 0.49791 | A: 1.09(0.86, 1.39) | 527, 660 |
| RS1148360 | 05: 134940625 | CXCL14 | 0.00290 | !C,C: 1.42(1.13, 1.8) | 0.00070 | T: 1.4(1.16, 1.7) | 527, 660 |
| RS2003491 | 05: 137272210 | PKD2L2 | 0.01339 | A,G: 1.43(1.13, 1.79) | 0.86822 | G: 1.02(0.86, 1.19) | 527, 660 |
| RS4835761 | 05: 137445768 | WNT8A | 0.01266 | A,G: 1.43(1.13, 1.79) | 0.93389 | A: 1.01(0.86, 1.19) | 527, 660 |
| RS6596422 | 05: 137454346 | WNT8A | 0.01052 | A,G: 1.44(1.14, 1.81) | 1.00000 | A: 1(0.85, 1.18) | 526, 659 |
| RS3860103 | 05: 137455173 | WNT8A | 0.01032 | C,G: 1.44(1.14, 1.81) | 0.96692 | C: 1(0.85, 1.18) | 527, 660 |
| RS3813321 | 05: 137827923 | EGR1 | 0.05500 | !C,C: 1.38(1.07, 1.77) | 0.03472 | T: 1.19(1.01, 1.4) | 527, 660 |
| RS171632 | 05: 139921502 | APBB3 | 0.03346 | !T,T: 1.65(1.08, 2.53) | 0.01238 | C: 1.26(1.05, 1.51) | 527, 660 |
| RS250429 | 05: 139925010 | SLC35A4, | 0.06257 | !A,A: 1.54(1.02, 2.32) | 0.02135 | G: 1.23(1.03, 1.48) | 527, 660 |
| | | APBB3 | | | | | |
| RS2563298 | 05: 139991499 | CD14 | 0.08939 | C,C: 1.25(1, 1.58) | 0.02505 | C: 1.23(1.03, 1.47) | 527, 660 |
| RS3776100 | 05: 140482361 | PCDHB4 | 0.06604 | C,C: 1.28(1.01, 1.61) | 0.02064 | C: 1.25(1.04, 1.52) | 527, 660 |
| RS7709485 | 05: 145875089 | GPR151 | 0.10996 | !G,G: 1.24(0.97, 1.58) | 0.03976 | C: 1.24(1.01, 1.53) | 527, 660 |
| RS1864962 | 05: 146761077 | DPYSL3 | 0.02847 | T,T: 1.4(1.1, 1.79) | 0.04476 | T: 1.18(1, 1.4) | 526, 659 |
| RS1973877 | 05: 147852159 | HTR4 | 0.03802 | T,T: 1.4(1.05, 1.86) | 0.31785 | T: 1.09(0.92, 1.28) | 527, 660 |
| RS216151 | 05: 149428620 | CSF1R | 0.05471 | !C,C: 1.34(1.06, 1.68) | 0.01839 | T: 1.25(1.04, 1.5) | 526, 660 |
| RS3776084 | 05: 149548286 | SLC6A7 | 0.04631 | G,G: 1.35(1.06, 1.71) | 0.01416 | G: 1.23(1.04, 1.45) | 527, 658 |
| RS2240794 | 05: 149563529 | SLC6A7 | 0.07056 | !C,C: 1.34(1.05, 1.71) | 0.03136 | T: 1.27(1.03, 1.56) | 526, 659 |
| RS7728652 | 05: 149763887 | CD74 | 0.14886 | !G,G: 1.38(1.01, 1.89) | 0.04784 | A: 1.36(1.01, 1.83) | 536, 667 |
| RS1895245 | 05: 151752248 | NMUR2 | 0.11156 | G,G: 1.28(1.01, 1.63) | 0.03084 | G: 1.25(1.02, 1.53) | 527, 660 |
| RS4958535 | 05: 151755257 | NMUR2 | 0.10850 | C,C: 1.29(1.01, 1.64) | 0.03074 | C: 1.25(1.02, 1.54) | 527, 659 |
| RS716257 | 05: 151757659 | NMUR2 | 0.11331 | T,T: 1.28(1.01, 1.63) | 0.03083 | T: 1.25(1.02, 1.53) | 526, 659 |
| RS730691 | 05: 158688805 | IL12B | 0.03036 | !T,T: 1.55(1.1, 2.2) | 0.24221 | C: 1.11(0.94, 1.31) | 527, 659 |
| RS2617503 | 05: 160697784 | GABRB2 | 0.08533 | !G,G: 1.28(1.01, 1.62) | 0.02394 | A: 1.22(1.03, 1.44) | 527, 659 |
| RS2194159 | 05: 160704868 | GABRB2 | 0.04355 | C,C: 1.84(1.07, 3.15) | 0.01816 | C: 1.27(1.04, 1.54) | 527, 660 |
| RS209358 | 05: 161452457 | GABRG2 | 0.14331 | !T,T: 1.25(0.98, 1.61) | 0.04596 | C: 1.18(1.01, 1.39) | 527, 660 |
| RS299295 | 05: 162842294 | HMMR | 0.02160 | C,T: 1.41(1.11, 1.79) | 0.13822 | T: 1.15(0.96, 1.39) | 525, 660 |
| RS1063310 | 05: 170171690 | GABRP | 0.04212 | !C,C: 1.36(1.08, 1.71) | 0.01533 | A: 1.27(1.05, 1.53) | 527, 660 |
| RS248180 | 05: 178672676 | ADAMTS2 | 0.03069 | !T,T: 1.73(1.12, 2.68) | 0.33142 | C: 1.1(0.92, 1.32) | 527, 660 |
| RS3111515 | 05: 179627958 | MAPK9 | 0.05794 | C,C: 1.35(1.06, 1.7) | 0.03462 | C: 1.2(1.01, 1.41) | 527, 660 |
| RS2242216 | 05: 179974097 | FLT4 | 0.04559 | !A,G: 1.35(1.07, 1.7) | 0.77261 | G: 1.02(0.87, 1.2) | 527, 660 |
| RS3799562 | 06: 006115271 | F13A1 | 0.04911 | !A,A: 1.35(1.07, 1.7) | 0.01950 | T: 1.26(1.04, 1.52) | 527, 660 |
| RS2208840 | 06: 011291370 | NEDD9 | 0.03727 | !G,G: 2.17(1.09, 4.32) | 0.01953 | A: 1.29(1.04, 1.59) | 527, 660 |
| RS3756966 | 06: 011310590 | NEDD9 | 0.12949 | !A,A: 1.25(0.99, 1.57) | 0.04373 | G: 1.22(1.01, 1.48) | 527, 660 |
| RS3798743 | 06: 011320945 | NEDD9 | 0.10693 | !G,G: 1.25(0.99, 1.58) | 0.03896 | A: 1.23(1.01, 1.49) | 527, 660 |
| RS3734404 | 06: 011321818 | NEDD9 | 0.03117 | C,C: 1.49(1.12, 1.99) | 0.04592 | C: 1.18(1, 1.39) | 527, 660 |
| RS- | 06: 031470048 | LOC442200 | 0.06232 | G,G: 1.73(1.01, 2.98) | 0.02737 | G: 1.24(1.03, 1.51) | 523, 638 |
| GSK8113708 | | | | | | | |
| RS1800629 | 06: 031651010 | LTA | 0.01511 | !A,A: 2.16(1.14, 4.09) | 0.00554 | G: 1.34(1.09, 1.66) | 527, 660 |
| RS2844479 | 06: 031680935 | | 0.02987 | !T,T: 1.35(1.07, 1.69) | 0.00589 | G: 1.28(1.08, 1.53) | 527, 660 |
| RS2259571 | 06: 031691806 | AIF1 | 0.05764 | !A,A: 1.32(1.05, 1.66) | 0.01846 | C: 1.24(1.04, 1.47) | 527, 656 |
| RS3117582 | 06: 031728499 | APOM, BAT3 | 0.00225 | A,A: 1.59(1.21, 2.08) | 0.00030 | A: 1.56(1.22, 1.99) | 537, 668 |
| RS3131383 | 06: 031812273 | CLIC1 | 0.00294 | C,C: 1.55(1.18, 2.03) | 0.00053 | C: 1.54(1.2, 1.97) | 526, 660 |
| RS3020644 | 06: 032002605 | C2 | 0.04827 | !A,A: 1.35(1.07, 1.7) | 0.03171 | G: 1.21(1.02, 1.43) | 527, 660 |
| RS1270942 | 06: 032026839 | CFB, RDBP | 0.00118 | T,T: 1.61(1.23, 2.12) | 0.00017 | T: 1.59(1.25, 2.04) | 527, 660 |
| RS2072633 | 06: 032027557 | CFB, RDBP | 0.02217 | !T,T: 1.43(1.1, 1.87) | 0.11638 | C: 1.14(0.97, 1.34) | 527, 659 |
| RS437179 | 06: 032036993 | SKIV2L | 0.05121 | !T,T: 1.53(1.08, 2.17) | 0.02212 | G: 1.22(1.03, 1.45) | 527, 660 |
| RS1150754 | 06: 032158736 | TNXB | 0.01497 | G,G: 1.46(1.14, 1.88) | 0.00497 | G: 1.37(1.1, 1.71) | 527, 660 |
| RS204995 | 06: 032262263 | PBX2 | 0.00240 | T,T: 1.41(1.12, 1.77) | 0.00042 | T: 1.38(1.15, 1.65) | 527, 660 |
| RS241424 | 06: 032912912 | TAP2 | 0.09672 | C,C: 1.26(0.99, 1.61) | 0.02671 | C: 1.21(1.02, 1.42) | 527, 660 |
| RS2239701 | 06: 032913027 | TAP2 | 0.04896 | !G,G: 1.39(1.04, 1.85) | 0.01133 | A: 1.23(1.05, 1.45) | 527, 660 |
| RS3763364 | 06: 032915498 | TAP2 | 0.05576 | !A,A: 1.42(1.05, 1.91) | 0.01966 | T: 1.22(1.03, 1.43) | 527, 660 |
| RS206786 | 06: 033043157 | BRD2, | 0.06653 | !A,A: 1.33(1.05, 1.69) | 0.03019 | C: 1.21(1.02, 1.42) | 527, 660 |
| | | LOC651864 | | | | | |
| RS683208 | 06: 033045879 | BRD2, | 0.03857 | A,G: 1.35(1.07, 1.7) | 0.06813 | G: 1.18(0.99, 1.4) | 527, 660 |
| | | LOC651864 | | | | | |
| RS5030979 | 06: 033270206 | COL11A2 | 0.12280 | !T,T: 1.36(1.02, 1.82) | 0.04157 | A: 1.33(1.02, 1.73) | 525, 639 |
| RS4711774 | 06: 044247469 | CAPN11 | 0.00932 | !C,G: 1.45(1.15, 1.84) | 0.11242 | C: 1.16(0.97, 1.4) | 527, 660 |
| RS4276501 | 06: 052365477 | PAQR8 | 0.02313 | T,T: 2.05(1.16, 3.61) | 0.01387 | T: 1.28(1.05, 1.56) | 527, 660 |
| RS2140510 | 06: 069574603 | BAI3 | 0.05247 | C,C: 1.31(1.03, 1.65) | 0.01347 | C: 1.28(1.05, 1.54) | 527, 660 |
| RS2350386 | 06: 073944327 | KCNQ5 | 0.06601 | C,C: 1.33(1.05, 1.67) | 0.04022 | C: 1.2(1.01, 1.43) | 527, 660 |
| RS914479 | 06: 089986927 | GABRR1 | 0.03109 | A,G: 1.37(1.09, 1.73) | 0.61416 | G: 1.05(0.89, 1.23) | 527, 660 |
| RS292246 | 06: 090652570 | CX62 | 0.15064 | G,G: 1.28(1, 1.65) | 0.04732 | G: 1.25(1.01, 1.56) | 527, 660 |
| RS1145726 | 06: 091273036 | MAP3K7 | 0.02257 | A,C: 1.35(1.07, 1.7) | 0.89390 | C: 1.01(0.85, 1.2) | 527, 659 |
| RS1475917 | 06: 102089483 | GRIK2 | 0.06671 | G,G: 1.3(1.03, 1.64) | 0.01791 | G: 1.23(1.04, 1.46) | 527, 660 |
| RS1223042 | 06: 102520922 | GRIK2 | 0.09446 | A,A: 1.39(1.01, 1.92) | 0.02978 | A: 1.2(1.02, 1.42) | 527, 660 |
| RS500972 | 06: 116394336 | FRK | 0.02575 | !C,T: 1.35(1.07, 1.71) | 0.93039 | C: 1.01(0.85, 1.2) | 527, 660 |
| RS2756117 | 06: 153108601 | VIP | 0.13282 | !G,G: 1.36(1.01, 1.82) | 0.04668 | A: 1.31(1.01, 1.72) | 527, 657 |
| RS8031 | 06: 160020630 | SOD2 | 0.11826 | A,A: 1.27(0.98, 1.65) | 0.03178 | A: 1.2(1.02, 1.41) | 527, 660 |
| RS377551 | 06: 160701723 | SLC22A3 | 0.00343 | !G,G: 1.49(1.18, 1.87) | 0.00087 | A: 1.36(1.14, 1.62) | 527, 660 |
| RS3127574 | 06: 160711360 | SLC22A3 | 0.07279 | C,C: 1.32(1.02, 1.72) | 0.02074 | C: 1.21(1.03, 1.42) | 527, 659 |
| RS10499314 | 06: 160735700 | SLC22A3 | 0.00903 | !C,C: 2.41(1.26, 4.64) | 0.00498 | A: 1.34(1.09, 1.65) | 527, 659 |
| RS4708867 | 06: 160762715 | SLC22A3 | 0.06336 | !G,G: | 0.03634 | A: 1.36(1.03, 1.79) | 526, 660 |
| | | | | 3.12(0.96, 10.16) | | | |
| RS1950562 | 06: 161043175 | PLG | 0.08822 | !A,A: 1.39(1.03, 1.89) | 0.03696 | G: 1.19(1.01, 1.4) | 527, 660 |
| RS2235297 | 06: 166746749 | RPS6KA2 | 0.00027 | T,T: 1.99(1.38, 2.89) | 0.00011 | T: 1.4(1.18, 1.67) | 527, 660 |
| RS10946164 | 06: 166751684 | RPS6KA2 | 0.11233 | C,C: 1.26(0.98, 1.62) | 0.03805 | C: 1.19(1.01, 1.4) | 527, 660 |
| RS3799630 | 06: 166885426 | RPS6KA2 | 0.04997 | !C,C: 1.33(1.06, 1.67) | 0.01309 | T: 1.25(1.05, 1.49) | 527, 660 |
| RS971152 | 06: 166889926 | RPS6KA2 | 0.00979 | G,G: 1.46(1.14, 1.88) | 0.00210 | G: 1.41(1.13, 1.76) | 527, 660 |
| RS1543802 | 06: 166892863 | RPS6KA2 | 0.03931 | !G,G: 1.53(1.06, 2.21) | 0.01578 | A: 1.23(1.04, 1.46) | 526, 659 |
| RS3799612 | 06: 166901630 | RPS6KA2 | 0.01960 | !G,G: 1.4(1.11, 1.76) | 0.00972 | C: 1.27(1.06, 1.52) | 526, 660 |
| RS845647 | 06: 167129757 | RPS6KA2 | 0.00323 | !T,T: 3.59(1.61, 8.01) | 0.10725 | C: 1.2(0.97, 1.48) | 527, 660 |
| RS1099652 | 06: 167150514 | RPS6KA2 | 0.02754 | !A,A: 3.09(1.29, 7.39) | 0.15489 | G: 1.17(0.94, 1.45) | 527, 660 |
| RS1099656 | 06: 167160489 | RPS6KA2 | 0.03319 | !G,G: 2.52(1.2, 5.3) | 0.05791 | A: 1.23(1, 1.51) | 522, 639 |
| RS2267719 | 07: 030922976 | AQP1 | 0.01830 | !C,C: 1.58(1.1, 2.27) | 0.00612 | T: 1.62(1.15, 2.29) | 527, 660 |
| RS6462302 | 07: 031782981 | PDE1C | 0.07924 | !G,G: 1.35(1.04, 1.76) | 0.02072 | A: 1.33(1.05, 1.68) | 527, 660 |
| RS11768234 | 07: 032112436 | PDE1C | 0.03938 | !C,C: 1.33(1.04, 1.71) | 0.00889 | G: 1.25(1.06, 1.48) | 517, 607 |
| RS32319 | 07: 032149169 | PDE1C | 0.11797 | T,T: 1.27(1.01, 1.61) | 0.03830 | T: 1.2(1.01, 1.42) | 527, 660 |
| RS7786797 | 07: 032239480 | PDE1C | 0.14314 | !A,A: 1.34(1, 1.8) | 0.03918 | C: 1.33(1.02, 1.74) | 527, 658 |
| RS1052969 | 07: 037913072 | SFRP4 | 0.04409 | !C,T: 1.36(1.08, 1.73) | 0.09322 | T: 1.18(0.97, 1.42) | 526, 660 |
| RS2075070 | 07: 044121777 | POLD2, | 0.02258 | C,T: 1.38(1.1, 1.74) | 0.90051 | T: 1.01(0.86, 1.19) | 527, 660 |
| | | AEBP1 | | | | | |
| RS2041547 | 07: 044167409 | GCK | 0.00741 | A,G: 1.46(1.16, 1.84) | 0.83639 | A: 1.02(0.87, 1.2) | 527, 660 |
| RS2284776 | 07: 044183279 | GCK | 0.02568 | A,G: 1.38(1.1, 1.74) | 0.64966 | A: 1.04(0.88, 1.22) | 527, 660 |
| RS217429 | 07: 044521724 | NPC1L1 | 0.01821 | G,G: 1.99(1.18, 3.35) | 0.62819 | G: 1.05(0.87, 1.27) | 527, 660 |
| RS1267 | 07: 055468742 | LANCL2 | 0.09523 | A,A: 1.29(1.02, 1.63) | 0.02995 | A: 1.21(1.02, 1.43) | 527, 660 |
| RS3966180 | 07: 056128615 | PHKG1 | 0.04051 | !C,C: 1.37(1.08, 1.74) | 0.02571 | T: 1.26(1.03, 1.55) | 521, 633 |
| RS3757598 | 07: 075283313 | CCL24 | 0.01264 | T,T: 1.93(1.21, 3.08) | 0.42760 | T: 1.08(0.9, 1.29) | 526, 659 |
| RS38559 | 07: 081722923 | CACNA2D1 | 0.03698 | !A,G: 1.46(1.09, 1.96) | 0.05724 | G: 1.29(0.99, 1.67) | 527, 660 |
| RS445 | 07: 092246306 | CDK6 | 0.03960 | !C,T: 1.43(1.05, 1.95) | 0.13857 | C: 1.24(0.94, 1.64) | 527, 660 |
| RS11489600 | 07: 102735276 | PMPCB | 0.03861 | C,T: 1.31(1.04, 1.65) | 0.82953 | C: 1.02(0.86, 1.21) | 527, 660 |
| RS2283064 | 07: 125912289 | GRM8 | 0.04309 | C,C: 1.43(1.05, 1.94) | 0.35391 | C: 1.08(0.92, 1.28) | 527, 660 |
| RS886176 | 07: 126134129 | GRM8 | 0.14561 | !C,C: 1.25(0.99, 1.59) | 0.04309 | T: 1.19(1.01, 1.4) | 527, 660 |
| RS1048531 | 07: 127885307 | HIG2 | 0.12253 | !G,G: 1.27(0.99, 1.61) | 0.04029 | A: 1.25(1.01, 1.54) | 527, 660 |
| RS968404 | 07: 129809277 | CPA1 | 0.03449 | !C,C: 1.41(1.09, 1.81) | 0.01353 | T: 1.32(1.06, 1.65) | 527, 659 |
| RS1990786 | 07: 129912737 | MEST | 0.07843 | C,C: 1.35(1.05, 1.74) | 0.02650 | C: 1.29(1.03, 1.62) | 527, 660 |
| RS10863 | 07: 129932825 | COPG2, | 0.06196 | G,G: 1.37(1.06, 1.76) | 0.02265 | G: 1.31(1.04, 1.64) | 527, 659 |
| | | MEST | | | | | |
| RS1131883 | 07: 142798653 | ZYX, EPHA1 | 0.01915 | G,G: 1.77(1.09, 2.87) | 0.02131 | G: 1.73(1.08, 2.79) | 526, 660 |
| RS753482 | 07: 150337316 | NOS3 | 0.02681 | !G,T: 1.42(1.1, 1.82) | 0.02876 | T: 1.26(1.03, 1.55) | 527, 660 |
| RS6990306 | 08: 006863504 | DEFA3 | 0.01000 | C,G: 1.52(1.16, 1.98) | 0.01534 | G: 1.35(1.06, 1.7) | 527, 660 |
| RS2976440 | 08: 024864142 | NEFL | 0.06977 | T,T: 1.32(1.05, 1.66) | 0.03966 | T: 1.2(1.01, 1.43) | 527, 660 |
| RS7831584 | 08: 024868236 | NEFL | 0.08024 | T,T: 1.47(1.05, 2.06) | 0.03082 | T: 1.42(1.03, 1.96) | 527, 660 |
| RS2976437 | 08: 024872047 | NEFL | 0.06585 | A,A: 1.33(1.05, 1.67) | 0.02864 | A: 1.21(1.02, 1.44) | 527, 659 |
| RS965130 | 08: 025343473 | KCTD9 | 0.10588 | C,C: 1.27(1, 1.6) | 0.03591 | C: 1.2(1.01, 1.42) | 527, 660 |
| RS6557933 | 08: 026560892 | DPYSL2 | 0.04314 | !A,A: 1.42(1.05, 1.9) | 0.29947 | G: 1.09(0.93, 1.29) | 527, 660 |
| RS2292977 | 08: 027374552 | PTK2B, | 0.02284 | C,C: 2.63(1.25, 5.55) | 0.60918 | C: 1.06(0.85, 1.33) | 527, 660 |
| | | CHRNA2 | | | | | |
| RS4733302 | 08: 032137312 | NRG1 | 0.04668 | C,C: 13.84(0.76, 250.77) | 0.59865 | C: 1.1(0.78, 1.55) | 536, 668 |
| RS9693898 | 08: 037938333 | ADRB3 | 0.02268 | !A,A: 1.52(1.11, 2.07) | 0.01310 | G: 1.46(1.09, 1.96) | 527, 660 |
| RS4997 | 08: 037941926 | ADRB3 | 0.01896 | !G,G: 1.54(1.13, 2.11) | 0.00978 | T: 1.48(1.1, 1.99) | 527, 660 |
| RS4994 | 08: 037942955 | ADRB3 | 0.00827 | !T,T: 1.6(1.17, 2.19) | 0.00500 | C: 1.53(1.14, 2.06) | 527, 660 |
| RS2288696 | 08: 038405382 | FGFR1 | 0.05178 | !C,C: 1.34(1.06, 1.69) | 0.03641 | T: 1.23(1.01, 1.48) | 527, 659 |
| RS3242 | 08: 041238711 | SFRP1 | 0.04320 | !C,C: 1.35(1.07, 1.7) | 0.05060 | T: 1.19(1, 1.41) | 527, 660 |
| RS892413 | 08: 042733535 | CHRNA6 | 0.04354 | !A,A: 2.21(1.14, 4.27) | 0.43015 | C: 1.09(0.89, 1.34) | 527, 659 |
| RS1440363 | 08: 073740057 | KCNB2 | 0.02043 | !T,T: 2.27(1.18, 4.38) | 0.76023 | C: 1.03(0.85, 1.26) | 527, 660 |
| RS1440332 | 08: 073897644 | KCNB2 | 0.01256 | !C,T: 1.42(1.13, 1.79) | 0.76856 | C: 1.03(0.87, 1.21) | 526, 659 |
| RS1054135 | 08: 082553315 | FABP4 | 0.11091 | G,G: 1.37(1.03, 1.82) | 0.04122 | G: 1.32(1.02, 1.72) | 527, 659 |
| RS1824717 | 08: 089145095 | MMP16 | 0.00839 | A,A: 1.44(1.11, 1.87) | 0.00193 | A: 1.3(1.1, 1.52) | 527, 660 |
| RS2664361 | 08: 089200925 | MMP16 | 0.07146 | A,A: 1.34(1.04, 1.73) | 0.02067 | A: 1.21(1.03, 1.43) | 527, 660 |
| RS1996637 | 08: 089227035 | MMP16 | 0.04874 | T,T: 1.36(1.06, 1.76) | 0.01315 | T: 1.23(1.05, 1.45) | 527, 660 |
| RS405734 | 08: 090837565 | RIPK2 | 0.03007 | !A,G: 1.36(1.08, 1.71) | 0.13639 | G: 1.14(0.96, 1.35) | 516, 643 |
| RS39499 | 08: 090839744 | RIPK2 | 0.03521 | !A,G: 1.36(1.08, 1.71) | 0.22353 | G: 1.11(0.94, 1.31) | 527, 660 |
| RS263255 | 08: 131893479 | ADCY8 | 0.04887 | A,G: 1.34(1.07, 1.69) | 0.64401 | A: 1.04(0.88, 1.23) | 525, 659 |
| RS3793391 | 08: 131903685 | ADCY8 | 0.06467 | G,G: 1.26(1, 1.6) | 0.02239 | G: 1.26(1.03, 1.53) | 527, 658 |
| RS1509843 | 08: 131910843 | ADCY8 | 0.02256 | !G,G: 1.89(1.08, 3.31) | 0.00846 | A: 1.3(1.07, 1.58) | 527, 660 |
| RS2100644 | 08: 133375983 | KCNQ3 | 0.07274 | !C,C: 1.31(1.02, 1.69) | 0.02233 | T: 1.21(1.03, 1.43) | 526, 658 |
| RS4128450 | 08: 143628473 | BAI1 | 0.06628 | !A,A: 2.02(1.01, 4.05) | 0.02616 | G: 1.28(1.03, 1.6) | 527, 660 |
| RS6144 | 09: 002635557 | VLDLR | 0.04154 | A,T: 1.39(1.07, 1.79) | 0.17187 | T: 1.17(0.94, 1.44) | 527, 660 |
| RS2734602 | 09: 033790934 | PRSS3 | 0.00192 | !T,T: 9.55(1.79, 50.78) | 0.40977 | C: 1.11(0.88, 1.4) | 527, 660 |
| RS1323361 | 09: 076366054 | RORB | 0.12559 | G,G: 1.28(0.97, 1.71) | 0.03791 | G: 1.19(1.01, 1.4) | 527, 660 |
| RS1327832 | 09: 076520742 | TRPM6 | 0.11467 | !G,G: 1.28(1.01, 1.61) | 0.03308 | A: 1.2(1.02, 1.43) | 527, 660 |
| RS10746718 | 09: 085501603 | UBQLN1 | 0.03156 | G,G: 1.38(1.09, 1.74) | 0.00883 | G: 1.29(1.07, 1.57) | 527, 660 |
| RS7874980 | 09: 085514724 | LOC645542, | 0.02988 | C,C: 1.38(1.09, 1.74) | 0.00761 | C: 1.3(1.07, 1.58) | 527, 660 |
| | | UBQLN1 | | | | | |
| RS4877368 | 09: 089419393 | DAPK1 | 0.04018 | !T,T: 1.96(1.15, 3.33) | 0.19173 | C: 1.14(0.94, 1.37) | 527, 660 |
| RS3118873 | 09: 089454146 | DAPK1 | 0.13169 | !G,G: 1.31(0.98, 1.76) | 0.04608 | A: 1.18(1, 1.39) | 527, 660 |
| RS2149380 | 09: 089484394 | DAPK1 | 0.07671 | T,T: 1.39(1.05, 1.83) | 0.03818 | T: 1.19(1.01, 1.4) | 527, 660 |
| RS3128494 | 09: 089502397 | DAPK1 | 0.08647 | G,G: 1.28(1.02, 1.62) | 0.02392 | G: 1.22(1.03, 1.44) | 527, 660 |
| RS1056719 | 09: 089511843 | DAPK1 | 0.11125 | !G,G: 1.35(0.97, 1.9) | 0.03665 | A: 1.2(1.01, 1.42) | 527, 659 |
| RS1405209 | 09: 101625366 | NR4A3 | 0.07292 | !A,A: 1.33(1.05, 1.68) | 0.04763 | G: 1.19(1, 1.41) | 527, 660 |
| RS1526267 | 09: 101629566 | NR4A3 | 0.01918 | !A,A: 1.88(1.15, 3.07) | 0.00964 | G: 1.28(1.06, 1.54) | 527, 660 |
| RS1323411 | 09: 103468920 | GRIN3A | 0.11702 | !C,C: 1.25(0.98, 1.58) | 0.03204 | T: 1.2(1.02, 1.41) | 526, 660 |
| RS1323434 | 09: 103472114 | GRIN3A | 0.11441 | !G,G: 1.25(0.98, 1.59) | 0.03210 | A: 1.2(1.02, 1.41) | 527, 660 |
| RS2515618 | 09: 106718618 | ABCA1 | 0.08455 | !G,G: 1.28(1.02, 1.61) | 0.02360 | A: 1.25(1.03, 1.5) | 527, 660 |
| RS2776 | 09: 112046109 | TXN | 0.04677 | A,A: 1.43(1.09, 1.88) | 0.03508 | A: 1.19(1.01, 1.4) | 527, 660 |
| RS3001131 | 09: 112474655 | MUSK | 0.05712 | G,G: 1.36(1.04, 1.77) | 0.01857 | G: 1.22(1.03, 1.43) | 527, 660 |
| RS1007000 | 09: 112702502 | EDG2 | 0.08303 | !C,C: 1.31(1.03, 1.66) | 0.02347 | T: 1.26(1.04, 1.53) | 527, 660 |
| RS8046 | 09: 124195229 | PTGS1 | 0.04403 | G,G: 8.82(0.45, 171.03) | 0.40716 | A: 1.18(0.82, 1.7) | 527, 660 |
| RS3802353 | 09: 133728596 | CRSP8 | 0.08999 | T,T: 1.31(1.04, 1.65) | 0.03984 | T: 1.2(1.01, 1.42) | 527, 660 |
| RS4266768 | 09: 133881946 | CRSP8 | 0.02447 | C,C: 1.69(1.16, 2.46) | 0.12072 | C: 1.15(0.97, 1.37) | 526, 660 |
| RS4617289 | 09: 133916779 | CRSP8 | 0.04204 | T,T: 1.62(1.12, 2.34) | 0.13293 | T: 1.14(0.96, 1.36) | 527, 660 |
| RS1611114 | 09: 135490024 | DBH | 0.03080 | T,T: 2.02(1.21, 3.36) | 0.09305 | T: 1.18(0.97, 1.42) | 527, 660 |
| RS6479643 | 09: 135504489 | DBH | 0.06647 | !C,C: 1.43(1.04, 1.97) | 0.02223 | G: 1.22(1.03, 1.44) | 526, 660 |
| RS2519078 | 09: 135906177 | BRD3 | 0.01497 | !C,C: 1.62(1.16, 2.26) | 0.12594 | T: 1.14(0.97, 1.35) | 527, 660 |
| RS2519081 | 09: 135914321 | LOC649659, | 0.02792 | !G,G: 1.59(1.13, 2.23) | 0.09625 | C: 1.15(0.98, 1.36) | 527, 658 |
| | | BRD3 | | | | | |
| RS1556417 | 09: 137812473 | KCNT1 | 0.12425 | A,A: 1.58(0.97, 2.59) | 0.04586 | A: 1.22(1.01, 1.48) | 527, 660 |
| RS12238250 | 09: 139166503 | GRIN1 | 0.03418 | !A,A: 1.48(1.06, 2.06) | 0.01450 | C: 1.49(1.08, 2.05) | 527, 660 |
| RS4880782 | 10: 001209153 | LOC642927 | 0.00694 | !G,G: 1.47(1.15, 1.87) | 0.00164 | A: 1.3(1.11, 1.54) | 527, 660 |
| RS1392827 | 10: 001234414 | ADARB2 | 0.02926 | !A,A: 1.38(1.09, 1.73) | 0.01776 | G: 1.25(1.04, 1.51) | 527, 660 |
| RS3793734 | 10: 001410963 | ADARB2 | 0.08905 | !G,G: 1.35(1, 1.82) | 0.02724 | A: 1.2(1.02, 1.42) | 525, 658 |
| RS2892334 | 10: 001416613 | ADARB2 | 0.12593 | !T,T: 1.32(0.98, 1.77) | 0.04144 | G: 1.19(1.01, 1.4) | 526, 660 |
| RS1986608 | 10: 001426204 | ADARB2 | 0.09905 | !T,T: 1.32(0.98, 1.78) | 0.03029 | C: 1.2(1.02, 1.41) | 527, 659 |
| RS2387952 | 10: 001657365 | ADARB2 | 0.00716 | A,A: 1.59(1.19, 2.11) | 0.00085 | A: 1.54(1.19, 1.98) | 526, 660 |
| RS3750685 | 10: 001702209 | ADARB2 | 0.13889 | !C,C: 1.24(0.99, 1.56) | 0.04671 | T: 1.21(1.01, 1.46) | 527, 660 |
| RS2892394 | 10: 001730278 | ADARB2 | 0.04405 | C,C: 1.81(1.1, 3) | 0.02273 | C: 1.25(1.03, 1.51) | 527, 660 |
| RS590467 | 10: 006516741 | PRKCQ | 0.00671 | !A,G: 1.72(1.16, 2.54) | 0.01671 | A: 1.59(1.09, 2.31) | 527, 660 |
| RS10508307 | 10: 006537313 | PRKCQ | 0.00037 | !C,T: 1.6(1.24, 2.07) | 0.09022 | C: 1.21(0.97, 1.5) | 527, 660 |
| RS604663 | 10: 006544132 | PRKCQ | 0.04439 | A,A: 1.37(1.06, 1.76) | 0.10587 | A: 1.14(0.97, 1.35) | 526, 660 |
| RS472317 | 10: 006572184 | PRKCQ | 0.05498 | T,T: 1.47(1.07, 2) | 0.02487 | T: 1.21(1.03, 1.43) | 526, 659 |
| RS648778 | 10: 006574598 | PRKCQ | 0.02625 | T,T: 1.66(1.16, 2.37) | 0.02893 | T: 1.21(1.02, 1.44) | 527, 660 |
| RS943451 | 10: 006661779 | PRKCQ, | 0.11805 | A,A: 1.44(0.98, 2.11) | 0.04647 | A: 1.19(1, 1.42) | 527, 660 |
| | | LOC439949 | | | | | |
| RS7074893 | 10: 012446905 | CAMK1D | 0.01098 | T,T: 1.61(1.16, 2.24) | 0.00189 | T: 1.63(1.19, 2.21) | 523, 656 |
| RS7073375 | 10: 012473948 | CAMK1D | 0.04622 | !C,C: 1.38(1.08, 1.78) | 0.01814 | T: 1.31(1.05, 1.64) | 527, 660 |
| RS2768451 | 10: 012530792 | CAMK1D | 0.11490 | C,C: 1.37(0.99, 1.91) | 0.03585 | C: 1.4(1.03, 1.92) | 526, 658 |
| RS2104588 | 10: 012537587 | CAMK1D | 0.11316 | C,C: 1.32(0.99, 1.77) | 0.03415 | C: 1.34(1.03, 1.74) | 527, 660 |
| RS1543595 | 10: 012555829 | CAMK1D | 0.04544 | !C,C: 2.55(1.17, 5.58) | 0.09849 | T: 1.19(0.97, 1.47) | 527, 660 |
| RS2768429 | 10: 012564354 | CAMK1D | 0.13333 | !C,C: 1.32(0.99, 1.76) | 0.04715 | T: 1.32(1.01, 1.72) | 527, 660 |
| RS7913187 | 10: 012570304 | CAMK1D | 0.07860 | !G,G: 1.33(1.04, 1.7) | 0.03453 | C: 1.26(1.02, 1.56) | 527, 660 |
| RS2768438 | 10: 012575062 | CAMK1D | 0.10642 | C,C: 1.23(0.98, 1.55) | 0.03603 | C: 1.21(1.01, 1.45) | 526, 660 |
| RS2768463 | 10: 012613112 | CAMK1D | 0.06531 | !G,G: 1.54(1.06, 2.24) | 0.03530 | A: 1.21(1.02, 1.43) | 526, 660 |
| RS2648708 | 10: 012641445 | CAMK1D | 0.01878 | !C,C: 1.56(1.14, 2.15) | 0.00691 | T: 1.52(1.12, 2.05) | 527, 659 |
| RS1001486 | 10: 018782483 | CACNB2 | 0.06943 | !A,A: 1.28(1.02, 1.61) | 0.02320 | G: 1.23(1.03, 1.47) | 527, 660 |
| RS11259812 | 10: 047039236 | CTSLL2, | 0.15987 | A,A: 1.27(0.97, 1.65) | 0.04872 | A: 1.27(1, 1.61) | 526, 655 |
| | | FAM21D, | | | | | |
| | | LOC338598 | | | | | |
| RS2574957 | 10: 047564039 | RP11-98I6.3, | 0.05829 | T,T: 1.41(1.07, 1.87) | 0.02750 | T: 1.21(1.02, 1.42) | 509, 614 |
| | | ASAH2 | | | | | |
| RS3810950 | 10: 050494625 | CHAT | 0.04762 | !A,G: 1.35(1.07, 1.71) | 0.14394 | G: 1.15(0.95, 1.38) | 527, 660 |
| RS293252 | 10: 052874515 | PRKG1 | 0.00166 | A,G: 1.49(1.18, 1.88) | 0.96549 | A: 1(0.85, 1.19) | 527, 660 |
| RS10508938 | 10: 052887821 | PRKG1 | 0.02276 | C,T: 1.38(1.08, 1.77) | 0.20847 | C: 1.14(0.93, 1.4) | 527, 660 |
| RS7100647 | 10: 053276294 | PRKG1 | 0.02286 | G,G: 1.57(1.12, 2.2) | 0.24528 | G: 1.11(0.94, 1.31) | 527, 660 |
| RS1937711 | 10: 053285285 | PRKG1 | 0.08865 | !C,C: 1.45(1.03, 2.04) | 0.04492 | G: 1.19(1.01, 1.41) | 527, 659 |
| RS7071831 | 10: 053297839 | PRKG1 | 0.03100 | T,T: 1.78(1.06, 3.01) | 0.03352 | T: 1.75(1.05, 2.94) | 527, 660 |
| RS10823960 | 10: 053446370 | PRKG1 | 0.12932 | A,A: 1.4(1.01, 1.95) | 0.04169 | A: 1.38(1.01, 1.9) | 527, 660 |
| RS10762554 | 10: 053516749 | PRKG1 | 0.04669 | A,A: 1.42(1.07, 1.88) | 0.12451 | A: 1.14(0.97, 1.34) | 529, 649 |
| RS17561995 | 10: 053536601 | PRKG1 | 0.06468 | !A,A: 1.29(1.01, 1.65) | 0.02207 | G: 1.28(1.04, 1.59) | 527, 660 |
| RS4747082 | 10: 072136936 | ADAMTS14 | 0.03683 | A,A: | 0.87333 | A: 1.03(0.75, 1.41) | 527, 660 |
| | | | | 13.91(0.77, 252.04) | | | |
| RS2249814 | 10: 073253526 | PSAP | 0.08108 | !G,G: 1.75(1, 3.08) | 0.03866 | C: 1.23(1.01, 1.49) | 527, 660 |
| RS1873695 | 10: 078364219 | KCNMA1 | 0.05853 | G,G: 1.38(1.06, 1.79) | 0.01580 | G: 1.34(1.06, 1.69) | 527, 660 |
| RS4933813 | 10: 083679105 | NRG3 | 0.07881 | !T,T: 1.46(1.01, 2.09) | 0.02622 | C: 1.22(1.02, 1.44) | 527, 660 |
| RS1474 | 10: 083964622 | NRG3 | 0.05510 | !G,G: 1.31(1.03, 1.67) | 0.01444 | C: 1.23(1.04, 1.45) | 527, 659 |
| RS10509454 | 10: 084250023 | NRG3 | 0.09411 | G,G: 1.35(1.03, 1.78) | 0.02163 | G: 1.34(1.04, 1.71) | 527, 660 |
| RS2184577 | 10: 084442753 | NRG3 | 0.08679 | !A,A: 1.3(1.04, 1.64) | 0.04858 | G: 1.19(1, 1.42) | 527, 660 |
| RS7902829 | 10: 084566021 | NRG3 | 0.02656 | A,C: 1.49(1.05, 2.1) | 0.03219 | A: 1.45(1.04, 2.02) | 523, 637 |
| RS10509522 | 10: 087363843 | GRID1 | 0.06804 | T,T: 1.28(1, 1.63) | 0.02146 | T: 1.28(1.04, 1.57) | 527, 660 |
| RS733758 | 10: 087402105 | GRID1 | 0.00577 | !C,T: 1.76(1.15, 2.69) | 0.03870 | T: 1.53(1.03, 2.29) | 527, 660 |
| RS7912559 | 10: 087724804 | GRID1 | 0.04543 | G,G: 1.47(1.06, 2.04) | 0.34992 | G: 1.09(0.92, 1.28) | 527, 660 |
| RS1870168 | 10: 087930977 | GRID1 | 0.02203 | !A,G: 1.38(1.1, 1.75) | 0.04588 | A: 1.2(1.01, 1.43) | 526, 660 |
| RS2607829 | 10: 087941666 | GRID1 | 0.03807 | !A,G: 1.38(1.09, 1.77) | 0.10038 | A: 1.18(0.97, 1.44) | 525, 659 |
| RS10887578 | 10: 088086027 | GRID1 | 0.00751 | !C,C: 1.55(1.18, 2.03) | 0.00335 | G: 1.28(1.09, 1.5) | 527, 660 |
| RS10509530 | 10: 088091347 | GRID1 | 0.13101 | A,A: 1.63(0.92, 2.9) | 0.04894 | A: 1.24(1.01, 1.52) | 527, 659 |
| RS3814614 | 10: 088118261 | GRID1 | 0.03572 | !T,T: 1.45(1.1, 1.91) | 0.04297 | C: 1.18(1.01, 1.39) | 527, 660 |
| RS1234225 | 10: 089663549 | PTEN | 0.04108 | A,G: 1.34(1.07, 1.69) | 0.76099 | A: 1.03(0.87, 1.22) | 527, 660 |
| RS532678 | 10: 089713322 | PTEN | 0.04108 | C,T: 1.34(1.07, 1.69) | 0.76099 | T: 1.03(0.87, 1.22) | 527, 660 |
| RS982764 | 10: 090759978 | FAS | 0.07458 | !A,A: 1.32(1.05, 1.66) | 0.04553 | G: 1.19(1.01, 1.41) | 527, 660 |
| RS1832197 | 10: 094288311 | IDE | 0.02410 | A,G: 1.38(1.1, 1.74) | 0.18555 | A: 1.12(0.95, 1.33) | 523, 659 |
| RS1853208 | 10: 096653809 | | 0.03573 | !T,T: 1.45(1.08, 1.94) | 0.01272 | C: 1.23(1.05, 1.45) | 527, 660 |
| RS9332104 | 10: 096688680 | CYP2C9 | 0.04091 | !T,T: 1.36(1.08, 1.71) | 0.01385 | C: 1.28(1.05, 1.55) | 527, 660 |
| RS2253635 | 10: 096690527 | CYP2C9 | 0.01950 | !G,G: 1.51(1.07, 2.12) | 0.00502 | A: 1.27(1.08, 1.5) | 525, 660 |
| RS1856908 | 10: 096722721 | CYP2C9 | 0.02012 | !T,T: 1.5(1.06, 2.1) | 0.00507 | G: 1.27(1.07, 1.5) | 527, 660 |
| RS1934969 | 10: 096738485 | CYP2C9 | 0.04478 | !A,A: 1.44(1.06, 1.95) | 0.01539 | T: 1.23(1.04, 1.45) | 527, 659 |
| RS7923726 | 10: 101953289 | CHUK | 0.07427 | !G,G: 1.33(1.01, 1.76) | 0.02048 | T: 1.21(1.03, 1.43) | 527, 660 |
| RS4919613 | 10: 103895769 | PPRC1 | 0.02202 | G,G: 1.42(1.11, 1.81) | 0.01200 | G: 1.31(1.06, 1.63) | 527, 660 |
| RS521674 | 10: 112825580 | ADRA2A | 0.00965 | A,A: 1.4(1.12, 1.77) | 0.00188 | A: 1.33(1.11, 1.59) | 527, 660 |
| RS553668 | 10: 112829569 | ADRA2A | 0.00093 | C,C: 1.62(1.26, 2.08) | 0.00061 | C: 1.46(1.18, 1.82) | 527, 660 |
| RS10885348 | 10: 114197470 | VTI1A, | 0.08915 | !G,G: 2.11(0.9, 4.94) | 0.04085 | A: 1.27(1.01, 1.59) | 527, 660 |
| | | ZDHHC6 | | | | | |
| RS7075980 | 10: 114198266 | VTI1A, | 0.02397 | A,T: 1.35(1.07, 1.72) | 0.52377 | T: 1.07(0.88, 1.29) | 527, 660 |
| | | ZDHHC6 | | | | | |
| RS10509964 | 10: 114260464 | VTI1A | 0.06907 | !A,A: 1.34(1.01, 1.78) | 0.02305 | T: 1.36(1.05, 1.76) | 527, 660 |
| RS7894915 | 10: 114267029 | VTI1A | 0.06907 | !C,C: 1.34(1.01, 1.78) | 0.02305 | G: 1.36(1.05, 1.76) | 527, 660 |
| RS7081965 | 10: 114274633 | VTI1A | 0.03431 | !A,A: 1.37(1.09, 1.72) | 0.01822 | T: 1.25(1.04, 1.51) | 527, 660 |
| RS11196056 | 10: 114435698 | VTI1A | 0.03603 | !G,G: 2.38(1.17, 4.85) | 0.03933 | A: 1.26(1.01, 1.56) | 519, 651 |
| RS6585171 | 10: 114445495 | VTI1A | 0.00473 | C,G: 1.81(1.21, 2.69) | 0.01505 | G: 1.61(1.1, 2.35) | 527, 660 |
| RS3802554 | 10: 114455760 | VTI1A | 0.04722 | C,T: 1.56(1.03, 2.37) | 0.10191 | T: 1.4(0.94, 2.09) | 527, 660 |
| RS7088796 | 10: 114476611 | VTI1A | 0.01489 | A,G: 1.75(1.17, 2.62) | 0.02532 | A: 1.55(1.06, 2.27) | 524, 657 |
| RS7901695 | 10: 114744078 | TCF7L2 | 0.01114 | !T,T: 1.36(1.08, 1.71) | 0.00241 | C: 1.32(1.1, 1.57) | 527, 660 |
| RS7903146 | 10: 114748339 | TCF7L2 | 0.01834 | !C,C: 1.36(1.08, 1.71) | 0.00445 | T: 1.3(1.09, 1.56) | 527, 660 |
| RS4132670 | 10: 114757761 | TCF7L2 | 0.01464 | T,T: 1.63(1.1, 2.43) | 0.00341 | T: 1.3(1.09, 1.55) | 527, 659 |
| RS12255372 | 10: 114798892 | TCF7L2 | 0.13790 | !G,G: 1.23(0.98, 1.54) | 0.04627 | T: 1.21(1.01, 1.44) | 527, 660 |
| RS11196213 | 10: 114811544 | TCF7L2 | 0.10892 | !C,C: 1.27(0.99, 1.62) | 0.02486 | T: 1.21(1.02, 1.42) | 527, 660 |
| RS10509969 | 10: 114903549 | TCF7L2 | 0.08967 | A,A: 1.29(1.02, 1.64) | 0.02071 | A: 1.26(1.04, 1.54) | 527, 660 |
| RS10509970 | 10: 114904903 | TCF7L2 | 0.08267 | T,T: 1.28(1.01, 1.61) | 0.02140 | T: 1.26(1.04, 1.53) | 536, 666 |
| RS2240776 | 10: 119297514 | EMX2 | 0.02671 | A,T: 1.36(1.08, 1.71) | 0.07054 | A: 1.17(0.99, 1.38) | 527, 660 |
| RS10788116 | 10: 122473397 | FGFR2 | 0.01136 | !C,T: 1.43(1.13, 1.81) | 0.08215 | C: 1.16(0.98, 1.38) | 526, 659 |
| RS2250266 | 10: 122887903 | FGFR2 | 0.01296 | !A,G: 1.43(1.13, 1.8) | 0.30790 | A: 1.09(0.93, 1.29) | 527, 659 |
| RS7085142 | 10: 122929364 | FGFR2 | 0.03240 | !C,T: 1.35(1.07, 1.7) | 0.07101 | C: 1.17(0.99, 1.38) | 527, 660 |
| RS4752556 | 10: 123169856 | FGFR2 | 0.04507 | !G,G: 1.3(1.03, 1.64) | 0.01090 | A: 1.28(1.06, 1.55) | 527, 657 |
| RS2981427 | 10: 123307721 | FGFR2 | 0.00531 | !G,G: 1.45(1.15, 1.84) | 0.00078 | A: 1.33(1.13, 1.57) | 527, 660 |
| RS1278354 | 10: 127763965 | ADAM12 | 0.01357 | !G,G: 1.44(1.13, 1.82) | 0.00924 | A: 1.25(1.06, 1.48) | 527, 660 |
| RS1878030 | 10: 127776499 | ADAM12 | 0.06788 | !A,A: 1.34(1.05, 1.7) | 0.02877 | G: 1.2(1.02, 1.42) | 527, 659 |
| RS1278302 | 10: 127788941 | ADAM12 | 0.09230 | !C,C: 1.33(1.01, 1.76) | 0.02530 | A: 1.21(1.02, 1.42) | 527, 660 |
| RS1278329 | 10: 127813141 | ADAM12 | 0.13695 | !G,G: 1.32(0.97, 1.79) | 0.04971 | T: 1.18(1, 1.39) | 527, 660 |
| RS2995313 | 10: 134933703 | ADAM8 | 0.00985 | !T,T: 1.38(1.08, 1.76) | 0.00384 | C: 1.37(1.11, 1.7) | 524, 657 |
| RS2275725 | 10: 134939025 | ADAM8 | 0.01680 | !G,G: 1.47(1.11, 1.95) | 0.00457 | A: 1.45(1.12, 1.88) | 527, 660 |
| RS11042484 | 11: 001274888 | TOLLIP | 0.04583 | !G,G: 1.51(1.08, 2.11) | 0.01428 | A: 1.5(1.09, 2.06) | 522, 658 |
| RS231915 | 11: 002705591 | KCNQ1 | 0.03801 | C,T: 1.34(1.06, 1.68) | 0.89954 | T: 1.01(0.86, 1.19) | 527, 660 |
| RS2075583 | 11: 006380206 | APBB1 | 0.01569 | !G,G: 1.71(1.11, 2.62) | 0.68416 | C: 1.04(0.87, 1.24) | 527, 660 |
| RS2890 | 11: 006577801 | KIAA0409 | 0.04149 | !T,T: 1.54(1.1, 2.16) | 0.00943 | C: 1.53(1.11, 2.09) | 527, 660 |
| RS6256 | 11: 013470629 | PTH | 0.08957 | !C,C: 1.34(1.04, 1.73) | 0.02679 | A: 1.29(1.03, 1.62) | 527, 660 |
| RS2299637 | 11: 017732315 | KCNC1 | 0.00945 | !T,T: 1.87(1.24, 2.83) | 0.14063 | G: 1.14(0.96, 1.36) | 527, 660 |
| RS211109 | 11: 017990774 | SERGEF | 0.04883 | T,T: 1.28(1.02, 1.62) | 0.01272 | T: 1.26(1.05, 1.51) | 527, 660 |
| RS2237907 | 11: 018005769 | L29306, | 0.00177 | G,G: 1.81(1.32, 2.5) | 0.00954 | G: 1.25(1.06, 1.48) | 527, 660 |
| | | TPH1 | | | | | |
| RS172423 | 11: 018012801 | L29306, | 0.06626 | T,T: 1.31(1.04, 1.65) | 0.01792 | T: 1.25(1.04, 1.5) | 527, 660 |
| | | TPH1 | | | | | |
| RS1824017 | 11: 018119521 | MRGPRX3 | 0.11959 | C,C: 1.27(1, 1.6) | 0.03343 | C: 1.23(1.02, 1.49) | 526, 660 |
| RS2468832 | 11: 018120458 | MRGPRX3 | 0.09762 | G,G: 1.29(1.02, 1.62) | 0.02522 | G: 1.24(1.03, 1.5) | 527, 660 |
| RS3758807 | 11: 020595107 | SLC6A5 | 0.02079 | !A,G: 1.34(1.07, 1.69) | 0.61145 | A: 1.05(0.89, 1.24) | 527, 660 |
| RS7046 | 11: 033683556 | CD59 | 0.05286 | !C,C: 1.36(1.07, 1.73) | 0.03577 | T: 1.2(1.01, 1.42) | 513, 649 |
| RS831637 | 11: 033694843 | CD59 | 0.06129 | !A,A: 1.94(0.99, 3.8) | 0.02526 | G: 1.28(1.03, 1.58) | 536, 666 |
| RS831630 | 11: 033699595 | CD59 | 0.03556 | G,G: 1.37(1.09, 1.72) | 0.01467 | G: 1.25(1.05, 1.48) | 527, 660 |
| RS704700 | 11: 033700110 | CD59 | 0.08050 | !C,C: 1.93(0.98, 3.77) | 0.03677 | T: 1.26(1.02, 1.55) | 527, 660 |
| RS831625 | 11: 033703012 | CD59 | 0.05672 | !C,C: 1.35(1.06, 1.71) | 0.02485 | G: 1.21(1.03, 1.43) | 527, 660 |
| RS2273121 | 11: 033714346 | CD59 | 0.03176 | C,C: 1.37(1.09, 1.72) | 0.01280 | C: 1.26(1.05, 1.51) | 535, 667 |
| RS831600 | 11: 033714517 | CD59 | 0.10917 | !G,G: 1.26(1, 1.58) | 0.03390 | A: 1.21(1.02, 1.44) | 526, 660 |
| RS831603 | 11: 033715214 | CD59 | 0.14414 | !A,A: 1.24(0.98, 1.57) | 0.04529 | G: 1.19(1.01, 1.4) | 527, 660 |
| RS1718067 | 11: 033716246 | CD59 | 0.09910 | !T,T: 1.27(1.01, 1.6) | 0.03039 | C: 1.21(1.02, 1.44) | 526, 660 |
| RS331455 | 11: 036497125 | TRAF6 | 0.04234 | !C,C: 1.79(1.14, 2.82) | 0.06721 | T: 1.19(0.99, 1.43) | 526, 660 |
| RS3781625 | 11: 047399656 | PSMC3 | 0.03296 | !G,G: 1.55(1.06, 2.27) | 0.00792 | A: 1.27(1.06, 1.51) | 527, 660 |
| RS544446 | 11: 057227420 | MED19 | 0.01072 | !C,T: 1.43(1.12, 1.83) | 0.25444 | T: 1.12(0.92, 1.36) | 527, 660 |
| RS9420 | 11: 057266870 | C11ORF31, | 0.03566 | A,A: 1.57(1.08, 2.28) | 0.45730 | A: 1.07(0.9, 1.27) | 527, 660 |
| | | LOC643376, | | | | | |
| | | LOC648278, | | | | | |
| | | TXNDC14 | | | | | |
| RS6591561 | 11: 059826752 | MS4A4A | 0.05194 | !A,A: 1.3(1.03, 1.64) | 0.01322 | G: 1.25(1.05, 1.5) | 527, 660 |
| RS11230563 | 11: 060532785 | CD6 | 0.00420 | T,T: 1.89(1.31, 2.75) | 0.01415 | T: 1.24(1.05, 1.47) | 527, 660 |
| RS2074227 | 11: 060533357 | CD6 | 0.00097 | C,C: 2.04(1.41, 2.96) | 0.01794 | C: 1.23(1.04, 1.46) | 532, 650 |
| RS12360861 | 11: 060533649 | CD6 | 0.03664 | A,A: 2.26(1.18, 4.36) | 0.03647 | A: 1.25(1.01, 1.54) | 527, 660 |
| RS2510066 | 11: 063809023 | GPR137, | 0.01623 | !G,G: 1.42(1.12, 1.8) | 0.02192 | A: 1.22(1.03, 1.44) | 527, 660 |
| | | BAD | | | | | |
| RS645078 | 11: 063891874 | RPS6KA4 | 0.04679 | !A,A: 1.35(1.06, 1.71) | 0.06234 | C: 1.17(0.99, 1.38) | 527, 660 |
| RS475032 | 11: 063897313 | RPS6KA4 | 0.04912 | !C,C: 1.35(1.06, 1.71) | 0.05664 | G: 1.18(1, 1.39) | 527, 660 |
| RS673224 | 11: 064304591 | SF1, | 0.12928 | A,A: 1.3(0.97, 1.75) | 0.04614 | A: 1.32(1.01, 1.74) | 527, 660 |
| | | LOC644613 | | | | | |
| RS679946 | 11: 064334689 | MEN1 | 0.04144 | !T,T: 1.35(1.03, 1.76) | 0.00805 | A: 1.25(1.06, 1.47) | 524, 659 |
| RSS35111 | 11: 065241913 | HTATIP, | 0.13885 | G,G: 1.29(0.97, 1.73) | 0.04631 | G: 1.18(1, 1.39) | 527, 660 |
| | | AYP1 | | | | | |
| RS1151519 | 11: 065402407 | CTSW | 0.04935 | !C,C: 1.35(1.07, 1.71) | 0.02890 | G: 1.25(1.03, 1.52) | 527, 660 |
| RS658524 | 11: 065403836 | CTSW | 0.04935 | !G,G: 1.35(1.07, 1.71) | 0.02890 | A: 1.25(1.03, 1.52) | 527, 660 |
| RS687672 | 11: 065406560 | CTSW, FIBP | 0.07077 | !C,C: 1.33(1.05, 1.69) | 0.03650 | T: 1.24(1.02, 1.51) | 522, 660 |
| RS507062 | 11: 065418232 | FOSL1 | 0.03004 | C,T: 1.4(1.09, 1.78) | 0.02610 | C: 1.25(1.03, 1.53) | 527, 660 |
| RS622614 | 11: 065419815 | FOSL1 | 0.02706 | C,T: 1.4(1.1, 1.79) | 0.02588 | T: 1.26(1.03, 1.53) | 527, 659 |
| RS11227756 | 11: 066790842 | ADRBK1 | 0.01113 | A,C: 1.72(1.18, 2.51) | 0.01808 | C: 1.54(1.08, 2.2) | 527, 660 |
| RS7480390 | 11: 066926175 | PPP1CA, | 0.03005 | C,G: 1.66(1.14, 2.42) | 0.03137 | G: 1.49(1.05, 2.11) | 527, 660 |
| | | TBC1D10C | | | | | |
| RS587397 | 11: 067923999 | LRP5 | 0.00449 | C,G: 1.66(1.21, 2.27) | 0.01971 | G: 1.4(1.06, 1.86) | 527, 659 |
| RS4988321 | 11: 067930765 | LRP5 | 0.03583 | A,G: 1.56(1.05, 2.33) | 0.09468 | A: 1.41(0.96, 2.06) | 537, 668 |
| RS314750 | 11: 067938604 | LRP5 | 0.11177 | G,G: 1.44(0.98, 2.11) | 0.04009 | G: 1.2(1.01, 1.43) | 527, 660 |
| RS556442 | 11: 067949266 | LRP5 | 0.06367 | G,G: 1.59(1.08, 2.34) | 0.03981 | G: 1.2(1.01, 1.43) | 527, 660 |
| RS3136541 | 11: 068214519 | GAL | 0.02929 | C,C: 1.53(1.09, 2.15) | 0.00996 | C: 1.25(1.06, 1.48) | 527, 660 |
| RS1042577 | 11: 068215046 | GAL | 0.02564 | A,A: 1.51(1.07, 2.13) | 0.00751 | A: 1.26(1.07, 1.49) | 526, 660 |
| RS3136543 | 11: 068215779 | GAL | 0.01996 | G,G: 1.53(1.09, 2.16) | 0.00512 | G: 1.27(1.08, 1.51) | 527, 660 |
| RS2278908 | 11: 068280486 | CPT1A | 0.04898 | A,A: 1.43(1.04, 1.97) | 0.01383 | A: 1.23(1.04, 1.46) | 527, 660 |
| RS1135029 | 11: 071966939 | PDE2A | 0.12770 | !A,A: 1.29(0.97, 1.71) | 0.04219 | G: 1.19(1.01, 1.4) | 526, 659 |
| RS341049 | 11: 071975856 | PDE2A | 0.08155 | G,G: 1.27(1, 1.63) | 0.02206 | G: 1.21(1.03, 1.43) | 527, 660 |
| RS366068 | 11: 072014212 | PDE2A | 0.03059 | !G,G: 1.71(1.13, 2.59) | 0.01887 | A: 1.24(1.04, 1.48) | 527, 660 |
| RS7111188 | 11: 072018414 | PDE2A | 0.03434 | !G,G: 1.32(1.02, 1.69) | 0.01213 | A: 1.33(1.07, 1.66) | 527, 660 |
| RS1009708 | 11: 072022993 | PDE2A | 0.05185 | !G,G: 1.35(1.04, 1.76) | 0.01416 | A: 1.35(1.07, 1.7) | 527, 660 |
| RS3781923 | 11: 072032871 | PDE2A | 0.03579 | !G,G: 1.65(1.13, 2.43) | 0.02997 | A: 1.22(1.02, 1.45) | 527, 660 |
| RS1685355 | 11: 073390871 | UCP3 | 0.02088 | !C,C: 1.45(1.11, 1.88) | 0.07573 | T: 1.16(0.99, 1.36) | 527, 660 |
| RS3781730 | 11: 075585731 | WNT11 | 0.00343 | !T,T: 5.04(1.62, 15.68) | 0.01672 | C: 1.32(1.05, 1.66) | 527, 660 |
| RS1367537 | 11: 083911429 | DLG2 | 0.11548 | !C,C: 1.5(0.95, 2.36) | 0.03554 | G: 1.23(1.02, 1.48) | 527, 660 |
| RS1939110 | 11: 086192720 | PRSS23 | 0.02480 | !T,T: 1.74(1.14, 2.64) | 0.01193 | C: 1.26(1.05, 1.51) | 527, 659 |
| RS2155081 | 11: 086198400 | PRSS23 | 0.06578 | !A,A: 1.8(1.01, 3.21) | 0.02618 | G: 1.26(1.03, 1.53) | 526, 660 |
| RS1024117 | 11: 087896251 | GRM5 | 0.11019 | C,C: 1.29(1, 1.67) | 0.03172 | C: 1.2(1.02, 1.41) | 527, 660 |
| RS2927493 | 11: 087980312 | GRM5 | 0.04422 | T,T: 1.34(1.07, 1.69) | 0.01343 | T: 1.24(1.05, 1.47) | 537, 668 |
| RS673869 | 11: 093179649 | CRSP6 | 0.02019 | !A,A: 1.39(1.1, 1.76) | 0.03129 | G: 1.2(1.02, 1.43) | 527, 660 |
| RS1870019 | 11: 100406683 | PGR | 0.02687 | T,T: 1.38(1.1, 1.74) | 0.01585 | T: 1.26(1.05, 1.51) | 526, 660 |
| RS1042838 | 11: 100438622 | PGR | 0.08920 | !G,G: 1.32(1.03, 1.69) | 0.02926 | T: 1.28(1.03, 1.59) | 526, 660 |
| RS507141 | 11: 100508322 | PGR | 0.10044 | !C,C: 1.31(1.02, 1.67) | 0.02936 | T: 1.28(1.03, 1.59) | 527, 660 |
| RS7931399 | 11: 100828343 | TRPC6 | 0.07642 | !A,A: 1.31(1.03, 1.66) | 0.02222 | C: 1.26(1.04, 1.54) | 527, 660 |
| RS10444351 | 11: 100830056 | TRPC6 | 0.07642 | !G,G: 1.31(1.03, 1.66) | 0.02222 | A: 1.26(1.04, 1.54) | 527, 660 |
| RS7948300 | 11: 100849909 | TRPC6 | 0.00685 | G,G: 2.08(1.2, 3.62) | 0.00240 | G: 1.35(1.11, 1.64) | 526, 660 |
| RS11224782 | 11: 100855026 | TRPC6 | 0.02131 | T,T: 2.02(1.1, 3.72) | 0.00731 | T: 1.33(1.08, 1.63) | 527, 660 |
| RS7938416 | 11: 100859651 | TRPC6 | 0.04210 | !A,A: 1.32(1.05, 1.67) | 0.01034 | G: 1.27(1.06, 1.52) | 527, 660 |
| RS7131328 | 11: 100918232 | TRPC6 | 0.12433 | G,G: 1.28(1.02, 1.62) | 0.04815 | G: 1.19(1, 1.41) | 527, 660 |
| RS7936514 | 11: 100938563 | TRPC6 | 0.04426 | A,A: 1.44(1.08, 1.93) | 0.14555 | A: 1.13(0.96, 1.33) | 527, 660 |
| RS1616349 | 11: 101979677 | MMP20 | 0.00715 | !T,T: 1.48(1.16, 1.88) | 0.00531 | G: 1.34(1.09, 1.65) | 527, 660 |
| RS470344 | 11: 102140448 | MMP10 | 0.01661 | A,T: 1.41(1.12, 1.77) | 0.06334 | A: 1.18(0.99, 1.4) | 526, 660 |
| RS470168 | 11: 102146592 | MMP10 | 0.04012 | !G,G: 1.34(1.07, 1.69) | 0.04714 | A: 1.19(1, 1.42) | 527, 660 |
| RS486055 | 11: 102155634 | MMP10 | 0.13769 | !C,C: 1.3(1.01, 1.66) | 0.04780 | T: 1.24(1, 1.54) | 527, 660 |
| RS470504 | 11: 102163899 | MMP1 | 0.04552 | !T,T: 3(1.05, 8.56) | 0.02988 | C: 1.33(1.03, 1.71) | 537, 668 |
| RS498186 | 11: 102174855 | MMP1 | 0.10559 | A,A: 1.28(1, 1.64) | 0.03214 | A: 1.2(1.02, 1.42) | 522, 637 |
| RS683878 | 11: 102210868 | MMP3 | 0.15121 | G,G: 1.21(0.96, 1.52) | 0.04556 | G: 1.21(1, 1.45) | 527, 660 |
| RS507879 | 11: 104383137 | CASP5 | 0.05013 | G,G: 1.43(1.08, 1.89) | 0.03142 | G: 1.2(1.02, 1.41) | 527, 660 |
| RS1503390 | 11: 104501247 | HIPHUM_302 | 0.09335 | C,C: 1.3(1.03, 1.64) | 0.04587 | C: 1.19(1.01, 1.42) | 527, 660 |
| RS7125415 | 11: 112815891 | DRD2 | 0.01376 | !C,C: 1.56(1.16, 2.11) | 0.00350 | T: 1.52(1.15, 2) | 527, 660 |
| RS1729411 | 11: 116168885 | APOA5 | 0.12216 | C,C: 1.29(0.98, 1.7) | 0.03928 | C: 1.3(1.01, 1.66) | 527, 659 |
| RS620263 | 11: 129556032 | ST14 | 0.04203 | !A,A: 1.35(1.07, 1.7) | 0.01504 | G: 1.25(1.05, 1.5) | 526, 660 |
| RS1548133 | 11: 129821890 | ADAMTS15 | 0.08607 | !A,A: 1.27(1, 1.6) | 0.02357 | G: 1.22(1.03, 1.44) | 527, 659 |
| RS2010851 | 12: 001626903 | WNT5B | 0.01250 | A,C: 1.38(1.1, 1.75) | 0.89378 | C: 1.02(0.85, 1.21) | 526, 659 |
| RS767870 | 12: 001760084 | ADIPOR2 | 0.04436 | !C,C: 3.04(1.18, 7.85) | 0.09827 | T: 1.22(0.96, 1.54) | 527, 660 |
| RS2239068 | 12: 002404727 | CACNA1C | 0.01999 | C,T: 1.34(1.06, 1.7) | 0.84965 | C: 1.02(0.85, 1.23) | 527, 660 |
| RS1076343 | 12: 002423837 | CACNA1C | 0.00455 | !G,G: 2.21(1.26, 3.88) | 0.00129 | A: 1.39(1.14, 1.7) | 527, 659 |
| RS1076344 | 12: 002424249 | CACNA1C | 0.00789 | !C,C: 2.18(1.26, 3.77) | 0.00400 | T: 1.34(1.1, 1.63) | 527, 660 |
| RS1990240 | 12: 002509517 | CACNA1C | 0.03269 | G,G: 1.37(1.09, 1.73) | 0.01174 | G: 1.27(1.05, 1.53) | 527, 659 |
| RS758534 | 12: 004789528 | KCNA6 | 0.05885 | A,A: 1.31(1.03, 1.67) | 0.01534 | A: 1.23(1.04, 1.45) | 527, 660 |
| RS1860419 | 12: 005030124 | KCNA5 | 0.15064 | T,T: 1.25(0.99, 1.57) | 0.04829 | T: 1.21(1, 1.45) | 527, 660 |
| RS2041375 | 12: 006343810 | SCNN1A | 0.03261 | !G,G: 1.36(1.08, 1.72) | 0.00808 | T: 1.27(1.07, 1.52) | 527, 660 |
| RS7301709 | 12: 006949476 | PHB2, EMG1 | 0.02647 | !G,G: 2.15(1.15, 4) | 0.01339 | T: 2.2(1.19, 4.05) | 527, 660 |
| RS12314349 | 12: 012179088 | BCL2L14, | 0.00490 | A,A: 1.53(1.19, 1.98) | 0.00277 | A: 1.4(1.13, 1.75) | 511, 629 |
| | | LRP6 | | | | | |
| RS11054721 | 12: 012222594 | LRP6, | 0.08215 | T,T: 1.31(1.02, 1.69) | 0.02806 | T: 1.2(1.02, 1.41) | 527, 660 |
| | | BCL2L14 | | | | | |
| RS10492120 | 12: 012224619 | BCL2L14, | 0.08762 | T,T: 1.3(1.03, 1.65) | 0.03299 | T: 1.2(1.02, 1.42) | 527, 660 |
| | | LRP6 | | | | | |
| RS1181332 | 12: 012253186 | LRP6, | 0.00925 | A,A: 1.47(1.15, 1.87) | 0.00606 | A: 1.34(1.09, 1.66) | 527, 660 |
| | | BCL2L14 | | | | | |
| RS759748 | 12: 012701921 | GPR19 | 0.00024 | !A,G: 1.72(1.33, 2.23) | 0.00044 | A: 1.48(1.19, 1.83) | 527, 660 |
| RS890 | 12: 013606575 | GRIN2B | 0.02680 | G,T: 1.38(1.1, 1.73) | 0.93395 | G: 1.01(0.86, 1.19) | 527, 660 |
| RS995803 | 12: 013729742 | GRIN2B | 0.02890 | C,T: 1.38(1.1, 1.73) | 0.35524 | T: 1.08(0.92, 1.27) | 527, 660 |
| RS219904 | 12: 013984155 | GRIN2B | 0.07315 | T,T: 1.32(1.01, 1.71) | 0.01844 | T: 1.22(1.04, 1.43) | 526, 659 |
| RS219934 | 12: 014004982 | GRIN2B | 0.05756 | A,A: 1.33(1.02, 1.73) | 0.01320 | A: 1.23(1.05, 1.45) | 527, 660 |
| RS1981781 | 12: 014016750 | GRIN2B | 0.14994 | A,A: 1.25(1, 1.57) | 0.04696 | A: 1.2(1, 1.43) | 527, 660 |
| RS2062165 | 12: 026711004 | ITPR2 | 0.07536 | C,C: 1.34(1.05, 1.72) | 0.02784 | C: 1.28(1.03, 1.59) | 527, 660 |
| RS11173979 | 12: 038537577 | C12ORF40, | 0.04008 | !C,C: 1.53(1.07, 2.18) | 0.02316 | T: 1.49(1.06, 2.09) | 536, 667 |
| | | SLC2A13 | | | | | |
| RS874714 | 12: 042060714 | ADAMTS20 | 0.07528 | !G,G: 1.41(1.04, 1.9) | 0.03411 | A: 1.19(1.01, 1.4) | 527, 660 |
| RS1949083 | 12: 042157496 | ADAMTS20 | 0.03237 | T,T: 6.34(1.09, 36.71) | 0.04039 | T: 1.4(1.01, 1.92) | 527, 660 |
| RS2189480 | 12: 046550095 | VDR | 0.00167 | A,A: 1.87(1.32, 2.65) | 0.00146 | A: 1.32(1.11, 1.56) | 527, 659 |
| RS2849266 | 12: 048652567 | AQP6 | 0.00732 | T,T: 1.71(1.22, 2.41) | 0.00199 | T: 1.66(1.2, 2.3) | 527, 660 |
| RS6580800 | 12: 049854841 | TFCP2 | 0.03826 | !G,G: 1.49(1.04, 2.12) | 0.00883 | A: 1.26(1.06, 1.5) | 527, 660 |
| RS744691 | 12: 050727817 | NR4A1 | 0.03817 | !C,C: 1.31(1.03, 1.65) | 0.01000 | A: 1.29(1.06, 1.57) | 527, 659 |
| RS2272301 | 12: 051874619 | ITGB7 | 0.02414 | G,G: 3.04(1.21, 7.66) | 0.01977 | G: 1.33(1.05, 1.69) | 526, 658 |
| RS3825084 | 12: 051880198 | ITGB7 | 0.01695 | C,C: 3.6(1.35, 9.6) | 0.03989 | C: 1.29(1.02, 1.63) | 527, 660 |
| RS6580942 | 12: 051948891 | ESPL1 | 0.00308 | A,A: 1.5(1.19, 1.89) | 0.00512 | A: 1.28(1.08, 1.53) | 526, 659 |
| RS4758963 | 12: 051950415 | ESPL1 | 0.13090 | T,T: 1.27(0.97, 1.66) | 0.03842 | T: 1.19(1.01, 1.4) | 526, 657 |
| RS1110720 | 12: 051968593 | ESPL1 | 0.00623 | A,A: 1.48(1.17, 1.86) | 0.00647 | A: 1.27(1.07, 1.5) | 527, 660 |
| RS3214023 | 12: 051969253 | ESPL1 | 0.11578 | !T,T: 1.26(1, 1.61) | 0.03453 | C: 1.2(1.02, 1.41) | 526, 660 |
| RS1716966 | 12: 052168141 | MAP3K12 | 0.06421 | C,C: 1.37(1.06, 1.76) | 0.03479 | C: 1.19(1.01, 14) | 527, 660 |
| RS784567 | 12: 052180732 | MAP3K12, | 0.00315 | C,C: 1.6(1.23, 2.09) | 0.00294 | C: 1.28(1.09, 1.5) | 527, 659 |
| | | TARBP2 | | | | | |
| RS784568 | 12: 052213821 | ATF7 | 0.00116 | C,C: 1.68(1.29, 2.21) | 0.00193 | C: 1.3(1.1, 1.52) | 527, 660 |
| RS703817 | 12: 055776095 | NAB2, | 0.13752 | G,G: 1.28(0.98, 1.67) | 0.03477 | G: 1.19(1.01, 1.4) | 527, 659 |
| | | STAT6 | | | | | |
| RS7299766 | 12: 063761468 | WIF1 | 0.12867 | C,C: 1.33(0.99, 1.79) | 0.04504 | C: 1.18(1, 1.39) | 527, 660 |
| RS11108452 | 12: 095197895 | PCTK2 | 0.00760 | !C,C: 2.27(1.18, 4.38) | 0.91734 | C: 1.01(0.83, 1.24) | 527, 660 |
| RS7965892 | 12: 095219307 | PCTK2 | 0.02138 | !T,T: | 0.58236 | T: 1.11(0.78, 1.59) | 494,591 |
| | | | | 10.98(0.62, 195.38) | | | |
| RS2342920 | 12: 123451782 | NCOR2 | 0.12387 | C,C: 1.28(1.01, 1.61) | 0.04639 | C: 1.19(1, 1.41) | 527, 660 |
| RS1244053 | 12: 123456165 | NCOR2 | 0.03495 | C,C: 1.42(1.09, 1.85) | 0.01556 | C: 1.34(1.06, 1.69) | 527, 660 |
| RS2049568 | 12: 130055280 | GPR133 | 0.04574 | !G,G: 1.56(1.1, 2.22) | 0.03462 | C: 1.2(1.02, 1.43) | 520, 651 |
| RS1725804 | 12: 130151491 | GPR133 | 0.04398 | !A,G: 1.37(1.07, 1.74) | 0.07467 | G: 1.19(0.98, 1.44) | 527, 660 |
| RS1041028 | 13: 019621698 | GJA3 | 0.08697 | G,G: 1.27(0.99, 1.62) | 0.02476 | G: 1.21(1.03, 1.42) | 527, 660 |
| RS729137 | 13: 023293662 | MIPEP | 0.08149 | !G,G: 1.3(1.04, 1.64) | 0.02676 | A: 1.22(1.03, 1.46) | 526, 660 |
| RS636171 | 13: 027970703 | FLT1 | 0.10033 | !G,G: 1.3(1.03, 1.63) | 0.03447 | C: 1.23(1.02, 1.48) | 526, 660 |
| RS1747157 | 13: 037265158 | TRPC4 | 0.05808 | C,C: 1.42(1.07, 1.9) | 0.02716 | C: 1.21(1.02, 1.42) | 527, 659 |
| RS4620866 | 13: 048182126 | CYSLTR2 | 0.01409 | !A,A: 1.52(1.14, 2.02) | 0.09798 | G: 1.15(0.98, 1.35) | 527, 660 |
| RS1571360 | 13: 050492202 | GUCY1B2 | 0.09155 | T,T: 1.27(1, 1.62) | 0.02922 | T: 1.2(1.02, 1.42) | 527, 660 |
| RS1328361 | 13: 050492742 | GUCY1B2 | 0.09033 | G,G: 1.26(0.99, 1.61) | 0.02912 | G: 1.2(1.02, 1.42) | 527, 660 |
| RS2031234 | 13: 075038498 | UCHL3 | 0.04743 | !A,G: 1.39(1.07, 1.79) | 0.02891 | A: 1.28(1.03, 1.59) | 527, 660 |
| RS614800 | 13: 100564874 | VGCNL1 | 0.04279 | !A,A: 1.36(1.08, 1.72) | 0.01869 | G: 1.23(1.04, 1.45) | 527, 660 |
| RS2152324 | 13: 100868161 | VGCNL1 | 0.01795 | !A,A: 1.37(1.05, 1.79) | 0.00827 | T: 1.38(1.09, 1.76) | 526, 658 |
| RS1998957 | 13: 102064784 | TPP2 | 0.06234 | G,G: 1.33(1.04, 1.71) | 0.01599 | G: 1.31(1.05, 1.63) | 527, 660 |
| RS1582188 | 13: 104934457 | G30, DAOA | 0.02946 | T,T: 1.33(1.03, 1.7) | 0.00710 | T: 1.34(1.08, 1.66) | 527, 660 |
| RS488703 | 13: 112818877 | F7 | 0.00961 | !C,T: 1.61(1.18, 2.18) | 0.00937 | C: 1.44(1.09, 1.89) | 527, 660 |
| RS3783436 | 14: 022675813 | SLC7A8 | 0.04909 | !T,T: 1.29(1.02, 1.62) | 0.01323 | C: 1.24(1.05, 1.47) | 527, 660 |
| RS999165 | 14: 022677706 | SLC7A8 | 0.01487 | A,A: 1.35(1.07, 1.7) | 0.00296 | A: 1.33(1.1, 1.6) | 527, 660 |
| RS1953722 | 14: 029300389 | PRKD1 | 0.02108 | !C,C: 1.79(1.15, 2.8) | 0.35638 | T: 1.09(0.91, 1.31) | 527, 660 |
| RS17096068 | 14: 029334384 | PRKD1 | 0.04496 | G,G: 4.83(1.19, 19.53) | 0.13539 | G: 1.23(0.94, 1.6) | 527, 660 |
| RS1816628 | 14: 051819186 | PTGDR | 0.06505 | !G,G: 1.36(1.04, 1.79) | 0.01850 | C: 1.22(1.03, 1.43) | 527, 660 |
| RS1254579 | 14: 051866596 | PTGER2 | 0.12255 | !T,T: 1.25(0.99, 1.58) | 0.03833 | C: 1.23(1.01, 1.49) | 527, 660 |
| RS1041644 | 14: 062250857 | KCNH5 | 0.05810 | A,A: 1.36(1.05, 1.75) | 0.01827 | A: 1.22(1.04, 1.43) | 525, 659 |
| RS1048315 | 14: 063762218 | SYNE2 | 0.09280 | T,T: 1.38(1, 1.9) | 0.02841 | T: 1.4(1.04, 1.9) | 526, 660 |
| RS1286920 | 14: 089609412 | KCNK13 | 0.00109 | !C,G: 1.53(1.22, 1.93) | 0.77026 | C: 1.03(0.87, 1.21) | 527, 659 |
| RS2236402 | 14: 089804131 | PSMC1 | 0.04252 | C,G: 1.36(1.08, 1.71) | 0.13478 | C: 1.15(0.96, 1.37) | 527, 660 |
| RS2228575 | 14: 092468764 | CHGA | 0.02801 | G,G: 5.67(1.02, 31.47) | 0.02836 | G: 5.63(1.02, 31.2) | 527, 660 |
| RS6647 | 14: 093917168 | SERPINA1 | 0.08312 | !C,C: 1.75(1.01, 3.03) | 0.03899 | T: 1.23(1.01, 1.49) | 527, 660 |
| RS3748312 | 14: 093924017 | SERPINA1 | 0.00078 | !A,A: 5.03(1.62, 15.65) | 0.00097 | G: 1.46(1.17, 1.84) | 526, 660 |
| RS6116 | 14: 094128215 | SERPINA5 | 0.04033 | !C,C: 1.44(1.09, 1.91) | 0.07507 | A: 1.16(0.99, 1.37) | 527, 659 |
| RS4934 | 14: 094150556 | SERPINA3 | 0.11774 | G,G: 1.27(0.98, 1.63) | 0.03484 | G: 1.19(1.01, 1.4) | 527, 660 |
| RS2268336 | 14: 094152869 | SERPINA3 | 0.00743 | C,C: 1.71(1.21, 2.41) | 0.00469 | C: 1.28(1.08, 1.51) | 527, 660 |
| RS945036 | 14: 095802516 | BDKRB1 | 0.02443 | C,C: 1.39(1.1, 1.75) | 0.01604 | C: 1.24(1.04, 1.48) | 527, 660 |
| RS11704 | 14: 101878408 | C14ORF131 | 0.09746 | C,C: 1.5(1.02, 2.21) | 0.04647 | C: 1.19(1, 1.42) | 527, 660 |
| RS2498794 | 14: 104316296 | AKT1 | 0.10775 | !T,T: 1.32(1.02, 1.71) | 0.04280 | C: 1.18(1.01, 1.39) | 526, 660 |
| RS1557871 | 15: 023132306 | UBE3A | 0.02469 | !C,C: | 0.14449 | T: 1.21(0.94, 1.55) | 527, 660 |
| | | | | 6.77(1.24, 36.94) | | | |
| RS11855196 | 15: 023150130 | UBE3A | 0.00767 | !A,A: 2.88(1.43, 5.77) | 0.11078 | G: 1.18(0.97, 1.45) | 526, 660 |
| RS2340625 | 15: 023190278 | UBE3A | 0.02858 | !C,C: 1.37(1.09, 1.72) | 0.00700 | G: 1.29(1.08, 1.55) | 527, 660 |
| RS1426219 | 15: 024406892 | GABRB3 | 0.01217 | G,G: 1.38(1.08, 1.76) | 0.00240 | G: 1.29(1.1, 1.52) | 527, 660 |
| RS2889395 | 15: 024897145 | GABRG3 | 0.09017 | !A,A: 1.28(1.01, 1.62) | 0.03202 | G: 1.25(1.02, 1.52) | 527, 660 |
| RS1432126 | 15: 024904962 | GABRG3 | 0.06267 | !T,T: 1.31(1.04, 1.66) | 0.02044 | C: 1.27(1.04, 1.55) | 527, 660 |
| RS1429751 | 15: 031904150 | RYR3 | 0.03910 | C,C: 1.51(1.1, 2.07) | 0.02268 | C: 1.22(1.03, 1.43) | 527, 660 |
| RS2306479 | 15: 038326856 | PAK6 | 0.00065 | A,G: 1.53(1.22, 1.93) | 0.26184 | G: 1.1(0.93, 1.29) | 525, 654 |
| RS1912404 | 15: 053999881 | NEDD4 | 0.16141 | !G,G: 1.24(0.99, 1.56) | 0.04928 | A: 1.2(1, 1.44) | 527, 660 |
| RS1509408 | 15: 054073605 | NEDD4 | 0.09476 | A,A: 1.28(1.02, 1.61) | 0.02167 | A: 1.24(1.03, 1.48) | 527, 658 |
| RS7163836 | 15: 058427520 | ANXA2 | 0.05160 | C,C: 1.36(1.07, 1.74) | 0.04954 | C: 1.18(1, 1.39) | 527, 660 |
| RS8024024 | 15: 058471418 | ANXA2 | 0.03364 | !A,A: 1.42(1.1, 1.85) | 0.01369 | C: 1.34(1.07, 1.69) | 508, 618 |
| RS920205 | 15: 058613068 | RORA | 0.14071 | !T,T: 1.27(1, 1.6) | 0.04523 | C: 1.19(1, 1.4) | 527, 660 |
| RS8684 | 15: 064570664 | SNAPC5, | 0.06921 | !C,C: 1.4(1, 1.94) | 0.03593 | T: 1.41(1.03, 1.94) | 525, 659 |
| | | MAP2K1 | | | | | |
| RS1504617 | 15: 067450976 | PAQR5 | 0.01990 | !A,A: 1.73(1.15, 2.62) | 0.00822 | G: 1.71(1.15, 2.55) | 527, 660 |
| RS937722 | 15: 067468887 | PAQR5 | 0.00989 | !C,C: 1.84(1.21, 2.8) | 0.00503 | T: 1.81(1.21, 2.72) | 527, 660 |
| RS3784361 | 15: 067479772 | PAQR5 | 0.00008 | !C,C: 3.41(1.84, 6.32) | 0.00003 | G: 3.41(1.86, 6.28) | 527, 660 |
| RS1499063 | 15: 074061567 | NRG4 | 0.02714 | !A,G: 1.37(1.09, 1.73) | 0.04421 | G: 1.2(1, 1.43) | 527, 660 |
| RS4531696 | 15: 079346671 | IL16 | 0.04219 | A,G: 1.36(1.08, 1.71) | 0.19401 | A: 1.13(0.94, 1.34) | 527, 660 |
| RS1051168 | 15: 083001524 | NMB | 0.04763 | C,C: 1.35(1.06, 1.71) | 0.00999 | C: 1.3(1.06, 1.58) | 527, 660 |
| RS2285527 | 15: 089543804 | SV2B | 0.01674 | !G,T: 1.41(1.12, 1.78) | 0.28348 | T: 1.1(0.93, 1.3) | 527, 660 |
| RS2684778 | 15: 097223362 | IGF1R | 0.04638 | C,T: 1.34(1.06, 1.7) | 0.35237 | T: 1.1(0.91, 1.33) | 527, 660 |
| RS2858002 | 16: 000281886 | AXIN1 | 0.04762 | !C,T: 1.37(1.07, 1.76) | 0.15343 | C: 1.16(0.95, 1.43) | 527, 660 |
| RS758033 | 16: 000337045 | AXIN1 | 0.14367 | G,G: 1.25(0.99, 1.58) | 0.04006 | G: 1.22(1.01, 1.47) | 527, 660 |
| RS929457 | 16: 002592441 | PDPK1, | 0.12948 | T,T: 1.32(1.01, 1.72) | 0.04281 | T: 1.28(1.01, 1.62) | 525, 659 |
| | | LOC124216 | | | | | |
| RS2230739 | 16: 003973437 | ADCY9 | 0.04275 | !G,G: 1.65(1.1, 2.46) | 0.24333 | A: 1.11(0.93, 1.33) | 527, 659 |
| RS1967309 | 16: 004005584 | ADCY9 | 0.07484 | C,C: 1.3(1.03, 1.66) | 0.02095 | C: 1.22(1.03, 1.43) | 526, 660 |
| RS1861191 | 16: 009815567 | GRIN2A | 0.03900 | A,C: 1.31(1.04, 1.66) | 0.68003 | C: 1.04(0.87, 1.24) | 527, 660 |
| RS2173685 | 16: 009960691 | GRIN2A | 0.02897 | C,C: 1.37(1.08, 1.72) | 0.00941 | C: 1.26(1.06, 1.5) | 527, 660 |
| RS2283508 | 16: 016180009 | ABCC6 | 0.03709 | C,T: 1.33(1.06, 1.67) | 0.21363 | C: 1.11(0.94, 1.31) | 524, 659 |
| RS4783361 | 16: 022128305 | EEF2K | 0.00334 | C,C: 1.5(1.19, 1.9) | 0.00383 | C: 1.33(1.1, 1.62) | 527, 660 |
| RS754290 | 16: 022131010 | EEF2K | 0.10578 | A,A: 1.26(1.01, 1.59) | 0.03097 | A: 1.22(1.02, 1.45) | 527, 660 |
| RS11649104 | 16: 022151389 | EEF2K | 0.03071 | G,G: 1.3(1.04, 1.64) | 0.00820 | G: 1.28(1.07, 1.53) | 527, 660 |
| RS2303188 | 16: 022163399 | EEF2K | 0.12260 | G,G: 1.36(1.02, 1.81) | 0.04630 | G: 1.32(1.01, 1.71) | 526, 660 |
| RS4783452 | 16: 022171169 | EEF2K | 0.04220 | !A,A: 1.64(1.05, 2.58) | 0.01513 | G: 1.25(1.05, 1.5) | 527, 660 |
| RS2239339 | 16: 024137386 | PRKCB1 | 0.09773 | G,G: 1.32(1.03, 1.69) | 0.03564 | G: 1.26(1.02, 1.56) | 527, 660 |
| RS4459557 | 16: 031269144 | ITGAX | 0.13637 | C,C: 1.22(0.97, 1.53) | 0.04691 | C: 1.2(1.01, 1.42) | 527, 660 |
| RS170364 | 16: 055967435 | CX3CL1 | 0.04496 | G,T: 1.35(1.07, 1.71) | 0.31803 | T: 1.1(0.91, 1.33) | 525, 660 |
| RS170361 | 16: 055972124 | CX3CL1 | 0.01595 | A,G: 1.44(1.12, 1.86) | 0.12292 | A: 1.18(0.96, 1.45) | 527, 660 |
| RS1004363 | 16: 056153500 | GPR114 | 0.02425 | A,G: 1.35(1.07, 1.7) | 0.89224 | G: 1.02(0.85, 1.21) | 527, 657 |
| RS733463 | 16: 056239845 | GPR56 | 0.04022 | T,T: 1.41(1.03, 1.94) | 0.00964 | T: 1.25(1.06, 1.47) | 526, 660 |
| RS1874625 | 16: 083501642 | CRISPLD2 | 0.03671 | A,C: 1.37(1.08, 1.73) | 0.08580 | C: 1.17(0.98, 1.41) | 537, 668 |
| RS322937 | 17: 003393635 | TRPV3 | 0.13862 | !A,A: 1.27(1, 1.62) | 0.04775 | G: 1.18(1, 1.39) | 524, 660 |
| RS177985 | 17: 003712348 | CAMKK1 | 0.12792 | !G,G: 1.26(0.99, 1.61) | 0.03671 | C: 1.25(1.02, 1.54) | 527, 660 |
| RS2532947 | 17: 003715428 | CAMKK1 | 0.12462 | C,C: 1.37(1, 1.89) | 0.04492 | C: 1.19(1.01, 1.41) | 527, 660 |
| RS2325788 | 17: 003765579 | P2RX1 | 0.02861 | !A,A: 1.52(1.11, 2.07) | 0.11989 | G: 1.14(0.97, 1.35) | 526, 660 |
| RS1807183 | 17: 022976416 | KSR1 | 0.06385 | C,C: 1.34(1.05, 1.7) | 0.02610 | C: 1.25(1.03, 1.53) | 526, 658 |
| RS9897022 | 17: 023578902 | PYY2 | 0.11251 | !C,C: 1.29(1.02, 1.63) | 0.04633 | T: 1.22(1.01, 1.48) | 527, 660 |
| RS434230 | 17: 028547108 | ACCN1 | 0.05777 | !T,T: 1.33(1.04, 1.69) | 0.02069 | A: 1.22(1.03, 1.44) | 527, 660 |
| RS2015086 | 17: 031415730 | CCL18 | 0.01312 | C,T: 1.48(1.13, 1.93) | 0.08441 | C: 1.23(0.97, 1.55) | 527, 660 |
| RS712042 | 17: 031416993 | CCL18 | 0.01374 | A,G: 1.47(1.13, 1.92) | 0.09617 | G: 1.22(0.97, 1.54) | 527, 658 |
| RS9972917 | 17: 031442684 | CCL3 | 0.00718 | !A,C: 1.55(1.16, 2.08) | 0.06788 | C: 1.28(0.99, 1.66) | 525, 658 |
| RS9896211 | 17: 031454941 | CCL4 | 0.00704 | !A,G: 1.55(1.16, 2.08) | 0.06793 | G: 1.28(0.99, 1.66) | 527, 660 |
| RS868340 | 17: 031906733 | LOC441792 | 0.01754 | !T,T: 1.46(1.12, 1.92) | 0.09015 | A: 1.15(0.98, 1.35) | 526, 659 |
| RS9912576 | 17: 037572260 | KCNH4 | 0.01128 | !A,G: 1.56(1.17, 2.08) | 0.01143 | A: 1.39(1.08, 1.79) | 527, 660 |
| RS4792847 | 17: 040739267 | MAP3K14 | 0.02016 | !A,G: 1.39(1.11, 1.75) | 0.27808 | G: 1.1(0.93, 1.29) | 527, 657 |
| RS916888 | 17: 042218292 | WNT3 | 0.00522 | !C,T: 1.5(1.18, 1.91) | 0.02374 | T: 1.25(1.03, 1.51) | 527, 660 |
| RS2074404 | 17: 042220599 | WNT3 | 0.04712 | !G,T: 1.36(1.07, 1.73) | 0.09399 | T: 1.18(0.97, 1.42) | 527, 660 |
| RS1530364 | 17: 042306776 | WNT9B | 0.04379 | !A,A: 1.85(1.13, 3.02) | 0.06748 | G: 1.19(0.99, 1.42) | 527, 660 |
| RS4968282 | 17: 042313936 | WNT9B | 0.04639 | !G,G: 1.8(1.12, 2.89) | 0.05588 | A: 1.19(1, 1.43) | 526, 660 |
| RS304296 | 17: 054189228 | PPM1E | 0.00589 | !C,C: 1.69(1.22, 2.34) | 0.05713 | A: 1.18(1, 1.39) | 527, 659 |
| RS4076229 | 17: 054292038 | PPM1E | 0.00389 | !C,C: 1.72(1.24, 2.38) | 0.04711 | T: 1.18(1, 1.4) | 527, 660 |
| RS2058194 | 17: 059374080 | SCN4A | 0.05393 | T,T: 1.41(1.07, 1.84) | 0.02558 | T: 1.2(1.02, 1.42) | 527, 660 |
| RS11868547 | 17: 060954065 | AXIN2 | 0.00106 | !G,G: 1.55(1.19, 2.01) | 0.00022 | C: 1.36(1.15, 1.59) | 536, 666 |
| RS7591 | 17: 060955544 | AXIN2 | 0.01970 | !A,A: 1.5(1.08, 2.09) | 0.00439 | T: 1.28(1.08, 1.51) | 526, 660 |
| RS4076118 | 17: 060957556 | AXIN2 | 0.01097 | G,G: 1.36(1.07, 1.72) | 0.00220 | G: 1.3(1.1, 1.54) | 525, 656 |
| RS11079570 | 17: 060962637 | AXIN2 | 0.05055 | G,G: 1.32(1.05, 1.67) | 0.01336 | G: 1.24(1.05, 1.47) | 527, 659 |
| RS11867417 | 17: 060968360 | AXIN2 | 0.11767 | C,C: 1.28(1.02, 1.61) | 0.04823 | C: 1.2(1, 1.43) | 527, 660 |
| RS11079571 | 17: 060979143 | AXIN2 | 0.08915 | G,G: 1.33(1.04, 1.69) | 0.03537 | G: 1.26(1.02, 1.56) | 537, 668 |
| RS3923086 | 17: 060979950 | AXIN2 | 0.00117 | G,G: 1.58(1.24, 2.02) | 0.00647 | G: 1.26(1.07, 1.49) | 527, 660 |
| RS2240308 | 17: 060985053 | AXIN2 | 0.01092 | !A,G: 1.39(1.11, 1.75) | 0.13593 | A: 1.13(0.96, 1.33) | 527, 659 |
| RS12949046 | 17: 060985609 | AXIN2 | 0.00407 | G,G: 1.52(1.19, 1.95) | 0.01223 | G: 1.23(1.05, 1.45) | 526, 660 |
| RS232099 | 17: 061144332 | CCDC46 | 0.08519 | !C,C: 1.31(1.03, 1.67) | 0.02845 | T: 1.21(1.02, 1.42) | 527, 660 |
| RS1420804 | 17: 061322612 | CCDC46 | 0.10694 | !T,T: 1.31(1.01, 1.68) | 0.03805 | C: 1.19(1.01, 1.4) | 527, 660 |
| RS4790921 | 17: 061394976 | CCDC46 | 0.00254 | !A,A: 1.59(1.23, 2.07) | 0.00814 | G: 1.25(1.06, 1.47) | 527, 660 |
| RS3764402 | 17: 061727915 | PRKCA | 0.03577 | A,G: 1.38(1.09, 1.75) | 0.06740 | G: 1.2(0.99, 1.44) | 527, 660 |
| RS8075066 | 17: 061800371 | PRKCA | 0.00813 | !G,G: 1.38(1.1, 1.74) | 0.00174 | A: 1.33(1.11, 1.59) | 526, 660 |
| RS4261587 | 17: 061824672 | PRKCA, | 0.12988 | !A,A: 1.31(1, 1.71) | 0.04016 | G: 1.29(1.02, 1.65) | 527, 660 |
| | | L0C645710 | | | | | |
| RS9789061 | 17: 061849132 | PRKCA | 0.02010 | !T,T: 1.36(1.08, 1.71) | 0.00394 | C: 1.29(1.09, 1.54) | 527, 660 |
| RS10512512 | 17: 061965255 | PRKCA | 0.06443 | A,A: 1.37(1.05, 1.77) | 0.01672 | A: 1.33(1.05, 1.67) | 527, 660 |
| RS7211269 | 17: 061987236 | PRKCA | 0.01111 | T,T: | 0.88282 | C: 1.02(0.77, 1.37) | 527, 660 |
| | | | | 16.47(0.93, 292.94) | | | |
| RS11871468 | 17: 061987874 | PRKCA | 0.01652 | T,T: 1.52(1.14, 2.01) | 0.00386 | T: 1.46(1.13, 1.89) | 527, 660 |
| RS8077294 | 17: 061994228 | PRKCA | 0.03518 | C,C: 1.39(1.09, 1.77) | 0.01978 | C: 1.28(1.04, 1.58) | 526, 659 |
| RS8067797 | 17: 061999442 | PRKCA | 0.02654 | !C,T: 1.36(1.08, 1.71) | 0.15617 | T: 1.13(0.96, 1.33) | 527, 658 |
| RS9896134 | 17: 062029903 | PRKCA | 0.06185 | T,T: 1.32(1.05, 1.66) | 0.04647 | T: 1.21(1, 1.45) | 526, 660 |
| RS9901261 | 17: 062211937 | PRKCA | 0.04445 | C,C: 1.77(1.11, 2.8) | 0.40239 | C: 1.08(0.9, 1.3) | 527, 660 |
| RS12451435 | 17: 069881299 | GPR142 | 0.07399 | A,A: 1.41(1.04, 1.92) | 0.02870 | A: 1.21(1.02, 1.42) | 527, 660 |
| RS1320040 | 17: 069881996 | GPR142 | 0.00478 | !A,G: 1.51(1.18, 1.93) | 0.05038 | G: 1.22(1, 1.49) | 526, 660 |
| RS2689005 | 18: 000903665 | ADCYAP1 | 0.06791 | !A,A: 1.35(1.05, 1.74) | 0.02528 | C: 1.21(1.03, 1.42) | 527, 659 |
| RS948333 | 18: 013884260 | MC2R | 0.04265 | C,C: 1.37(1.07, 1.74) | 0.01379 | C: 1.23(1.04, 1.45) | 527, 660 |
| RS1421125 | 18: 020311909 | HRH4 | 0.10071 | A,A: 1.34(0.98, 1.84) | 0.03108 | A: 1.2(1.02, 1.42) | 527, 660 |
| RS626217 | 18: 042683180 | PIAS2 | 0.08854 | !T,T: 1.31(0.99, 1.73) | 0.02679 | G: 1.21(1.02, 1.42) | 518, 643 |
| RS1873191 | 18: 044722249 | SMAD7 | 0.09770 | !A,A: 1.29(1.02, 1.62) | 0.02871 | G: 1.22(1.02, 1.45) | 527, 660 |
| RS1893489 | 18: 046449659 | MAPK4 | 0.04504 | C,T: 1.33(1.06, 1.67) | 0.38557 | T: 1.08(0.92, 1.27) | 527, 660 |
| RS504904 | 18: 053918066 | NEDD4L | 0.03326 | !G,G: 1.53(1.11, 2.11) | 0.00853 | C: 1.5(1.11, 2.03) | 527, 660 |
| RS2062011 | 18: 059027094 | BCL2 | 0.07082 | T,T: 1.27(1.01, 1.61) | 0.01833 | T: 1.26(1.04, 1.52) | 527, 659 |
| RS3852908 | 19: 000545599 | HCN2 | 0.05498 | !C,C: 1.47(1.06, 2.05) | 0.01856 | T: 1.23(1.04, 1.45) | 526, 659 |
| RS687077 | 19: 000780408 | AZU1 | 0.06672 | !A,A: 1.36(1.06, 1.76) | 0.04212 | G: 1.19(1.01, 1.39) | 526, 660 |
| RS1640275 | 19: 002762293 | THOP1 | 0.11181 | A,A: 1.29(1.01, 1.64) | 0.03329 | A: 1.2(1.02, 1.41) | 527, 659 |
| RS1008076 | 19: 003919605 | DAPK3 | 0.10506 | !A,A: 1.37(0.98, 1.93) | 0.03743 | C: 1.2(1.01, 1.41) | 527, 657 |
| RS2885734 | 19: 004641259 | DPP9 | 0.06529 | A,A: 1.29(1.02, 1.62) | 0.01552 | A: 1.26(1.04, 1.51) | 527, 660 |
| RS4401152 | 19: 004659102 | DPP9 | 0.00626 | C,C: 1.46(1.14, 1.87) | 0.00095 | C: 1.43(1.16, 1.77) | 527, 660 |
| RS7253024 | 19: 006313724 | CLPP | 0.07861 | C,C: 1.45(1.05, 1.99) | 0.03089 | C: 1.39(1.03, 1.88) | 527, 660 |
| RS330877 | 19: 006847483 | EMR1 | 0.03147 | A,A: 1.59(1.06, 2.38) | 0.71746 | A: 1.03(0.87, 1.23) | 525, 658 |
| RS3745369 | 19: 007641475 | RETN | 0.09582 | !G,G: 1.34(1.02, 1.75) | 0.03504 | C: 1.19(1.01, 1.4) | 526, 660 |
| RS2287868 | 19: 007670772 | FCER2 | 0.06048 | !C,C: 1.37(1.06, 1.76) | 0.02296 | T: 1.21(1.03, 1.42) | 527, 660 |
| RS8105483 | 19: 007715283 | CD209 | 0.07138 | C,C: 1.3(1, 1.71) | 0.02709 | C: 1.32(1.03, 1.68) | 526, 659 |
| RS8105572 | 19: 007715327 | CD209 | 0.06299 | C,C: 1.32(1, 1.72) | 0.02333 | C: 1.33(1.04, 1.69) | 527, 660 |
| RS7252229 | 19: 007718181 | CD209 | 0.04286 | G,G: 1.33(1.02, 1.74) | 0.01530 | G: 1.35(1.06, 1.72) | 526, 660 |
| RS15723 | 19: 010064171 | FLJ11286, | 0.03556 | A,G: 1.5(1.1, 2.03) | 0.03839 | G: 1.35(1.02, 1.78) | 527, 660 |
| | | ANGPTL6 | | | | | |
| RS8109578 | 19: 010074154 | ANGPTL6 | 0.07579 | A,G: 1.43(1.06, 1.94) | 0.03954 | A: 1.33(1.01, 1.73) | 536, 668 |
| RS1056538 | 19: 010263938 | ICAM5 | 0.14881 | T,T: 1.32(0.96, 1.81) | 0.04645 | T: 1.18(1, 1.4) | 527, 660 |
| RS- | 19: 010477403 | EDG8 | 0.12109 | !A,A: 1.28(1.01, 1.63) | 0.03851 | G: 1.25(1.01, 1.53) | 524, 647 |
| GSK8116076 | | | | | | | |
| RS895819 | 19: 013808292 | LOC284454 | 0.00777 | C,T: 1.37(1.09, 1.72) | 0.44078 | T: 1.07(0.9, 1.27) | 526, 656 |
| RS3181163 | 19: 015027162 | CASP14 | 0.06362 | T,T: 1.35(1.06, 1.72) | 0.02765 | T: 1.26(1.03, 1.55) | 527, 660 |
| RS2288540 | 19: 017198344 | FLJ22709 | 0.01130 | !A,A: 1.42(1.09, 1.86) | 0.00321 | C: 1.44(1.13, 1.82) | 513, 645 |
| RS2288539 | 19: 017212535 | NR2F6 | 0.01586 | T,T: 3.51(1.19, 10.38) | 0.01082 | T: 1.39(1.08, 1.78) | 526, 654 |
| RS2238646 | 19: 018199101 | PDE4C | 0.03041 | A,G: 1.55(1.12, 2.15) | 0.02596 | A: 1.42(1.05, 1.91) | 527, 660 |
| RS7252583 | 19: 040990435 | PRODH2 | 0.10061 | A,A: 1.25(0.98, 1.59) | 0.03139 | A: 1.25(1.02, 1.53) | 527, 660 |
| RS3848666 | 19: 040995504 | PRODH2 | 0.07434 | !C,C: 1.97(1.03, 3.76) | 0.04168 | G: 1.25(1.01, 1.55) | 527, 660 |
| RS2163823 | 19: 043684018 | RYR1 | 0.02554 | A,A: 1.91(1.16, 3.15) | 0.43762 | A: 1.08(0.89, 1.3) | 527, 660 |
| RS1469699 | 19: 043685478 | RYR1 | 0.03044 | G,G: 1.7(1.06, 2.75) | 0.92411 | G: 1.01(0.84, 1.22) | 527, 660 |
| RS12978914 | 19: 043915557 | CAPN12 | 0.01070 | C,T: 1.56(1.06, 2.29) | 0.22738 | T: 1.27(0.88, 1.82) | 527, 660 |
| RS10410544 | 19: 044077372 | SIRT2 | 0.12441 | !C,C: 1.28(0.99, 1.66) | 0.03853 | T: 1.19(1.01, 1.4) | 527, 659 |
| RS3810386 | 19: 044998406 | DYRK1B | 0.03146 | T,T: 1.46(1.1, 1.93) | 0.08168 | T: 1.16(0.98, 1.36) | 526, 660 |
| RS718066 | 19: 045013680 | DYRK1B | 0.03337 | A,A: 1.46(1.1, 1.93) | 0.07508 | A: 1.16(0.99, 1.36) | 527, 660 |
| RS10404456 | 19: 052504740 | C5AR1 | 0.04539 | T,T: 1.35(1.07, 1.7) | 0.02916 | T: 1.23(1.02, 1.49) | 526, 659 |
| RS11670330 | 19: 052505005 | C5AR1 | 0.03065 | G,G: 1.37(1.09, 1.73) | 0.02150 | G: 1.25(1.03, 1.51) | 527, 660 |
| RS431234 | 19: 053279960 | PLA2G4C | 0.03423 | C,C: 1.76(1.13, 2.75) | 0.35996 | C: 1.09(0.91, 1.3) | 534, 667 |
| RS156631 | 19: 053290635 | PLA2G4C | 0.04000 | C,C: 1.51(1.09, 2.09) | 0.18832 | C: 1.12(0.95, 1.32) | 527, 660 |
| RS3760798 | 19: 053649309 | KCNJ14, | 0.05246 | !C,T: 1.5(1.07, 2.11) | 0.03804 | T: 1.41(1.03, 1.95) | 526, 658 |
| | | GRWD1 | | | | | |
| RS1799257 | 19: 053664351 | PSCD2 | 0.09337 | C,C: 1.32(0.99, 1.75) | 0.03110 | C: 1.33(1.03, 1.73) | 527, 660 |
| RS1062708 | 19: 054205085 | RUVBL2 | 0.08136 | T,T: 1.35(1.03, 1.77) | 0.02833 | T: 1.2(1.02, 1.41) | 527, 660 |
| RS753307 | 19: 054210296 | RUVBL2, | 0.02316 | T,T: 1.47(1.12, 1.94) | 0.05173 | T: 1.18(1, 1.39) | 526, 659 |
| | | LHB | | | | | |
| RS722036 | 19: 055922302 | CLEC11A | 0.09044 | !T,T: 1.24(0.99, 1.56) | 0.02699 | C: 1.22(1.02, 1.46) | 527, 660 |
| RS266851 | 19: 056028151 | KLK15, | 0.02485 | T,T: 2.1(1.14, 3.85) | 0.01018 | T: 1.32(1.07, 1.62) | 527, 660 |
| | | LOC646115 | | | | | |
| RS6045868 | 20: 001915278 | PDYN | 0.09048 | A,A: 1.59(0.97, 2.62) | 0.03370 | A: 1.23(1.02, 1.49) | 526, 660 |
| RS2281285 | 20: 001920460 | PDYN | 0.05031 | !T,T: 1.39(1.07, 1.81) | 0.01771 | C: 1.34(1.05, 1.69) | 527, 660 |
| RS3830064 | 20: 001923679 | PDYN | 0.08451 | !T,T: 1.33(1.03, 1.72) | 0.02896 | G: 1.29(1.03, 1.63) | 526, 660 |
| RS1010608 | 20: 003732593 | CDC25B | 0.05411 | C,C: 1.34(1.06, 1.69) | 0.03100 | C: 1.24(1.02, 1.5) | 527, 659 |
| RS910655 | 20: 003735550 | CDC25B | 0.00315 | !A,G: 1.5(1.18, 1.9) | 0.17614 | A: 1.14(0.95, 1.36) | 526, 660 |
| RS11570030 | 20: 003736194 | CDC25B | 0.12970 | !C,C: 1.35(1.02, 1.8) | 0.04931 | T: 1.31(1.01, 1.7) | 527, 660 |
| RS2234395 | 20: 005221745 | PROKR2 | 0.02776 | C,C: 1.39(1.07, 1.81) | 0.00815 | C: 1.25(1.06, 1.47) | 526, 660 |
| RS927104 | 20: 005227654 | PROKR2 | 0.02980 | C,C: 1.38(1.09, 1.74) | 0.00874 | C: 1.3(1.07, 1.57) | 527, 660 |
| RS3178250 | 20: 006708201 | BMP2 | 0.11262 | T,T: 1.27(1.01, 1.62) | 0.03005 | T: 1.25(1.02, 1.52) | 527, 658 |
| RS2423410 | 20: 009602944 | PAK7 | 0.05883 | !C,C: 1.35(1.06, 1.73) | 0.02414 | A: 1.21(1.03, 1.42) | 527, 660 |
| RS2423421 | 20: 009615956 | PAK7 | 0.13574 | T,T: 1.25(0.99, 1.58) | 0.04126 | T: 1.22(1.01, 1.48) | 527, 660 |
| RS2423425 | 20: 009632756 | PAK7 | 0.03703 | !T,T: 1.81(1.1, 3) | 0.01847 | C: 1.25(1.04, 1.51) | 527, 660 |
| RS2423432 | 20: 009640033 | PAK7 | 0.05398 | !T,T: 1.34(1.06, 1.69) | 0.03266 | C: 1.21(1.02, 1.43) | 527, 660 |
| RS2423437 | 20: 009651740 | PAK7 | 0.02565 | !A,A: 1.38(1.1, 1.74) | 0.00796 | G: 1.27(1.06, 1.5) | 525, 655 |
| RS2423456 | 20: 009668742 | PAK7 | 0.07931 | A,A: 1.26(1, 1.59) | 0.02565 | A: 1.24(1.03, 1.49) | 527, 660 |
| RS910969 | 20: 013455872 | TASP1 | 0.03442 | A,A: 1.59(1.12, 2.26) | 0.16004 | A: 1.14(0.96, 1.35) | 527, 660 |
| RS9927 | 20: 025225244 | ABHD12, | 0.01147 | G,G: 1.44(1.11, 1.87) | 0.00256 | G: 1.29(1.09, 1.51) | 527, 660 |
| | | PYGB | | | | | |
| RS2223745 | 20: 039147283 | TOP1 | 0.03360 | A,A: 1.44(1.08, 1.92) | 0.19738 | A: 1.12(0.95, 1.31) | 527, 660 |
| RS2284271 | 20: 043038835 | STK4 | 0.02482 | !C,C: 1.44(1.07, 1.94) | 0.00999 | T: 1.45(1.09, 1.91) | 527, 660 |
| RS553359 | 20: 043964245 | PLTP | 0.05531 | A,A: 1.34(1.06, 1.69) | 0.02326 | A: 1.22(1.03, 1.45) | 524, 659 |
| RS2250889 | 20: 044075813 | MMP9 | 0.00133 | C,G: 2.05(1.35, 3.13) | 0.00257 | G: 1.89(1.26, 2.84) | 527, 660 |
| RS6073991 | 20: 044089519 | SLC12A5 | 0.07326 | A,A: 1.33(1.05, 1.69) | 0.02882 | A: 1.26(1.03, 1.55) | 525, 660 |
| RS2297201 | 20: 044118385 | SLC12A5 | 0.04584 | !C,C: 1.44(1.03, 2.01) | 0.03807 | T: 1.42(1.03, 1.96) | 527, 660 |
| RS2179706 | 20: 055573277 | PCK1 | 0.12209 | T,T: 1.29(0.97, 1.73) | 0.03741 | T: 1.19(1.01, 1.4) | 527, 660 |
| RS149265 | 20: 057000032 | TH1L | 0.04105 | !C,T: 1.3(1.03, 1.63) | 0.69845 | C: 1.03(0.87, 1.22) | 526, 658 |
| RS9760 | 20: 057005158 | CTSZ, TH1L | 0.03390 | !A,G: 1.3(1.03, 1.64) | 0.66703 | A: 1.04(0.88, 1.23) | 527, 660 |
| RS260743 | 20: 057306280 | EDN3 | 0.00710 | !T,T: 1.41(1.09, 1.82) | 0.00123 | C: 1.31(1.11, 1.54) | 527, 660 |
| RS1044396 | 20: 061451578 | CHRNA4 | 0.06094 | !C,C: 1.39(1.05, 1.84) | 0.02020 | T: 1.22(1.03, 1.43) | 527, 660 |
| RS186332 | 20: 061629912 | PTK6 | 0.11020 | !G,G: 1.42(1, 2.01) | 0.04864 | A: 1.41(1.01, 1.96) | 527, 660 |
| RS310639 | 20: 061637126 | PTK6 | 0.04045 | !A,A: 1.5(1.02, 2.21) | 0.02759 | C: 1.52(1.05, 2.21) | 524, 659 |
| RS2027700 | 21: 026304672 | APP | 0.04692 | T,T: 1.34(1.06, 1.68) | 0.04348 | T: 1.21(1.01, 1.46) | 527, 659 |
| RS2248989 | 21: 029865858 | GRIK1 | 0.02288 | A,A: 1.68(1.1, 2.57) | 0.00617 | A: 1.29(1.08, 1.55) | 527, 660 |
| RS455804 | 21: 030068040 | GRIK1 | 0.00482 | !T,T: 2.88(1.44, 5.78) | 0.29973 | G: 1.12(0.91, 1.37) | 527, 660 |
| RS456466 | 21: 030107163 | GRIK1 | 0.06122 | !T,T: 1.28(1.01, 1.62) | 0.01589 | C: 1.27(1.05, 1.55) | 527, 660 |
| RS2211781 | 21: 032171165 | HUNK | 0.12064 | !A,A: 1.27(1.01, 1.61) | 0.03565 | C: 1.2(1.01, 1.42) | 527, 660 |
| RS2833552 | 21: 032203438 | HUNK | 0.09154 | A,A: 1.51(1.01, 2.25) | 0.03182 | A: 1.22(1.02, 1.45) | 527, 660 |
| RS2833554 | 21: 032206206 | HUNK | 0.08986 | !T,T: 1.26(0.99, 1.58) | 0.02775 | C: 1.24(1.03, 1.51) | 527, 660 |
| RS2833556 | 21: 032207171 | HUNK | 0.03131 | !G,G: 1.38(1.09, 1.75) | 0.00878 | A: 1.31(1.07, 1.6) | 527, 660 |
| RS2833557 | 21: 032207812 | HUNK | 0.04849 | C,G: 1.58(1, 2.48) | 0.05250 | C: 1.55(0.99, 2.42) | 526, 660 |
| RS11910494 | 21: 032217984 | HUNK | 0.09719 | !A,A: 1.28(1.02, 1.62) | 0.02966 | G: 1.22(1.02, 1.45) | 527, 659 |
| RS8126742 | 21: 032226361 | HUNK | 0.10982 | !G,G: 1.27(1.01, 1.6) | 0.03159 | A: 1.22(1.02, 1.46) | 527, 660 |
| RS743303 | 21: 032243857 | HUNK | 0.09421 | !C,C: 1.25(1, 1.58) | 0.02354 | T: 1.23(1.03, 1.48) | 527, 660 |
| RS- | 21: 034743691 | KCNE1 | 0.10404 | !G,G: 1.29(1.02, 1.63) | 0.03985 | A: 1.2(1.01, 1.42) | 524, 659 |
| GSK59473977 | | | | | | | |
| RS2249650 | 21: 035102856 | RUNX1 | 0.00844 | !A,A: 1.57(1.18, 2.07) | 0.02035 | G: 1.22(1.03, 1.43) | 526, 659 |
| RS2249884 | 21: 035104029 | RUNX1 | 0.05942 | !G,G: 1.33(1.06, 1.68) | 0.04371 | A: 1.19(1.01, 1.42) | 527, 660 |
| RS2834642 | 21: 035108189 | RUNX1 | 0.03996 | !G,G: 1.38(1.08, 1.76) | 0.02579 | A: 1.21(1.03, 1.43) | 527, 660 |
| RS2834644 | 21: 035108617 | RUNX1 | 0.03312 | !T,T: 1.4(1.09, 1.78) | 0.02343 | C: 1.21(1.03, 1.43) | 527, 660 |
| RS2243988 | 21: 035124310 | RUNX1 | 0.00774 | !C,C: 1.44(1.14, 1.81) | 0.02770 | T: 1.21(1.02, 1.43) | 527, 660 |
| RS2051394 | 21: 035128012 | RUNX1 | 0.02788 | C,G: 1.36(1.08, 1.71) | 0.22900 | G: 1.11(0.94, 1.3) | 527, 660 |
| RS2284612 | 21: 035135749 | RUNX1 | 0.02242 | C,T: 1.38(1.1, 1.74) | 0.09265 | T: 1.16(0.98, 1.37) | 527, 660 |
| RS8134380 | 21: 035144009 | RUNX1 | 0.01424 | !T,T: 1.51(1.13, 2) | 0.08924 | A: 1.15(0.98, 1.36) | 527, 660 |
| RS2834664 | 21: 035197309 | RUNX1 | 0.01680 | T,T: 1.49(1.14, 1.95) | 0.00343 | T: 1.43(1.12, 1.81) | 527, 660 |
| RS2834666 | 21: 035198545 | RUNX1 | 0.04837 | G,G: 1.47(1.08, 1.99) | 0.01118 | G: 1.44(1.08, 1.91) | 526, 658 |
| RS1475840 | 21: 035200812 | RUNX1 | 0.12280 | A,A: 1.3(0.99, 1.7) | 0.04290 | A: 1.18(1.01, 1.39) | 527, 659 |
| RS2834726 | 21: 035301918 | RUNX1 | 0.00746 | G,G: 16.5(0.93, 293.5) | 0.71729 | A: 1.06(0.8, 1.41) | 526, 660 |
| RS2242890 | 21: 035316502 | RUNX1 | 0.13656 | T,T: 1.31(0.96, 1.78) | 0.04290 | T: 1.19(1.01, 1.4) | 526, 659 |
| RS2294163 | 21: 035329260 | RUNX1 | 0.07332 | !C,C: 1.33(1.05, 1.69) | 0.03905 | T: 1.24(1.01, 1.53) | 526, 660 |
| RS2834739 | 21: 035330783 | RUNX1 | 0.08020 | A,A: 1.39(1.03, 1.89) | 0.02870 | A: 1.21(1.02, 1.42) | 527, 660 |
| | | C21ORF96 | | | | | |
| RS2835833 | 21: 037909767 | KCNJ6 | 0.00122 | !A,A: 2.12(1.39, 3.24) | 0.00145 | G: 1.33(1.12, 1.58) | 527, 659 |
| RS1709835 | 21: 037959506 | KCNJ6 | 0.01618 | !T,T: 1.48(1.09, 2.01) | 0.00464 | C: 1.27(1.08, 1.49) | 527, 659 |
| RS2070995 | 21: 038008835 | KCNJ6 | 0.10496 | A,A: 1.6(0.96, 2.66) | 0.03702 | A: 1.23(1.01, 1.49) | 527, 660 |
| RS1964927 | 21: 041575107 | BACE2 | 0.03085 | !G,G: 1.38(1.09, 1.74) | 0.01977 | A: 1.22(1.03, 1.45) | 527, 660 |
| RS427716 | 21: 042575991 | ABCG1 | 0.00944 | C,T: 1.44(1.14, 1.81) | 0.45648 | T: 1.07(0.91, 1.25) | 525, 660 |
| RS3788007 | 21: 042579845 | ABCG1 | 0.04478 | A,A: 2.13(1.08, 4.23) | 0.02207 | A: 1.28(1.04, 1.59) | 527, 660 |
| RS186529 | 21: 042617387 | ABCG1, TFF3 | 0.00392 | !G,G: 1.63(1.23, 2.16) | 0.00552 | T: 1.26(1.07, 1.48) | 526, 660 |
| RS3814896 | 21: 042644780 | TFF2 | 0.11207 | !A,A: 1.29(1.02, 1.62) | 0.03657 | G: 1.23(1.01, 1.48) | 527, 659 |
| RS225433 | 21: 042682487 | TMPRSS3 | 0.03132 | !G,G: 1.84(1.14, 2.98) | 0.37094 | C: 1.09(0.91, 1.31) | 527, 660 |
| RS2235133 | 21: 045145600 | ITGB2 | 0.05676 | !A,A: 1.3(1.01, 1.67) | 0.01425 | G: 1.23(1.04, 1.45) | 526, 660 |
| RS2006272 | 21: 045163757 | ITGB2 | 0.03035 | C,C: 2.04(1.21, 3.44) | 0.10292 | C: 1.17(0.97, 1.41) | 527, 660 |
| RS1987449 | 22: 015439560 | LOC644784, | 0.07817 | C,T: 1.54(1.06, 2.24) | 0.04705 | C: 1.44(1.02, 2.05) | 527, 660 |
| | | LOC648299 | | | | | |
| RS2904551 | 22: 017285934 | PRODH | 0.00376 | A,A: 3.91(1.41, 10.87) | 0.00393 | A: 3.86(1.39, 10.7) | 527, 660 |
| RS2073732 | 22: 017884470 | CDC45L | 0.03567 | !A,G: 1.3(1.03, 1.64) | 0.96362 | A: 1.01(0.84, 1.2) | 527, 660 |
| RS6269 | 22: 018329952 | COMT | 0.03325 | A,G: 1.37(1.09, 1.72) | 0.38115 | G: 1.08(0.91, 1.27) | 527, 660 |
| RS740601 | 22: 018330763 | COMT | 0.02583 | A,C: 1.38(1.1, 1.74) | 0.38101 | C: 1.08(0.91, 1.27) | 526, 660 |
| RS4818 | 22: 018331207 | COMT | 0.04646 | C,G: 1.34(1.07, 1.69) | 0.38017 | G: 1.08(0.92, 1.27) | 527, 660 |
| RS2275985 | 22: 022960093 | GGTLA1 | 0.05133 | A,A: 2.9(1.06, 7.95) | 0.04162 | A: 1.29(1.01, 1.64) | 526, 660 |
| RS1884817 | 22: 027436945 | CHEK2 | 0.07600 | G,G: 1.34(1.04, 1.73) | 0.02100 | G: 1.31(1.04, 1.65) | 526, 659 |
| RS165821 | 22: 028207625 | NEFH | 0.03688 | !A,G: 1.41(1.09, 1.82) | 0.03465 | A: 1.27(1.02, 1.58) | 527, 660 |
| RS2284013 | 22: 035420289 | CACNG2 | 0.02373 | A,A: 1.74(1.13, 2.67) | 0.46488 | A: 1.07(0.89, 1.28) | 527, 660 |
| RS3218322 | 22: 035858044 | IL2RB | 0.04548 | G,G: 1.35(1.07, 1.7) | 0.01879 | G: 1.24(1.04, 1.47) | 525, 658 |
| RS2235330 | 22: 035869659 | IL2RB | 0.03410 | C,T: 1.4(1.09, 1.8) | 0.05474 | C: 1.23(1, 1.52) | 527, 660 |
| RS132948 | 22: 036878791 | PLA2G6 | 0.01137 | !C,C: 3.09(1.29, 7.39) | 0.00914 | A: 1.36(1.08, 1.71) | 527, 660 |
| RS1534891 | 22: 037025045 | CSNK1E | 0.08649 | C,C: 1.36(1.03, 1.81) | 0.02602 | C: 1.35(1.04, 1.75) | 527, 660 |
| RS6001093 | 22: 037031339 | CSNK1E | 0.05360 | !C,T: 1.37(1.07, 1.77) | 0.04199 | T: 1.25(1.01, 1.54) | 527, 658 |
| RS135757 | 22: 037033599 | CSNK1E | 0.12047 | G,G: 1.29(1.02, 1.62) | 0.04737 | G: 1.21(1, 1.47) | 527, 660 |
| RS1008677 | 22: 038339282 | CACNA1I | 0.04184 | !C,C: 1.34(1.06, 1.68) | 0.01150 | T: 1.25(1.05, 1.48) | 527, 660 |
| RS714031 | 22: 038400180 | CACNA1I | 0.08259 | C,C: 1.37(1.03, 1.81) | 0.03103 | C: 1.2(1.02, 1.41) | 527, 659 |
| RS909680 | 22: 038403731 | CACNA1I | 0.02502 | !C,T: 1.39(1.1, 1.75) | 0.29411 | T: 1.1(0.93, 1.3) | 527, 660 |
| RS20551 | 22: 039877954 | EP300 | 0.10953 | G,G: 1.5(0.99, 2.28) | 0.04618 | G: 1.2(1, 1.43) | 527, 660 |
| RS6971 | 22: 041888870 | TSPO | 0.10498 | !T,T: 1.45(0.98, 2.16) | 0.03583 | C: 1.21(1.01, 1.44) | 527, 660 |
| RS2017222 | 22: 045153859 | CELSR1 | 0.00662 | !A,G: 1.44(1.05, 1.99) | 0.15343 | G: 1.25(0.92, 1.68) | 527, 659 |
| RS1883189 | 22: 045313477 | CELSR1 | 0.01717 | !C,T: 1.42(1.12, 1.79) | 0.19383 | C: 1.13(0.94, 1.34) | 526, 660 |
| RS3817816 | 22: 048951780 | PANX2 | 0.04147 | A,A: 1.8(1.04, 3.13) | 0.01166 | A: 1.31(1.07, 1.6) | 527, 660 |
| RS722801 | X: 012127416 | FRMPD4 | 0.12821 | !G,G: 1.77(0.98, 3.21) | 0.04138 | T: 1.29(1.05, 1.59) | 527, 660 |
| RS17328072 | X: 012130304 | FRMPD4 | 0.03671 | A,G: 1.89(1.18, 3.03) | 0.04516 | A: 1.55(1.21, 1.99) | 537, 667 |
| RS5933978 | X: 012199858 | FRMPD4 | 0.08945 | T,T: 1.64(1.04, 2.6) | 0.02264 | T: 1.29(1.01, 1.64) | 527, 660 |
| RS5935314 | X: 012298101 | FRMPD4 | 0.04033 | T,T: 2.11(1.2, 3.73) | 0.04982 | T: 1.03(0.84, 1.27) | 526, 660 |
| RS1874040 | X: 012341603 | FRMPD4 | 0.01223 | T,T: 2.15(1.28, 3.63) | 0.00279 | T: 1.1(0.9, 1.35) | 527, 660 |
| RS4240149 | X: 012612639 | FRMPD4 | 0.03237 | !T,T: 4.13(1.28, 13.31) | 0.08384 | C: 1.01(0.81, 1.27) | 526, 660 |
| RS3827468 | X: 012649215 | FRMPD4 | 0.03400 | !A,A: 1.71(1.09, 2.69) | 0.00807 | G: 1.17(0.94, 1.46) | 527, 660 |
| RS1989323 | X: 021794759 | MBTPS2 | 0.06766 | !T,T: 1.79(1, 3.23) | 0.04686 | C: 1.13(0.79, 1.62) | 527, 660 |
| RS6525471 | X: 070268563 | MED12 | 0.04655 | C,T: 1.93(1.16, 3.21) | 0.08199 | T: 1.29(0.96, 1.71) | 527, 660 |
| RS6318 | X: 113871991 | HTR2C | 0.01687 | C,C: | 0.71134 | G: 1.29(0.96, 1.74) | 527, 660 |
| | | | | 12.06(0.64, 225.88) | | | |
| RS5910591 | X: 118487578 | SLC25A5 | 0.04136 | A,G: 1.92(1.09, 3.39) | 0.25626 | G: 1.03(0.76, 1.4) | 527, 660 |
| RS5910593 | X: 118489695 | SLC25A5 | 0.04136 | A,G: 1.92(1.09, 3.39) | 0.25626 | G: 1.02(0.76, 1.38) | 526, 660 |
| RS2353720 | X: 122140083 | GRIA3 | 0.03084 | !C,C: 2.94(1.11, 7.79) | 0.00627 | G: 1.28(1, 1.63) | 527, 660 |
| RS2266797 | X: 132283809 | GPC4 | 0.04448 | !A,A: 3.19(1.1, 9.21) | 0.51908 | G: 1.12(0.89, 1.41) | 526, 660 |
| RS2223639 | X: 132299423 | GPC4 | 0.03335 | !A,A: 3.39(1.18, 9.74) | 0.40885 | G: 1.14(0.91, 1.44) | 526, 660 |
| RS2266807 | X: 132314889 | GPC4 | 0.03327 | !C,C: 2.62(1.17, 5.88) | 0.29945 | A: 1.15(0.93, 1.43) | 526, 660 |
| RS2267514 | X: 132823033 | GPC3 | 0.09047 | A,A: 1.65(1.04, 2.61) | 0.02343 | A: 1.33(1.04, 1.69) | 526, 659 |
| RS11797732 | X: 151129417 | GABRA3 | 0.05603 | T,T: 1.69(1.03, 2.76) | 0.03696 | T: 1.44(1.09, 1.9) | 527, 660 |
| RS994423 | X: 151132851 | GABRA3 | 0.05603 | T,T: 1.69(1.03, 2.76) | 0.03696 | T: 1.44(1.09, 1.9) | 527, 660 |
| RS5925191 | X: 151560198 | GABRQ | 0.06141 | !C,C: 1.71(1.08, 2.71) | 0.02165 | A: 1.17(0.92, 1.5) | 527, 660 |
| RS5924750 | X: 151564225 | GABRQ | 0.07823 | !A,A: 1.66(1.05, 2.63) | 0.02767 | G: 1.17(0.91, 1.5) | 527, 660 |

1. PolyName are RS identifiers. SNPs without an RS prefix were identified at GSK.
2. Key denoting strength of association 2.78E−05 < p-value ≦ 0.0005 in bold underline 0.0005 < p-value ≦ 0.005 in bold 0.005 < p-value ≦ 0.05 in italic
3. For example, if a SNP had genotypes AA, AG and GG, 3 chi-square tests were performed contrasting cases and controls: 1) AA vs AG + GG, 2) AG vs AA + GG and 3) GG vs AA + AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.
4. See Table 6 for descriptions for genes listed in this table

TABLE 5
SNPs with a Genotypic or Allelic Fishers Exact P ≦ 0.05 in both Munich and Aberdeen Collections

| | | | Munich | | Munich | Munich Risk Allele | Aberdeen | Aberdeen Risk | Aberdeen | Aberdeen Risk | Concordance |
|---|---|---|---|---|---|---|---|---|---|---|---|
| | NCBI 36 | Gene | Genotypic | Munich Risk Genotype | Allelic | & Odd Ratio (95% | Genotypic | Genotype & Odd | Allelic | Allele & Odd Ratio | between |
| Poly Name | position | Symbols | Exact P | & Odd Ratio (95% CI) | Exact P | CI) | Exact P | Ratio (95% CI) | Exact P | (95% CI) | Collections |
| RS- | 01: 149026497 | CTSK | 0.0258 | G,G: 2.57(1.09, 6.06) | 0.0268 | G: 2.54(1.08, 5.96) | 0.0242 | G,G: 2.2(1.11, 4.39) | 0.0255 | G: 2.17(1.1, 4.31) | YES |
| GSK8111961 | | | | | | | | | | | |
| RS1446966 | 01: 157830107 | APCS | 0.0077 | !G,G: 1.47(1.13, 1.91) | 0.0018 | A: 1.35(1.12, 1.63) | 0.0892 | G,G: 1.28(1.01, 1.62) | 0.0252 | G: 1.21(1.02, 1.43) | NO |
| RS2296618 | 01: 196932855 | PTPRC | 0.0097 | !G,G: 8.1(1.47, 44.6) | 0.3259 | A: 1.14(0.88, 1.48) | 0.1201 | !A,A: 1.32(1.02, 1.71) | 0.0339 | G: 1.28(1.02, 1.61) | NO |
| RS2046490 | 02: 163147732 | KCNH7 | 0.0127 | A,A: 1.56(1.16, 2.09) | 0.0202 | A: 1.24(1.04, 1.48) | 0.0241 | A,G: 1.37(1.09, 1.73) | 0.6196 | G: 1.05(0.89, 1.23) | NO |
| RS1376865 | 02: 175332843 | CHRNA1 | 0.0845 | A,A: 1.4(1.04, 1.88) | 0.0348 | A: 1.34(1.03, 1.75) | 0.0261 | !G,G: 2.45(1.06, 5.64) | 0.0083 | A: 1.39(1.09, 1.78) | YES |
| RS889895 | 02: 208107174 | CREB1 | 0.0394 | C,T: 1.32(1.01, 1.72) | 0.7814 | C: 1.03(0.83, 1.28) | 0.0541 | !T,T: 1.34(1.05, 1.71) | 0.0124 | C: 1.3(1.06, 1.61) | NO |
| RS977162 | 03: 020166510 | PCAF | 0.0125 | G,G: 1.47(1.14, 1.91) | 0.0093 | G: 1.34(1.08, 1.67) | 0.1039 | !G,G: 1.28(1.01, 1.63) | 0.0268 | A: 1.26(1.03, 1.54) | NO |
| RS610902 | 03: 120734475 | CD80 | 0.0570 | C,C: 1.35(1.04, 1.75) | 0.0224 | C: 1.24(1.03, 1.49) | 0.0885 | C,C: 1.31(1.04, 1.66) | 0.0411 | C: 1.19(1.01, 1.41) | YES |
| RS2244291 | 04: 053163236 | USP46 | 0.0358 | !A,G: 1.52(1.08, 2.14) | 0.0593 | A: 1.35(0.99, 1.84) | 0.0094 | A,A: 1.56(1.17, 2.08) | 0.0016 | A: 1.53(1.17, 1.99) | YES |
| RS1047530 | 05: 075947155 | IQGAP2, | 0.0298 | A,G: 1.4(1.08, 1.8) | 0.6159 | G: 1.05(0.88, 1.25) | 0.0055 | !A,A: 1.58(1.2, 2.08) | 0.0148 | G: 1.23(1.04, 1.44) | YES |
| | | F2RL2 | | | | | | | | | |
| RS2072633 | 06: 032027557 | CFB, RDBP | 0.0324 | !T,T: 1.52(1.05, 2.21) | 0.0129 | C: 1.27(1.05, 1.52) | 0.0222 | !T,T: 1.43(1.1, 1.87) | 0.1164 | C: 1.14(0.97, 1.34) | YES |
| RS38559 | 07: 081722923 | CACNA2D1 | 0.0370 | G,G: 1.45(1.04, 2.02) | 0.0152 | G: 1.47(1.08, 2) | 0.0370 | !A,G: 1.46(1.09, 1.96) | 0.0572 | G: 1.29(0.99, 1.67) | YES |
| RS2976440 | 08: 024864142 | NEFL | 0.0426 | T,T: 1.34(1.04, 1.74) | 0.0121 | T: 1.27(1.06, 1.53) | 0.0698 | T,T: 1.32(1.05, 1.66) | 0.0397 | T: 1.2(1.01, 1.43) | YES |
| RS2976437 | 08: 024872047 | NEFL | 0.0304 | A,A: 1.36(1.05, 1.76) | 0.0081 | A: 1.29(1.07, 1.55) | 0.0659 | A,A: 1.33(1.05, 1.67) | 0.0286 | A: 1.21(1.02, 1.44) | YES |
| RS1440332 | 08: 073897644 | KCNB2 | 0.0034 | C,T: 1.6(1.22, 2.1) | 0.4254 | T: 1.09(0.89, 1.32) | 0.0126 | !C,T: 1.42(1.13, 1.79) | 0.7686 | C: 1.03(0.87, 1.21) | NO |
| RS2664361 | 08: 089200925 | MMP16 | 0.1018 | A,A: 1.3(0.99, 1.71) | 0.0310 | A: 1.22(1.02, 1.46) | 0.0715 | A,A: 1.34(1.04, 1.73) | 0.0207 | A: 1.21(1.03, 1.43) | YES |
| RS1996637 | 08: 089227035 | MMP16 | 0.0794 | T,T: 1.33(1.01, 1.74) | 0.0240 | T: 1.23(1.03, 1.48) | 0.0487 | T,T: 1.36(1.06, 1.76) | 0.0131 | T: 1.23(1.05, 1.45) | YES |
| RS6144 | 09: 002635557 | VLDLR | 0.0836 | A,A: 1.36(1.03, 1.78) | 0.0259 | A: 1.3(1.03, 1.63) | 0.0415 | A,T: 1.39(1.07, 1.79) | 0.1719 | T: 1.17(0.94, 1.44) | YES |
| RS4747082 | 10: 072136936 | ADAMTS14 | 0.0318 | !A,G: 1.55(1.07, 2.25) | 0.0769 | G: 1.38(0.98, 1.96) | 0.0368 | A,A: 3.91(0.77, 252.04) | 0.8733 | A: 1.03(0.75, 1.41) | YES |
| RS3781730 | 11: 075585731 | WNT11 | 0.0169 | !C,T: 1.61(1.15, 2.24) | 0.0151 | C: 1.42(1.07, 1.89) | 0.0034 | !T,T: 5.04(1.62, 15.68) | 0.0167 | C: 1.32(1.05, 1.66) | YES |
| RS2927493 | 11: 087980312 | GRM5 | 0.0306 | !C,C: 1.66(1.11, 2.49) | 0.2631 | T: 1.12(0.93, 1.36) | 0.0442 | T,T: 1.34(1.07, 1.69) | 0.0134 | T: 1.24(1.05, 1.47) | YES |
| RS498186 | 11: 102174855 | MMP1 | 0.0153 | !A,A: 1.41(1.08, 1.85) | 0.0047 | C: 1.3(1.08, 1.56) | 0.1056 | A,A: 1.28(1, 1.64) | 0.0321 | A: 1.2(1.02, 1.42) | NO |
| RS1548133 | 11: 129821890 | ADAMTS15 | 0.0573 | G,G: 1.46(1.03, 2.07) | 0.0202 | G: 1.24(1.03, 1.49) | 0.0861 | !A,A: 1.27(1, 1.6) | 0.0236 | G: 1.22(1.03, 1.44) | YES |
| RS2189480 | 12: 046550095 | VDR | 0.0663 | C,C: 1.34(1.03, 1.74) | 0.0224 | C: 1.24(1.03, 1.5) | 0.0017 | A,A: 1.87(1.32, 2.65) | 0.0015 | A: 1.32(1.11, 1.56) | NO |
| RS1582188 | 13: 104934457 | G30, DAOA | 0.0212 | !C,T: 1.48(1.11, 1.98) | 0.1120 | T: 1.22(0.96, 1.55) | 0.0295 | T,T: 1.33(1.03, 1.7) | 0.0071 | T: 1.34(1.08, 1.66) | YES |
| RS1816628 | 14: 051819186 | PTGDR | 0.0454 | G,G: 1.41(1.04, 1.9) | 0.0157 | G: 1.25(1.05, 1.49) | 0.0650 | !G,G: 1.36(1.04, 1.79) | 0.0185 | C: 1.22(1.03, 1.43) | NO |
| RS1286920 | 14: 089609412 | KCNK13 | 0.0806 | !C,C: 1.4(1.02, 1.91) | 0.0443 | G: 1.21(1.01, 1.44) | 0.0011 | !C,G: 1.53(1.22, 1.93) | 0.7703 | C: 1.03(0.87, 1.21) | YES |
| RS2230739 | 16: 003973437 | ADCY9 | 0.0219 | !A,A: 1.4(1.08, 1.81) | 0.0052 | G: 1.33(1.09, 1.61) | 0.0428 | !G,G: 1.65(1.1, 2.46) | 0.2433 | A: 1.11(0.93, 1.33) | YES |
| RS1873191 | 18: 044722249 | SMAD7 | 0.0519 | !A,A: 1.29(1, 1.67) | 0.0233 | G: 1.26(1.03, 1.54) | 0.0977 | !A,A: 1.29(1.02, 1.62) | 0.0287 | G: 1.22(1.02, 1.45) | YES |
| RS3760798 | 19: 053649309 | KCNJ14, | 0.0444 | C,C: 6.21(1.11, 34.94) | 0.1727 | C: 1.27(0.91, 1.77) | 0.0525 | !C,T: 1.5(1.07, 2.11) | 0.0380 | T: 1.41(1.03, 1.95) | NO |
| | | GRWD1 | | | | | | | | | |
| RS2234395 | 20: 005221745 | PROKR2 | 0.0137 | !C,C: 1.5(1.12, 2) | 0.0041 | T: 1.3(1.09, 1.56) | 0.0278 | C,C: 1.39(1.07, 1.81) | 0.0081 | C: 1.25(1.06, 1.47) | NO |
| RS2423410 | 20: 009602944 | PAK7 | 0.0028 | !A,C: 1.49(1.15, 1.93) | 0.6041 | A: 1.05(0.88, 1.27) | 0.0588 | !C,C: 1.35(1.06, 1.73) | 0.0241 | A: 1.21(1.03, 1.42) | YES |
| RS6318 | X: 113871991 | HTR2C | 0.0114 | !C,C: 5.88(1.04, 33.31) | 0.0035 | G: 1.58(1.16, 2.14) | 0.0169 | C,C: 2.06(0.64, 225.88) | 0.7113 | G: 1.29(0.96, 1.74) | NO |
| RS994423 | X: 151132851 | GABRA3 | 0.0028 | !G,T: 2.22(1.33, 3.71) | 0.0792 | T: 1.32(0.98, 1.78) | 0.0560 | T,T: 1.69(1.03, 2.76) | 0.0370 | T: 1.44(1.09, 1.9) | YES |

1. PolyName are RS identifiers. SNPs without an RS prefix were identified at GSK.
2. Key denoting strength of association
2.78E−05 < p-value ≦ 0.0005 in bold underline
0.0005 < p-value ≦ 0.005 in bold
0.005 < p-value ≦ 0.05 in italic
3. For example, if a SNP had genotypes AA, AG and GG, 3 chi-square tests were performed contrasting cases and controls: 1) AA vs AG + GG, 2) AG vs AA + GG and 3) GG vs AA + AG. An odds ratio was then calculated for the test with the largest chi-square statistic. If the odds ratio was > 1, this genotype was reported as the risk genotype. If the odds ratio was < 1, then 1) the risk genotype was reported as “!” (“!” means “not”) this genotype and 2) a new odds ratio was calculated as the inverse of the original odds ratio. This new odds ratio was reported.
4. Concordance was assessed by combining the Munich and Aberdeen collections into a single set and conducting genotypic and allelic association on the combined set. Concordance was defined as ‘YES’ if the genotypic and/or allelic p-value ≦ 0.05. Concordance was defined as ‘NO’ if both the genotypic and/or allelic p-values were > 0.05.
5. See Table 6 for descriptions for genes listed in this table

TABLE 6
Gene Description for Genes Listed in Tables 3 to 5.

| | | | Entrez | | |
|---|---|---|---|---|---|
| Gene | Map | HUGO | Gene | | Collection |
| Symbol | Location | Gene¹ | ID² | Gene Description | Associated In³ |
| AAA1 | 7p14.3 | FALSE | 404744 | AAA1 protein | Munich |
| ABCA1 | 9q31.1 | TRUE | 19 | ATP-binding cassette, sub-family A (ABC1), member 1 | Aberdeen |
| ABCA1 | 9q31.1 | TRUE | 19 | ATP-binding cassette, sub-family A (ABC1), member 1 | Munich |
| ABCB11 | 2q24 | TRUE | 8647 | ATP-binding cassette, sub-family B (MDR/TAP), member 11 | Munich |
| ABCC1 | 16p13.1 | TRUE | 4363 | ATP-binding cassette, sub-family C (CFTR/MRP), member 1 | Munich |
| ABCC3 | 17q22 | TRUE | 8714 | ATP-binding cassette, sub-family C (CFTR/MRP), member 3 | Munich |
| ABCC4 | 13q32 | TRUE | 10257 | ATP-binding cassette, sub-family C (CFTR/MRP), member 4 | Munich |
| ABCC6 | 16p13.1 | TRUE | 368 | ATP-binding cassette, sub-family C (CFTR/MRP), member 6 | Aberdeen |
| ABCC8 | 11p15.1 | TRUE | 6833 | ATP-binding cassette, sub-family C (CFTR/MRP), member 8 | Munich |
| ABCC9 | 12p12.1 | TRUE | 10060 | ATP-binding cassette, sub-family C (CFTR/MRP), member 9 | Munich |
| ABCG1 | 21q22.3 | TRUE | 9619 | ATP-binding cassette, sub-family G (WHITE), member 1 | Aberdeen |
| ABCG5 | 2p21 | TRUE | 64240 | ATP-binding cassette, sub-family G (WHITE), member 5 | Aberdeen |
| | | | | (sterolin 1) | |
| ABCG5 | 2p21 | TRUE | 64240 | ATP-binding cassette, sub-family G (WHITE), member 5 | Munich |
| | | | | (sterolin 1) | |
| ABCG8 | 2p21 | TRUE | 64241 | ATP-binding cassette, sub-family G (WHITE), member 8 | Aberdeen |
| | | | | (sterolin 2) | |
| ABCG8 | 2p21 | TRUE | 64241 | ATP-binding cassette, sub-family G (WHITE), member 8 | Munich |
| | | | | (sterolin 2) | |
| ABHD12 | 20p11.21 | TRUE | 26090 | abhydrolase domain containing 12 | Aberdeen |
| ABL2 | 1q24-q25 | TRUE | 27 | v-abl Abelson murine leukemia viral oncogene homolog 2 (arg, | Aberdeen |
| | | | | Abelson-related gene) | |
| ACCN1 | 17q11.2-q12 | TRUE | 40 | amiloride-sensitive cation channel 1, neuronal (degenerin) | Aberdeen |
| ACCN4 | 2q35 | TRUE | 55515 | amiloride-sensitive cation channel 4, pituitary | Aberdeen |
| ACLY | 17q12-q21 | TRUE | 47 | ATP citrate lyase | Munich |
| ACSL5 | 10q25.1-q25.2 | TRUE | 51703 | acyl-CoA synthetase long-chain family member 5 | Munich |
| ADAM12 | 10q26.3 | TRUE | 8038 | ADAM metallopeptidase domain 12 (meltrin alpha) | Aberdeen |
| ADAM15 | 1q21.3 | TRUE | 8751 | ADAM metallopeptidase domain 15 (metargidin) | Aberdeen |
| ADAM19 | 5q32-q33 | TRUE | 8728 | ADAM metallopeptidase domain 19 (meltrin beta) | Munich |
| ADAM23 | 2q33 | TRUE | 8745 | ADAM metallopeptidase domain 23 | Munich |
| ADAM8 | 10q26.3 | TRUE | 101 | ADAM metallopeptidase domain 8 | Aberdeen |
| ADAMTS14 | 10q2 | TRUE | 140766 | ADAM metallopeptidase with thrombospondin type 1 motif, 14 | Both Sets |
| ADAMTS14 | 10q2 | TRUE | 140766 | ADAM metallopeptidase with thrombospondin type 1 motif, 14 | Aberdeen |
| ADAMTS14 | 10q2 | TRUE | 140766 | ADAM metallopeptidase with thrombospondin type 1 motif, 14 | Munich |
| ADAMTS15 | 11q25 | TRUE | 170689 | ADAM metallopeptidase with thrombospondin type 1 motif, 15 | Both Sets |
| ADAMTS15 | 11q25 | TRUE | 170689 | ADAM metallopeptidase with thrombospondin type 1 motif, 15 | Aberdeen |
| ADAMTS15 | 11q25 | TRUE | 170689 | ADAM metallopeptidase with thrombospondin type 1 motif, 15 | Munich |
| ADAMTS16 | 5p15 | TRUE | 170690 | ADAM metallopeptidase with thrombospondin type 1 motif, 16 | Munich |
| ADAMTS17 | 15q24 | TRUE | 170691 | ADAM metallopeptidase with thrombospondin type 1 motif, 17 | Munich |
| ADAMTS2 | 5qter | TRUE | 9509 | ADAM metallopeptidase with thrombospondin type 1 motif, 2 | Aberdeen |
| ADAMTS20 | 12q12 | TRUE | 80070 | ADAM metallopeptidase with thrombospondin type 1 motif, 20 | Aberdeen |
| ADAMTS3 | 4q13.3 | TRUE | 9508 | ADAM metallopeptidase with thrombospondin type 1 motif, 3 | Aberdeen |
| ADAMTS7 | 15q24.2 | TRUE | 11173 | ADAM metallopeptidase with thrombospondin type 1 motif, 7 | Munich |
| ADARB1 | 21q22.3 | TRUE | 104 | adenosine deaminase, RNA-specific, B1 (RED1 homolog rat) | Munich |
| ADARB2 | 10p15.3 | TRUE | 105 | adenosine deaminase, RNA-specific, B2 (RED2 homolog rat) | Aberdeen |
| ADARB2 | 10p15.3 | TRUE | 105 | adenosine deaminase, RNA-specific, B2 (RED2 homolog rat) | Munich |
| ADCY8 | 8q24 | TRUE | 114 | adenylate cyclase 8 (brain) | Aberdeen |
| ADCY8 | 8q24 | TRUE | 114 | adenylate cyclase 8 (brain) | Munich |
| ADCY9 | 16p13.3 | TRUE | 115 | adenylate cyclase 9 | Both Sets |
| ADCY9 | 16p13.3 | TRUE | 115 | adenylate cyclase 9 | Aberdeen |
| ADCY9 | 16p13.3 | TRUE | 115 | adenylate cyclase 9 | Munich |
| ADCYAP1 | 18p11 | TRUE | 116 | adenylate cyclase activating polypeptide 1 (pituitary) | Aberdeen |
| ADH7 | 4q23-q24 | TRUE | 131 | alcohol dehydrogenase 7 (class IV), mu or sigma polypeptide | Munich |
| ADIPOQ | 3q27 | TRUE | 9370 | adiponectin, C1Q and collagen domain containing | Aberdeen |
| ADIPOR2 | 12p13.31 | TRUE | 79602 | adiponectin receptor 2 | Aberdeen |
| ADMR | 12q13.3 | TRUE | 11318 | adrenomedullin receptor | Munich |
| ADORA1 | 1q32.1 | TRUE | 134 | adenosine A1 receptor | Munich |
| ADORA3 | 1p13.2 | TRUE | 140 | adenosine A3 receptor | Aberdeen |
| ADRA1B | 5q23-q32 | TRUE | 147 | adrenergic, alpha-1B-, receptor | Munich |
| ADRA2A | 10q24-q26 | TRUE | 150 | adrenergic, alpha-2A-, receptor | Aberdeen |
| ADRB3 | 8p12-p11.2 | TRUE | 155 | adrenergic, beta-3-, receptor | Aberdeen |
| ADRBK1 | 11q13 | TRUE | 156 | adrenergic, beta, receptor kinase 1 | Aberdeen |
| AEBP1 | 7p13 | TRUE | 165 | AE binding protein 1 | Aberdeen |
| AFF1 | 4q21 | TRUE | 4299 | AF4/FMR2 family, member 1 | Aberdeen |
| AGTR1 | 3q21-q25 | TRUE | 185 | angiotensin II receptor, type 1 | Munich |
| AIF1 | 6p21.3 | TRUE | 199 | allograft inflammatory factor 1 | Aberdeen |
| AK057159 | | FALSE | | SPTREMBL Q96MC9 KNOWN | Aberdeen |
| AKT1 | 14q32.32\| | TRUE | 207 | v-akt murine thymoma viral oncogene homolog 1 | Aberdeen |
| | 14q32.32 | | | | |
| ALAS1 | 3p21.1 | TRUE | 211 | aminolevulinate, delta-, synthase 1 | Munich |
| ALCAM | 3q13.1 | TRUE | 214 | activated leukocyte cell adhesion molecule | Aberdeen |
| ALK | 2p23 | TRUE | 238 | anaplastic lymphoma kinase (Ki-1) | Aberdeen |
| ALOX5AP | 13q12 | TRUE | 241 | arachidonate 5-lipoxygenase-activating protein | Munich |
| ANGPTL3 | 1p31.1-p22.3 | TRUE | 27329 | angiopoietin-like 3 | Munich |
| ANGPTL6 | 19p13.2 | TRUE | 83854 | angiopoietin-like 6 | Aberdeen |
| ANKRD35 | 1q21.1 | TRUE | 148741 | ankyrin repeat domain 35 | Aberdeen |
| ANKRD40 | 17q21.33 | TRUE | 91369 | ankyrin repeat domain 40 | Munich |
| ANXA2 | 15q21-q22 | TRUE | 302 | annexin A2 | Aberdeen |
| ANXA2 | 15q21-q22 | TRUE | 302 | annexin A2 | Munich |
| AP1S1 | 7q22.1 | TRUE | 1174 | adaptor-related protein complex 1, sigma 1 subunit | Munich |
| AP2M1 | 3q28 | TRUE | 1173 | adaptor-related protein complex 2, mu 1 subunit | Aberdeen |
| APBB1 | 11p15 | TRUE | 322 | amyloid beta (A4) precursor protein-binding, family B, member 1 | Aberdeen |
| | | | | (Fe65) | |
| APBB3 | 5q31 | TRUE | 10307 | amyloid beta (A4) precursor protein-binding, family B, member 3 | Aberdeen |
| APCS | 1q21-q23 | TRUE | 325 | amyloid P component, serum | Both Sets |
| APCS | 1q21-q23 | TRUE | 325 | amyloid P component, serum | Aberdeen |
| APCS | 1q21-q23 | TRUE | 325 | amyloid P component, serum | Munich |
| APLN | Xq25-26.3 | TRUE | 8862 | apelin, AGTRL1 ligand | Munich |
| APOA5 | 11q23 | TRUE | 116519 | apolipoprotein A-V | Aberdeen |
| APOM | 6p21.33 | TRUE | 55937 | apolipoprotein M | Aberdeen |
| APP | 21q21.2\|21q21.3 | TRUE | 351 | amyloid beta (A4) precursor protein (peptidase nexin-II, | Aberdeen |
| | | | | Alzheimer disease) | |
| APP | 21q21.2\|21q21.3 | TRUE | 351 | amyloid beta (A4) precursor protein (peptidase nexin-II, | Munich |
| | | | | Alzheimer disease) | |
| AQP1 | 7p14 | TRUE | 358 | aquaporin 1 (Colton blood group) | Aberdeen |
| AQP3 | 9p13 | TRUE | 360 | aquaporin 3 (Gill blood group) | Munich |
| AQP4 | 18q11.2-q12.1 | TRUE | 361 | aquaporin 4 | Munich |
| AQP6 | 12q13 | TRUE | 363 | aquaporin 6, kidney specific | Aberdeen |
| AQP9 | 15q22.1-22.2 | TRUE | 366 | aquaporin 9 | Munich |
| ARF4 | 3p21.2-p21.1 | TRUE | 378 | ADP-ribosylation factor 4 | Munich |
| ARHGAP10 | 4q31.23 | TRUE | 79658 | Rho GTPase activating protein 10 | Aberdeen |
| ASAH2 | 10q11.21 | TRUE | 56624 | N-acylsphingosine amidohydrolase (non-lysosomal ceramidase) 2 | Aberdeen |
| ASAH2 | 10q11.21 | TRUE | 56624 | N-acylsphingosine amidohydrolase (non-lysosomal ceramidase) 2 | Munich |
| ATF2 | 2q32 | TRUE | 1386 | activating transcription factor 2 | Aberdeen |
| ATF7 | 12q13 | TRUE | 11016 | activating transcription factor 7 | Aberdeen |
| ATG4B | 2q37.3 | TRUE | 23192 | ATG4 autophagy related 4 homolog B (S. cerevisiae) | Aberdeen |
| ATG4B | 2q37.3 | TRUE | 23192 | ATG4 autophagy related 4 homolog B (S. cerevisiae) | Munich |
| ATG4D | 19p13.2 | TRUE | 84971 | ATG4 autophagy related 4 homolog D (S. cerevisiae) | Munich |
| AVP | 20p13 | TRUE | 551 | arginine vasopressin (neurophysin II, antidiuretic hormone, | Munich |
| | | | | diabetes insipidus, neurohypophyseal) | |
| AVPR1B | 1q32 | TRUE | 553 | arginine vasopressin receptor 1B | Munich |
| AXIN1 | 16p13.3 | TRUE | 8312 | axin 1 | Aberdeen |
| AXIN2 | 17q23-q24 | TRUE | 8313 | axin 2 (conductin, axil) | Aberdeen |
| AXL | 19q13.1 | TRUE | 558 | AXL receptor tyrosine kinase | Munich |
| AYP1 | 11q13.1 | FALSE | 84153 | AYP1 protein | Aberdeen |
| AZU1 | 19p13.3 | TRUE | 566 | azurocidin 1 (cationic antimicrobial protein 37) | Aberdeen |
| BACE2 | 21q22.3 | TRUE | 25825 | beta-site APP-cleaving enzyme 2 | Aberdeen |
| BAD | 11q13.1 | TRUE | 572 | BCL2-antagonist of cell death | Aberdeen |
| BAI1 | 8q24 | TRUE | 575 | brain-specific angiogenesis inhibitor 1 | Aberdeen |
| BAI3 | 6q12 | TRUE | 577 | brain-specific angiogenesis inhibitor 3 | Aberdeen |
| BAI3 | 6q12 | TRUE | 577 | brain-specific angiogenesis inhibitor 3 | Munich |
| BAT1 | 6p21.3 | TRUE | 7919 | HLA-B associated transcript 1 | Munich |
| BAT3 | 6p21.3 | TRUE | 7917 | HLA-B associated transcript 3 | Aberdeen |
| BAX | 19q13.3-q13.4 | TRUE | 581 | BCL2-associated X protein | Munich |
| BAZ1B | 7q11.23 | TRUE | 9031 | bromodomain adjacent to zinc finger domain, 1B | Munich |
| BCL2 | 18q21.33\| | TRUE | 596 | B-cell CLL/lymphoma 2 | Aberdeen |
| | 18q21.3 | | | | |
| BCL2 | 18q21.33\| | TRUE | 596 | B-cell CLL/lymphoma 2 | Munich |
| | 18q21.3 | | | | |
| BCL2L14 | 12p13-p12 | TRUE | 79370 | BCL2-like 14 (apoptosis facilitator) | Aberdeen |
| BDKRB1 | 14q32.1-q32.2 | TRUE | 623 | bradykinin receptor B1 | Aberdeen |
| BLK | 8p23-p22 | TRUE | 640 | B lymphoid tyrosine kinase | Munich |
| BMP2 | 20p12 | TRUE | 650 | bone morphogenetic protein 2 | Aberdeen |
| BMPR1B | 4q22-q24 | TRUE | 658 | bone morphogenetic protein receptor, type IB | Aberdeen |
| BRD2 | 6p21.3 | TRUE | 6046 | bromodomain containing 2 | Aberdeen |
| BRD3 | 9q34 | TRUE | 8019 | bromodomain containing 3 | Aberdeen |
| C10ORF6 | 10q24.31 | TRUE | 55719 | chromosome 10 open reading frame 6 | Munich |
| C11ORF31 | 11q12.1 | TRUE | 280636 | chromosome 11 open reading frame 31 | Aberdeen |
| C11ORF36 | 11p15.4 | TRUE | 283303 | chromosome 11 open reading frame 36 | Munich |
| C12ORF40 | 12q12 | TRUE | 283461 | chromosome 12 open reading frame 40 | Aberdeen |
| C14ORF131 | 14q32.31 | TRUE | 55778 | chromosome 14 open reading frame 131 | Aberdeen |
| C14ORF156 | 14q24.3 | TRUE | 81892 | chromosome 14 open reading frame 156 | Munich |
| C14ORF21 | 14q12 | TRUE | 161424 | chromosome 14 open reading frame 21 | Munich |
| C18ORF16 | 18q11.2 | TRUE | 147429 | chromosome 18 open reading frame 16 | Munich |
| C1D | 2p13-p12 | FALSE | 10438 | nuclear DNA-binding protein | Munich |
| C1ORF107 | 1q32.2 | TRUE | 27042 | chromosome 1 open reading frame 107 | Aberdeen |
| C1ORF147 | 1q32.1 | TRUE | 574431 | chromosome 1 open reading frame 147 | Aberdeen |
| C1QTNF3 | | TRUE | 114899 | C1q and tumor necrosis factor related protein 3 | Aberdeen |
| C1QTNF5 | 11q23.3 | TRUE | 114902 | C1q and tumor necrosis factor related protein 5 | Munich |
| C1QTNF7 | 4p16-p15 | TRUE | 114905 | C1q and tumor necrosis factor related protein 7 | Aberdeen |
| C2 | 6p21.3 | TRUE | 717 | complement component 2 | Aberdeen |
| C21ORF96 | 21q22.12 | TRUE | 80215 | chromosome 21 open reading frame 96 | Aberdeen |
| C3ORF42 | 3p26-p25 | TRUE | 84657 | chromosome 3 open reading frame 42 | Aberdeen |
| C5 | 9q33-q34 | TRUE | 727 | complement component 5 | Munich |
| C5AR1 | 19q13.3-q13.4 | TRUE | 728 | complement component 5a receptor 1 | Aberdeen |
| C6ORF125 | 6p21.31 | TRUE | 84300 | chromosome 6 open reading frame 125 | Munich |
| CA2 | 8q22 | TRUE | 760 | carbonic anhydrase II | Munich |
| CACNA1C | 12p13.3 | TRUE | 775 | calcium channel, voltage-dependent, L type, alpha 1C subunit | Aberdeen |
| CACNA1D | 3p14.3 | TRUE | 776 | calcium channel, voltage-dependent, L type, alpha 1D subunit | Aberdeen |
| CACNA1E | 1q25-q31 | TRUE | 777 | calcium channel, voltage-dependent, alpha 1E subunit | Munich |
| CACNA1I | 22q13.1 | TRUE | 8911 | calcium channel, voltage-dependent, alpha 1I subunit | Aberdeen |
| CACNA1S | 1q32 | TRUE | 779 | calcium channel, voltage-dependent, L type, alpha 1S subunit | Aberdeen |
| CACNA2D1 | 7q21-q22 | TRUE | 781 | calcium channel, voltage-dependent, alpha 2/delta subunit 1 | Both Sets |
| CACNA2D1 | 7q21-q22 | TRUE | 781 | calcium channel, voltage-dependent, alpha 2/delta subunit 1 | Aberdeen |
| CACNA2D1 | 7q21-q22 | TRUE | 781 | calcium channel, voltage-dependent, alpha 2/delta subunit 1 | Munich |
| CACNA2D3 | 3p21.1 | TRUE | 55799 | calcium channel, voltage-dependent, alpha 2/delta 3 subunit | Aberdeen |
| CACNB2 | 10p12 | TRUE | 783 | calcium channel, voltage-dependent, beta 2 subunit | Aberdeen |
| CACNB2 | 10p12 | TRUE | 783 | calcium channel, voltage-dependent, beta 2 subunit | Munich |
| CACNG2 | 22q13.1 | TRUE | 10369 | calcium channel, voltage-dependent, gamma subunit 2 | Aberdeen |
| CAMK1D | 10p13 | TRUE | 57118 | calcium/calmodulin-dependent protein kinase ID | Aberdeen |
| CAMK2D | 4q26 | TRUE | 817 | calcium/calmodulin-dependent protein kinase (CaM kinase) II | Aberdeen |
| | | | | delta | |
| CAMKK1 | 17p13.2 | TRUE | 84254 | calcium/calmodulin-dependent protein kinase kinase 1, alpha | Aberdeen |
| CAPN11 | 6p12 | TRUE | 11131 | calpain 11 | Aberdeen |
| CAPN12 | 19q13.2 | TRUE | 147968 | calpain 12 | Aberdeen |
| CAPN13 | 2p22-p21 | TRUE | 92291 | calpain 13 | Aberdeen |
| CAPN5 | 11q14 | TRUE | 726 | calpain 5 | Munich |
| CAPN7 | 3p24 | TRUE | 23473 | calpain 7 | Aberdeen |
| CAPN9 | 1q42.11-q42.3 | TRUE | 10753 | calpain 9 | Munich |
| CART | 5q13.2 | FALSE | 9607 | cocaine- and amphetamine-regulated transcript | Munich |
| CASP14 | 19p13.1 | TRUE | 23581 | caspase 14, apoptosis-related cysteine peptidase | Aberdeen |
| CASP5 | 11q22.2-q22.3 | TRUE | 838 | caspase 5, apoptosis-related cysteine peptidase | Aberdeen |
| CASP6 | 4q25 | TRUE | 839 | caspase 6, apoptosis-related cysteine peptidase | Aberdeen |
| CASR | 3q13 | TRUE | 846 | calcium-sensing receptor (hypocalciuric hypercalcemia 1, | Aberdeen |
| | | | | severe neonatal hyperparathyroidism) | |
| CAST | 5q15 | TRUE | 831 | calpastatin | Aberdeen |
| CCDC46 | 17q24.1 | TRUE | 201134 | coiled-coil domain containing 46 | Aberdeen |
| CCL18 | 17q11.2 | TRUE | 6362 | chemokine (C-C motif) ligand 18 (pulmonary and activation- | Aberdeen |
| | | | | regulated) | |
| CCL2 | 17q11.2-q12 | TRUE | 6347 | chemokine (C-C motif) ligand 2 | Munich |
| CCL24 | 7q11.23 | TRUE | 6369 | chemokine (C-C motif) ligand 24 | Aberdeen |
| CCL27 | 9p13 | TRUE | 10850 | chemokine (C-C motif) ligand 27 | Munich |
| CCL3 | 17q11-q21 | TRUE | 6348 | chemokine (C-C motif) ligand 3 | Aberdeen |
| CCL4 | 17q12 | TRUE | 6351 | chemokine (C-C motif) ligand 4 | Aberdeen |
| CCR3 | 3p21.3 | TRUE | 1232 | chemokine (C-C motif) receptor 3 | Munich |
| CD14 | 5q22-q32\| | TRUE | 929 | CD14 molecule | Aberdeen |
| | 5q31.1 | | | | |
| CD209 | 19p13 | TRUE | 30835 | CD209 molecule | Aberdeen |
| CD36 | 7q11.2 | TRUE | 948 | CD36 molecule (thrombospondin receptor) | Munich |
| CD59 | 11p13 | TRUE | 966 | CD59 molecule, complement regulatory protein | Aberdeen |
| CD6 | 11q13 | TRUE | 923 | CD6 molecule | Aberdeen |
| CD74 | 5q32 | TRUE | 972 | CD74 molecule, major histocompatibility complex, class II | Aberdeen |
| | | | | invariant chain | |
| CD80 | 3q13.3-q21 | TRUE | 941 | CD80 molecule | Both Sets |
| CD80 | 3q13.3-q21 | TRUE | 941 | CD80 molecule | Aberdeen |
| CD80 | 3q13.3-q21 | TRUE | 941 | CD80 molecule | Munich |
| CD86 | 3q21 | TRUE | 942 | CD86 molecule | Munich |
| CDC25B | 20p13 | TRUE | 994 | cell division cycle 25B | Aberdeen |
| CDC25B | 20p13 | TRUE | 994 | cell division cycle 25B | Munich |
| CDC45L | 22q11.21 | TRUE | 8318 | CDC45 cell division cycle 45-like (S. cerevisiae) | Aberdeen |
| CDK6 | 7q21-q22 | TRUE | 1021 | cyclin-dependent kinase 6 | Aberdeen |
| CDK7 | 5q12.1 | TRUE | 1022 | cyclin-dependent kinase 7 (MO15 homolog, Xenopus laevis, | Aberdeen |
| | | | | cdk-activating kinase) | |
| CDKN1A | 6p21.2 | TRUE | 1026 | cyclin-dependent kinase inhibitor 1A (p21, Cip1) | Munich |
| CELSR1 | 22q13.3 | TRUE | 9620 | cadherin, EGF LAG seven-pass G-type receptor 1 (flamingo | Aberdeen |
| | | | | homolog, Drosophila) | |
| CELSR1 | 22q13.3 | TRUE | 9620 | cadherin, EGF LAG seven-pass G-type receptor 1 (flamingo | Munich |
| | | | | homolog, Drosophila) | |
| CELSR2 | 1p21 | TRUE | 1952 | cadherin, EGF LAG seven-pass G-type receptor 2 (flamingo | Aberdeen |
| | | | | homolog, Drosophila) | |
| CELSR3 | 3p24.1-p21.2 | TRUE | 1951 | cadherin, EGF LAG seven-pass G-type receptor 3 (flamingo | Munich |
| | | | | homolog, Drosophila) | |
| CENTB5 | | TRUE | 116983 | centaurin, beta 5 | Aberdeen |
| CES1 | 16q13-q22.1 | TRUE | 1066 | carboxylesterase 1 (monocyte/macrophage serine esterase 1) | Munich |
| CFB | 6p21.3 | TRUE | 629 | complement factor B | Both Sets |
| CFB | 6p21.3 | TRUE | 629 | complement factor B | Aberdeen |
| CFB | 6p21.3 | TRUE | 629 | complement factor B | Munich |
| CFI | 4q25 | TRUE | 3426 | complement factor I | Aberdeen |
| CFTR | 7q31.2 | TRUE | 1080 | cystic fibrosis transmembrane conductance regulator, ATP- | Munich |
| | | | | binding cassette (sub-family C, member 7) | |
| CHAT | 10q11.2 | TRUE | 1103 | choline acetyltransferase | Aberdeen |
| CHEK2 | 22q11\|22q12.1 | TRUE | 11200 | CHK2 checkpoint homolog (S. pombe) | Aberdeen |
| CHGA | 14q32 | TRUE | 1113 | chromogranin A (parathyroid secretory protein 1) | Aberdeen |
| CHRM3 | 1q43 | TRUE | 1131 | cholinergic receptor, muscarinic 3 | Munich |
| CHRNA1 | 2q24-q32 | TRUE | 1134 | cholinergic receptor, nicotinic, alpha 1 (muscle) | Both Sets |
| CHRNA1 | 2q24-q32 | TRUE | 1134 | cholinergic receptor, nicotinic, alpha 1 (muscle) | Aberdeen |
| CHRNA1 | 2q24-q32 | TRUE | 1134 | cholinergic receptor, nicotinic, alpha 1 (muscle) | Munich |
| CHRNA2 | 8p21 | TRUE | 1135 | cholinergic receptor, nicotinic, alpha 2 (neuronal) | Aberdeen |
| CHRNA4 | 20q13.2-q13.3 | TRUE | 1137 | cholinergic receptor, nicotinic, alpha 4 | Aberdeen |
| CHRNA6 | 8p11.21 | TRUE | 8973 | cholinergic receptor, nicotinic, alpha 6 | Aberdeen |
| CHRNA7 | 15q14 | TRUE | 1139 | cholinergic receptor, nicotinic, alpha 7 | Munich |
| CHRNA9 | 4p14 | TRUE | 55584 | cholinergic receptor, nicotinic, alpha 9 | Aberdeen |
| CHRND | 2q33-q34 | TRUE | 1144 | cholinergic receptor, nicotinic, delta | Munich |
| CHUK | 10q24-q25 | TRUE | 1147 | conserved helix-loop-helix ubiquitous kinase | Aberdeen |
| CHUK | 10q24-q25 | TRUE | 1147 | conserved helix-loop-helix ubiquitous kinase | Munich |
| CIDEB | 14q12 | TRUE | 27141 | cell death-inducing DFFA-like effector b | Munich |
| CLCA4 | 1p31-p22 | TRUE | 22802 | chloride channel, calcium activated, family member 4 | Munich |
| CLCN1 | 7q32-qter\| | TRUE | 1180 | chloride channel 1, skeletal muscle (Thomsen disease, | Munich |
| | 7q35 | | | autosomal dominant) | |
| CLCN3 | 4q33 | TRUE | 1182 | chloride channel 3 | Aberdeen |
| CLCN4 | Xp22.3 | TRUE | 1183 | chloride channel 4 | Munich |
| CLEC11A | 19q13.3 | TRUE | 6320 | C-type lectin domain family 11, member A | Aberdeen |
| CLIC1 | 6p22.1-p21.2 | TRUE | 1192 | chloride intracellular channel 1 | Aberdeen |
| CLPP | 19p13.3 | TRUE | 8192 | CIpP caseinolytic peptidase, ATP-dependent, proteolytic subunit | Aberdeen |
| | | | | homolog (E. coli) | |
| CMKLR1 | 12q24.1 | TRUE | 1240 | chemokine-like receptor 1 | Munich |
| CNGB1 | 16q13 | TRUE | 1258 | cyclic nucleotide gated channel beta 1 | Munich |
| COG2 | 1q42.2 | TRUE | 22796 | component of oligomeric golgi complex 2 | Aberdeen |
| COL11A2 | 6p21.3 | TRUE | 1302 | collagen, type XI, alpha 2 | Aberdeen |
| COL17A1 | 10q24.3 | TRUE | 1308 | collagen, type XVII, alpha 1 | Munich |
| COMT | 22q11.21-q11.23\| | TRUE | 1312 | catechol-O-methyltransferase | Aberdeen |
| | 22q11.21 | | | | |
| COPG2 | 7q32 | TRUE | 26958 | coatomer protein complex, subunit gamma 2 | Aberdeen |
| CPA1 | 7q32 | TRUE | 1357 | carboxypeptidase A1 (pancreatic) | Aberdeen |
| CPA3 | 3q21-q25 | TRUE | 1359 | carboxypeptidase A3 (mast cell) | Munich |
| CPE | 4q32.3 | TRUE | 1363 | carboxypeptidase E | Aberdeen |
| CPSF3L | 1p36.33 | TRUE | 54973 | cleavage and polyadenylation specific factor 3-like | Aberdeen |
| CPT1A | 11q13.1-q13.2 | TRUE | 1374 | carnitine palmitoyltransferase 1A (liver) | Aberdeen |
| CPVL | 7p15-p14 | TRUE | 54504 | carboxypeptidase, vitellogenic-like | Munich |
| CREB1 | 2q34 | TRUE | 1385 | cAMP responsive element binding protein 1 | Both Sets |
| CREB1 | 2q34 | TRUE | 1385 | cAMP responsive element binding protein 1 | Aberdeen |
| CREB1 | 2q34 | TRUE | 1385 | cAMP responsive element binding protein 1 | Munich |
| CREBBP | 16p13.3 | TRUE | 1387 | CREB binding protein (Rubinstein-Taybi syndrome) | Munich |
| CRH | 8q13 | TRUE | 1392 | corticotropin releasing hormone | Munich |
| CRHR1 | 17q12-q22 | TRUE | 1394 | corticotropin releasing hormone receptor 1 | Munich |
| CRHR2 | 7p15.1 | TRUE | 1395 | corticotropin releasing hormone receptor 2 | Munich |
| CRISP2 | 6p21-qter | TRUE | 7180 | cysteine-rich secretory protein 2 | Munich |
| CRISPLD2 | 16q24.1 | TRUE | 83716 | cysteine-rich secretory protein LCCL domain containing 2 | Aberdeen |
| CRISPLD2 | 16q24.1 | TRUE | 83716 | cysteine-rich secretory protein LCCL domain containing 2 | Munich |
| CRMP1 | 4p16.1-p15 | TRUE | 1400 | collapsin response mediator protein 1 | Aberdeen |
| CRMP1 | 4p16.1-p15 | TRUE | 1400 | collapsin response mediator protein 1 | Munich |
| CRSP2 | Xp11.4-p11.2 | TRUE | 9282 | cofactor required for Sp1 transcriptional activation, subunit 2, | Munich |
| | | | | 150 kDa | |
| CRSP6 | 11q14 | TRUE | 9440 | cofactor required for Sp1 transcriptional activation, subunit 6, | Aberdeen |
| | | | | 77 kDa | |
| CRSP8 | 9q34.1-q34.3 | TRUE | 9442 | cofactor required for Sp1 transcriptional activation, subunit 8, | Aberdeen |
| | | | | 34 kDa | |
| CSF1R | 5q33-q35 | TRUE | 1436 | colony stimulating factor 1 receptor, formerly McDonough feline | Aberdeen |
| | | | | sarcoma viral (v-fms) oncogene homolog | |
| CSNK1E | 22q13.1 | TRUE | 1454 | casein kinase 1, epsilon | Aberdeen |
| CSPG2 | 5q14.3 | TRUE | 1462 | chondroitin sulfate proteoglycan 2 (versican) | Munich |
| CTLA4 | 2q33 | TRUE | 1493 | cytotoxic T-lymphocyte-associated protein 4 | Aberdeen |
| CTSC | 11q14.1-q14.3 | TRUE | 1075 | cathepsin C | Munich |
| CTSK | 1q21 | TRUE | 1513 | cathepsin K (pycnodysostosis) | Both Sets |
| CTSK | 1q21 | TRUE | 1513 | cathepsin K (pycnodysostosis) | Aberdeen |
| CTSK | 1q21 | TRUE | 1513 | Cathepsin K (pycnodysostosis) | Munich |
| CTSLL2 | 10q | TRUE | 1517 | cathepsin L-like 2 | Aberdeen |
| CTSO | 4q31-q32 | TRUE | 1519 | cathepsin O | Aberdeen |
| CTSW | 11q13.1 | TRUE | 1521 | cathepsin W (lymphopain) | Aberdeen |
| CTSZ | 20q13 | TRUE | 1522 | cathepsin Z | Aberdeen |
| CX3CL1 | 16q13 | TRUE | 6376 | chemokine (C—X3—C motif) ligand 1 | Aberdeen |
| CX62 | 6q15-q16 | FALSE | 84694 | connexin 62 | Aberdeen |
| CXCL10 | 4q21 | TRUE | 3627 | chemokine (C—X—C motif) ligand 10 | Munich |
| CXCL11 | 4q21.2 | TRUE | 6373 | chemokine (C—X—C motif) ligand 11 | Munich |
| CXCL14 | 5q31 | TRUE | 9547 | chemokine (C—X—C motif) ligand 14 | Aberdeen |
| CXCL16 | 17p13 | TRUE | 58191 | chemokine (C—X—C motif) ligand 16 | Munich |
| CXCL9 | 4q21 | TRUE | 4283 | chemokine (C—X—C motif) ligand 9 | Munich |
| CXCR4 | 2q21 | TRUE | 7852 | chemokine (C—X—C motif) receptor 4 | Aberdeen |
| CXCR4 | 2q21 | TRUE | 7852 | chemokine (C—X—C motif) receptor 4 | Munich |
| CYP2C9 | 10q24 | TRUE | 1559 | cytochrome P450, family 2, subfamily C, polypeptide 9 | Aberdeen |
| CYP7A1 | 8q11-q12 | TRUE | 1581 | cytochrome P450, family 7, subfamily A, polypeptide 1 | Munich |
| CYSLTR2 | 13q14.12-q21.1 | TRUE | 57105 | cysteinyl leukotriene receptor 2 | Aberdeen |
| DAOA | 13q33.2\|13q34 | TRUE | 267012 | D-amino acid oxidase activator | Both Sets |
| DAOA | 13q33.2\|13q34 | TRUE | 267012 | D-amino acid oxidase activator | Aberdeen |
| DAOA | 13q33.2\|13q34 | TRUE | 267012 | D-amino acid oxidase activator | Munich |
| DAPK1 | 9q34.1 | TRUE | 1612 | death-associated protein kinase 1 | Aberdeen |
| DAPK3 | 19p13.3 | TRUE | 1613 | death-associated protein kinase 3 | Aberdeen |
| DBH | 9q34 | TRUE | 1621 | dopamine beta-hydroxylase (dopamine beta-monooxygenase) | Aberdeen |
| DCBLD2 | 3q12.1\|3 | TRUE | 131566 | discoidin, CUB and LCCL domain containing 2 | Aberdeen |
| DCBLD2 | 3q12.1\|3 | TRUE | 131566 | discoidin, CUB and LCCL domain containing 2 | Munich |
| DEFA3 | 8pter-p23.3 | TRUE | 1668 | defensin, alpha 3, neutrophil-specific | Aberdeen |
| DKFZP547C195 | 11q23.3 | FALSE | 257160 | hypothetical protein DKFZp547C195 | Munich |
| DLG2 | 11q14.1 | TRUE | 1740 | discs, large homolog 2, chapsyn-110 (Drosophila) | Aberdeen |
| DLG2 | 11q14.1 | TRUE | 1740 | discs, large homolog 2, chapsyn-110 (Drosophila) | Munich |
| DMP1 | 4q21 | TRUE | 1758 | dentin matrix acidic phosphoprotein | Aberdeen |
| DOCK7 | 1p31.3 | TRUE | 85440 | dedicator of cytokinesis 7 | Munich |
| DPP4 | 2q24.3 | TRUE | 1803 | dipeptidyl-peptidase 4 (CD26, adenosine deaminase | Munich |
| | | | | complexing protein 2) | |
| DPP9 | 19p13.3 | TRUE | 91039 | dipeptidyl-peptidase 9 | Aberdeen |
| DPYSL2 | 8p22-p21 | TRUE | 1808 | dihydropyrimidinase-like 2 | Aberdeen |
| DPYSL3 | 5q32 | TRUE | 1809 | dihydropyrimidinase-like 3 | Aberdeen |
| DPYSL5 | 2p23.3 | TRUE | 56896 | dihydropyrimidinase-like 5 | Aberdeen |
| DRD2 | 11q23 | TRUE | 1813 | dopamine receptor D2 | Aberdeen |
| DVL3 | 3q27 | TRUE | 1857 | dishevelled, dsh homolog 3 (Drosophila) | Aberdeen |
| DYRK1B | 19q12-13.1 | TRUE | 9149 | dual-specificity tyrosine-(Y)-phosphorylation regulated kinase 1B | Aberdeen |
| EBI2 | 13q32.3 | TRUE | 1880 | Epstein-Barr virus induced gene 2 (lymphocyte-specific G | Munich |
| | | | | protein-coupled receptor) | |
| EDG2 | 9q31.3 | TRUE | 1902 | endothelial differentiation, lysophosphatidic acid G-protein- | Aberdeen |
| | | | | coupled receptor, 2 | |
| EDG2 | 9q31.3 | TRUE | 1902 | endothelial differentiation, lysophosphatidic acid G-protein- | Munich |
| | | | | coupled receptor, 2 | |
| EDG4 | 19p12 | TRUE | 9170 | endothelial differentiation, lysophosphatidic acid G-protein- | Munich |
| | | | | coupled receptor, 4 | |
| EDG8 | 19p13.2 | TRUE | 53637 | endothelial differentiation, sphingolipid G-protein-coupled | Aberdeen |
| | | | | receptor, 8 | |
| EDIL3 | 5q14 | TRUE | 10085 | EGF-like repeats and discoidin I-like domains 3 | Aberdeen |
| EDIL3 | 5q14 | TRUE | 10085 | EGF-like repeats and discoidin I-like domains 3 | Munich |
| EDN1 | 6p24.1 | TRUE | 1906 | endothelin 1 | Munich |
| EDN3 | 20q13.2-q13.3 | TRUE | 1908 | endothelin 3 | Aberdeen |
| EEF2K | 16p12.1 | TRUE | 29904 | eukaryotic elongation factor-2 kinase | Aberdeen |
| EGF | 4q25 | TRUE | 1950 | epidermal growth factor (beta-urogastrone) | Munich |
| EGFR | 7p12 | TRUE | 1956 | epidermal growth factor receptor (erythroblastic leukemia viral | Munich |
| | | | | (v-erb-b) oncogene homolog, avian) | |
| EGR1 | 5q31.1 | TRUE | 1958 | early growth response 1 | Aberdeen |
| ELTD1 | 1p33-p32 | TRUE | 64123 | EGF, latrophilin and seven transmembrane domain containing 1 | Aberdeen |
| EMG1 | 12p13 | TRUE | 10436 | EMG1 nucleolar protein homolog (S. cerevisiae) | Aberdeen |
| EMR1 | 19p13.3 | TRUE | 2015 | egf-like module containing, mucin-like, hormone receptor-like 1 | Aberdeen |
| EMX2 | 10q26.1 | TRUE | 2018 | empty spiracles homolog 2 (Drosophila) | Aberdeen |
| ENSG00000148805 | 10q26.3 | FALSE | | Formerly hypothetical gene LOC118709. | Munich |
| EP300 | 22q13.2 | TRUE | 2033 | E1A binding protein p300 | Aberdeen |
| EPHA1 | 7q34 | TRUE | 2041 | EPH receptor A1 | Aberdeen |
| EPHA2 | 1p36 | TRUE | 1969 | EPH receptor A2 | Aberdeen |
| EPHA4 | 2q36.1 | TRUE | 2043 | EPH receptor A4 | Munich |
| EPHA5 | 4q13.1 | TRUE | 2044 | EPH receptor A5 | Munich |
| EPHA6 | 3q11.2 | TRUE | 285220 | EPH receptor A6 | Aberdeen |
| EPHB2 | 1p36.1-p35 | TRUE | 2048 | EPH receptor B2 | Aberdeen |
| ERBB3 | 12q13 | TRUE | 2065 | v-erb-b2 erythroblastic leukemia viral oncogene homolog 3 | Munich |
| | | | | (avian) | |
| ERBB4 | 2q33.3-q34 | TRUE | 2066 | v-erb-a erythroblastic leukemia viral oncogene homolog 4 | Aberdeen |
| | | | | (avian) | |
| ESM1 | 5q11.2 | TRUE | 11082 | endothelial cell-specific molecule 1 | Munich |
| ESPL1 | 12q | TRUE | 9700 | extra spindle poles like 1 (S. cerevisiae) | Aberdeen |
| ESR1 | 6q25.1 | TRUE | 2099 | estrogen receptor 1 | Munich |
| ESRRB | 14q24.3 | TRUE | 2103 | estrogen-related receptor beta | Munich |
| ESRRG | 1q41 | TRUE | 2104 | estrogen-related receptor gamma | Aberdeen |
| ESRRG | 1q41 | TRUE | 2104 | estrogen-related receptor gamma | Munich |
| EVC | 4p16 | TRUE | 2121 | Ellis van Creveld syndrome | Munich |
| F13A1 | 6p25.3-p24.3 | TRUE | 2162 | coagulation factor XIII, A1 polypeptide | Aberdeen |
| F2 | 11p11-q12 | TRUE | 2147 | coagulation factor II (thrombin) | Munich |
| F2RL2 | 5q13 | TRUE | 2151 | coagulation factor II (thrombin) receptor-like 2 | Both Sets |
| F2RL2 | 5q13 | TRUE | 2151 | coagulation factor II (thrombin) receptor-like 2 | Aberdeen |
| F2RL2 | 5q13 | TRUE | 2151 | coagulation factor II (thrombin) receptor-like 2 | Munich |
| F7 | 13q34 | TRUE | 2155 | coagulation factor VII (serum prothrombin conversion | Aberdeen |
| | | | | accelerator) | |
| F9 | Xq27.1-q27.2 | TRUE | 2158 | coagulation factor IX (plasma thromboplastic component, | Munich |
| | | | | Christmas disease, hemophilia B) | |
| FABP4 | 8q21 | TRUE | 2167 | fatty acid binding protein 4, adipocyte | Aberdeen |
| FAM20A | 17q24.2 | TRUE | 54757 | family with sequence similarity 20, member A | Munich |
| FAM21D | 10q11.23 | TRUE | 439973 | family with sequence similarity 21, member D | Aberdeen |
| FARP2 | 2q37.3 | TRUE | 9855 | FERM, RhoGEF and pleckstrin domain protein 2 | Aberdeen |
| FAS | 10q24.1 | TRUE | 355 | Fas (TNF receptor superfamily, member 6) | Aberdeen |
| FCER2 | 19p13.3 | TRUE | 2208 | Fc fragment of IgE, low affinity II, receptor for (CD23) | Aberdeen |
| FER | 5q21 | TRUE | 2241 | fer (fps/fes related) tyrosine kinase (phosphoprotein NCP94) | Aberdeen |
| FGF2 | 4q26-q27 | TRUE | 2247 | fibroblast growth factor 2 (basic) | Aberdeen |
| FGF2 | 4q26-q27 | TRUE | 2247 | fibroblast growth factor 2 (basic) | Munich |
| FGFR1 | 8p11.2-p11.1 | TRUE | 2260 | fibroblast growth factor receptor 1 (fms-related tyrosine kinase | Aberdeen |
| | | | | 2, Pfeiffer syndrome) | |
| FGFR2 | 10q26 | TRUE | 2263 | fibroblast growth factor receptor 2 (bacteria-expressed kinase, | Aberdeen |
| | | | | keratinocyte growth factor receptor, craniofacial dysostosis 1, | |
| | | | | Crouzon syndrome, Pfeiffer syndrome, Jackson-Weiss | |
| | | | | syndrome) | |
| FGFR3 | 4p16.3 | TRUE | 2261 | fibroblast growth factor receptor 3 (achondroplasia, | Aberdeen |
| | | | | thanatophoric dwarfism) | |
| FGR | 1p36.2-p36.1 | TRUE | 2268 | Gardner-Rasheed feline sarcoma viral (v-fgr) oncogene | Aberdeen |
| | | | | homolog | |
| FIBP | 11q13.1 | TRUE | 9158 | fibroblast growth factor (acidic) intracellular binding protein | Aberdeen |
| FLJ11286 | 19p13.2 | FALSE | 55337 | hypothetical protein FLJ11286 | Aberdeen |
| FLJ11850 | 4q11 | FALSE | 64752 | hypothetical protein FLJ11850 | Aberdeen |
| FLJ16331 | 11q13.1 | FALSE | 440044 | FLJ16331 protein | Munich |
| FLJ22709 | 19p13.11 | FALSE | 79629 | hypothetical protein FLJ22709 | Aberdeen |
| FLT1 | 13q12 | TRUE | 2321 | fms-related tyrosine kinase 1 (vascular endothelial growth | Aberdeen |
| | | | | factor/vascular permeability factor receptor) | |
| FLT1 | 13q12 | TRUE | 2321 | fms-related tyrosine kinase 1 (vascular endothelial growth | Munich |
| | | | | factor/vascular permeability factor receptor) | |
| FLT3 | 13q12 | TRUE | 2322 | fms-related tyrosine kinase 3 | Munich |
| FLT4 | 5q34-q35 | TRUE | 2324 | fms-related tyrosine kinase 4 | Aberdeen |
| FOSL1 | 11q13 | TRUE | 8061 | FOS-like antigen 1 | Aberdeen |
| FRK | 6q21-q22.3 | TRUE | 2444 | fyn-related kinase | Aberdeen |
| FRMPD4 | Xp22.2 | TRUE | 9758 | FERM and PDZ domain containing 4 | Aberdeen |
| FRMPD4 | Xp22.2 | TRUE | 9758 | FERM and PDZ domain containing 4 | Munich |
| FRZB | 2qter | TRUE | 2487 | frizzled-related protein | Aberdeen |
| FSHR | 2p21-p16 | TRUE | 2492 | follicle stimulating hormone receptor | Munich |
| FYN | 6q21 | TRUE | 2534 | FYN oncogene related to SRC, FGR, YES | Munich |
| FZD2 | 17q21.1 | TRUE | 2535 | frizzled homolog 2 (Drosophila) | Munich |
| FZD4 | 11q14.2 | TRUE | 8322 | frizzled homolog 4 (Drosophila) | Munich |
| G30 | 13q33.2\|13q34 | FALSE | 282706 | putative protein LG30 | Both Sets |
| G30 | 13q33.2\|13q34 | FALSE | 282706 | putative protein LG30 | Aberdeen |
| G30 | 13q33.2\|13q34 | FALSE | 282706 | putative protein LG30 | Munich |
| GABBR2 | 9q22.1-q22.3 | TRUE | 9568 | gamma-aminobutyric acid (GABA) B receptor, 2 | Munich |
| GABRA3 | Xq28 | TRUE | 2556 | gamma-aminobutyric acid (GABA) A receptor, alpha 3 | Both Sets |
| GABRA3 | Xq28 | TRUE | 2556 | gamma-aminobutyric acid (GABA) A receptor, alpha 3 | Aberdeen |
| GABRA3 | Xq28 | TRUE | 2556 | gamma-aminobutyric acid (GABA) A receptor, alpha 3 | Munich |
| GABRA5 | 15q11.2-q12 | TRUE | 2558 | gamma-aminobutyric acid (GABA) A receptor, alpha 5 | Munich |
| GABRB2 | 5q34 | TRUE | 2561 | gamma-aminobutyric acid (GABA) A receptor, beta 2 | Aberdeen |
| GABRB3 | 15q11.2-q12 | TRUE | 2562 | gamma-aminobutyric acid (GABA) A receptor, beta 3 | Aberdeen |
| GABRG2 | 5q31.1-q33.1 | TRUE | 2566 | gamma-aminobutyric acid (GABA) A receptor, gamma 2 | Aberdeen |
| GABRG3 | 15q12 | TRUE | 2567 | gamma-aminobutyric acid (GABA) A receptor, gamma 3 | Aberdeen |
| GABRP | 5q33-q34 | TRUE | 2568 | gamma-aminobutyric acid (GABA) A receptor, pi | Aberdeen |
| GABRQ | Xq28 | TRUE | 55879 | gamma-aminobutyric acid (GABA) receptor, theta | Aberdeen |
| GABRR1 | 6q14-q21\| | TRUE | 2569 | gamma-aminobutyric acid (GABA) receptor, rho 1 | Aberdeen |
| | 6q13-q16.3 | | | | |
| GAK | 4p16 | TRUE | 2580 | cyclin G associated kinase | Aberdeen |
| GAL | 11q13.2 | TRUE | 51083 | galanin | Aberdeen |
| GATA4 | 8p23.1-p22 | TRUE | 2626 | GATA binding protein 4 | Munich |
| GCK | 7p15.3-p15.1 | TRUE | 2645 | glucokinase (hexokinase 4, maturity onset diabetes of the young | Aberdeen |
| | | | | 2) | |
| GGTLA1 | 22q11.23 | TRUE | 2687 | gamma-glutamyltransferase-like activity 1 | Aberdeen |
| GHR | 5p13-p12 | TRUE | 2690 | growth hormone receptor | Munich |
| GHRL | 3p26-p25 | TRUE | 51738 | ghrelin/obestatin preprohormone | Aberdeen |
| GJA3 | 13q11-q12 | TRUE | 2700 | gap junction protein, alpha 3, 46 kDa (connexin 46) | Aberdeen |
| GLRA1 | 5q32 | TRUE | 2741 | glycine receptor, alpha 1 (startle disease/hyperekplexia, stiff | Munich |
| | | | | man syndrome) | |
| GMIP | 19p12-p11 | TRUE | 51291 | GEM interacting protein | Munich |
| GNAI1 | 7q21 | TRUE | 2770 | guanine nucleotide binding protein (G protein), alpha inhibiting | Munich |
| | | | | activity polypeptide 1 | |
| GNAI2 | 3p21 | TRUE | 2771 | guanine nucleotide binding protein (G protein), alpha inhibiting | Aberdeen |
| | | | | activity polypeptide 2 | |
| GNAL | 18p11.22-p11.21 | TRUE | 2774 | guanine nucleotide binding protein (G protein), alpha activating | Munich |
| | | | | activity polypeptide, olfactory type | |
| GNRH1 | 8p21-p11.2 | TRUE | 2796 | gonadotropin-releasing hormone 1 (luteinizing-releasing | Munich |
| | | | | hormone) | |
| GNRHR | 4q21.2 | TRUE | 2798 | gonadotropin-releasing hormone receptor | Munich |
| GPC3 | Xq26.1 | TRUE | 2719 | glypican 3 | Aberdeen |
| GPC4 | Xq26.1 | TRUE | 2239 | glypican 4 | Aberdeen |
| GPC5 | 13q32 | TRUE | 2262 | glypican 5 | Munich |
| GPR113 | 2p23.3 | TRUE | 165082 | G protein-coupled receptor 113 | Munich |
| GPR114 | 16q13 | TRUE | 221188 | G protein-coupled receptor 114 | Aberdeen |
| GPR114 | 16q13 | TRUE | 221188 | G protein-coupled receptor 114 | Munich |
| GPR116 | 6p12.3 | TRUE | 221395 | G protein-coupled receptor 116 | Munich |
| GPR126 | 6q24.1 | TRUE | 57211 | G protein-coupled receptor 126 | Munich |
| GPR128 | 3q12.2 | TRUE | 84873 | G protein-coupled receptor 128 | Aberdeen |
| GPR133 | 12q24.33 | TRUE | 283383 | G protein-coupled receptor 133 | Aberdeen |
| GPR137 | 11cen-q22.3 | TRUE | 56834 | G protein-coupled receptor 137 | Aberdeen |
| GPR142 | 17q25.1 | TRUE | 350383 | G protein-coupled receptor 142 | Aberdeen |
| GPR142 | 17q25.1 | TRUE | 350383 | G protein-coupled receptor 142 | Munich |
| GPR146 | 7p22.3 | TRUE | 115330 | G protein-coupled receptor 146 | Munich |
| GPR150 | 5q15 | TRUE | 285601 | G protein-coupled receptor 150 | Munich |
| GPR151 | 5q32 | TRUE | 134391 | G protein-coupled receptor 151 | Aberdeen |
| GPR160 | 3q26.2-q27 | TRUE | 26996 | G protein-coupled receptor 160 | Munich |
| GPR171 | 3q25.1 | TRUE | 29909 | G protein-coupled receptor 171 | Munich |
| GPR173 | Xp11 | TRUE | 54328 | G protein-coupled receptor 173 | Munich |
| GPR19 | 12p12.3 | TRUE | 2842 | G protein-coupled receptor 19 | Aberdeen |
| GPR20 | 8q24.2-q24.3 | TRUE | 2843 | G protein-coupled receptor 20 | Munich |
| GPR35 | 2q37.3 | TRUE | 2859 | G protein-coupled receptor 35 | Munich |
| GPR56 | 16q12.2-q21 | TRUE | 9289 | G protein-coupled receptor 56 | Aberdeen |
| GPR61 | 1p13.3 | TRUE | 83873 | G protein-coupled receptor 61 | Munich |
| GPR64 | Xp22.13 | TRUE | 10149 | G protein-coupled receptor 64 | Munich |
| GPR98 | 5q13 | TRUE | 84059 | G protein-coupled receptor 98 | Aberdeen |
| GPR98 | 5q13 | TRUE | 84059 | G protein-coupled receptor 98 | Munich |
| GPRC5B | 16p12 | TRUE | 51704 | G protein-coupled receptor, family C, group 5, member B | Munich |
| GRIA1 | 5q33\|5q31.1 | TRUE | 2890 | glutamate receptor, ionotropic, AMPA 1 | Munich |
| GRIA3 | Xq25-q26 | TRUE | 2892 | glutamate receptor, ionotrophic, AMPA 3 | Aberdeen |
| GRID1 | 10q22 | TRUE | 2894 | glutamate receptor, ionotropic, delta 1 | Aberdeen |
| GRID2 | 4q22 | TRUE | 2895 | glutamate receptor, ionotropic, delta 2 | Aberdeen |
| GRIK1 | 21q22.11 | TRUE | 2897 | glutamate receptor, ionotropic, kainate 1 | Aberdeen |
| GRIK2 | 6q16.3-q21 | TRUE | 2898 | glutamate receptor, ionotropic, kainate 2 | Aberdeen |
| GRIN1 | 9q34.3 | TRUE | 2902 | glutamate receptor, ionotropic, N-methyl D-aspartate 1 | Aberdeen |
| GRIN2A | 16p13.2 | TRUE | 2903 | glutamate receptor, ionotropic, N-methyl D-aspartate 2A | Aberdeen |
| GRIN2B | 12p12 | TRUE | 2904 | glutamate receptor, ionotropic, N-methyl D-aspartate 2B | Aberdeen |
| GRIN2B | 12p12 | TRUE | 2904 | glutamate receptor, ionotropic, N-methyl D-aspartate 2B | Munich |
| GRIN3A | 9q31.1 | TRUE | 116443 | glutamate receptor, ionotropic, N-methyl-D-aspartate 3A | Aberdeen |
| GRM2 | 3p21.1 | TRUE | 2912 | glutamate receptor, metabotropic 2 | Aberdeen |
| GRM3 | 7q21.1-q21.2 | TRUE | 2913 | glutamate receptor, metabotropic 3 | Munich |
| GRM5 | 11q14.2-q14.3 | TRUE | 2915 | glutamate receptor, metabotropic 5 | Both Sets |
| GRM5 | 11q14.2-q14.3 | TRUE | 2915 | glutamate receptor, metabotropic 5 | Aberdeen |
| GRM5 | 11q14.2-q14.3 | TRUE | 2915 | glutamate receptor, metabotropic 5 | Munich |
| GRM7 | 3p26.1-p25.1 | TRUE | 2917 | glutamate receptor, metabotropic 7 | Aberdeen |
| GRM7 | 3p26.1-p25.1 | TRUE | 2917 | glutamate receptor, metabotropic 7 | Munich |
| GRM8 | 7q31.3-q32.1 | TRUE | 2918 | glutamate receptor, metabotropic 8 | Aberdeen |
| GRWD1 | 19q13.32 | TRUE | 83743 | glutamate-rich WD repeat containing 1 | Both Sets |
| GRWD1 | 19q13.32 | TRUE | 83743 | glutamate-rich WD repeat containing 1 | Aberdeen |
| GRWD1 | 19q13.32 | TRUE | 83743 | glutamate-rich WD repeat containing 1 | Munich |
| GUCA2A | 1p35-p34 | TRUE | 2980 | guanylate cyclase activator 2A (guanylin) | Munich |
| GUCY1B2 | 13q14.2-q14.3 | TRUE | 2974 | guanylate cyclase 1, soluble, beta 2 | Aberdeen |
| GZMH | 14q11.2 | TRUE | 2999 | granzyme H (cathepsin G-like 2, protein h-CCPX) | Munich |
| HAAO | 2p21 | TRUE | 23498 | 3-hydroxyanthranilate 3,4-dioxygenase | Aberdeen |
| HCN2 | 19p13.3 | TRUE | 610 | hyperpolarization activated cyclic nucleotide-gated potassium | Aberdeen |
| | | | | channel 2 | |
| HDAC4 | 2q37.2 | TRUE | 9759 | histone deacetylase 4 | Aberdeen |
| HDAC4 | 2q37.2 | TRUE | 9759 | histone deacetylase 4 | Munich |
| HFE | 6p21.3 | TRUE | 3077 | hemochromatosis | Munich |
| HIG2 | 7q32.1 | FALSE | 29923 | hypoxia-inducible protein 2 | Aberdeen |
| HIPHUM_302 | 11q22 | FALSE | | Cysteine protease, caspase | Aberdeen |
| HMMR | 5q33.2-qter | TRUE | 3161 | hyaluronan-mediated motility receptor (RHAMM) | Aberdeen |
| HRH1 | 3p25 | TRUE | 3269 | histamine receptor H1 | Munich |
| HRH3 | 20q13.33 | TRUE | 11255 | histamine receptor H3 | Munich |
| HRH4 | 18q11.2 | TRUE | 59340 | histamine receptor H4 | Aberdeen |
| HSD3B1 | 1p13.1 | TRUE | 3283 | hydroxy-delta-5-steroid dehydrogenase, 3 beta- and steroid | Munich |
| | | | | delta-isomerase 1 | |
| HTATIP | 11q13 | TRUE | 10524 | HIV-1 Tat interacting protein, 60 kDa | Aberdeen |
| HTR2A | 13q14-q21 | TRUE | 3356 | 5-hydroxytryptamine (serotonin) receptor 2A | Munich |
| HTR2B | 2q36.3-q37.1 | TRUE | 3357 | 5-hydroxytryptamine (serotonin) receptor 2B | Aberdeen |
| HTR2C | Xq24 | TRUE | 3358 | 5-hydroxytryptamine (serotonin) receptor 2C | Both Sets |
| HTR2C | Xq24 | TRUE | 3358 | 5-hydroxytryptamine (serotonin) receptor 2C | Aberdeen |
| HTR2C | Xq24 | TRUE | 3358 | 5-hydroxytryptamine (serotonin) receptor 2C | Munich |
| HTR3A | 11q23.1 | TRUE | 3359 | 5-hydroxytryptamine (serotonin) receptor 3A | Munich |
| HTR4 | 5q31-q33 | TRUE | 3360 | 5-hydroxytryptamine (serotonin) receptor 4 | Aberdeen |
| HTR4 | 5q31-q33 | TRUE | 3360 | 5-hydroxytryptamine (serotonin) receptor 4 | Munich |
| HUNK | 21q22.1 | TRUE | 30811 | hormonally upregulated Neu-associated kinase | Aberdeen |
| ICAM1 | 19p13.3-p13.2 | TRUE | 3383 | intercellular adhesion molecule 1 (CD54), human rhinovirus | Munich |
| | | | | receptor | |
| ICAM4 | 19p13.2-cen | TRUE | 3386 | intercellular adhesion molecule 4 (Landsteiner-Wiener blood | Munich |
| | | | | group) | |
| ICAM5 | 19p13.2 | TRUE | 7087 | intercellular adhesion molecule 5, telencephalin | Aberdeen |
| IDE | 10q23-q25 | TRUE | 3416 | insulin-degrading enzyme | Aberdeen |
| IFI16 | 1q22 | TRUE | 3428 | interferon, gamma-inducible protein 16 | Aberdeen |
| IGF1R | 15q26.3 | TRUE | 3480 | insulin-like growth factor 1 receptor | Aberdeen |
| IKBKAP | 9q31 | TRUE | 8518 | inhibitor of kappa light polypeptide gene enhancer in B-cells, | Munich |
| | | | | kinase complex-associated protein | |
| IKBKE | 1q32.1 | TRUE | 9641 | inhibitor of kappa light polypeptide gene enhancer in B-cells, | Aberdeen |
| | | | | kinase epsilon | |
| IKBKE | 1q32.1 | TRUE | 9641 | inhibitor of kappa light polypeptide gene enhancer in B-cells, | Munich |
| | | | | kinase epsilon | |
| IL10 | 1q31-q32 | TRUE | 3586 | interleukin 10 | Aberdeen |
| IL12B | 5q31.1-q33.1 | TRUE | 3593 | interleukin 12B (natural killer cell stimulatory factor 2, cytotoxic | Aberdeen |
| | | | | lymphocyte maturation factor 2, p40) | |
| IL15 | 4q31 | TRUE | 3600 | interleukin 15 | Aberdeen |
| IL15RA | 10p15-p14 | TRUE | 3601 | interleukin 15 receptor, alpha | Munich |
| IL16 | 15q26.3 | TRUE | 3603 | interleukin 16 (lymphocyte chemoattractant factor) | Aberdeen |
| IL1B | 2q14 | TRUE | 3553 | interleukin 1, beta | Munich |
| IL1R1 | 2q12 | TRUE | 3554 | interleukin 1 receptor, type I | Aberdeen |
| IL2 | 4q26-q27 | TRUE | 3558 | interleukin 2 | Aberdeen |
| IL2RB | 22q13\|22q13.1 | TRUE | 3560 | interleukin 2 receptor, beta | Aberdeen |
| IL4 | 5q31.1 | TRUE | 3565 | interleukin 4 | Aberdeen |
| IL6R | 1q21 | TRUE | 3570 | interleukin 6 receptor | Munich |
| IL6ST | 5q11 | TRUE | 3572 | interleukin 6 signal transducer (gp130, oncostatin M receptor) | Munich |
| ILK | 11p15.5-p15.4 | TRUE | 3611 | integrin-linked kinase | Munich |
| INSR | 19p13.3-p13.2 | TRUE | 3643 | insulin receptor | Munich |
| IQGAP2 | 5q13.3 | TRUE | 10788 | IQ motif containing GTPase activating protein 2 | Both Sets |
| IQGAP2 | 5q13.3 | TRUE | 10788 | IQ motif containing GTPase activating protein 2 | Aberdeen |
| IQGAP2 | 5q13.3 | TRUE | 10788 | IQ motif containing GTPase activating protein 2 | Munich |
| IRAK2 | 3p25.3 | TRUE | 3656 | interleukin-1 receptor-associated kinase 2 | Aberdeen |
| IRF2 | 4q34.1-q35.1 | TRUE | 3660 | interferon regulatory factor 2 | Aberdeen |
| IRS2 | 13q34 | TRUE | 8660 | insulin receptor substrate 2 | Munich |
| ITGA1 | 5q11.2 | TRUE | 3672 | integrin, alpha 1 | Aberdeen |
| ITGA2 | 5q23-q31 | TRUE | 3673 | integrin, alpha 2 (CD49B, alpha 2 subunit of VLA-2 receptor) | Munich |
| ITGA9 | 3p21.3 | TRUE | 3680 | integrin, alpha 9 | Aberdeen |
| ITGAV | 2q31-q32 | TRUE | 3685 | integrin, alpha V (vitronectin receptor, alpha polypeptide, | Aberdeen |
| | | | | antigen CD51) | |
| ITGAX | 16p11.2 | TRUE | 3687 | integrin, alpha X (complement component 3 receptor 4 subunit) | Aberdeen |
| ITGB1 | 10p11.2 | TRUE | 3688 | integrin, beta 1 (fibronectin receptor, beta polypeptide, antigen | Munich |
| | | | | CD29 includes MDF2, MSK12) | |
| ITGB2 | 21q22.3 | TRUE | 3689 | integrin, beta 2 (complement component 3 receptor 3 and 4 | Aberdeen |
| | | | | subunit) | |
| ITGB3 | 17q21.32 | TRUE | 3690 | integrin, beta 3 (platelet glycoprotein IIIa, antigen CD61) | Munich |
| ITGB6 | 2q24.2 | TRUE | 3694 | integrin, beta 6 | Aberdeen |
| ITGB7 | 12q13.13 | TRUE | 3695 | integrin, beta 7 | Aberdeen |
| ITK | 5q31-q32 | TRUE | 3702 | IL2-inducible T-cell kinase | Munich |
| ITPR1 | 3p26-p25 | TRUE | 3708 | inositol 1,4,5-triphosphate receptor, type 1 | Aberdeen |
| ITPR2 | 12p11 | TRUE | 3709 | inositol 1,4,5-triphosphate receptor, type 2 | Aberdeen |
| ITPR2 | 12p11 | TRUE | 3709 | inositol 1,4,5-triphosphate receptor, type 2 | Munich |
| JAK1 | 1p32.3-p31.3 | TRUE | 3716 | Janus kinase 1 (a protein tyrosine kinase) | Aberdeen |
| JAK1 | 1p32.3-p31.3 | TRUE | 3716 | Janus kinase 1 (a protein tyrosine kinase) | Munich |
| JMJD1C | 10q21.2 | TRUE | 221037 | jumonji domain containing 1C | Munich |
| KCNA5 | 12p13 | TRUE | 3741 | potassium voltage-gated channel, shaker-related subfamily, | Aberdeen |
| | | | | member 5 | |
| KCNA5 | 12p13 | TRUE | 3741 | potassium voltage-gated channel, shaker-related subfamily, | Munich |
| | | | | member 5 | |
| KCNA6 | 12p13 | TRUE | 3742 | potassium voltage-gated channel, shaker-related subfamily, | Aberdeen |
| | | | | member 6 | |
| KCNAB1 | 3q26.1 | TRUE | 7881 | potassium voltage-gated channel, shaker-related subfamily, | Munich |
| | | | | beta member 1 | |
| KCNAB2 | 1p36.3 | TRUE | 8514 | potassium voltage-gated channel, shaker-related subfamily, | Aberdeen |
| | | | | beta member 2 | |
| KCNB2 | 8q13.2 | TRUE | 9312 | potassium voltage-gated channel, Shab-related subfamily, | Both Sets |
| | | | | member 2 | |
| KCNB2 | 8q13.2 | TRUE | 9312 | potassium voltage-gated channel, Shab-related subfamily, | Aberdeen |
| | | | | member 2 | |
| KCNB2 | 8q13.2 | TRUE | 9312 | potassium voltage-gated channel, Shab-related subfamily, | Munich |
| | | | | member 2 | |
| KCNC1 | 11p15 | TRUE | 3746 | potassium voltage-gated channel, Shaw-related subfamily, | Aberdeen |
| | | | | member 1 | |
| KCND3 | 1p13.3 | TRUE | 3752 | potassium voltage-gated channel, Shal-related subfamily, | Aberdeen |
| | | | | member 3 | |
| KCND3 | 1p13.3 | TRUE | 3752 | potassium voltage-gated channel, Shal-related subfamily, | Munich |
| | | | | member 3 | |
| KCNE1 | 21q22.1-q22.2\| | TRUE | 3753 | potassium voltage-gated channel, lsk-related family, member 1 | Aberdeen |
| | 21q22.12 | | | | |
| KCNG3 | 2p21 | TRUE | 170850 | potassium voltage-gated channel, subfamily G, member 3 | Aberdeen |
| KCNG4 | 16q24.1 | TRUE | 93107 | potassium voltage-gated channel, subfamily G, member 4 | Munich |
| KCNH1 | 1q32-q41 | TRUE | 3756 | potassium voltage-gated channel, subfamily H (eag-related), | Aberdeen |
| | | | | member 1 | |
| KCNH1 | 1q32-q41 | TRUE | 3756 | potassium voltage-gated channel, subfamily H (eag-related), | Munich |
| | | | | member 1 | |
| KCNH4 | 17q21.2 | TRUE | 23415 | potassium voltage-gated channel, subfamily H (eag-related), | Aberdeen |
| | | | | member 4 | |
| KCNH4 | 17q21.2 | TRUE | 23415 | potassium voltage-gated channel, subfamily H (eag-related), | Munich |
| | | | | member 4 | |
| KCNH5 | 14q23.1 | TRUE | 27133 | potassium voltage-gated channel, subfamily H (eag-related), | Aberdeen |
| | | | | member 5 | |
| KCNH5 | 14q23.1 | TRUE | 27133 | potassium voltage-gated channel, subfamily H (eag-related), | Munich |
| | | | | member 5 | |
| KCNH7 | 2q24.2 | TRUE | 90134 | potassium voltage-gated channel, subfamily H (eag-related), | Both Sets |
| | | | | member 7 | |
| KCNH7 | 2q24.2 | TRUE | 90134 | potassium voltage-gated channel, subfamily H (eag-related), | Aberdeen |
| | | | | member 7 | |
| KCNH7 | 2q24.2 | TRUE | 90134 | potassium voltage-gated channel, subfamily H (eag-related), | Munich |
| | | | | member 7 | |
| KCNIP3 | 2q21.1 | TRUE | 30818 | Kv channel interacting protein 3, calsenilin | Munich |
| KCNJ14 | 19q13 | TRUE | 3770 | potassium inwardly-rectifying channel, subfamily J, member 14 | Both Sets |
| KCNJ14 | 19q13 | TRUE | 3770 | potassium inwardly-rectifying channel, subfamily J, member 14 | Aberdeen |
| KCNJ14 | 19q13 | TRUE | 3770 | potassium inwardly-rectifying channel, subfamily J, member 14 | Munich |
| KCNJ16 | 17q23.1-q24.2 | TRUE | 3773 | potassium inwardly-rectifying channel, subfamily J, member 16 | Munich |
| KCNJ6 | 21q22.1\|21q22.13-q22.2 | TRUE | 3763 | potassium inwardly-rectifying channel, subfamily J, member 6 | Aberdeen |
| KCNJ6 | 21q22.1\|21q22.13-q22.2 | TRUE | 3763 | potassium inwardly-rectifying channel, subfamily J, member 6 | Munich |
| KCNK1 | 1q42-q43 | TRUE | 3775 | potassium channel, subfamily K, member 1 | Aberdeen |
| KCNK12 | 2p22-p21 | TRUE | 56660 | potassium channel, subfamily K, member 12 | Aberdeen |
| KCNK13 | 14q31-q32 | TRUE | 56659 | potassium channel, subfamily K, member 13 | Both Sets |
| KCNK13 | 14q31-q32 | TRUE | 56659 | potassium channel, subfamily K, member 13 | Aberdeen |
| KCNK13 | 14q31-q32 | TRUE | 56659 | potassium channel, subfamily K, member 13 | Munich |
| KCNK15 | 20q12-20q13 | TRUE | 60598 | potassium channel, subfamily K, member 15 | Munich |
| KCNK9 | 8q24.3 | TRUE | 51305 | potassium channel, subfamily K, member 9 | Munich |
| KCNMA1 | 10q22.3 | TRUE | 3778 | potassium large conductance calcium-activated channel, | Aberdeen |
| | | | | subfamily M, alpha member 1 | |
| KCNMB4 | 12q | TRUE | 27345 | potassium large conductance calcium-activated channel, | Munich |
| | | | | subfamily M, beta member 4 | |
| KCNN2 | 5q22.3 | TRUE | 3781 | potassium intermediate/small conductance calcium-activated | Aberdeen |
| | | | | channel, subfamily N, member 2 | |
| KCNN3 | 1q21.3 | TRUE | 3782 | potassium intermediate/small conductance calcium-activated | Aberdeen |
| | | | | channel, subfamily N, member 3 | |
| KCNN3 | 1q21.3 | TRUE | 3782 | potassium intermediate/small conductance calcium-activated | Munich |
| | | | | channel, subfamily N, member 3 | |
| KCNQ1 | 11p15.5 | TRUE | 3784 | potassium voltage-gated channel, KQT-like subfamily, member 1 | Aberdeen |
| KCNQ3 | 8q24 | TRUE | 3786 | potassium voltage-gated channel, KQT-like subfamily, member 3 | Aberdeen |
| KCNQ5 | 6q14 | TRUE | 56479 | potassium voltage-gated channel, KQT-like subfamily, member 5 | Aberdeen |
| KCNT1 | 9q34.3 | TRUE | 57582 | potassium channel, subfamily T, member 1 | Aberdeen |
| KCTD9 | 8p21.1 | TRUE | 54793 | potassium channel tetramerisation domain containing 9 | Aberdeen |
| KIAA0319L | 1p34.2 | TRUE | 79932 | KIAA0319-like | Aberdeen |
| KIAA0409 | 11p15.4 | TRUE | 23378 | KIAA0409 | Aberdeen |
| KIAA1245 | 1q21.2 | TRUE | 149013 | KIAA1245 | Aberdeen |
| KIRREL3 | 11q24 | TRUE | 84623 | kin of IRRE like 3 (Drosophila) | Munich |
| KIT | 4q11-q12 | TRUE | 3815 | v-kit Hardy-Zuckerman 4 feline sarcoma viral oncogene | Aberdeen |
| | | | | homolog | |
| KLK15 | 19q13.41 | TRUE | 55554 | kallikrein 15 | Aberdeen |
| KMO | 1q42-q44 | TRUE | 8564 | kynurenine 3-monooxygenase (kynurenine 3-hydroxylase) | Aberdeen |
| KMO | 1q42-q44 | TRUE | 8564 | kynurenine 3-monooxygenase (kynurenine 3-hydroxylase) | Munich |
| KNG1 | 3q27 | TRUE | 3827 | kininogen 1 | Aberdeen |
| KSR1 | 17q11.1 | TRUE | 8844 | kinase suppressor of ras 1 | Aberdeen |
| L29306 | | FALSE | | | Aberdeen |
| LANCL2 | 7q31.1-q31.33 | TRUE | 55915 | LanC lantibiotic synthetase component C-like 2 (bacterial) | Aberdeen |
| LGR4 | 11p14-p13 | TRUE | 55366 | leucine-rich repeat-containing G protein-coupled receptor 4 | Munich |
| LGR6 | 1q32.1 | TRUE | 59352 | leucine-rich repeat-containing G protein-coupled receptor 6 | Aberdeen |
| LHB | 19q13.32 | TRUE | 3972 | luteinizing hormone beta polypeptide | Aberdeen |
| LNPEP | 5q15 | TRUE | 4012 | leucyl/cystinyl aminopeptidase | Munich |
| LOC123862 | 16q24.1 | FALSE | 123862 | similar to Interferon-induced transmembrane protein 3 | Munich |
| | | | | (Interferon-inducible protein 1-8U) | |
| LOC124216 | 16p13.3 | FALSE | 124216 | hypothetical LOC124216 | Aberdeen |
| LOC284454 | 19p13.13 | FALSE | 284454 | hypothetical protein LOC284454 | Aberdeen |
| LOC338598 | 10q11.22 | FALSE | 338598 | hypothetical LOC338598 | Aberdeen |
| LOC439949 | 10p15.1 | FALSE | 439949 | hypothetical gene supported by AY007155 | Aberdeen |
| LOC441792 | 17q12 | FALSE | 441792 | similar to Nitric oxide synthase, inducible (NOS type II) | Aberdeen |
| | | | | (Inducible NOS) (iNOS) (Hepatocyte NOS) (HEP-NOS) | |
| LOC442200 | 6p21.33 | FALSE | 442200 | similar to Fibroblast growth factor receptor 3 precursor (FGFR- | Aberdeen |
| | | | | 3) | |
| LOC642927 | 10p15.3 | FALSE | 642927 | similar to alpha 3 type IV collagen isoform 1, precursor | Aberdeen |
| LOC643376 | 11q12.1 | FALSE | 643376 | similar to Kelch-like protein 2 | Aberdeen |
| LOC643524 | 5q21.3 | FALSE | 643524 | hypothetical protein LOC643524 | Aberdeen |
| LOC644248 | 4q22.1 | FALSE | 644248 | hypothetical protein LOC644248 | Aberdeen |
| LOC644613 | 11q13.1 | FALSE | 644613 | hypothetical protein LOC644613 | Aberdeen |
| LOC644784 | 22q11.1 | FALSE | 644784 | similar to Phosphatidylinositol-4,5-bisphosphate 3-kinase | Aberdeen |
| | | | | catalytic subunit, alpha isoform (PI3-kinase p110 subunit alpha) | |
| | | | | (PtdIns-3-kinase p110) (PI3K) | |
| LOC645542 | 9q21.32 | FALSE | 645542 | similar to SET domain and mariner transposase fusion gene | Aberdeen |
| LOC645710 | 17q24.2 | FALSE | 645710 | hypothetical protein LOC645710 | Aberdeen |
| LOC646115 | 19q13.33 | FALSE | 646115 | hypothetical protein LOC646115 | Aberdeen |
| LOC646960 | 2q37.1 | FALSE | 646960 | similar to transmembrane protease, serine 9 | Munich |
| LOC647747 | | FALSE | 647747 | hypothetical protein LOC647747 | Aberdeen |
| LOC648278 | | FALSE | 648278 | similar to Kelch-like protein 2 | Aberdeen |
| LOC648299 | | FALSE | 648299 | similar to Phosphatidylinositol-4,5-bisphosphate 3-kinase | Aberdeen |
| | | | | catalytic subunit, alpha isoform (PI3-kinase p110 subunit alpha) | |
| | | | | (PtdIns-3-kinase p110) (PI3K) | |
| LOC649659 | | FALSE | 649659 | hypothetical protein LOC649659 | Aberdeen |
| LOC651864 | | FALSE | 651864 | hypothetical protein LOC651864 | Aberdeen |
| LOC654433 | | FALSE | 654433 | hypothetical LOC654433 | Munich |
| LPHN3 | 4q13.1 | TRUE | 23284 | latrophilin 3 | Munich |
| LPL | 8p22 | TRUE | 4023 | lipoprotein lipase | Munich |
| LRP5 | 11q13.4 | TRUE | 4041 | low density lipoprotein receptor-related protein 5 | Aberdeen |
| LRP6 | 12p11-p13 | TRUE | 4040 | low density lipoprotein receptor-related protein 6 | Aberdeen |
| LTA | 6p21.3 | TRUE | 4049 | lymphotoxin alpha (TNF superfamily, member 1) | Aberdeen |
| LTA4H | 12q22 | TRUE | 4048 | leukotriene A4 hydrolase | Munich |
| LTB4R2 | 14q11.2-q12 | TRUE | 56413 | leukotriene B4 receptor 2 | Munich |
| MAP2K1 | 15q22.1-q22.33 | TRUE | 5604 | mitogen-activated protein kinase kinase 1 | Aberdeen |
| MAP3K10 | 19q13.2 | TRUE | 4294 | mitogen-activated protein kinase kinase kinase 10 | Munich |
| MAP3K12 | 12q13 | TRUE | 7786 | mitogen-activated protein kinase kinase kinase 12 | Aberdeen |
| MAP3K14 | 17q21 | TRUE | 9020 | mitogen-activated protein kinase kinase kinase 14 | Aberdeen |
| MAP3K7 | 6q16.1-q16.3 | TRUE | 6885 | mitogen-activated protein kinase kinase kinase 7 | Aberdeen |
| MAP3K8 | 10p11.23 | TRUE | 1326 | mitogen-activated protein kinase kinase kinase 8 | Munich |
| MAP4K3 | 2p22.1 | TRUE | 8491 | mitogen-activated protein kinase kinase kinase kinase 3 | Aberdeen |
| MAP4K4 | 2q11.2-q12 | TRUE | 9448 | mitogen-activated protein kinase kinase kinase kinase 4 | Aberdeen |
| MAPK4 | 18q12-q21 | TRUE | 5596 | mitogen-activated protein kinase 4 | Aberdeen |
| MAPK9 | 5q35 | TRUE | 5601 | mitogen-activated protein kinase 9 | Aberdeen |
| MAS1L | 6p21 | TRUE | 116511 | MAS1 oncogene-like | Munich |
| MBTPS1 | 16\|16q24 | TRUE | 8720 | membrane-bound transcription factor peptidase, site 1 | Munich |
| MBTPS2 | Xp22.1-p22.2 | TRUE | 51360 | membrane-bound transcription factor peptidase, site 2 | Aberdeen |
| MC1R | 16q24.3 | TRUE | 4157 | melanocortin 1 receptor (alpha melanocyte stimulating hormone | Munich |
| | | | | receptor) | |
| MC2R | 18p11.2 | TRUE | 4158 | melanocortin 2 receptor (adrenocorticotropic hormone) | Aberdeen |
| MED12 | Xq13 | TRUE | 9968 | mediator of RNA polymerase II transcription, subunit 12 | Aberdeen |
| | | | | homolog (S. cerevisiae) | |
| MED12L | 3q25.1 | TRUE | 116931 | mediator of RNA polymerase II transcription, subunit 12 | Munich |
| | | | | homolog (S. cerevisiae)-like | |
| MED19 | 11q12.1 | TRUE | 219541 | mediator of RNA polymerase II transcription, subunit 19 | Aberdeen |
| | | | | homolog (S. cerevisiae) | |
| MEN1 | 11q13 | TRUE | 4221 | multiple endocrine neoplasia I | Aberdeen |
| MEPE | 4q21.1 | TRUE | 56955 | matrix, extracellular phosphoglycoprotein with ASARM motif | Aberdeen |
| | | | | (bone) | |
| MEST | 7q32 | TRUE | 4232 | mesoderm specific transcript homolog (mouse) | Aberdeen |
| MFRP | 11q23 | TRUE | 83552 | membrane frizzled-related protein | Munich |
| MGC10334 | 1p36.33 | FALSE | 80772 | hypothetical protein MGC10334 | Aberdeen |
| MGC11257 | 7p22.3 | FALSE | 84310 | hypothetical protein MGC11257 | Munich |
| MGC4618 | 4p16.3 | FALSE | 84286 | hypothetical protein MGC4618 | Aberdeen |
| MICA | 6p21.3 | TRUE | 4276 | MHC class I polypeptide-related sequence A | Munich |
| MIPEP | 13q12 | TRUE | 4285 | mitochondrial intermediate peptidase | Aberdeen |
| MKNK1 | 1p33 | TRUE | 8569 | MAP kinase interacting serine/threonine kinase 1 | Munich |
| MMP1 | 11q22.3 | TRUE | 4312 | matrix metallopeptidase 1 (interstitial collagenase) | Both Sets |
| MMP1 | 11q22.3 | TRUE | 4312 | matrix metallopeptidase 1 (interstitial collagenase) | Aberdeen |
| MMP1 | 11q22.3 | TRUE | 4312 | matrix metallopeptidase 1 (interstitial collagenase) | Munich |
| MMP10 | 11q22.3 | TRUE | 4319 | matrix metallopeptidase 10 (stromelysin 2) | Aberdeen |
| MMP16 | 8q21 | TRUE | 4325 | matrix metallopeptidase 16 (membrane-inserted) | Both Sets |
| MMP16 | 8q21 | TRUE | 4325 | matrix metallopeptidase 16 (membrane-inserted) | Aberdeen |
| MMP16 | 8q21 | TRUE | 4325 | matrix metallopeptidase 16 (membrane-inserted) | Munich |
| MMP20 | 11q22.3 | TRUE | 9313 | matrix metallopeptidase 20 (enamelysin) | Aberdeen |
| MMP28 | 17q11-q21.1 | TRUE | 79148 | matrix metallopeptidase 28 | Munich |
| MMP3 | 11q22.3 | TRUE | 4314 | matrix metallopeptidase 3 (stromelysin 1, progelatinase) | Aberdeen |
| MMP9 | 20q11.2-q13.1 | TRUE | 4318 | matrix metallopeptidase 9 (gelatinase B, 92 kDa gelatinase, | Aberdeen |
| | | | | 92 kDa type IV collagenase) | |
| MON1A | 3p21.31 | TRUE | 84315 | MON1 homolog A (yeast) | Aberdeen |
| MRGPRD | 11q13.2 | TRUE | 116512 | MAS-related GPR, member D | Munich |
| MRGPRG | 11p15.4 | TRUE | 386746 | MAS-related GPR, member G | Munich |
| MRGPRX2 | 11p15.1 | TRUE | 117194 | MAS-related GPR, member X2 | Munich |
| MRGPRX3 | 11p15.1 | TRUE | 117195 | MAS-related GPR, member X3 | Aberdeen |
| MS4A4A | 11q12 | TRUE | 51338 | membrane-spanning 4-domains, subfamily A, member 4 | Aberdeen |
| MS4A6E | 11q12.2 | TRUE | 245802 | membrane-spanning 4-domains, subfamily A, member 6E | Munich |
| MS4A8B | 11q12.2 | TRUE | 83661 | membrane-spanning 4-domains, subfamily A, member 8B | Munich |
| MTNR1A | 4q35.1 | TRUE | 4543 | melatonin receptor 1A | Aberdeen |
| MUSK | 9q31.3-q32 | TRUE | 4593 | muscle, skeletal, receptor tyrosine kinase | Aberdeen |
| MUSK | 9q31.3-q32 | TRUE | 4593 | muscle, skeletal, receptor tyrosine kinase | Munich |
| NAB2 | 12q13.3-q14.1 | TRUE | 4665 | NGFI-A binding protein 2 (EGR1 binding protein 2) | Aberdeen |
| NAPSB | 19q13.33 | TRUE | 256236 | napsin B aspartic peptidase pseudogene | Munich |
| NCOR2 | 12q24 | TRUE | 9612 | nuclear receptor co-repressor 2 | Aberdeen |
| NCOR2 | 12q24 | TRUE | 9612 | nuclear receptor co-repressor 2 | Munich |
| NEDD4 | 15q | TRUE | 4734 | neural precursor cell expressed, developmentally down- | Aberdeen |
| | | | | regulated 4 | |
| NEDD4L | 18q21 | TRUE | 23327 | neural precursor cell expressed, developmentally down- | Aberdeen |
| | | | | regulated 4-like | |
| NEDD9 | 6p25-p24 | TRUE | 4739 | neural precursor cell expressed, developmentally down- | Aberdeen |
| | | | | regulated 9 | |
| NEFH | 22q12.2 | TRUE | 4744 | neurofilament, heavy polypeptide 200 kDa | Aberdeen |
| NEFL | 8p21 | TRUE | 4747 | neurofilament, light polypeptide 68 kDa | Both Sets |
| NEFL | 8p21 | TRUE | 4747 | neurofilament, light polypeptide 68 kDa | Aberdeen |
| NEFL | 8p21 | TRUE | 4747 | neurofilament, light polypeptide 68 kDa | Munich |
| NFATC2 | 20q13.2-q13.3 | TRUE | 4773 | nuclear factor of activated T-cells, cytoplasmic, calcineurin- | Munich |
| | | | | dependent 2 | |
| NFKB1 | 4q24 | TRUE | 4790 | nuclear factor of kappa light polypeptide gene enhancer in B- | Aberdeen |
| | | | | cells 1 (p105) | |
| NFKBIB | 19q13.1 | TRUE | 4793 | nuclear factor of kappa light polypeptide gene enhancer in B- | Munich |
| | | | | cells inhibitor, beta | |
| NFKBIL1 | 6p21.3 | TRUE | 4795 | nuclear factor of kappa light polypeptide gene enhancer in B- | Munich |
| | | | | cells inhibitor-like 1 | |
| NLN | 5q12.3 | TRUE | 57486 | neurolysin (metallopeptidase M3 family) | Aberdeen |
| NMB | 15q22-qter | TRUE | 4828 | neuromedin B | Aberdeen |
| NMUR2 | 5q33.1 | TRUE | 56923 | neuromedin U receptor 2 | Aberdeen |
| NOLC1 | 10q24.32 | TRUE | 9221 | nucleolar and coiled-body phosphoprotein 1 | Munich |
| NOS2A | 17q11.2-q12 | TRUE | 4843 | nitric oxide synthase 2A (inducible, hepatocytes) | Munich |
| NOS3 | 7q36 | TRUE | 4846 | nitric oxide synthase 3 (endothelial cell) | Aberdeen |
| NPC1L1 | 7p13 | TRUE | 29881 | NPC1 (Niemann-Pick disease, type C1, gene)-like 1 | Aberdeen |
| NPPC | 2q24-qter | TRUE | 4880 | natriuretic peptide precursor C | Aberdeen |
| NPSR1 | 7p14.3 | TRUE | 387129 | neuropeptide S receptor 1 | Munich |
| NPY1R | 4q31.3-q32 | TRUE | 4886 | neuropeptide Y receptor Y1 | Aberdeen |
| NR0B2 | 1p36.1 | TRUE | 8431 | nuclear receptor subfamily 0, group B, member 2 | Munich |
| NR2F6 | 19p13.1 | TRUE | 2063 | nuclear receptor subfamily 2, group F, member 6 | Aberdeen |
| NR3C1 | 5q31.3 | TRUE | 2908 | nuclear receptor subfamily 3, group C, member 1 (glucocorticoid | Munich |
| | | | | receptor) | |
| NR3C2 | 4q31.1 | TRUE | 4306 | nuclear receptor subfamily 3, group C, member 2 | Munich |
| NR4A1 | 12q13 | TRUE | 3164 | nuclear receptor subfamily 4, group A, member 1 | Aberdeen |
| NR4A3 | 9q22 | TRUE | 8013 | nuclear receptor subfamily 4, group A, member 3 | Aberdeen |
| NRG1 | 8p21-p12 | TRUE | 3084 | neuregulin 1 | Aberdeen |
| NRG3 | 10q22-q23 | TRUE | 10718 | neuregulin 3 | Aberdeen |
| NRG4 | 15q24.2 | TRUE | 145957 | neuregulin 4 | Aberdeen |
| NTF3 | 12p13 | TRUE | 4908 | neurotrophin 3 | Munich |
| NUDT18 | 8p21.3 | TRUE | 79873 | nudix (nucleoside diphosphate linked moiety X)-type motif 18 | Munich |
| NUDT6 | 4q26 | TRUE | 11162 | nudix (nucleoside diphosphate linked moiety X)-type motif 6 | Munich |
| OXER1 | 2p21 | TRUE | 165140 | oxoeicosanoid (OXE) receptor 1 | Aberdeen |
| OXGR1 | 13q32.1 | TRUE | 27199 | oxoglutarate (alpha-ketoglutarate) receptor 1 | Munich |
| P2RX1 | 17p13.3 | TRUE | 5023 | purinergic receptor P2X, ligand-gated ion channel, 1 | Aberdeen |
| P2RX4 | 12q24.32 | TRUE | 5025 | purinergic receptor P2X, ligand-gated ion channel, 4 | Munich |
| P2RX7 | 12q24 | TRUE | 5027 | purinergic receptor P2X, ligand-gated ion channel, 7 | Munich |
| P2RY2 | 11q13.5-q14.1 | TRUE | 5029 | purinergic receptor P2Y, G-protein coupled, 2 | Munich |
| PADI2 | 1p35.2-p35.1 | TRUE | 11240 | peptidyl arginine deiminase, type II | Munich |
| PAK2 | 3q29 | TRUE | 5062 | p21 (CDKN1A)-activated kinase 2 | Aberdeen |
| PAK6 | 15q14 | TRUE | 56924 | p21(CDKN1A)-activated kinase 6 | Aberdeen |
| PAK7 | 20p12 | TRUE | 57144 | p21(CDKN1A)-activated kinase 7 | Both Sets |
| PAK7 | 20p12 | TRUE | 57144 | p21(CDKN1A)-activated kinase 7 | Aberdeen |
| PAK7 | 20p12 | TRUE | 57144 | p21(CDKN1A)-activated kinase 7 | Munich |
| PANX2 | 22q13.33 | TRUE | 56666 | pannexin 2 | Aberdeen |
| PAQR5 | 15q23 | TRUE | 54852 | progestin and adipoQ receptor family member V | Aberdeen |
| PAQR8 | 6p12.1 | TRUE | 85315 | progestin and adipoQ receptor family member VIII | Aberdeen |
| PAX8 | 2q12-q14 | TRUE | 7849 | paired box gene 8 | Aberdeen |
| PAX8 | 2q12-q14 | TRUE | 7849 | paired box gene 8 | Munich |
| PBX2 | 6p21.3 | TRUE | 5089 | pre-B-cell leukemia transcription factor 2 | Aberdeen |
| PCAF | 3p24 | TRUE | 8850 | p300/CBP-associated factor | Both Sets |
| PCAF | 3p24 | TRUE | 8850 | p300/CBP-associated factor | Aberdeen |
| PCAF | 3p24 | TRUE | 8850 | p300/CBP-associated factor | Munich |
| PCDHB4 | 5q31 | TRUE | 56131 | protocadherin beta 4 | Aberdeen |
| PCK1 | 20q13.31 | TRUE | 5105 | phosphoenolpyruvate carboxykinase 1 (soluble) | Aberdeen |
| PCSK9 | 1p32.3 | TRUE | 255738 | proprotein convertase subtilisin/kexin type 9 | Aberdeen |
| PCSK9 | 1p32.3 | TRUE | 255738 | proprotein convertase subtilisin/kexin type 9 | Munich |
| PCTK2 | 12q23.1 | TRUE | 5128 | PCTAIRE protein kinase 2 | Aberdeen |
| PDE1C | 7p15.1-p14.3 | TRUE | 5137 | phosphodiesterase 1C, calmodulin-dependent 70 kDa | Aberdeen |
| PDE2A | 11q13.4 | TRUE | 5138 | phosphodiesterase 2A, cGMP-stimulated | Aberdeen |
| PDE4B | 1p31 | TRUE | 5142 | phosphodiesterase 4B, cAMP-specific (phosphodiesterase E4 | Aberdeen |
| | | | | dunce homolog, Drosophila) | |
| PDE4B | 1p31 | TRUE | 5142 | phosphodiesterase 4B, cAMP-specific (phosphodiesterase E4 | Munich |
| | | | | dunce homolog, Drosophila) | |
| PDE4C | 19p13.11 | TRUE | 5143 | phosphodiesterase 4C, cAMP-specific (phosphodiesterase E1 | Aberdeen |
| | | | | dunce homolog, Drosophila) | |
| PDE4D | 5q12 | TRUE | 5144 | phosphodiesterase 4D, cAMP-specific (phosphodiesterase E3 | Aberdeen |
| | | | | dunce homolog, Drosophila) | |
| PDE7A | 8q13 | TRUE | 5150 | phosphodiesterase 7A | Munich |
| PDGFRA | 4q11-q13 | TRUE | 5156 | platelet-derived growth factor receptor, alpha polypeptide | Aberdeen |
| PDK1 | 2q31.1 | TRUE | 5163 | pyruvate dehydrogenase kinase, isozyme 1 | Aberdeen |
| PDPK1 | 16p13.3 | TRUE | 5170 | 3-phosphoinositide dependent protein kinase-1 | Aberdeen |
| PDYN | 20pter-p12 | TRUE | 5173 | prodynorphin | Aberdeen |
| PEO1 | 10q23.3-24.3 | TRUE | 56652 | progressive external ophthalmoplegia 1 | Munich |
| PGC | 6p21.3-p21.1 | TRUE | 5225 | progastricsin (pepsinogen C) | Munich |
| PGR | 11q22-q23 | TRUE | 5241 | progesterone receptor | Aberdeen |
| PHB2 | 12p13 | TRUE | 11331 | prohibitin 2 | Aberdeen |
| PHEX | Xp22.2-p22.1 | TRUE | 5251 | phosphate regulating endopeptidase homolog, X-linked | Munich |
| | | | | (hypophosphatemia, vitamin D resistant rickets) | |
| PHGDHL1 | 13q32.3 | TRUE | 337867 | phosphoglycerate dehydrogenase like 1 | Munich |
| PHKG1 | 7p11.2 | TRUE | 5260 | phosphorylase kinase, gamma 1 (muscle) | Aberdeen |
| PIAS2 | 18q21.1 | TRUE | 9063 | protein inhibitor of activated STAT, 2 | Aberdeen |
| PIM2 | Xp11.23 | TRUE | 11040 | pim-2 oncogene | Munich |
| PKD2L2 | 5q31 | TRUE | 27039 | polycystic kidney disease 2-like 2 | Aberdeen |
| PLA2G4A | 1q25 | TRUE | 5321 | phospholipase A2, group IVA (cytosolic, calcium-dependent) | Aberdeen |
| PLA2G4C | 19q13.3 | TRUE | 8605 | phospholipase A2, group IVC (cytosolic, calcium-independent) | Aberdeen |
| PLA2G6 | 22q13.1 | TRUE | 8398 | phospholipase A2, group VI (cytosolic, calcium-independent) | Aberdeen |
| PLG | 6q26 | TRUE | 5340 | plasminogen | Aberdeen |
| PLK4 | 4q27-q28 | TRUE | 10733 | polo-like kinase 4 (Drosophila) | Aberdeen |
| PLTP | 20q12-q13.1 | TRUE | 5360 | phospholipid transfer protein | Aberdeen |
| PLTP | 20q12-q13.1 | TRUE | 5360 | phospholipid transfer protein | Munich |
| PMPCB | 7q22.1 | TRUE | 9512 | peptidase (mitochondrial processing) beta | Aberdeen |
| POLD2 | 7p13 | TRUE | 5425 | polymerase (DNA directed), delta 2, regulatory subunit 50 kDa | Aberdeen |
| POLRMT | 19p13.3 | TRUE | 5442 | polymerase (RNA) mitochondrial (DNA directed) | Munich |
| PPARBP | 17q12-q21.1 | TRUE | 5469 | PPAR binding protein | Munich |
| PPARGC1A | 4p15.1 | TRUE | 10891 | peroxisome proliferative activated receptor, gamma, coactivator | Aberdeen |
| | | | | 1, alpha | |
| PPM1B | 2p21 | TRUE | 5495 | protein phosphatase 1B (formerly 2C), magnesium-dependent, | Aberdeen |
| | | | | beta isoform | |
| PPM1E | 17q22 | TRUE | 22843 | protein phosphatase 1E (PP2C domain containing) | Aberdeen |
| PPM1G | 2p23.3 | TRUE | 5496 | protein phosphatase 1G (formerly 2C), magnesium-dependent, | Aberdeen |
| | | | | gamma isoform | |
| PPP1CA | 11q13 | TRUE | 5499 | protein phosphatase 1, catalytic subunit, alpha isoform | Aberdeen |
| PPRC1 | 10q24.32 | TRUE | 23082 | peroxisome proliferative activated receptor, gamma, coactivator- | Aberdeen |
| | | | | related 1 | |
| PREP | 6q22 | TRUE | 5550 | prolyl endopeptidase | Munich |
| PRKAB1 | 12q24.1 | TRUE | 5564 | protein kinase, AMP-activated, beta 1 non-catalytic subunit | Munich |
| PRKAB2 | 1q21.1 | TRUE | 5565 | protein kinase, AMP-activated, beta 2 non-catalytic subunit | Munich |
| PRKAR1A | 17q23-q24 | TRUE | 5573 | protein kinase, cAMP-dependent, regulatory, type I, alpha | Munich |
| | | | | (tissue specific extinguisher 1) | |
| PRKCA | 17q22-q23.2 | TRUE | 5578 | protein kinase C, alpha | Aberdeen |
| PRKCB1 | 16p11.2 | TRUE | 5579 | protein kinase C, beta 1 | Aberdeen |
| PRKCE | 2p21 | TRUE | 5581 | protein kinase C, epsilon | Munich |
| PRKCQ | 10p15 | TRUE | 5588 | protein kinase C, theta | Aberdeen |
| PRKD1 | 14q11 | TRUE | 5587 | protein kinase D1 | Aberdeen |
| PRKG1 | 10q11.2 | TRUE | 5592 | protein kinase, cGMP-dependent, type I | Aberdeen |
| PRLHR | 10q26.13 | TRUE | 2834 | prolactin releasing hormone receptor | Munich |
| PRND | 20pter-p12 | TRUE | 23627 | prion protein 2 (dublet) | Munich |
| | p12 | | | | |
| PRODH | 22q11.21 | TRUE | 5625 | proline dehydrogenase (oxidase) 1 | Aberdeen |
| PRODH2 | 19q13.1 | TRUE | 58510 | proline dehydrogenase (oxidase) 2 | Aberdeen |
| PROKR2 | 20p12.3 | TRUE | 128674 | prokineticin receptor 2 | Both Sets |
| PROKR2 | 20p12.3 | TRUE | 128674 | prokineticin receptor 2 | Aberdeen |
| PROKR2 | 20p12.3 | TRUE | 128674 | prokineticin receptor 2 | Munich |
| PRSS22 | 16p13.3 | TRUE | 64063 | protease, serine, 22 | Munich |
| PRSS23 | 11q14.1 | TRUE | 11098 | protease, serine, 23 | Aberdeen |
| PRSS3 | 9p11.2 | TRUE | 5646 | protease, serine, 3 (mesotrypsin) | Aberdeen |
| PRSS3 | 9p11.2 | TRUE | 5646 | protease, serine, 3 (mesotrypsin) | Munich |
| PSAP | 10q21-q22 | TRUE | 5660 | prosaposin (variant Gaucher disease and variant metachromatic | Aberdeen |
| | | | | leukodystrophy) | |
| PSCD2 | 19q13.3 | TRUE | 9266 | pleckstrin homology, Sec7 and coiled-coil domains 2 (cytohesin- | Aberdeen |
| | | | | 2) | |
| PSD4 | 2q13 | TRUE | 23550 | pleckstrin and Sec7 domain containing 4 | Aberdeen |
| PSMA5 | 1p13 | TRUE | 5686 | proteasome (prosome, macropain) subunit, alpha type, 5 | Aberdeen |
| PSMB1 | 6q27 | TRUE | 5689 | proteasome (prosome, macropain) subunit, beta type, 1 | Munich |
| PSMB6 | 17p13 | TRUE | 5694 | proteasome (prosome, macropain) subunit, beta type, 6 | Munich |
| PSMB7 | 9q34.11-q34.12 | TRUE | 5695 | proteasome (prosome, macropain) subunit, beta type, 7 | Munich |
| PSMC1 | 14q32.11 | TRUE | 5700 | proteasome (prosome, macropain) 26S subunit, ATPase, 1 | Aberdeen |
| PSMC3 | 11p12-p13 | TRUE | 5702 | proteasome (prosome, macropain) 26S subunit, ATPase, 3 | Aberdeen |
| PSMD1 | 2q37.1 | TRUE | 5707 | proteasome (prosome, macropain) 26S subunit, non-ATPase, 1 | Aberdeen |
| PTAFR | 1p35-p34.3 | TRUE | 5724 | platelet-activating factor receptor | Aberdeen |
| PTEN | 10q23.3 | TRUE | 5728 | phosphatase and tensin homolog (mutated in multiple advanced | Aberdeen |
| | | | | cancers 1) | |
| PTGDR | 14q22.1 | TRUE | 5729 | prostaglandin D2 receptor (DP) | Both Sets |
| PTGDR | 14q22.1 | TRUE | 5729 | prostaglandin D2 receptor (DP) | Aberdeen |
| PTGDR | 14q22.1 | TRUE | 5729 | prostaglandin D2 receptor (DP) | Munich |
| PTGER2 | 14q22 | TRUE | 5732 | prostaglandin E receptor 2 (subtype EP2), 53 kDa | Aberdeen |
| PTGER3 | 1p31.2 | TRUE | 5733 | prostaglandin E receptor 3 (subtype EP3) | Munich |
| PTGS1 | 9q32-q33.3 | TRUE | 5742 | prostaglandin-endoperoxide synthase 1 (prostaglandin G/H | Aberdeen |
| | | | | synthase and cyclooxygenase) | |
| PTH | 11p15.3-p15.1 | TRUE | 5741 | parathyroid hormone | Aberdeen |
| PTH | 11p15.3-p15.1 | TRUE | 5741 | parathyroid hormone | Munich |
| PTK2 | 8q24-qter | TRUE | 5747 | PTK2 protein tyrosine kinase 2 | Munich |
| PTK2B | 8p21.1 | TRUE | 2185 | PTK2B protein tyrosine kinase 2 beta | Aberdeen |
| PTK6 | 20q13.3 | TRUE | 5753 | PTK6 protein tyrosine kinase 6 | Aberdeen |
| PTPN13 | 4q21.3 | TRUE | 5783 | protein tyrosine phosphatase, non-receptor type 13 (APO- | Munich |
| | | | | 1/CD95 (Fas)-associated phosphatase) | |
| PTPN22 | 1p13.3-p13.1 | TRUE | 26191 | protein tyrosine phosphatase, non-receptor type 22 (lymphoid) | Aberdeen |
| PTPRC | 1q31-q32 | TRUE | 5788 | protein tyrosine phosphatase, receptor type, C | Both Sets |
| PTPRC | 1q31-q32 | TRUE | 5788 | protein tyrosine phosphatase, receptor type, C | Aberdeen |
| PTPRC | 1q31-q32 | TRUE | 5788 | protein tyrosine phosphatase, receptor type, C | Munich |
| PYGB | 20p11.2-p11.1 | TRUE | 5834 | phosphorylase, glycogen; brain | Aberdeen |
| PYY2 | 17q11 | TRUE | 23615 | peptide YY, 2 (seminalplasmin) | Aberdeen |
| QSCN6 | 1q24 | TRUE | 5768 | quiescin Q6 | Aberdeen |
| RAB5B | 12q13 | TRUE | 5869 | RAB5B, member RAS oncogene family | Munich |
| RARB | 3p24 | TRUE | 5915 | retinoic acid receptor, beta | Aberdeen |
| RDBP | 6p21.3 | TRUE | 7936 | RD RNA binding protein | Both Sets |
| RDBP | 6p21.3 | TRUE | 7936 | RD RNA binding protein | Aberdeen |
| RDBP | 6p21.3 | TRUE | 7936 | RD RNA binding protein | Munich |
| RECK | 9p13-p12 | TRUE | 8434 | reversion-inducing-cysteine-rich protein with kazal motifs | Munich |
| RETN | 19p13.2 | TRUE | 56729 | resistin | Aberdeen |
| RIMS4 | 20q13.12 | TRUE | 140730 | regulating synaptic membrane exocytosis 4 | Munich |
| RIPK2 | 8q21 | TRUE | 8767 | receptor-interacting serine-threonine kinase 2 | Aberdeen |
| RNF175 | 4q31.3 | TRUE | 285533 | ring finger protein 175 | Aberdeen |
| RNF26 | 11q23 | TRUE | 79102 | ring finger protein 26 | Munich |
| ROCK1 | 18q11.1 | TRUE | 6093 | Rho-associated, coiled-coil containing protein kinase 1 | Munich |
| ROR1 | 1p32-p31 | TRUE | 4919 | receptor tyrosine kinase-like orphan receptor 1 | Aberdeen |
| RORA | 15q21-q22 | TRUE | 6095 | RAR-related orphan receptor A | Aberdeen |
| RORB | 9q22 | TRUE | 6096 | RAR-related orphan receptor B | Aberdeen |
| RP11- | 10q11.22 | FALSE | 653365 | N-acylsphingosine amidohydrolase (non-lysosomal ceramidase) | Aberdeen |
| 98I6.3 | | | | 2C | |
| RPS6KA1 | 1p | TRUE | 6195 | ribosomal protein S6 kinase, 90 kDa, polypeptide 1 | Aberdeen |
| RPS6KA2 | 6q27 | TRUE | 6196 | ribosomal protein S6 kinase, 90 kDa, polypeptide 2 | Aberdeen |
| RPS6KA4 | 11q11-q13 | TRUE | 8986 | ribosomal protein S6 kinase, 90 kDa, polypeptide 4 | Aberdeen |
| RUNX1 | 21q22.3 | TRUE | 861 | runt-related transcription factor 1 (acute myeloid leukemia 1; | Aberdeen |
| | | | | aml1 oncogene) | |
| RUVBL2 | 19q13.3 | TRUE | 10856 | RuvB-like 2 (E. coli) | Aberdeen |
| RXRG | 1q22-q23 | TRUE | 6258 | retinoid X receptor, gamma | Munich |
| RYR1 | 19q13.1 | TRUE | 6261 | ryanodine receptor 1 (skeletal) | Aberdeen |
| RYR2 | 1q42.1-q43 | TRUE | 6262 | ryanodine receptor 2 (cardiac) | Aberdeen |
| RYR2 | 1q42.1-q43 | TRUE | 6262 | ryanodine receptor 2 (cardiac) | Munich |
| RYR3 | 15q14-q15 | TRUE | 6263 | ryanodine receptor 3 | Aberdeen |
| RYR3 | 15q14-q15 | TRUE | 6263 | ryanodine receptor 3 | Munich |
| S100A9 | 1q21 | TRUE | 6280 | S100 calcium binding protein A9 (calgranulin B) | Aberdeen |
| SCD5 | 4q21.22 | TRUE | 79966 | stearoyl-CoA desaturase 5 | Aberdeen |
| SCD5 | 4q21.22 | TRUE | 79966 | stearoyl-CoA desaturase 5 | Munich |
| SCN1A | 2q24.3 | TRUE | 6323 | sodium channel, voltage-gated, type I, alpha | Aberdeen |
| SCN1B | 19q13.1 | TRUE | 6324 | sodium channel, voltage-gated, type I, beta | Munich |
| SCN4A | 17q23-q25.3 | TRUE | 6329 | sodium channel, voltage-gated, type IV, alpha | Aberdeen |
| SCN7A | 2q21-q23 | TRUE | 6332 | sodium channel, voltage-gated, type VII, alpha | Aberdeen |
| SCNN1A | 12p13 | TRUE | 6337 | sodium channel, nonvoltage-gated 1 alpha | Aberdeen |
| SCTR | 2q14.1 | TRUE | 6344 | secretin receptor | Aberdeen |
| SCTR | 2q14.1 | TRUE | 6344 | secretin receptor | Munich |
| SEC11L1 | 15q25.3 | TRUE | 23478 | SEC11-like 1 (S. cerevisiae) | Munich |
| SEC13L1 | 3p25-p24 | TRUE | 6396 | SEC13-like 1 (S. cerevisiae) | Aberdeen |
| SEMA3A | 7p12.1 | TRUE | 10371 | sema domain, immunoglobulin domain (Ig), short basic domain, | Munich |
| | | | | secreted, (semaphorin) 3A | |
| SENP1 | 12q13.1 | TRUE | 29843 | SUMO1/sentrin specific peptidase 1 | Munich |
| SERGEF | 11p14.3 | TRUE | 26297 | secretion regulating guanine nucleotide exchange factor | Aberdeen |
| SERPINA1 | 14q32.1 | TRUE | 5265 | serpin peptidase inhibitor, clade A (alpha-1 antiproteinase, | Aberdeen |
| | | | | antitrypsin), member 1 | |
| SERPINA3 | 14q32.1 | TRUE | 12 | serpin peptidase inhibitor, clade A (alpha-1 antiproteinase, | Aberdeen |
| | | | | antitrypsin), member 3 | |
| SERPINA5 | 14q32.1 | TRUE | 5104 | serpin peptidase inhibitor, clade A (alpha-1 antiproteinase, | Aberdeen |
| | | | | antitrypsin), member 5 | |
| SERPINI1 | 3q26.1 | TRUE | 5274 | serpin peptidase inhibitor, clade I (neuroserpin), member 1 | Aberdeen |
| SF1 | 11q13 | TRUE | 7536 | splicing factor 1 | Aberdeen |
| SFRP1 | 8p12-p11.1 | TRUE | 6422 | secreted frizzled-related protein 1 | Aberdeen |
| SFRP4 | 7p14.1 | TRUE | 6424 | secreted frizzled-related protein 4 | Aberdeen |
| SGK | 6q23 | TRUE | 6446 | serum/glucocorticoid regulated kinase | Munich |
| SGK2 | 20q13.2 | TRUE | 10110 | serum/glucocorticoid regulated kinase 2 | Munich |
| SH3D19 | 4q31.3 | FALSE | 152503 | SH3 domain protein D19 | Munich |
| SIRT2 | 19q13 | TRUE | 22933 | sirtuin (silent mating type information regulation 2 homolog) 2 | Aberdeen |
| | | | | (S. cerevisiae) | |
| SIRT2 | 19q13 | TRUE | 22933 | sirtuin (silent mating type information regulation 2 homolog) 2 | Munich |
| | | | | (S. cerevisiae) | |
| SKIV2L | 6p21 | TRUE | 6499 | superkiller viralicidic activity 2-like (S. cerevisiae) | Aberdeen |
| SLC12A2 | 5q23.3 | TRUE | 6558 | solute carrier family 12 (sodium/potassium/chloride | Aberdeen |
| | | | | transporters), member 2 | |
| SLC12A5 | 20q13.12 | TRUE | 57468 | solute carrier family 12, (potassium-chloride transporter) | Aberdeen |
| | | | | member 5 | |
| SLC22A3 | 6q26-q27 | TRUE | 6581 | solute carrier family 22 (extraneuronal monoamine transporter), | Aberdeen |
| | | | | member 3 | |
| SLC25A5 | Xq24-q26 | TRUE | 292 | solute carrier family 25 (mitochondrial carrier; adenine | Aberdeen |
| | | | | nucleotide translocator), member 5 | |
| SLC2A13 | 12q12 | TRUE | 114134 | solute carrier family 2 (facilitated glucose transporter), member | Aberdeen |
| | | | | 13 | |
| SLC30A3 | 2p23.3 | TRUE | 7781 | solute carrier family 30 (zinc transporter), member 3 | Munich |
| SLC30A5 | 5q12.1 | TRUE | 64924 | solute carrier family 30 (zinc transporter), member 5 | Munich |
| SLC35A2 | Xp11.23-p11.22 | TRUE | 7355 | solute carrier family 35 (UDP-galactose transporter), member | Munich |
| | | | | A2 | |
| SLC35A4 | 5q31.3 | TRUE | 113829 | solute carrier family 35, member A4 | Aberdeen |
| SLC6A1 | 3p25-p24 | TRUE | 6529 | solute carrier family 6 (neurotransmitter transporter, GABA), | Aberdeen |
| | | | | member 1 | |
| SLC6A3 | 5p15.3 | TRUE | 6531 | solute carrier family 6 (neurotransmitter transporter, dopamine), | Aberdeen |
| | | | | member 3 | |
| SLC6A5 | 11p15.2-p15.1 | TRUE | 9152 | solute carrier family 6 (neurotransmitter transporter, glycine), | Aberdeen |
| | | | | member 5 | |
| SLC6A5 | 11p15.2-p15.1 | TRUE | 9152 | solute carrier family 6 (neurotransmitter transporter, glycine), | Munich |
| | | | | member 5 | |
| SLC6A7 | 5q31-q32 | TRUE | 6534 | solute carrier family 6 (neurotransmitter transporter, L-proline), | Aberdeen |
| | | | | member 7 | |
| SLC7A8 | 14q11.2 | TRUE | 23428 | solute carrier family 7 (cationic amino acid transporter, y+ | Aberdeen |
| | | | | system), member 8 | |
| SLC7A8 | 14q11.2 | TRUE | 23428 | solute carrier family 7 (cationic amino acid transporter, y+ | Munich |
| | | | | system), member 8 | |
| SLC8A3 | 14q24.1 | TRUE | 6547 | solute carrier family 8 (sodium-calcium exchanger), member 3 | Munich |
| SLK | 10q25.1 | TRUE | 9748 | STE20-like kinase (yeast) | Munich |
| SMAD7 | 18q21.1 | TRUE | 4092 | SMAD, mothers against DPP homolog 7 (Drosophila) | Both Sets |
| SMAD7 | 18q21.1 | TRUE | 4092 | SMAD, mothers against DPP homolog 7 (Drosophila) | Aberdeen |
| SMAD7 | 18q21.1 | TRUE | 4092 | SMAD, mothers against DPP homolog 7 (Drosophila) | Munich |
| SNAPC5 | 15q22.31 | TRUE | 10302 | small nuclear RNA activating complex, polypeptide 5, 19 kDa | Aberdeen |
| SNCA | 4q21 | TRUE | 6622 | synuclein, alpha (non A4 component of amyloid precursor) | Aberdeen |
| SNW1 | 14q24.3 | TRUE | 22938 | SNW domain containing 1 | Munich |
| SOD2 | 6q25.3 | TRUE | 6648 | superoxide dismutase 2, mitochondrial | Aberdeen |
| SOX2 | 3q26.3-q27 | TRUE | 6657 | SRY (sex determining region Y)-box 2 | Munich |
| SPFH1 | 10q21-q22 | TRUE | 10613 | SPFH domain family, member 1 | Munich |
| SPOCK3 | 4q32.3 | TRUE | 50859 | sparc/osteonectin, cwcv and kazal-like domains proteoglycan | Aberdeen |
| | | | | (testican) 3 | |
| SPOCK3 | 4q32.3 | TRUE | 50859 | sparc/osteonectin, cwcv and kazal-like domains proteoglycan | Munich |
| | | | | (testican) 3 | |
| SRI | 7q21.1 | TRUE | 6717 | sorcin | Munich |
| SST | 3q28 | TRUE | 6750 | somatostatin | Aberdeen |
| ST14 | 11q24-q25 | TRUE | 6768 | suppression of tumorigenicity 14 (colon carcinoma) | Aberdeen |
| ST3GAL6 | 3q12.1 | TRUE | 10402 | ST3 beta-galactoside alpha-2,3-sialyltransferase 6 | Munich |
| STARD3 | 17q11-q12 | TRUE | 10948 | START domain containing 3 | Munich |
| STAT6 | 12q13 | TRUE | 6778 | signal transducer and activator of transcription 6, interleukin-4 | Aberdeen |
| | | | | induced | |
| STC1 | 8p21-p11.2 | TRUE | 6781 | stanniocalcin 1 | Munich |
| STK16 | 2q34-q37 | TRUE | 8576 | serine/threonine kinase 16 | Aberdeen |
| STK17B | 2q32.3 | TRUE | 9262 | serine/threonine kinase 17b (apoptosis-inducing) | Aberdeen |
| STK25 | 2q37.3 | TRUE | 10494 | serine/threonine kinase 25 (STE20 homolog, yeast) | Aberdeen |
| STK4 | 20q11.2-q13.2 | TRUE | 6789 | serine/threonine kinase 4 | Aberdeen |
| STK4 | 20q11.2-q13.2 | TRUE | 6789 | serine/threonine kinase 4 | Munich |
| STX17 | 9q31.1 | TRUE | 55014 | syntaxin 17 | Munich |
| SULT1A2 | 16p12.1 | TRUE | 6799 | sulfotransferase family, cytosolic, 1A, phenol-preferring, | Munich |
| | | | | member 2 | |
| SUPT6H | 17q11.2 | TRUE | 6830 | suppressor of Ty 6 homolog (S. cerevisiae) | Munich |
| SV2B | 15q26.1 | TRUE | 9899 | synaptic vesicle glycoprotein 2B | Aberdeen |
| SYK | 9q22 | TRUE | 6850 | spleen tyrosine kinase | Munich |
| SYNE2 | 14q23.2 | TRUE | 23224 | spectrin repeat containing, nuclear envelope 2 | Aberdeen |
| TAC3 | 12q13-q21 | TRUE | 6866 | tachykinin 3 (neuromedin K, neurokinin beta) | Munich |
| TACR1 | 2p13.1 | TRUE | 6869 | tachykinin receptor 1 | Munich |
| TAP2 | 6p21.3 | TRUE | 6891 | transporter 2, ATP-binding cassette, sub-family B (MDR/TAP) | Aberdeen |
| TAR5753 | 4q31.23-q31.3 | FALSE | | Serine protease, trypsin | Munich |
| TARBP2 | 12q12-q13 | TRUE | 6895 | Tar (HIV-1) RNA binding protein 2 | Aberdeen |
| TASP1 | 20p12.1 | TRUE | 55617 | taspase, threonine aspartase, 1 | Aberdeen |
| TBC1D10C | 11q13.1 | TRUE | 374403 | TBC1 domain family, member 10C | Aberdeen |
| TCF7L2 | 10q25.3 | TRUE | 6934 | transcription factor 7-like 2 (T-cell specific, HMG-box) | Aberdeen |
| TEK | 9p21 | TRUE | 7010 | TEK tyrosine kinase, endothelial (venous malformations, | Munich |
| | | | | multiple cutaneous and mucosal) | |
| TFCP2 | 12q13 | TRUE | 7024 | transcription factor CP2 | Aberdeen |
| TFEB | 6p21 | TRUE | 7942 | transcription factor EB | Munich |
| TFF2 | 21q22.3 | TRUE | 7032 | trefoil factor 2 (spasmolytic protein 1) | Aberdeen |
| TFF3 | 21q22.3 | TRUE | 7033 | trefoil factor 3 (intestinal) | Aberdeen |
| TFF3 | 21q22.3 | TRUE | 7033 | trefoil factor 3 (intestinal) | Munich |
| TFRC | 3q29 | TRUE | 7037 | transferrin receptor (p90, CD71) | Munich |
| TGFA | 2p13 | TRUE | 7039 | transforming growth factor, alpha | Aberdeen |
| TH1L | 20q13 | TRUE | 51497 | TH1-like (Drosophila) | Aberdeen |
| THOP1 | 19q13.3 | TRUE | 7064 | thimet oligopeptidase 1 | Aberdeen |
| THRAP3 | 1p34.3 | TRUE | 9967 | thyroid hormone receptor associated protein 3 | Aberdeen |
| TIE1 | 1p34-p33 | TRUE | 7075 | tyrosine kinase with immunoglobulin-like and EGF-like domains 1 | Aberdeen |
| TIG22692 | 1p13.2 | FALSE | | AD026 | Aberdeen |
| TIG41002 | | FALSE | | flj30839 | Munich |
| TLR7 | Xp22.3 | TRUE | 51284 | toll-like receptor 7 | Munich |
| TLR8 | Xp22 | TRUE | 51311 | toll-like receptor 8 | Munich |
| TLR9 | 3p21.3 | TRUE | 54106 | toll-like receptor 9 | Munich |
| TMEM63A | 1q42.12 | TRUE | 9725 | transmembrane protein 63A | Aberdeen |
| TMPRSS3 | 21q22.3 | TRUE | 64699 | transmembrane protease, serine 3 | Aberdeen |
| TNFRSF1B | 1p36.3-p36.2 | TRUE | 7133 | tumor necrosis factor receptor superfamily, member 1B | Aberdeen |
| TNIK | 3q26.2-q26.31 | TRUE | 23043 | TRAF2 and NCK interacting kinase | Aberdeen |
| TNK2 | 3q29 | TRUE | 10188 | tyrosine kinase, non-receptor, 2 | Aberdeen |
| TNNI3K | 1p31.1 | TRUE | 51086 | TNNI3 interacting kinase | Aberdeen |
| TNXB | 6p21.3 | TRUE | 7148 | tenascin XB | Aberdeen |
| TOLLIP | 11p15.5 | TRUE | 54472 | toll interacting protein | Aberdeen |
| TOP1 | 20q12-q13.1 | TRUE | 7150 | topoisomerase (DNA) I | Aberdeen |
| TP53BP2 | 1q42.1 | TRUE | 7159 | tumor protein p53 binding protein, 2 | Aberdeen |
| TPH1 | 11p15.3-p14 | TRUE | 7166 | tryptophan hydroxylase 1 (tryptophan 5-monooxygenase) | Aberdeen |
| TPP2 | 13q32-q33 | TRUE | 7174 | tripeptidyl peptidase II | Aberdeen |
| TRAF4 | 17q11-q12 | TRUE | 9618 | TNF receptor-associated factor 4 | Munich |
| TRAF6 | 11p12 | TRUE | 7189 | TNF receptor-associated factor 6 | Aberdeen |
| TREM1 | 6p21.1 | TRUE | 54210 | triggering receptor expressed on myeloid cells 1 | Munich |
| TRIP12 | 2q36.3 | TRUE | 9320 | thyroid hormone receptor interactor 12 | Aberdeen |
| TRPC4 | 13q13.1-q13.2 | TRUE | 7223 | transient receptor potential cation channel, subfamily C, | Aberdeen |
| | | | | member 4 | |
| TRPC4AP | 20q11.22 | TRUE | 26133 | transient receptor potential cation channel, subfamily C, | Munich |
| | | | | member 4 associated protein | |
| TRPC6 | 11q21-q22 | TRUE | 7225 | transient receptor potential cation channel, subfamily C, | Aberdeen |
| | | | | member 6 | |
| TRPM2 | 21q22.3 | TRUE | 7226 | transient receptor potential cation channel, subfamily M, | Munich |
| | | | | member 2 | |
| TRPM5 | 11p15.5 | TRUE | 29850 | transient receptor potential cation channel, subfamily M, | Munich |
| | | | | member 5 | |
| TRPM6 | 9q21.13 | TRUE | 140803 | transient receptor potential cation channel, subfamily M, | Aberdeen |
| | | | | member 6 | |
| TRPV3 | 17p13.3 | TRUE | 162514 | transient receptor potential cation channel, subfamily V, member 3 | Aberdeen |
| TRPV3 | 17p13.3 | TRUE | 162514 | transient receptor potential cation channel, subfamily V, member 3 | Munich |
| TRPV6 | 7q33-q34 | TRUE | 55503 | transient receptor potential cation channel, subfamily V, member 6 | Munich |
| TSPO | 22q13.31 | TRUE | 706 | translocator protein (18 kDa) | Aberdeen |
| TUBA1 | 2q35 | TRUE | 7277 | tubulin, alpha 1 (testis specific) | Aberdeen |
| TUBB3 | 16q24.3 | TRUE | 10381 | tubulin, beta 3 | Munich |
| TXN | 9q31 | TRUE | 7295 | thioredoxin | Aberdeen |
| TXN | 9q31 | TRUE | 7295 | thioredoxin | Munich |
| TXNDC14 | 11cen-q22.3 | TRUE | 51075 | thioredoxin domain containing 14 | Aberdeen |
| UBE3A | 15q11-q13 | TRUE | 7337 | ubiquitin protein ligase E3A (human papilloma virus E6- | Aberdeen |
| | | | | associated protein, Angelman syndrome) | |
| UBQLN1 | 9q22\|9q21.2-q21.3 | TRUE | 29979 | ubiquilin 1 | Aberdeen |
| UCHL3 | 13q22.2 | TRUE | 7347 | ubiquitin carboxyl-terminal esterase L3 (ubiquitin thiolesterase) | Aberdeen |
| UCP3 | 11q13 | TRUE | 7352 | uncoupling protein 3 (mitochondrial, proton carrier) | Aberdeen |
| ULK1 | 12q24.3 | TRUE | 8408 | unc-51-like kinase 1 (C. elegans) | Munich |
| USP29 | 19q13.43 | TRUE | 57663 | ubiquitin specific peptidase 29 | Munich |
| USP46 | 4q12 | TRUE | 64854 | ubiquitin specific peptidase 46 | Both Sets |
| USP46 | 4q12 | TRUE | 64854 | ubiquitin specific peptidase 46 | Aberdeen |
| USP46 | 4q12 | TRUE | 64854 | ubiquitin specific peptidase 46 | Munich |
| USP9X | Xp11.4 | TRUE | 8239 | ubiquitin specific peptidase 9, X-linked | Munich |
| VDAC1 | 5q31 | TRUE | 7416 | voltage-dependent anion channel 1 | Munich |
| VDR | 12q13.11 | TRUE | 7421 | vitamin D (1,25-dihydroxyvitamin D3) receptor | Both Sets |
| VDR | 12q13.11 | TRUE | 7421 | vitamin D (1,25-dihydroxyvitamin D3) receptor | Aberdeen |
| VDR | 12q13.11 | TRUE | 7421 | vitamin D (1,25-dihydroxyvitamin D3) receptor | Munich |
| VGCNL1 | 13q32.3 | TRUE | 259232 | voltage gated channel like 1 | Aberdeen |
| VGCNL1 | 13q32.3 | TRUE | 259232 | voltage gated channel like 1 | Munich |
| VGF | 7q22 | TRUE | 7425 | VGF nerve growth factor inducible | Munich |
| VIP | 6q25 | TRUE | 7432 | vasoactive intestinal peptide | Aberdeen |
| VLDLR | 9p24 | TRUE | 7436 | very low density lipoprotein receptor | Both Sets |
| VLDLR | 9p24 | TRUE | 7436 | very low density lipoprotein receptor | Aberdeen |
| VLDLR | 9p24 | TRUE | 7436 | very low density lipoprotein receptor | Munich |
| VTI1A | 10q25.2 | TRUE | 143187 | vesicle transport through interaction with t-SNAREs homolog 1A | Aberdeen |
| | | | | (yeast) | |
| WASF1 | 6q21-q22 | TRUE | 8936 | WAS protein family, member 1 | Munich |
| WIF1 | 12q14.3 | TRUE | 11197 | WNT inhibitory factor 1 | Aberdeen |
| WNT10A | 2q35 | TRUE | 80326 | wingless-type MMTV integration site family, member 10A | Aberdeen |
| WNT11 | 11q13.5 | TRUE | 7481 | wingless-type MMTV integration site family, member 11 | Both Sets |
| WNT11 | 11q13.5 | TRUE | 7481 | wingless-type MMTV integration site family, member 11 | Aberdeen |
| WNT11 | 11q13.5 | TRUE | 7481 | wingless-type MMTV integration site family, member 11 | Munich |
| WNT2B | 1p13 | TRUE | 7482 | wingless-type MMTV integration site family, member 2B | Aberdeen |
| WNT3 | 17q21 | TRUE | 7473 | wingless-type MMTV integration site family, member 3 | Aberdeen |
| WNT4 | 1p36.23-p35.1 | TRUE | 54361 | wingless-type MMTV integration site family, member 4 | Aberdeen |
| WNT5B | 12p13.3 | TRUE | 81029 | wingless-type MMTV integration site family, member 5B | Aberdeen |
| WNT7A | 3p25 | TRUE | 7476 | wingless-type MMTV integration site family, member 7A | Aberdeen |
| WNT8A | 5q31 | TRUE | 7478 | wingless-type MMTV integration site family, member 8A | Aberdeen |
| WNT9B | 17q21 | TRUE | 7484 | wingless-type MMTV integration site family, member 9B | Aberdeen |
| XM_065294 | 2q36.1 | FALSE | | similar to Transmembrane protease, serine 3 (Serine protease | Munich |
| | | | | TADG-12) (Tumor associated differentially-expressed gene-12 | |
| | | | | protein) | |
| ZDHHC6 | 10q25.2 | TRUE | 64429 | zinc finger, DHHC-type containing 6 | Aberdeen |
| ZIM3 | | TRUE | 114026 | zinc finger, imprinted 3 | Munich |
| ZNF238 | 1q44-qter | TRUE | 10472 | zinc finger protein 238 | Munich |
| ZNF513 | 2p23.3 | TRUE | 130557 | zinc finger protein 513 | Aberdeen |
| ZYX | 7q32 | TRUE | 7791 | zyxin | Aberdeen |

¹HUGO gene = TRUE indicates the gene symbol is an official, unique Human Genome name. http://www.gene.ucl.ac.uk/nomenclature/index.html
²ENTREZ Gene ID is the gene ID assigned by NCBI http://www.ncbi.nlm.nih.gov/Database/index.html
³Genes associated in Munich (genes in Table 3), Aberdeen (genes in Table 4), Both Sets (genes in Table 5)

### REFERENCES

Allison D B, Faith M S, and Nathan J S. (1996) Risch's Lambda Values for Human Schizophrenia. International Journal of Schizophrenia Related Metabolic Disorders 20 (11):990-999.
American Psychiatric Association (1994): Diagnostic and Statistical Manual of Mental Disorders (DSM-IV); 4^{th} Edition, Washington DC, APA.
Barbato A. Schizophrenia and public health WHO 1998
Baskin M L et al (2005) Prevalence of Schizophrenia in the United States. Schizophrenia Review 6 (1):5-7.
Cardno A G, Gottesman II. Twin studies of schizophrenia: From bow-and-arrow concordances to Star Wars Mx and functional genomics. Am J Med Genet. 2000 January;97(1):12-17.
Cardno A G, Marshall E J, Coid B, Macdonald A M, Ribchester T R, Davies N J, Venturi P, Jones L A, Lewis S W, Sham P C, Gottesman II, Farmer A E, McGuffin P, Reveley A M, Murray R M. Heritability estimates for psychotic disorders: the Maudsley twin psychosis series. Arch Gen Psychiatry. 1999 February;56(2):162-8
Comuzzie A G and Allison D B. (1998) The search for human Schizophrenia genes. Science 280 (5368):1374-1377.
Crandall, M A. The US Market for Schizophrenia Treatment and Weight Management. Theta Report. 2001.
Devlin B. Roeder K. Wassernan L. Genomic control, a new approach to genetic-based association studies. Theoretical Population Biology. 60(3):155-66, 2001 November.
Fleiss J, Levin B., Paik M C. (2003) Statistical Methods for Rates and Proportions. 3^{rd} Edition. Wiley.
The Genetics of Schizophrenia. 1994. Bouchard C (ed). Boca Raton: CRC Press.
McGue M, Gottesman II. A single dominant gene still cannot account for the transmission of schizophrenia. Arch Gen Psychiatry. 1989 May;46(5):478-80.
Meyer J M S A J (1994) Twin Studies of Human Schizophrenia. The Genetics of Schizophrenia. Boca Raton: CRC Press. Ref Type: Generic
Mehta, C. and Patel, N. (1983) A Network Algorithm for Performing Fisher's Exact Test in rXc contingency tables. Journal of the American Statistical Association 78:427-434.
Meng, Z. et al. (2003) Selection of Genetic Markers for Assoication Analyses, Using Linkage Disequilbrium and Haplotypes. American Journal of Human Genetics 71(1): 115-130.
National Institutes of Health. Clinical (1998) Guidelines on the Identification, Evaluation, and Treatment of Overweight and Schizophrenia in Adults. National Heart, Lung and Blood Institute in cooperation with The National Institute of Diabetes and Digestive and Kidney Diseases. NIH Publication No. 98-4083. Ref Type: Generic
Riley B, McGuffin P. Linkage and associated studies of schizophrenia. Am J Med Genet (Semin. Med. Genet.) 2000, 97:23-44
Risch N. Linkage strategies for genetically complex traits. I. Multilocus models. Am J Hum Genet. 1990 February;46(2):222-8
Roses A D., Bums D K., Chissoe S., Middleton L., St Jean P., (2005) Disease-specific target selction: A Critical First Step Down the Right Road. Drug Discovery Today 10: 177-189.
Sorensen T I A S A (1994) Overview of the adoption studies. The Genetics of Schizophrenia. Boca Raton: CRC Press.
Stunkard A J, Foch T T, and Hrubec Z (1986) A twin study of human Schizophrenia. JAMA 256 (1):51-54.
Taylor J D., Briley D., Nguyen Q., Long K., Iannone M A., Li M S., Ye F., Afshari A., Lai E., Wagner M., Chen J., Weiner M P. (2001) Flow cytometric platform for high-throughput single nucleotide polymorphism analysis. [Journal Article] Biotechniques. 30(3): 661-6, 668-9, March
Thaker G K, Carpenter W T Jr. Advances in schizophrenia. Nat Med. 2001 June;7(6):667-71
Weir, B S. (1996) Genetic Data Analysis II. Sinauer Associates, Inc., Sunderland, Mass., pp. 109-110.
Zaykin D V, Zhivotovsky L A, Weir B S (1995) Exact tests for association between alleles at arbitrary numbers of loci. Genetica 96: 169-178.

1. A method of screening a small molecule compound for use in treating schizophrenia, comprising screening a test compound against a target selected from the group consisting of the gene products encoded by ADAMTS14, ADAMTS15, ADCY9, APCS, CACNA2D1, CD80, CFB/RDBP, CHRNA1, CREB1, CTSK, G30/DAOA, GABRA3, GRM5, HTR2C, IQGAP2/F2RL2, KCNB2, KCNH7, KCNJ14/GRWD1, KCNK13, MMP1, MMP16, NEFL, PAK7, PCAF, PROKR2, PTGDR, PTPRC, SMAD7, USP46, VDR, LVDLR, and WNT11, where activity against said target indicates the test compound has potential use in treating schizophrenia.
